SEQUENCE LISTING (719 sequences)

SEQ ID NO: 1 - DNA, 20 bp, Artificial Sequence
  primer
cagcttgtccaactggtcgg

SEQ ID NO: 2 - DNA, 20 bp, artificial
  primer
agcacacaagcacgttaccc

SEQ ID NO: 3 - DNA, 20 bp, artificial
  primer
ggactgagagctgttgacac

SEQ ID NO: 4 - DNA, 17 bp, artificial
  primer
gaccgcgtctcacaccg

SEQ ID NO: 5 - DNA, 26 bp, artificial
  primer
ctgcgctccacgagcccgacgcaatg

SEQ ID NO: 6 - DNA, 26 bp, artificial
  primer
ctggcgtggtcgcgtgctcggcaatg

SEQ ID NO: 7 - DNA, 20 bp, artificial
  primer
gatcagggccgtctcgaaac

SEQ ID NO: 8 - DNA, 20 bp, artificial
  primer
ggactatcatatgcttaccg

SEQ ID NO: 9 - DNA, 20 bp, artificial
  primer
gacccagaattctcgcattc

SEQ ID NO: 10 - DNA, 89 bp, artificial
  primer
aatgatacggcgaccaccgagatctacactatagcctacactctttccctacacgacgctcttccgatcttgtggaaaggacgaaacac

SEQ ID NO: 11 - DNA, 86 bp, artificial
  primer
caagcagaagacggcatacgagatggagatgagtgactggagttcagacgtgtgctcttccgatcttgctaggaccggccttaaag

SEQ ID NO: 12 - DNA, 75 bp, artificial
  primer
aatgatacggcgaccaccgagatctacactctttccctacacgacgctcttccgatctgctggttccatggtgta

SEQ ID NO: 13 - DNA, 88 bp, artificial
  primer
caagcagaagacggcatacgagatcgagtaatgtgactggagttcagacgtgtgctcttccgatctccttagccgctaataggtgagc

SEQ ID NO: 14 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctaccttcacatggatggttactgaagtttgnnngcaggtgcaatggcaacgtcaaatccgtcgacgcattgcacctgcnntatgcaccctcaccttctacccagccgtttgaagagcgtttaaacgctcttcgccctgcccacaaagtatggcggtgtttcgagacggccctga

SEQ ID NO: 15 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcaatacagggatacccactagtttgnnngcaggtgcaatgaccattcgaacctttcggacgcattgcacctgcnntatgcatcatcctgcatgctagaccagtttgaagagcgtttaaacgctcttcgccctgttgagatcgccctttagcgtttcgagacggccctga

SEQ ID NO: 16 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctagatcatacagctgcccaacgtttgnnngcaggtgcaatgccaggcccaaaaagtagcagccattgcacctgcnntatgcaggagagccaacccctaccaggtttgaagagcgtttaaacgctcttcgccctcatcctgcatgctagaccagtttcgagacggccctga

SEQ ID NO: 17 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctaccttgtgatggtgaatattcacagtttgnnngcaggtgcaatgcgcgaggcaacatataaaccccattgcacctgcnntatgcagattccttccaataacacaagtttgaagagcgtttaaacgctcttcgcccctgaaattagacatcaaagcgtttcgagacggccctga

SEQ ID NO: 18 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctaccttgaatgaccagggtacctgcgtttgnnngcaggtgcaatgttccatgacattcccagtgcccattgcacctgcnntatgcatgcccacaaagtatggcggtgtttgaagagcgtttaaacgctcttcgcccagcacacaagcacgttacccgtttcgagacggccctga

SEQ ID NO: 19 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctagcacacaagcacgttacccgtttgnnngcaggtgcaatgctcaacccacgtatgacttcccattgcacctgcnntatgcaagctgtagtagagtccccatgtttgaagagcgtttaaacgctcttcgccccaatacagggatacccactagtttcgagacggccctga

SEQ ID NO: 20 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctacacaacactgatgaggtccgtttgnnngcaggtgcaatgcatacgcgcccattaacttgccattgcacctgcnntatgcagttacactctatctacacctgtttgaagagcgtttaaacgctcttcgccccagtgcaacttactattacggtttcgagacggccctga

SEQ ID NO: 21 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctaccttgcaagccttacctgtagcggtttgnnngcaggtgcaatgctgaatgctgatagacacccccattgcacctgcnntatgcactccacaaagacctgcggcggtttgaagagcgtttaaacgctcttcgccctcatcctgcatgctagaccagtttcgagacggccctga

SEQ ID NO: 22 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctcccaccaggatacgtatgatgtttgnnngcaggtgcaatgcgacccatttccttacccatccattgcacctgcnntatgcactccacaaagacctgcggcggtttgaagagcgtttaaacgctcttcgcccggaagggacactaacctggtgtttcgagacggccctga

SEQ ID NO: 23 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctcgtcaccaagccattgtaccgtttgnnngcaggtgcaatgactcccgcctattcgagttagcattgcacctgcnntatgcactgctagccaacaccactgagtttgaagagcgtttaaacgctcttcgcccttgttacagccttattacacgtttcgagacggccctga

SEQ ID NO: 24 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctactttccatgtcagccatacgtttgnnngcaggtgcaatgtttcccaatcgtccacttcggcattgcacctgcnntatgcacctatcatacgtatcctggtgtttgaagagcgtttaaacgctcttcgcccctgaaattagacatcaaagcgtttcgagacggccctga

SEQ ID NO: 25 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctaccttctgggcatgtgggtccggcgtttgnnngcaggtgcaatgagtctccagtaccccttgaaccattgcacctgcnntatgcatgtgatggtgaatattcacagtttgaagagcgtttaaacgctcttcgcccggagagccaacccctaccaggtttcgagacggccctga

SEQ ID NO: 26 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctacctatcatacgtatcctgggtttgnnngcaggtgcaatgctctatcctgcactgtccagtcattgcacctgcnntatgcatgttgagatcgccctttagcgtttgaagagcgtttaaacgctcttcgccctctgggcatgtgggtccggcgtttcgagacggccctga

SEQ ID NO: 27 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctagatcatacagctgcccaacgtttgnnngcaggtgcaatgcacagtgacgctatcccttaccattgcacctgcnntatgcacaaagacctgcggcgagggcgtttgaagagcgtttaaacgctcttcgcccgccctttagctggtatctgcgtttcgagacggccctga

SEQ ID NO: 28 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctatcagttctgagcaactcgtgtttgnnngcaggtgcaatggaacatagaaccgaccgaccacattgcacctgcnntatgcaacctatcatacgtatcctgggtttgaagagcgtttaaacgctcttcgccctgtgatggtgaatattcacagtttcgagacggccctga

SEQ ID NO: 29 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctaccttcacccataggagccacagcgtttgnnngcaggtgcaatgatgaccaccccaaggaccttccattgcacctgcnntatgcaccctcaccttctacccagccgtttgaagagcgtttaaacgctcttcgccccaagcgcgttagcggtcgtcgtttcgagacggccctga

SEQ ID NO: 30 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctacctacaggtgacccaaccttcaggtttgnnngcaggtgcaatgaattccgctacgaagccacctcattgcacctgcnntatgcacagcttgtccaactggtcgggtttgaagagcgtttaaacgctcttcgccctcggtgcgctcgctaaaatcgtttcgagacggccctga

SEQ ID NO: 31 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctgtgacttggtgtaagcacaagtttgnnngcaggtgcaatggcaagatccttcaagctaggccattgcacctgcnntatgcacagcttgtccaactggtcgggtttgaagagcgtttaaacgctcttcgcccgagcgatatacgcattgacggtttcgagacggccctga

SEQ ID NO: 32 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctaccttctgggcatgtgggtccggcgtttgnnngcaggtgcaatgatccccgaaacggcaaaacgccattgcacctgcnntatgcattcatagacctaatgtgactgtttgaagagcgtttaaacgctcttcgccccggcgccgcggtcaggtattgtttcgagacggccctga

SEQ ID NO: 33 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctacatagacccctgttgtaaggtttgnnngcaggtgcaatgcgacgagctcaattctatgcccattgcacctgcnntatgcatcacccataggagccacagcgtttgaagagcgtttaaacgctcttcgccccgtatggttcgtggatcgccgtttcgagacggccctga

SEQ ID NO: 34 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctcagtgcaacttactattacggtttgnnngcaggtgcaatgcacaaccgttcaagcgaagctcattgcacctgcnntatgcagaggcggtcaggatggctgcgtttgaagagcgtttaaacgctcttcgccctcgtaccgacgcacattaaggtttcgagacggccctga

SEQ ID NO: 35 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctacctgattccttccaataacacaagtttgnnngcaggtgcaatgcacaaccacacaggtaggccacattgcacctgcnntatgcaggccatttatagtgttgaccgtttgaagagcgtttaaacgctcttcgcccttccccggcgacactcgcgcgtttcgagacggccctga

SEQ ID NO: 36 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctaccttgtgatggtgaatattcacagtttgnnngcaggtgcaatggacgtaaatgccacctatgcccattgcacctgcnntatgcagttacactctatctacacctgtttgaagagcgtttaaacgctcttcgcccggatacgttcggacgccatcgtttcgagacggccctga

SEQ ID NO: 37 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctctgctagccaacaccactgagtttgnnngcaggtgcaatgttgccaacagaacaacgggctcattgcacctgcnntatgcatcacatggatggttactgaagtttgaagagcgtttaaacgctcttcgcccgcgtcatactatcgttcgtcgtttcgagacggccctga

SEQ ID NO: 38 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctctccacaaagacctgcggcggtttgnnngcaggtgcaatgccacgatacttacacgctccacattgcacctgcnntatgcattcatagacctaatgtgactgtttgaagagcgtttaaacgctcttcgcccctatcaaggtacgtccgttcgtttcgagacggccctga

SEQ ID NO: 39 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctaccttgttgggcagctgtatgatcgtttgnnngcaggtgcaatgccgctttgctttggttctacgcattgcacctgcnntatgcatgcccacaaagtatggcggtgtttgaagagcgtttaaacgctcttcgccctcgcgaccccgatattcacggtttcgagacggccctga

SEQ ID NO: 40 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctctggaaggtgaggcctaggcgtttgnnngcaggtgcaatggccaccagaactgaattgcgacattgcacctgcnntatgcagccctttagctggtatctgcgtttgaagagcgtttaaacgctcttcgccccgtcgttacagtctaccgttgtttcgagacggccctga

SEQ ID NO: 41 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctctggaaggtgaggcctaggcgtttgnnngcaggtgcaatggaatgcaccattggtcaccctcattgcacctgcnntatgcaagaagttggtgacgttctgcgtttgaagagcgtttaaacgctcttcgcccgcgcacaccgcgtcgtaccggtttcgagacggccctga

SEQ ID NO: 42 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctacaggtgacccaaccttcaggtttgnnngcaggtgcaatgcccttaagtcacatcagcccacattgcacctgcnntatgcaccctcaccttctacccagccgtttgaagagcgtttaaacgctcttcgcccggtcggcgcggcatttcgacgtttcgagacggccctga

SEQ ID NO: 43 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctctgcagttgagctggcaatcgtttgnnngcaggtgcaatgaccctgacagttagcgcgtcacattgcacctgcnntatgcactccacaaagacctgcggcggtttgaagagcgtttaaacgctcttcgcccgcgcgacgtaaattcgccttgtttcgagacggccctga

SEQ ID NO: 44 - DNA, 236 bp, artificial
  oligo
  misc_feature (93)..(95): n is a, c, g, or t
  misc_feature (141)..(142): n is a, c, g, or t
gaccgcgtctcacaccgtgttgagatcgccctttagcgtttggagaccgtttaaacggtctctacctccctcaccttctacccagccgtttgnnngcaggtgcaatgaatctaaaaccggcaccgtcccattgcacctgcnntatgcactccacaaagacctgcggcggtttgaagagcgtttaaacgctcttcgccccgcactcgacctacgataccgtttcgagacggccctga

SEQ ID NO: 45 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctgtatcctggtgggatttacagtttgnnngcaggtgcaatgctaaaccacagaatgcgccgacattgcacctgcnntatgcagccctttagctggtatctgcgtttgaagagcgtttaaacgctcttcgcccctagcggcgcaatacgtgctgtttcgagacggccctga

SEQ ID NO: 46 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcaacttcttcgtggtatctcgtttgnnngcaggtgcaatgccaaagtgcaagaatcgcggccattgcacctgcnntatgcaacaggtgacccaaccttcaggtttgaagagcgtttaaacgctcttcgcccgataccgacctctcgccgcggtttcgagacggccctga

SEQ ID NO: 47 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctagcacacaagcacgttacccgtttgnnngcaggtgcaatgcaatttctagcagcgctcccacattgcacctgcnntatgcatgttgagatcgccctttagcgtttgaagagcgtttaaacgctcttcgccccgctcgaccgatatagttacgtttcgagacggccctga

SEQ ID NO: 48 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctagcacacaagcacgttacccgtttgnnngcaggtgcaatgacagtaagaagcaccgcacgtcattgcacctgcnntatgcagccctttagctggtatctgcgtttgaagagcgtttaaacgctcttcgcccctccgcgttagcattaccgcgtttcgagacggccctga

SEQ ID NO: 49 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctcagtgcaacttactattacggtttgnnngcaggtgcaatggtgcactcatttaaggtcccgcattgcacctgcnntatgcagtacatcggacttcgcgtaggtttgaagagcgtttaaacgctcttcgcccgcgatgcaagtacgattcgagtttcgagacggccctga

SEQ ID NO: 50 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctcaggcacgatagatacaaaggtttgnnngcaggtgcaatgcattagcagcaacccgtcacccattgcacctgcnntatgcacccctgctcgtcgaggcgatgtttgaagagcgtttaaacgctcttcgcccccgaacctagtaccgcgttagtttcgagacggccctga

SEQ ID NO: 51 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctacatagacccctgttgtaaggtttgnnngcaggtgcaatgcaaaggagccccgactttagtcattgcacctgcnntatgcagtaccgtcaaccgtagcggtgtttgaagagcgtttaaacgctcttcgcccttacacgattaggcgagcacgtttcgagacggccctga

SEQ ID NO: 52 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctgttacactctatctacacctgtttgnnngcaggtgcaatggctcactcacaacgggtttagcattgcacctgcnntatgcagacgtgccattaacgctagggtttgaagagcgtttaaacgctcttcgcccaccgtagtcgtgcatccgtcgtttcgagacggccctga

SEQ ID NO: 53 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctaccttcacatggatggttactgaagtttgnnngcaggtgcaatgttaccgcagcatcgttcactgcattgcacctgcnntatgcactacgcgctaaccgcattgtgtttgaagagcgtttaaacgctcttcgcccgaatttacggcggtcaccgggtttcgagacggccctga

SEQ ID NO: 54 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctctgcagttgagctggcaatcgtttgnnngcaggtgcaatgtacaccctcgaaaggtcaagccattgcacctgcnntatgcaattcgcgttatcgaacacgtgtttgaagagcgtttaaacgctcttcgcccccctacgttacggctccggggtttcgagacggccctga

SEQ ID NO: 55 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctccctcaccttctacccagccgtttgnnngcaggtgcaatggtaactgcggcaccatacttccattgcacctgcnntatgcattgtacgcttcgggcgcgatgtttgaagagcgtttaaacgctcttcgcccctaacggatccccgcgccaggtttcgagacggccctga

SEQ ID NO: 56 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctttgttacagccttattacacgtttgnnngcaggtgcaatggcaggaagatgaagcatcacgcattgcacctgcnntatgcaggtttgcgctaacgtgtggcgtttgaagagcgtttaaacgctcttcgccctagcgaccgtaccgccgattgtttcgagacggccctga

SEQ ID NO: 57 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctaccttctgggcatgtgggtccggcgtttgnnngcaggtgcaatgagcaacttggaactaggcccccattgcacctgcnntatgcatcaggcacggtcgatacgaggtttgaagagcgtttaaacgctcttcgccccgtgattcacgacgatagcggtttcgagacggccctga

SEQ ID NO: 58 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctggactgagagctgttgacacgtttgnnngcaggtgcaatgcagccttaacacgtactagcgcattgcacctgcnntatgcaccggtcatgcggcgaaaccggtttgaagagcgtttaaacgctcttcgccctatctcgcgagcgagttcacgtttcgagacggccctga

SEQ ID NO: 59 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctctggaaggtgaggcctaggcgtttgnnngcaggtgcaatgttccatgcgacaccctcacaacattgcacctgcnntatgcaatttgcgaatccgccacgacgtttgaagagcgtttaaacgctcttcgccctcggagccctattagcgcgagtttcgagacggccctga

SEQ ID NO: 60 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctcccaccaggatacgtatgatgtttgnnngcaggtgcaatgaccaccttccgttactctagccattgcacctgcnntatgcagtggcgtcgagcctatgcgtgtttgaagagcgtttaaacgctcttcgccccgtcgtaataggcattggcggtttcgagacggccctga

SEQ ID NO: 61 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctacctaatcccttgccccagcacatgtttgnnngcaggtgcaatgatccactaatacttcgcgccccattgcacctgcnntatgcacacctaactacgcccgcgacgtttgaagagcgtttaaacgctcttcgccctctaacgtcggcgctctatagtttcgagacggccctga

SEQ ID NO: 62 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctaccttgtgatggtgaatattcacagtttgnnngcaggtgcaatgaccccgaacaaagaggtcagccattgcacctgcnntatgcagctacggtcgcgcaaaactagtttgaagagcgtttaaacgctcttcgccctgtcatattcgacgccgtacgtttcgagacggccctga

SEQ ID NO: 63 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctacctatcagttctgagcaactcgtgtttgnnngcaggtgcaatgtccagagccaccggaataatgcattgcacctgcnntatgcacgctacgttacgtccgggttgtttgaagagcgtttaaacgctcttcgccccaacgcgagatcgcatgcaagtttcgagacggccctga

SEQ ID NO: 64 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctggccgagtgcgtaaaaacgagtttgnnngcaggtgcaatggccataagatcctcttccagccattgcacctgcnntatgcagaggaagcttaacgcgcgctgtttgaagagcgtttaaacgctcttcgcccgacagcgcgaacgacctaacgtttcgagacggccctga

SEQ ID NO: 65 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctcggtacggttctccgctacggtttgnnngcaggtgcaatggaccccatagtcccgaattagcattgcacctgcnntatgcaccactcgcggtttataccgggtttgaagagcgtttaaacgctcttcgccctgcgcgatgatctaacgcacgtttcgagacggccctga

SEQ ID NO: 66 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctggttgaccgcgaccgttcgggtttgnnngcaggtgcaatgtcgcgaaagcaagacttagcccattgcacctgcnntatgcacccctcgccatacgcgtaaggtttgaagagcgtttaaacgctcttcgcccccgagtagaccgcgtatggtgtttcgagacggccctga

SEQ ID NO: 67 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctacgtatctcgtccaatcgcagtttgnnngcaggtgcaatgccagacgttagcacactaaggcattgcacctgcnntatgcagttcgaacatgctaccggcggtttgaagagcgtttaaacgctcttcgcccagccgtgccgtattgacgatgtttcgagacggccctga

SEQ ID NO: 68 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctctgtgggttaacgtcgcggtgtttgnnngcaggtgcaatgcccaactattccgcacgttcccattgcacctgcnntatgcaagcaacgaccgcgatcacgtgtttgaagagcgtttaaacgctcttcgcccgcgtcaccggcagactaaacgtttcgagacggccctga

SEQ ID NO: 69 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctgtcacggcggtatactgcgtgtttgnnngcaggtgcaatgtccgtataaagccagctcccacattgcacctgcnntatgcagtacatcgccccggtcgattgtttgaagagcgtttaaacgctcttcgccctcagcgggccgacatcgacggtttcgagacggccctga

SEQ ID NO: 70 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctccaagtaccttctccagcatgtttgnnngcaggtgcaatgcccctaaagggtacttagagccattgcacctgcnntatgcaggggcatgggccatgctgtagtttgaagagcgtttaaacgctcttcgcccgagaggtttccgaagactaagtttcgagacggccctga

SEQ ID NO: 71 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctacctatccccgtcgcagttgaactgtttgnnngcaggtgcaatggtatactgagcatcccactggcattgcacctgcnntatgcaaaccccaacatcagcaagcggtttgaagagcgtttaaacgctcttcgcccccaagtaccttctccagcatgtttcgagacggccctga

SEQ ID NO: 72 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctaaccccaacatcagcaagcggtttgnnngcaggtgcaatggtgcacgttgcatcacttggacattgcacctgcnntatgcattcctacaaccttctcgcaagtttgaagagcgtttaaacgctcttcgcccgtcacacctgagtaccactggtttcgagacggccctga

SEQ ID NO: 73 - DNA, 236 bp, artificial
  oligo
  misc_feature (93)..(95): n is a, c, g, or t
  misc_feature (141)..(142): n is a, c, g, or t
gaccgcgtctcacaccgatcttgcagatgctaatagcgtttggagaccgtttaaacggtctctacctccacagtactcacttggtccgtttgnnngcaggtgcaatgcgaccttcagagtatactcgccattgcacctgcnntatgcaaagcactacccctgccaaccgtttgaagagcgtttaaacgctcttcgcccaaagcagtcgcccctgcttggtttcgagacggccctga

SEQ ID NO: 74 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctgtcacacctgagtaccactggtttgnnngcaggtgcaatgagaatgacgccaactccagagcattgcacctgcnntatgcacagcttcaggaccccgatccgtttgaagagcgtttaaacgctcttcgccctacgctgcaaagtctctggcgtttcgagacggccctga

SEQ ID NO: 75 - DNA, 236 bp, artificial
  oligo
  misc_feature (93)..(95): n is a, c, g, or t
  misc_feature (141)..(142): n is a, c, g, or t
gaccgcgtctcacaccgcagcttcaggaccccgatccgtttggagaccgtttaaacggtctctacctgcaaagccgatgtcagcgccgtttgnnngcaggtgcaatgccactttccacagaacctccgcattgcacctgcnntatgcagtgtctgtatgacccacccggtttgaagagcgtttaaacgctcttcgccctattgcatggcgacaaccgggtttcgagacggccctga

SEQ ID NO: 76 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctaccttaccttgagactccaccctcgtttgnnngcaggtgcaatgccaccatcagataccagcgtacattgcacctgcnntatgcagtgtctgtatgacccacccggtttgaagagcgtttaaacgctcttcgcccctaccgcggtgtagcgcgaagtttcgagacggccctga

SEQ ID NO: 77 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctttcttctcagttcaagtaacgtttgnnngcaggtgcaatgcatcgagcacggcataaagtccattgcacctgcnntatgcataaccatagtacccagttgtgtttgaagagcgtttaaacgctcttcgcccctcgtcgcatagcgggtgaggtttcgagacggccctga

SEQ ID NO: 78 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctaaccccaacatcagcaagcggtttgnnngcaggtgcaatgcggtattcacagacctcacctcattgcacctgcnntatgcaaagcactacccctgccaaccgtttgaagagcgtttaaacgctcttcgcccggcgccgctatcggataagtgtttcgagacggccctga

SEQ ID NO: 79 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgtggaagtctgtgtcgggaagtttgnnngcaggtgcaatgccaaccacatcgttcgattcgcattgcacctgcnntatgcagagaggtttccgaagactaagtttgaagagcgtttaaacgctcttcgcccagtcgttcggcccgtctagtgtttcgagacggccctga

SEQ ID NO: 80 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctggggcatgggccatgctgtagtttgnnngcaggtgcaatgggcagctaattacatagcccccattgcacctgcnntatgcattcctacaaccttctcgcaagtttgaagagcgtttaaacgctcttcgcccacgtagataaccccgcgcgggtttcgagacggccctga

SEQ ID NO: 81 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctccacagtactcacttggtccgtttgnnngcaggtgcaatgtatcaactagcgcacgatcgccattgcacctgcnntatgcataagctgctctctagacggagtttgaagagcgtttaaacgctcttcgcccctcgtatcggaattgggaccgtttcgagacggccctga

SEQ ID NO: 82 - DNA, 236 bp, artificial
  oligo
  misc_feature (93)..(95): n is a, c, g, or t
  misc_feature (141)..(142): n is a, c, g, or t
gaccgcgtctcacaccggctgacatcggctttgctgcgtttggagaccgtttaaacggtctctacctgtggaagtctgtgtcgggaagtttgnnngcaggtgcaatggatccctatcatcctcggaagcattgcacctgcnntatgcagtagaccaggcacccaacctgtttgaagagcgtttaaacgctcttcgccccggaccgtcgcgtcgtttaagtttcgagacggccctga

SEQ ID NO: 83 - DNA, 236 bp, artificial
  oligo
  misc_feature (93)..(95): n is a, c, g, or t
  misc_feature (141)..(142): n is a, c, g, or t
gaccgcgtctcacaccggcaggtccttaccttgttccgtttggagaccgtttaaacggtctctacctgtcacacctgagtaccactggtttgnnngcaggtgcaatgtaactgcatttaccgcgacgccattgcacctgcnntatgcattcttctcagttcaagtaacgtttgaagagcgtttaaacgctcttcgccctctaccgagggtagcgtccggtttcgagacggccctga

SEQ ID NO: 84 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctacctgagaagcactatgggggcatgtttgnnngcaggtgcaatgtttatgcgcacgatcccagagcattgcacctgcnntatgcatcccacagttgtaccttatcgtttgaagagcgtttaaacgctcttcgccccgtcgttgataggtcgagcggtttcgagacggccctga

SEQ ID NO: 85 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctatcttgcagatgctaatagcgtttgnnngcaggtgcaatgccctattctgaacatcctggccattgcacctgcnntatgcattggattcacgcgtacgagagtttgaagagcgtttaaacgctcttcgcccggtcggttcgctcgtttcgagtttcgagacggccctga

SEQ ID NO: 86 - DNA, 236 bp, artificial
  oligo
  misc_feature (93)..(95): n is a, c, g, or t
  misc_feature (141)..(142): n is a, c, g, or t
gaccgcgtctcacaccggagaggtttccgaagactaagtttggagaccgtttaaacggtctctacctgagccgctgtgatcataccagtttgnnngcaggtgcaatgacgcagccttattaacacggccattgcacctgcnntatgcatgcgcggacgctggtagcgagtttgaagagcgtttaaacgctcttcgccccgattttgggtcgaccgcccgtttcgagacggccctga

SEQ ID NO: 87 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctaaccccaacatcagcaagcggtttgnnngcaggtgcaatgctgagccacacgatatgaacgcattgcacctgcnntatgcatcaatgcgccgatcgagatcgtttgaagagcgtttaaacgctcttcgcccacgaccatcgttagcggtgggtttcgagacggccctga

SEQ ID NO: 88 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctaccttaaccatagtacccagttgtgtttgnnngcaggtgcaatgcacccgagaactagccgaaagcattgcacctgcnntatgcagtccccggacattcgacgcggtttgaagagcgtttaaacgctcttcgcccgcgattacgcgtccgtttcggtttcgagacggccctga

SEQ ID NO: 89 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctccacagtactcacttggtccgtttgnnngcaggtgcaatgccgcaaagaatatcacctcgccattgcacctgcnntatgcaagtcgtcgatctctgacgacgtttgaagagcgtttaaacgctcttcgcccgcgcgacacatgggcggttcgtttcgagacggccctga

SEQ ID NO: 90 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctaagcactacccctgccaaccgtttgnnngcaggtgcaatgcgccactgatactcccttaaccattgcacctgcnntatgcatcgattggccactaacggttgtttgaagagcgtttaaacgctcttcgcccacgcgcgttgacgaagtactgtttcgagacggccctga

SEQ ID NO: 91 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctaacatgactccccggaggccgtttgnnngcaggtgcaatggaccccttgatttcacaaccgcattgcacctgcnntatgcaccgttatgtagattggtcgtgtttgaagagcgtttaaacgctcttcgcccacgctacgttatcgccgcgagtttcgagacggccctga

SEQ ID NO: 92 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctttattcttgtccaggcaagggtttgnnngcaggtgcaatgccccaactcgcgaaatgatgacattgcacctgcnntatgcaactcaacgcgcgttggtctagtttgaagagcgtttaaacgctcttcgccccggtcaggccaatttcggcggtttcgagacggccctga

SEQ ID NO: 93 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctaccttgctgtcgtgagtgccagccgtttgnnngcaggtgcaatgccaagactcccagccctaatccattgcacctgcnntatgcataggacgcgcatcgcgtggcgtttgaagagcgtttaaacgctcttcgcccacgatgcgatcggagcgtacgtttcgagacggccctga

SEQ ID NO: 94 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctacctttcttctcagttcaagtaacgtttgnnngcaggtgcaatgatgtatccagtaaggcccctccattgcacctgcnntatgcacgttcagtcgttagggtacggtttgaagagcgtttaaacgctcttcgccctacgacggaccgattcgatcgtttcgagacggccctga

SEQ ID NO: 95 - DNA, 236 bp, artificial
  oligo
  misc_feature (93)..(95): n is a, c, g, or t
  misc_feature (141)..(142): n is a, c, g, or t
gaccgcgtctcacaccgaaccccaacatcagcaagcggtttggagaccgtttaaacggtctctacctcctgcgatcgctcgtcgaaagtttgnnngcaggtgcaatggactacctcttaccaaggtcccattgcacctgcnntatgcagcccgcagcaatcgtacgcggtttgaagagcgtttaaacgctcttcgcccaccggaataactggcgcgtcgtttcgagacggccctga

SEQ ID NO: 96 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctgtgcaccgtataacgctcgcgtttgnnngcaggtgcaatgtacaccaagtaccaagcggtccattgcacctgcnntatgcacgacgagtttgtctcgataggtttgaagagcgtttaaacgctcttcgccctcccggataagtcggtcgtagtttcgagacggccctga

SEQ ID NO: 97 - DNA, 236 bp, artificial
  oligo
  misc_feature (93)..(95): n is a, c, g, or t
  misc_feature (141)..(142): n is a, c, g, or t
gaccgcgtctcacaccgccacagtactcacttggtccgtttggagaccgtttaaacggtctctacctcgcgcgttgtgtcggctctagtttgnnngcaggtgcaatgcgcaagacctagatcgtcaagcattgcacctgcnntatgcagtcgtggtaccgtttcgtcggtttgaagagcgtttaaacgctcttcgcccacgcgtagtccaggacgaccgtttcgagacggccctga

SEQ ID NO: 98 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctacctactaatccgaactgcgtccggtttgnnngcaggtgcaatgctacgaaaccgagagtatccccattgcacctgcnntatgcacccggacgcgcatcatccatgtttgaagagcgtttaaacgctcttcgccctcgagtccgtaatttaacgtgtttcgagacggccctga

SEQ ID NO: 99 - DNA, 236 bp, artificial
  oligo
  misc_feature (93)..(95): n is a, c, g, or t
  misc_feature (141)..(142): n is a, c, g, or t
gaccgcgtctcacaccgtacgctgcaaagtctctggcgtttggagaccgtttaaacggtctctacctcgtccggtctatcgactaccgtttgnnngcaggtgcaatgctcacagactgtcacttgacgcattgcacctgcnntatgcatgaacacgatacgcgcacgcgtttgaagagcgtttaaacgctcttcgcccataacgccgcgaccaacgtagtttcgagacggccctga

SEQ ID NO: 100 - DNA, 236 bp, artificial
  oligo
  misc_feature (93)..(95): n is a, c, g, or t
  misc_feature (141)..(142): n is a, c, g, or t
gaccgcgtctcacaccggctcaatctagggcgaagaggtttggagaccgtttaaacggtctctacctggccagaatacaggtcccgcgtttgnnngcaggtgcaatgcagaactcgctcagcacggtacattgcacctgcnntatgcagccttgagcccggtggggctgtttgaagagcgtttaaacgctcttcgccccggccatgcctgccagcttcgtttcgagacggccctga

SEQ ID NO: 101 - DNA, 236 bp, artificial
  oligo
  misc_feature (93)..(95): n is a, c, g, or t
  misc_feature (141)..(142): n is a, c, g, or t
gaccgcgtctcacaccgaacggcacgtacgccatcgcgtttggagaccgtttaaacggtctctacctgccttgagcccggtggggctgtttgnnngcaggtgcaatgagtgtccaccaaggatctacgcattgcacctgcnntatgcaccgagttacctcgagctttcgtttgaagagcgtttaaacgctcttcgccctgacaacgcggatttggtcggtttcgagacggccctga

SEQ ID NO: 102 - DNA, 236 bp, artificial
  oligo
  misc_feature (93)..(95): n is a, c, g, or t
  misc_feature (141)..(142): n is a, c, g, or t
gaccgcgtctcacaccgatcctccacgtgcggttcccgtttggagaccgtttaaacggtctctacctactcacggcagcgcacgcacgtttgnnngcaggtgcaatgtctcgattgacatcccactcccattgcacctgcnntatgcaaacggcacgtacgccatcgcgtttgaagagcgtttaaacgctcttcgcccccgttccacgacccccgttggtttcgagacggccctga

SEQ ID NO: 103 - DNA, 236 bp, artificial
  oligo
  misc_feature (93)..(95): n is a, c, g, or t
  misc_feature (141)..(142): n is a, c, g, or t
gaccgcgtctcacaccgctcgtagcttgccacactgtgtttggagaccgtttaaacggtctctaccttgataagcacctggtgctccgtttgnnngcaggtgcaatgcattcgacattccgctctacgcattgcacctgcnntatgcaaagaggcttcttatgtcggcgtttgaagagcgtttaaacgctcttcgccccgagttgcggcttcggtaacgtttcgagacggccctga

SEQ ID NO: 104 - DNA, 236 bp, artificial
  oligo
  misc_feature (93)..(95): n is a, c, g, or t
  misc_feature (141)..(142): n is a, c, g, or t
gaccgcgtctcacaccgcgcccagggagcgcaaacacgtttggagaccgtttaaacggtctctaccttatccggttacctcatagtcgtttgnnngcaggtgcaatgtccctcttgtaaccgaaagcgcattgcacctgcnntatgcacattgtatacgccttcctgggtttgaagagcgtttaaacgctcttcgccctacgcagccttacgcggtttgtttcgagacggccctga

SEQ ID NO: 105 - DNA, 236 bp, artificial
  oligo
  misc_feature (93)..(95): n is a, c, g, or t
  misc_feature (141)..(142): n is a, c, g, or t
gaccgcgtctcacaccgcttctatggcagcatctcgcgtttggagaccgtttaaacggtctctacctactgtcaggcagcaccactcgtttgnnngcaggtgcaatgatacgtaaccaccgcgaagcccattgcacctgcnntatgcagatcccgacgtatagcccgcgtttgaagagcgtttaaacgctcttcgcccgcggatcgcaatataacgaagtttcgagacggccctga

SEQ ID NO: 106 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctaccttcagaagcatgataagcaccgtttgnnngcaggtgcaatgagcacaactaaccgattggcccattgcacctgcnntatgcacatgagagcgacgatgcgtagtttgaagagcgtttaaacgctcttcgcccaccgccgaatcgagcctgcggtttcgagacggccctga

SEQ ID NO: 107 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctgcatccacattcttacaggcgtttgnnngcaggtgcaatggccccaatcacattcgttcaccattgcacctgcnntatgcagatatcgacggccgattgcggtttgaagagcgtttaaacgctcttcgcccatagggcgctcgattacgcagtttcgagacggccctga

SEQ ID NO: 108 - DNA, 236 bp, artificial
  oligo
  misc_feature (93)..(95): n is a, c, g, or t
  misc_feature (141)..(142): n is a, c, g, or t
gaccgcgtctcacaccgcgcgcaccatagcatcacgcgtttggagaccgtttaaacggtctctacctttctcggaagtcgaggacaggtttgnnngcaggtgcaatgtcccgacgcaatatatcccggcattgcacctgcnntatgcataacggctactgccgcgcgtgtttgaagagcgtttaaacgctcttcgccctacgttaacgctatgcggcggtttcgagacggccctga

SEQ ID NO: 109 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctacctgcgccagtgtttgcgctcccgtttgnnngcaggtgcaatgcccaccagttgataaacctcccattgcacctgcnntatgcacgttggggcacgtcgtcgaagtttgaagagcgtttaaacgctcttcgccccgtggcgaatcgcggctaaagtttcgagacggccctga

SEQ ID NO: 110 - DNA, 236 bp, artificial
  oligo
  misc_feature (93)..(95): n is a, c, g, or t
  misc_feature (141)..(142): n is a, c, g, or t
gaccgcgtctcacaccgtacttgtacctgaaagctcggtttggagaccgtttaaacggtctctacctcattgtatacgccttcctgggtttgnnngcaggtgcaatgaccgacattagtcacatgcgccattgcacctgcnntatgcaggcgggcgattgtcgtcgtagtttgaagagcgtttaaacgctcttcgcccgtacaattagtcgttaacgtgtttcgagacggccctga

SEQ ID NO: 111 - DNA, 236 bp, artificial
  oligo
  misc_feature (93)..(95): n is a, c, g, or t
  misc_feature (141)..(142): n is a, c, g, or t
gaccgcgtctcacaccgtcgggggacggttatttctggtttggagaccgtttaaacggtctctacctgttgtgctatatctacgcgtgtttgnnngcaggtgcaatggtcaacatccggcaattccgtcattgcacctgcnntatgcagtggacttcgccgatccgtcgtttgaagagcgtttaaacgctcttcgccctcggacacccgagattgccggtttcgagacggccctga

SEQ ID NO: 112 - DNA, 236 bp, artificial
  oligo
  misc_feature (93)..(95): n is a, c, g, or t
  misc_feature (141)..(142): n is a, c, g, or t
gaccgcgtctcacaccgaacggcacgtacgccatcgcgtttggagaccgtttaaacggtctctacctgggcggcgtatcgcgtactcgtttgnnngcaggtgcaatgacgaacctagccgtacgcaaacattgcacctgcnntatgcatcctacaaaggcgcgactccgtttgaagagcgtttaaacgctcttcgcccaacgctttaccgggtggaacgtttcgagacggccctga

SEQ ID NO: 113 - DNA, 236 bp, artificial
  oligo
  misc_feature (93)..(95): n is a, c, g, or t
  misc_feature (141)..(142): n is a, c, g, or t
gaccgcgtctcacaccgtgataagcacctggtgctccgtttggagaccgtttaaacggtctctacctgctatacgtcgcgctttaatgtttgnnngcaggtgcaatgcatcgcaggctaatcgctatgcattgcacctgcnntatgcagtataactagtcgcgtgcaagtttgaagagcgtttaaacgctcttcgcccgcttcgttgtcgacaagtcggtttcgagacggccctga

SEQ ID NO: 114 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctacctaacaaggcgccttagtcgtggtttgnnngcaggtgcaatgcctgataactaactagcgccccattgcacctgcnntatgcatcgagcgtaccgaggatactgtttgaagagcgtttaaacgctcttcgccccgcgttagcgtacgatagtagtttcgagacggccctga

SEQ ID NO: 115 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctagatcatacagctgcccaacgtttgnnngcaggtgcaatgcacgtacgaaccgagatgatgcattgcacctgcnntatgcaacatagacccctgttgtaaggtttgaagagcgtttaaacgctcttcgccctcacatggatggttactgaagtttcgagacggccctga

SEQ ID NO: 116 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctcaggcacgatagatacaaaggtttgnnngcaggtgcaatgcttcctgaacaccgagcacagcattgcacctgcnntatgcacaatacagggatacccactagtttgaagagcgtttaaacgctcttcgcccttgggactcctcggttatttgtttcgagacggccctga

SEQ ID NO: 117 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctaccttgtcgatggcaatagccaaggtttgnnngcaggtgcaatgacagttcaatcagcacgtccccattgcacctgcnntatgcagctcaaaccattacagaagggtttgaagagcgtttaaacgctcttcgcccgtgacttggtgtaagcacaagtttcgagacggccctga

SEQ ID NO: 118 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctacctttgggactcctcggttatttgtttgnnngcaggtgcaatgtgggcaaccagtcgactatgccattgcacctgcnntatgcagttacactctatctacacctgtttgaagagcgtttaaacgctcttcgccctgcccacaaagtatggcggtgtttcgagacggccctga

SEQ ID NO: 119 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctagatcatacagctgcccaacgtttgnnngcaggtgcaatgcgagaatccctgattacacgccattgcacctgcnntatgcattcatagacctaatgtgactgtttgaagagcgtttaaacgctcttcgccccaacttcttcgtggtatctcgtttcgagacggccctga

SEQ ID NO: 120 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctaccttgttgagatcgccctttagcgtttgnnngcaggtgcaatgtcgcacgaaatggaccgagaacattgcacctgcnntatgcacagtgcaacttactattacggtttgaagagcgtttaaacgctcttcgccccaacttcttcgtggtatctcgtttcgagacggccctga

SEQ ID NO: 121 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctacctcagtgcaacttactattacggtttgnnngcaggtgcaatggcattctcaacttagctcccgcattgcacctgcnntatgcactgaaattagacatcaaagcgtttgaagagcgtttaaacgctcttcgccccccaccaggatacgtatgatgtttcgagacggccctga

SEQ ID NO: 122 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctcaggcacgatagatacaaaggtttgnnngcaggtgcaatgcactttagccaacgtgtccaccattgcacctgcnntatgcaggactgagagctgttgacacgtttgaagagcgtttaaacgctcttcgccctctttgaaccattgtgttatgtttcgagacggccctga

SEQ ID NO: 123 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctgaggcggtcaggatggctgcgtttgnnngcaggtgcaatgaatgccatagcgcacacagcgcattgcacctgcnntatgcagtgacttggtgtaagcacaagtttgaagagcgtttaaacgctcttcgcccagaagttggtgacgttctgcgtttcgagacggccctga

SEQ ID NO: 124 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctaccttgggactcctcggttatttagtttgnnngcaggtgcaatgcgctttatactcaccacggagcattgcacctgcnntatgcacatcggcctgacccccatgcgtttgaagagcgtttaaacgctcttcgcccattcttctctgtgaatgtcggtttcgagacggccctga

SEQ ID NO: 125 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctggactgagagctgttgacacgtttgnnngcaggtgcaatgcccagcgaagtataacctgagcattgcacctgcnntatgcacccaccaggatacgtatgatgtttgaagagcgtttaaacgctcttcgcccctaaagggcgatctcaacaagtttcgagacggccctga

SEQ ID NO: 126 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctcctatcatacgtatcctggtgtttgnnngcaggtgcaatgtttccggaaacctatcgtcgccattgcacctgcnntatgcaacatagacccctgttgtaaggtttgaagagcgtttaaacgctcttcgcccccctcaccttctacccagccgtttcgagacggccctga

SEQ ID NO: 127 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctaccttcacccataggagccacagcgtttgnnngcaggtgcaatgtgtatatgtccagtcgggcggcattgcacctgcnntatgcaagatcatacagctgcccaacgtttgaagagcgtttaaacgctcttcgcccagctgtagtagagtccccatgtttcgagacggccctga

SEQ ID NO: 128 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctctgctagccaacaccactgagtttgnnngcaggtgcaatgccagtcctaactagcccgtttcattgcacctgcnntatgcacaacttcttcgtggtatctcgtttgaagagcgtttaaacgctcttcgccctgttgggcagctgtatgatcgtttcgagacggccctga

SEQ ID NO: 129 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctacctatcatacgtatcctgggtttgnnngcaggtgcaatgctcttacacccatgcactgcacattgcacctgcnntatgcagccctttagctggtatctgcgtttgaagagcgtttaaacgctcttcgcccagaagttggtgacgttctgcgtttcgagacggccctga

SEQ ID NO: 130 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctacctctgcagttgagctggcaatcgtttgnnngcaggtgcaatgcccgcctactaaggaacgaatcattgcacctgcnntatgcaacctatcatacgtatcctgggtttgaagagcgtttaaacgctcttcgcccggtcatcgatcgcctctagtgtttcgagacggccctga

SEQ ID NO: 131 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctggaagggacactaacctggtgtttgnnngcaggtgcaatgcgaccgacaccttaacccatgcattgcacctgcnntatgcatgttgggcagctgtatgatcgtttgaagagcgtttaaacgctcttcgccccctacgcagcgacgagtcttgtttcgagacggccctga

SEQ ID NO: 132 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctggaagggacactaacctggtgtttgnnngcaggtgcaatggcctaccaccgaaattcttcccattgcacctgcnntatgcaccctcaccttctacccagccgtttgaagagcgtttaaacgctcttcgcccgtcgcttacgaacgcttacggtttcgagacggccctga

SEQ ID NO: 133 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctcaaagacctgcggcgagggcgtttgnnngcaggtgcaatgcttggtcaaatctcgacgacccattgcacctgcnntatgcatgaatgaccagggtacctgcgtttgaagagcgtttaaacgctcttcgccctaggcgcccattcgacgacggtttcgagacggccctga

SEQ ID NO: 134 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctttcatagacctaatgtgactgtttgnnngcaggtgcaatggtgcaaacagtaaccgactcccattgcacctgcnntatgcactgctagccaacaccactgagtttgaagagcgtttaaacgctcttcgccccgtcggtcacgagtacatgcgtttcgagacggccctga

SEQ ID NO: 135 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctacctatcatacgtatcctgggtttgnnngcaggtgcaatgctactgtcaaccccggttaaccattgcacctgcnntatgcagattccttccaataacacaagtttgaagagcgtttaaacgctcttcgcccggttcgacagcgttaggcccgtttcgagacggccctga

SEQ ID NO: 136 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctgtgacttggtgtaagcacaagtttgnnngcaggtgcaatgcgtccgttaatccgactcactcattgcacctgcnntatgcacatcggcctgacccccatgcgtttgaagagcgtttaaacgctcttcgcccgcccgtatgcgagaatcgaggtttcgagacggccctga

SEQ ID NO: 137 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctaccttgcccacaaagtatggcggtgtttgnnngcaggtgcaatgcactttcactaccacagggaccattgcacctgcnntatgcattgttacagccttattacacgtttgaagagcgtttaaacgctcttcgcccagctcgggtaatgtccgttagtttcgagacggccctga

SEQ ID NO: 138 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctattcttctctgtgaatgtcggtttgnnngcaggtgcaatgccaacgcataaccgctatacccattgcacctgcnntatgcattgggactcctcggttatttgtttgaagagcgtttaaacgctcttcgccccgcggacgatatatcggctcgtttcgagacggccctga

SEQ ID NO: 139 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctttgttacagccttattacacgtttgnnngcaggtgcaatgccaacagacatgccccacagtcattgcacctgcnntatgcacagtgcaacttactattacggtttgaagagcgtttaaacgctcttcgccccggacgaatgaccaaccggggtttcgagacggccctga

SEQ ID NO: 140 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctggccatttatagtgttgaccgtttgnnngcaggtgcaatgagccaaaagatcgccgactctcattgcacctgcnntatgcacaatacagggatacccactagtttgaagagcgtttaaacgctcttcgccctgtcgtgggcagtagtatcggtttcgagacggccctga

SEQ ID NO: 141 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctgctcaaaccattacagaagggtttgnnngcaggtgcaatgcagcacagagagtacaaggcccattgcacctgcnntatgcaacctatcatacgtatcctgggtttgaagagcgtttaaacgctcttcgccccgcacgccgatatcattcgggtttcgagacggccctga

SEQ ID NO: 142 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctcaatacagggatacccactagtttgnnngcaggtgcaatgcacaaaaggaacgcacgactgcattgcacctgcnntatgcatcacccataggagccacagcgtttgaagagcgtttaaacgctcttcgccccgactaccccgggtgcgacagtttcgagacggccctga

SEQ ID NO: 143 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctgttacactctatctacacctgtttgnnngcaggtgcaatgctaatcgcgaccacgaagcatcattgcacctgcnntatgcatgaatgaccagggtacctgcgtttgaagagcgtttaaacgctcttcgccctagttcgttcgacggagcgtgtttcgagacggccctga

SEQ ID NO: 144 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctacctctgcagttgagctggcaatcgtttgnnngcaggtgcaatgtcgcgcaacctcgtcaaatcccattgcacctgcnntatgcacaacttcttcgtggtatctcgtttgaagagcgtttaaacgctcttcgcccattttagtacccgcgccgacgtttcgagacggccctga

SEQ ID NO: 145 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctaccttgttgagatcgccctttagcgtttgnnngcaggtgcaatgacgttcttacaccagcgcatgcattgcacctgcnntatgcacgtcaccaagccattgtaccgtttgaagagcgtttaaacgctcttcgcccacccgctccgacgacccaaggtttcgagacggccctga

SEQ ID NO: 146 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctaccttgttgagatcgccctttagcgtttgnnngcaggtgcaatggctaacttgcacgaatcgagccattgcacctgcnntatgcaattcttctctgtgaatgtcggtttgaagagcgtttaaacgctcttcgcccaacgatccggtaggtcggccgtttcgagacggccctga

SEQ ID NO: 147 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctacctgtatcctggtgggatttacagtttgnnngcaggtgcaatgcgaagattgcacacggtaagccattgcacctgcnntatgcaagcacacaagcacgttacccgtttgaagagcgtttaaacgctcttcgcccatctgtattaacgcggtacagtttcgagacggccctga

SEQ ID NO: 148 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctcgtcaccaagccattgtaccgtttgnnngcaggtgcaatgcgactcccttactcagacgaccattgcacctgcnntatgcagccctttagctggtatctgcgtttgaagagcgtttaaacgctcttcgccctcccgccgcgtacctatgtagtttcgagacggccctga

SEQ ID NO: 149 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctattcttctctgtgaatgtcggtttgnnngcaggtgcaatggacctattgataaccccagcgcattgcacctgcnntatgcagaggcggtcaggatggctgcgtttgaagagcgtttaaacgctcttcgcccagattctcatcgcgcggattgtttcgagacggccctga

SEQ ID NO: 150 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctagatcatacagctgcccaacgtttgnnngcaggtgcaatggcacgaccaacatcctcgaatcattgcacctgcnntatgcaagttgaacgcgtcaccgcgtgtttgaagagcgtttaaacgctcttcgccctagacctcgacgaacgctttgtttcgagacggccctga

SEQ ID NO: 151 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctaccttctttgaaccattgtgttatgtttgnnngcaggtgcaatggtccaacatgaacccgcttagcattgcacctgcnntatgcagcgagcttaccgactttccggtttgaagagcgtttaaacgctcttcgcccgcgagcgcggaaaattcggagtttcgagacggccctga

SEQ ID NO: 152 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctgattccttccaataacacaagtttgnnngcaggtgcaatgaccgacgttacatagacgacgcattgcacctgcnntatgcaaggggcgacatcgttttacggtttgaagagcgtttaaacgctcttcgccccgggctattataacgacggggtttcgagacggccctga

SEQ ID NO: 153 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctaatcccttgccccagcacatgtttgnnngcaggtgcaatgtcgtaacatacgcgcaatccccattgcacctgcnntatgcacgggcaacgaacgcgggcacgtttgaagagcgtttaaacgctcttcgccctagcatgcgctttgcggcgagtttcgagacggccctga

SEQ ID NO: 154 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctggaagggacactaacctggtgtttgnnngcaggtgcaatgcttcgaaaagccccagagctgcattgcacctgcnntatgcatacatgcacagcgcgcgacggtttgaagagcgtttaaacgctcttcgcccaattcgcgcgaattggcggggtttcgagacggccctga

SEQ ID NO: 155 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctaccttctgggcatgtgggtccggcgtttgnnngcaggtgcaatgcgatacagcaacgtccatgaccattgcacctgcnntatgcacgataggtacccgtagcgaagtttgaagagcgtttaaacgctcttcgcccttcccgcgcctactatcggcgtttcgagacggccctga

SEQ ID NO: 156 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctacctacacaacactgatgaggtccgtttgnnngcaggtgcaatgcagcagaggctacaattaccgcattgcacctgcnntatgcatctcgggcgacgccgaaatagtttgaagagcgtttaaacgctcttcgcccatctacgtaccgttagcggagtttcgagacggccctga

SEQ ID NO: 157 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctgttacactctatctacacctgtttgnnngcaggtgcaatgctacgtagaccaatgcaccaccattgcacctgcnntatgcagcgcggttccgcggatattcgtttgaagagcgtttaaacgctcttcgcccttacgcagcgacgtcttaatgtttcgagacggccctga

SEQ ID NO: 158 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctaccttctgggcatgtgggtccggcgtttgnnngcaggtgcaatgtcaatgaccggcacggaaacccattgcacctgcnntatgcacatcgggttaaggtcgtagtgtttgaagagcgtttaaacgctcttcgccccgcggaaatcgagcatcacggtttcgagacggccctga

SEQ ID NO: 159 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctacctggccatttatagtgttgaccgtttgnnngcaggtgcaatgcggcagacgaagaaaaggccacattgcacctgcnntatgcagcgctaaaaacggaccgttcgtttgaagagcgtttaaacgctcttcgccccaccgcgagctaatccgcgagtttcgagacggccctga

SEQ ID NO: 160 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctgaggcggtcaggatggctgcgtttgnnngcaggtgcaatgtaccttcccaccctactggaccattgcacctgcnntatgcacggcataactacgctcgctcgtttgaagagcgtttaaacgctcttcgcccgtcaacaccccgcgcgaatagtttcgagacggccctga

SEQ ID NO: 161 - DNA, 236 bp, artificial
  oligo
  misc_feature (93)..(95): n is a, c, g, or t
  misc_feature (141)..(142): n is a, c, g, or t
gaccgcgtctcacaccgcgtcaccaagccattgtaccgtttggagaccgtttaaacggtctctacctcctatcatacgtatcctggtgtttgnnngcaggtgcaatgacctacctctcaagtaccggccattgcacctgcnntatgcacgtaccgagaagtagggccggtttgaagagcgtttaaacgctcttcgcccgctacggtagacgtcgacaagtttcgagacggccctga

SEQ ID NO: 162 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctctgaaattagacatcaaagcgtttgnnngcaggtgcaatgccacactataaggcgtcacaccattgcacctgcnntatgcacgatacatgttcgtcggtaggtttgaagagcgtttaaacgctcttcgcccggtacgcgtaccatagtcgggtttcgagacggccctga

SEQ ID NO: 163 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctcaaagacctgcggcgagggcgtttgnnngcaggtgcaatgccccgacttaatgcaagagcgcattgcacctgcnntatgcatatcgactactacgaccgccgtttgaagagcgtttaaacgctcttcgcccctcgatcccgttcgacggccgtttcgagacggccctga

SEQ ID NO: 164 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctcatcggcctgacccccatgcgtttgnnngcaggtgcaatgagtatgccccagaccgatcaccattgcacctgcnntatgcatatcgcgaagtgccgtgagtgtttgaagagcgtttaaacgctcttcgccctcggtactttgaaaccgcgcgtttcgagacggccctga

SEQ ID NO: 165 - DNA, 236 bp, artificial
  oligo
  misc_feature (93)..(95): n is a, c, g, or t
  misc_feature (141)..(142): n is a, c, g, or t
gaccgcgtctcacaccggattccttccaataacacaagtttggagaccgtttaaacggtctctacctgttcgcgaagtgcccctagtgtttgnnngcaggtgcaatgagaacgggccgaagaatcgaacattgcacctgcnntatgcactcgcgcgcttagacaatttgtttgaagagcgtttaaacgctcttcgccccgcgacatgatggtacgcgtgtttcgagacggccctga

SEQ ID NO: 166 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctacctattgcgcgtttgaccgaatggtttgnnngcaggtgcaatgctctttcgcaggcccacaatacattgcacctgcnntatgcaccgaagtgtatcccgatccggtttgaagagcgtttaaacgctcttcgcccaatacattcgagcggcgtcggtttcgagacggccctga

SEQ ID NO: 167 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcgggtgcgtttactcgtctagtttgnnngcaggtgcaatgctaattcgtcgcaccatcgagcattgcacctgcnntatgcatcggtgcttcgattgtcggggtttgaagagcgtttaaacgctcttcgccccgcgcgcacataatgccttagtttcgagacggccctga

SEQ ID NO: 168 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctcgcattgaccgacccgtcgagtttgnnngcaggtgcaatgcctcacttacggaaaccccgacattgcacctgcnntatgcaatattcctcgctccgcgtaagtttgaagagcgtttaaacgctcttcgccctatcgcaatcgctccggaatgtttcgagacggccctga

SEQ ID NO: 169 - DNA, 236 bp, artificial
  oligo
  misc_feature (93)..(95): n is a, c, g, or t
  misc_feature (141)..(142): n is a, c, g, or t
gaccgcgtctcacaccggttacactctatctacacctgtttggagaccgtttaaacggtctctacctaatcttatcgattagcgggcgtttgnnngcaggtgcaatgcactcgctaaacgcagcactccattgcacctgcnntatgcatatggccgatgcaacgcaacgtttgaagagcgtttaaacgctcttcgcccggccccatatcggtacggcagtttcgagacggccctga

SEQ ID NO: 170 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctagtgaacggttgggtacgccgtttgnnngcaggtgcaatggcaacatgtcagtacccgttccattgcacctgcnntatgcagcgcgacgattatcacgttcgtttgaagagcgtttaaacgctcttcgccctccgcggaacatacctacgtgtttcgagacggccctga

SEQ ID NO: 171 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctgagaagcactatgggggcatgtttgnnngcaggtgcaatgtgggtagagacttacaacgcccattgcacctgcnntatgcagtagaccaggcacccaacctgtttgaagagcgtttaaacgctcttcgcccaaccccaacatcagcaagcggtttcgagacggccctga

SEQ ID NO: 172 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctcaagggcgtgaacagcgacggtttgnnngcaggtgcaatgcctataccaacctcggcatcgcattgcacctgcnntatgcagtgcatacgtacttcgtccagtttgaagagcgtttaaacgctcttcgcccccacagtactcacttggtccgtttcgagacggccctga

SEQ ID NO: 173 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctatcttgcagatgctaatagcgtttgnnngcaggtgcaatgcaacctagacaagacctcccgcattgcacctgcnntatgcagcagtgaccgtcacgacctggtttgaagagcgtttaaacgctcttcgcccaacatgactccccggaggccgtttcgagacggccctga

SEQ ID NO: 174 - DNA, 236 bp, artificial
  oligo
  misc_feature (93)..(95): n is a, c, g, or t
  misc_feature (141)..(142): n is a, c, g, or t
gaccgcgtctcacaccggttgccaggtgagcccgctcgtttggagaccgtttaaacggtctctacctcaagggcgtgaacagcgacggtttgnnngcaggtgcaatgaccgtgacaaactgactagcccattgcacctgcnntatgcagtcacacctgagtaccactggtttgaagagcgtttaaacgctcttcgccctcccacagttgtaccttatcgtttcgagacggccctga

SEQ ID NO: 175 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctgcagtgaccgtcacgacctggtttgnnngcaggtgcaatgtaacgacgaccaaggaccactcattgcacctgcnntatgcaccacagtactcacttggtccgtttgaagagcgtttaaacgctcttcgcccgcaaagccgatgtcagcgccgtttcgagacggccctga

SEQ ID NO: 176 - DNA, 236 bp, artificial
  oligo
  misc_feature (93)..(95): n is a, c, g, or t
  misc_feature (141)..(142): n is a, c, g, or t
gaccgcgtctcacaccggtagaccaggcacccaacctgtttggagaccgtttaaacggtctctacctgagaggtttccgaagactaagtttgnnngcaggtgcaatgtcggaaccacaacactgaccccattgcacctgcnntatgcataagctgctctctagacggagtttgaagagcgtttaaacgctcttcgcccatagcggggaacgcatcgtcgtttcgagacggccctga

SEQ ID NO: 177 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctgcaggtccttaccttgttccgtttgnnngcaggtgcaatgtgtgttaccacaatcgcgcgccattgcacctgcnntatgcaaagcactacccctgccaaccgtttgaagagcgtttaaacgctcttcgccccggccgttatcgagcaatatgtttcgagacggccctga

SEQ ID NO: 178 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctgttcactacgagtgtattccgtttgnnngcaggtgcaatgacaccgacgacattaggaagccattgcacctgcnntatgcaaccaggctgacagttatcacgtttgaagagcgtttaaacgctcttcgcccatatggcggtcgaacaatgcgtttcgagacggccctga

SEQ ID NO: 179 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctaccttaccttgagactccaccctcgtttgnnngcaggtgcaatggcgcaggacatttacgcagctcattgcacctgcnntatgcaaaccccaacatcagcaagcggtttgaagagcgtttaaacgctcttcgcccccgtgacttacgctatacaggtttcgagacggccctga

SEQ ID NO: 180 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctaccaggcacccaacctcggcgtttgnnngcaggtgcaatgtctccatcgtgaaatcccgcacattgcacctgcnntatgcatggcattggggacctcgggtgtttgaagagcgtttaaacgctcttcgcccggtagcgcgttaaccttggtgtttcgagacggccctga

SEQ ID NO: 181 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctacctacctgtggtggttatggggcgtttgnnngcaggtgcaatgcctaaagtccgtcaaagaccccattgcacctgcnntatgcatcctcagctgccatatgtgcgtttgaagagcgtttaaacgctcttcgccctatgcggcgcggatcgtgacgtttcgagacggccctga

SEQ ID NO: 182 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctacctgcaaagccgatgtcagcgccgtttgnnngcaggtgcaatgttgacctcagcgatcaacgcccattgcacctgcnntatgcaaccaggcacccaacctcggcgtttgaagagcgtttaaacgctcttcgcccaaaccgcgacgcactcctaggtttcgagacggccctga

SEQ ID NO: 183 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctcagcttcaggaccccgatccgtttgnnngcaggtgcaatgcgagatgtgctaccaacctcgcattgcacctgcnntatgcagcttgtggtagatagcaacggtttgaagagcgtttaaacgctcttcgcccttgcccgcccgatactgcgagtttcgagacggccctga

SEQ ID NO: 184 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctccaagtaccttctccagcatgtttgnnngcaggtgcaatgatgcgccaccaaagcacgatacattgcacctgcnntatgcaaaagcagtcgcccctgcttggtttgaagagcgtttaaacgctcttcgccctagaccgtcatcggaatccggtttcgagacggccctga

SEQ ID NO: 185 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctgagaagcactatgggggcatgtttgnnngcaggtgcaatgtgttatccccgttcacaccgacattgcacctgcnntatgcatcacacttggagttacagcagtttgaagagcgtttaaacgctcttcgcccttaacacgtcccggcgccgtgtttcgagacggccctga

SEQ ID NO: 186 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctgctgacatcggctttgctgcgtttgnnngcaggtgcaatgttgcatgaccacactctgacgcattgcacctgcnntatgcacgatcgtgtggctcgtcgaggtttgaagagcgtttaaacgctcttcgccccgtagcggggcgaacttttagtttcgagacggccctga

SEQ ID NO: 187 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgttcactacgagtgtattccgtttgnnngcaggtgcaatgtccacgtactatctcgtcccccattgcacctgcnntatgcacgtcctacgacccgtggcgcgtttgaagagcgtttaaacgctcttcgcccaaccggcggtacgcgcgtttgtttcgagacggccctga

SEQ ID NO: 188 - DNA, 236 bp, artificial
  oligo
  misc_feature (93)..(95): n is a, c, g, or t
  misc_feature (141)..(142): n is a, c, g, or t
gaccgcgtctcacaccggttcactacgagtgtattccgtttggagaccgtttaaacggtctctaccttaccttgagactccaccctcgtttgnnngcaggtgcaatggcctctaacactcactggaagcattgcacctgcnntatgcagtcccgcaccgacctttaatgtttgaagagcgtttaaacgctcttcgccccgctcgagaacacgtacatcgtttcgagacggccctga

SEQ ID NO: 189 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctacctgagaggtttccgaagactaagtttgnnngcaggtgcaatgagattcctctgccttatcccccattgcacctgcnntatgcatctcaacgtagcgcgacatcgtttgaagagcgtttaaacgctcttcgccctccactcgaacgagcggggtgtttcgagacggccctga

SEQ ID NO: 190 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctacctgtgcatacgtacttcgtccagtttgnnngcaggtgcaatgcttacacgtacagccactcgccattgcacctgcnntatgcagcgacgttagccctatcgcggtttgaagagcgtttaaacgctcttcgccctaccctcggcattcgaccgagtttcgagacggccctga

SEQ ID NO: 191 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctaccttgtgaaaaggactgcaggccgtttgnnngcaggtgcaatgcccatggatcattatccggcacattgcacctgcnntatgcacgccgaatattagcccgaacgtttgaagagcgtttaaacgctcttcgcccccacgccgaacgccgtcagtgtttcgagacggccctga

SEQ ID NO: 192 - DNA, 236 bp, artificial
  oligo
  misc_feature (93)..(95): n is a, c, g, or t
  misc_feature (141)..(142): n is a, c, g, or t
gaccgcgtctcacaccgtacgctgcaaagtctctggcgtttggagaccgtttaaacggtctctaccttaagctgctctctagacggagtttgnnngcaggtgcaatgcgcatcacatatctaggtccgcattgcacctgcnntatgcacggctatcggatagactagggtttgaagagcgtttaaacgctcttcgccccgacgacctacaatggcggtgtttcgagacggccctga

SEQ ID NO: 193 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctaccaggcacccaacctcggcgtttgnnngcaggtgcaatgcctccttcaataaacgcgcaccattgcacctgcnntatgcacgcgataagatttcgagcacgtttgaagagcgtttaaacgctcttcgccccaatatcccggcgcgaagcggtttcgagacggccctga

SEQ ID NO: 194 - DNA, 236 bp, artificial
  oligo
  misc_feature (93)..(95): n is a, c, g, or t
  misc_feature (141)..(142): n is a, c, g, or t
gaccgcgtctcacaccgtcacacttggagttacagcagtttggagaccgtttaaacggtctctacctgcttgtggtagatagcaacggtttgnnngcaggtgcaatgcaaatccgaccaagcggtactcattgcacctgcnntatgcagacgccttaatcatcgagatgtttgaagagcgtttaaacgctcttcgcccgttccgtcgttcctcgcgtcgtttcgagacggccctga

SEQ ID NO: 195 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctacctctagcaagccataacaaaacgtttgnnngcaggtgcaatgatgcccgatccttaccacatccattgcacctgcnntatgcagctcgcgcattcttacgtcggtttgaagagcgtttaaacgctcttcgccccgatcgccccttttcggtaagtttcgagacggccctga

SEQ ID NO: 196 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctgcgaggttcgccgtcgtgatgtttgnnngcaggtgcaatgcccattacccgcacttattgccattgcacctgcnntatgcacggatgttacctcggtagacgtttgaagagcgtttaaacgctcttcgccctcgtgtacactgatccggccgtttcgagacggccctga

SEQ ID NO: 197 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctaccttaccatccgacgcgctcgaagtttgnnngcaggtgcaatgtcatcgggaccacaacaaacgcattgcacctgcnntatgcaactcgttcgaccgcgtcgcggtttgaagagcgtttaaacgctcttcgcccgaccgcgtagcaattcgtatgtttcgagacggccctga

SEQ ID NO: 198 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctacctcgccaagtggattgccgtacgtttgnnngcaggtgcaatggaattcacagcccatccccttcattgcacctgcnntatgcatggaacggtcgacttcgggcgtttgaagagcgtttaaacgctcttcgcccctctcggcaattcgcgctcagtttcgagacggccctga

SEQ ID NO: 199 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctacctcggttcgaccgttacgttgtgtttgnnngcaggtgcaatgccgcatttcaaacgccttcaccattgcacctgcnntatgcatcgcgctccgtcgttttggtgtttgaagagcgtttaaacgctcttcgcccgtacgtttgtaactcggtaggtttcgagacggccctga

SEQ ID NO: 200 - DNA, 236 bp, artificial
  oligo
  misc_feature (93)..(95): n is a, c, g, or t
  misc_feature (141)..(142): n is a, c, g, or t
gaccgcgtctcacaccgatccccgtcgcagttgaactgtttggagaccgtttaaacggtctctacctatcagttcgagcgtgcgatggtttgnnngcaggtgcaatgactagcccctatacggaaccacattgcacctgcnntatgcaatattgccggcacgatagatgtttgaagagcgtttaaacgctcttcgccccccgaacggaataccccgtagtttcgagacggccctga

SEQ ID NO: 201 - DNA, 236 bp, artificial
  oligo
  misc_feature (93)..(95): n is a, c, g, or t
  misc_feature (141)..(142): n is a, c, g, or t
gaccgcgtctcacaccgccgagttacctcgagctttcgtttggagaccgtttaaacggtctctacctaagaggcttcttatgtcggcgtttgnnngcaggtgcaatgttcctcagagcaacgcctcttcattgcacctgcnntatgcatcgacaacccctacatcgtggtttgaagagcgtttaaacgctcttcgcccctcacggcagcgcacgcacagtttcgagacggccctga

SEQ ID NO: 202 - DNA, 236 bp, artificial
  oligo
  misc_feature (93)..(95): n is a, c, g, or t
  misc_feature (141)..(142): n is a, c, g, or t
gaccgcgtctcacaccggcatccacattcttacaggcgtttggagaccgtttaaacggtctctacctttctcggaagtcgaggacaggtttgnnngcaggtgcaatgaacaccgcagctataacagcccattgcacctgcnntatgcaaacggcacgtacgccatcgcgtttgaagagcgtttaaacgctcttcgccccgccggcgctctcaagtatcgtttcgagacggccctga

SEQ ID NO: 203 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctactcacggcagcgcacgcacgtttgnnngcaggtgcaatgtaatccaccagcggcacggaacattgcacctgcnntatgcacgcccagggagcgcaaacacgtttgaagagcgtttaaacgctcttcgcccgaactgtatacggctccgcggtttcgagacggccctga

SEQ ID NO: 204 - DNA, 236 bp, artificial
  oligo
  misc_feature (93)..(95): n is a, c, g, or t
  misc_feature (141)..(142): n is a, c, g, or t
gaccgcgtctcacaccgttacattgtatacgccttccgtttggagaccgtttaaacggtctctaccttatccggttacctcatagtcgtttgnnngcaggtgcaatgttctagcccaaccctatctcccattgcacctgcnntatgcatcgggggacggttatttctggtttgaagagcgtttaaacgctcttcgcccatagccttgcgtcgagtatggtttcgagacggccctga

SEQ ID NO: 205 - DNA, 236 bp, artificial
  oligo
  misc_feature (93)..(95): n is a, c, g, or t
  misc_feature (141)..(142): n is a, c, g, or t
gaccgcgtctcacaccggctcaatctagggcgaagaggtttggagaccgtttaaacggtctctacctgtgacccgaggaaccggtgcgtttgnnngcaggtgcaatgcccccacatcattatagccctcattgcacctgcnntatgcacgcgcaccatagcatcacgcgtttgaagagcgtttaaacgctcttcgcccacgtgaaatcgatgccggatgtttcgagacggccctga

SEQ ID NO: 206 - DNA, 236 bp, artificial
  oligo
  misc_feature (93)..(95): n is a, c, g, or t
  misc_feature (141)..(142): n is a, c, g, or t
gaccgcgtctcacaccgctcacggcagcgcacgcacagtttggagaccgtttaaacggtctctacctcgcgcaccatagcatcacgcgtttgnnngcaggtgcaatgttatttgcccagacacctcggcattgcacctgcnntatgcatcgaacgcgaagggtcgcgtgtttgaagagcgtttaaacgctcttcgccccgacgccgacggccgatgcagtttcgagacggccctga

SEQ ID NO: 207 - DNA, 236 bp, artificial
  oligo
  misc_feature (93)..(95): n is a, c, g, or t
  misc_feature (141)..(142): n is a, c, g, or t
gaccgcgtctcacaccgtatccggttacctcatagtcgtttggagaccgtttaaacggtctctacctctcgtagcttgccacactgtgtttgnnngcaggtgcaatgaccctacctcaagaatgtcggcattgcacctgcnntatgcatcgtaatagatccgcgtctagtttgaagagcgtttaaacgctcttcgccctcgatagttgacccgttcaagtttcgagacggccctga

SEQ ID NO: 208 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctacctctcacggcagcgcacgcacagtttgnnngcaggtgcaatggcaccttccagaatgttcccacattgcacctgcnntatgcatatggcgcgctcgattcagggtttgaagagcgtttaaacgctcttcgcccctacgggtacatacgcgcgtgtttcgagacggccctga

SEQ ID NO: 209 - DNA, 236 bp, artificial
  oligo
  misc_feature (93)..(95): n is a, c, g, or t
  misc_feature (141)..(142): n is a, c, g, or t
gaccgcgtctcacaccggtgacccgaggaaccggtgcgtttggagaccgtttaaacggtctctacctgaagcgagagaatctcctcggtttgnnngcaggtgcaatgataaccatccacacacgggagcattgcacctgcnntatgcaccgcggcggataacgtgtttgtttgaagagcgtttaaacgctcttcgcccggccctattagcgtcgcgacgtttcgagacggccctga

SEQ ID NO: 210 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctacctcttctatggcagcatctcgcgtttgnnngcaggtgcaatgcacaagccaaaacatccgacccattgcacctgcnntatgcaggtcgtaattatgcccttcggtttgaagagcgtttaaacgctcttcgcccacgtcgacccttcgacgcttgtttcgagacggccctga

SEQ ID NO: 211 - DNA, 236 bp, artificial
  oligo
  misc_feature (93)..(95): n is a, c, g, or t
  misc_feature (141)..(142): n is a, c, g, or t
gaccgcgtctcacaccgggggcacgatggcctttaccgtttggagaccgtttaaacggtctctacctgtgacccgaggaaccggtgcgtttgnnngcaggtgcaatgcggctactcacaattcaggtgcattgcacctgcnntatgcaataagtccaatcgggcgccagtttgaagagcgtttaaacgctcttcgccctaaaacgccgcgtcggaatggtttcgagacggccctga

SEQ ID NO: 212 - DNA, 236 bp, artificial
  oligo
  misc_feature (93)..(95): n is a, c, g, or t
  misc_feature (141)..(142): n is a, c, g, or t
gaccgcgtctcacaccgttgggggatactcacgggatgtttggagaccgtttaaacggtctctacctaaacgggacaacggccgcgtgtttgnnngcaggtgcaatggcacgtagacggacctatagccattgcacctgcnntatgcagcgagtcgaccgttggacgtgtttgaagagcgtttaaacgctcttcgcccggaacgcaatcgccggttgagtttcgagacggccctga

SEQ ID NO: 213 - DNA, 236 bp, artificial
  oligo
  misc_feature (93)..(95): n is a, c, g, or t
  misc_feature (141)..(142): n is a, c, g, or t
gaccgcgtctcacaccgcgcgcaccatagcatcacgcgtttggagaccgtttaaacggtctctacctcgaacgatatccgaatgcacgtttgnnngcaggtgcaatgccgctgtttatatcaccgacgcattgcacctgcnntatgcaccgcccacgatattggcggagtttgaagagcgtttaaacgctcttcgccctcgcttcgttcgcacgtattgtttcgagacggccctga

SEQ ID NO: 214 - DNA, 236 bp, artificial
  oligo
  misc_feature (93)..(95): n is a, c, g, or t
  misc_feature (141)..(142): n is a, c, g, or t
gaccgcgtctcacaccgttctcggaagtcgaggacaggtttggagaccgtttaaacggtctctacctgagcgcgtcggtatgcgtcggtttgnnngcaggtgcaatgcgcactcacctcacgtttaaccattgcacctgcnntatgcatgacgttcaccgaatacgacgtttgaagagcgtttaaacgctcttcgcccctttccttcgcgcgtaatcggtttcgagacggccctga

SEQ ID NO: 215 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctaccttagtggtcgcctgcgcgaatgtttgnnngcaggtgcaatggcctcttgaccacgaactatgcattgcacctgcnntatgcacgcgaggcgtttagcctagtgtttgaagagcgtttaaacgctcttcgccctacgcacgaggatcgcctatgtttcgagacggccctga

SEQ ID NO: 216 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctcaatacagggatacccactagtttgnnngcaggtgcaatggcagaaatcatatcggccacccattgcacctgcnntatgcaacctatcatacgtatcctgggtttgaagagcgtttaaacgctcttcgcccacacaacactgatgaggtccgtttcgagacggccctga

SEQ ID NO: 217 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctttgttacagccttattacacgtttgnnngcaggtgcaatgccggacacagaaaactccgtacattgcacctgcnntatgcaggccatttatagtgttgaccgtttgaagagcgtttaaacgctcttcgccctcatcctgcatgctagaccagtttcgagacggccctga

SEQ ID NO: 218 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctcaacttcttcgtggtatctcgtttgnnngcaggtgcaatggagccactaggaaatcttccccattgcacctgcnntatgcatcatcctgcatgctagaccagtttgaagagcgtttaaacgctcttcgccccagcttgtccaactggtcgggtttcgagacggccctga

SEQ ID NO: 219 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctaccttgttgggcagctgtatgatcgtttgnnngcaggtgcaatgcaccgtcagcactttaatcgccattgcacctgcnntatgcatctttgaaccattgtgttatgtttgaagagcgtttaaacgctcttcgcccattcttctctgtgaatgtcggtttcgagacggccctga

SEQ ID NO: 220 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctagatcatacagctgcccaacgtttgnnngcaggtgcaatgaccacgccgagcatttatcctcattgcacctgcnntatgcaggaagggacactaacctggtgtttgaagagcgtttaaacgctcttcgcccactttccatgtcagccatacgtttcgagacggccctga

SEQ ID NO: 221 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctcagcttgtccaactggtcgggtttgnnngcaggtgcaatgtccatgaacagtcccatgccacattgcacctgcnntatgcaggagagccaacccctaccaggtttgaagagcgtttaaacgctcttcgcccttgggactcctcggttatttgtttcgagacggccctga

SEQ ID NO: 222 - DNA, 236 bp, artificial
  oligo
  misc_feature (93)..(95): n is a, c, g, or t
  misc_feature (141)..(142): n is a, c, g, or t
gaccgcgtctcacaccgtcacatggatggttactgaagtttggagaccgtttaaacggtctctacctggactgagagctgttgacacgtttgnnngcaggtgcaatgcgacttgtacaacgatcccgacattgcacctgcnntatgcactggaaggtgaggcctaggcgtttgaagagcgtttaaacgctcttcgcccgaggcggtcaggatggctgcgtttcgagacggccctga

SEQ ID NO: 223 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctgattccttccaataacacaagtttgnnngcaggtgcaatgacctcagcacagacagtgacccattgcacctgcnntatgcacctatcatacgtatcctggtgtttgaagagcgtttaaacgctcttcgccctgcccacaaagtatggcggtgtttcgagacggccctga

SEQ ID NO: 224 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctggagagccaacccctaccaggtttgnnngcaggtgcaatggacctcacggcatcacaacagcattgcacctgcnntatgcattgttacagccttattacacgtttgaagagcgtttaaacgctcttcgcccgattccttccaataacacaagtttcgagacggccctga

SEQ ID NO: 225 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctacaggtgacccaaccttcaggtttgnnngcaggtgcaatgcctttcacattccggccaactcattgcacctgcnntatgcagccctttagctggtatctgcgtttgaagagcgtttaaacgctcttcgcccggagagccaacccctaccaggtttcgagacggccctga

SEQ ID NO: 226 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctgtatcctggtgggatttacagtttgnnngcaggtgcaatgtgtgatggggtggacctgttgcattgcacctgcnntatgcagctcaaaccattacagaagggtttgaagagcgtttaaacgctcttcgcccaatcccttgccccagcacatgtttcgagacggccctga

SEQ ID NO: 227 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctctccacaaagacctgcggcggtttgnnngcaggtgcaatgccagcaggccgtatgtatagtcattgcacctgcnntatgcaggccatttatagtgttgaccgtttgaagagcgtttaaacgctcttcgcccacaggtgacccaaccttcaggtttcgagacggccctga

SEQ ID NO: 228 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctctaaagggcgatctcaacaagtttgnnngcaggtgcaatggccaatcgtccgactaaacgccattgcacctgcnntatgcacatcggcctgacccccatgcgtttgaagagcgtttaaacgctcttcgccctcacccataggagccacagcgtttcgagacggccctga

SEQ ID NO: 229 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctctgctagccaacaccactgagtttgnnngcaggtgcaatgtcacccggagttcactacccacattgcacctgcnntatgcagttacactctatctacacctgtttgaagagcgtttaaacgctcttcgcccgctcaaaccattacagaagggtttcgagacggccctga

SEQ ID NO: 230 - DNA, 236 bp, artificial
  oligo
  misc_feature (93)..(95): n is a, c, g, or t
  misc_feature (141)..(142): n is a, c, g, or t
gaccgcgtctcacaccgcgtcaccaagccattgtaccgtttggagaccgtttaaacggtctctacctctggaaggtgaggcctaggcgtttgnnngcaggtgcaatgatgcatacaccgtcgctaaggcattgcacctgcnntatgcaattcttctctgtgaatgtcggtttgaagagcgtttaaacgctcttcgcccagctgtagtagagtccccatgtttcgagacggccctga

SEQ ID NO: 231 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctccctcaccttctacccagccgtttgnnngcaggtgcaatgtccctcgaaaatccccacaagcattgcacctgcnntatgcacagtgcaacttactattacggtttgaagagcgtttaaacgctcttcgcccgtgcgatagaccgtcccgatgtttcgagacggccctga

SEQ ID NO: 232 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctcaatacagggatacccactagtttgnnngcaggtgcaatgctaaaccgatagacgtacccccattgcacctgcnntatgcaacatagacccctgttgtaaggtttgaagagcgtttaaacgctcttcgccctcgatagttgcgtcgcatacgtttcgagacggccctga

SEQ ID NO: 233 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctaccttctttgaaccattgtgttatgtttgnnngcaggtgcaatggaaatatcagcgtccgctcaccattgcacctgcnntatgcatgttgagatcgccctttagcgtttgaagagcgtttaaacgctcttcgccccgcgaaaacggctgtgcgtagtttcgagacggccctga

SEQ ID NO: 234 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctaccttctttgaaccattgtgttatgtttgnnngcaggtgcaatgccccattgtaatgaccatccccattgcacctgcnntatgcacgtcaccaagccattgtaccgtttgaagagcgtttaaacgctcttcgcccggcgctcgataatactgtttgtttcgagacggccctga

SEQ ID NO: 235 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctaccttcacccataggagccacagcgtttgnnngcaggtgcaatggagaaacgttctgtccccaagcattgcacctgcnntatgcatcatcctgcatgctagaccagtttgaagagcgtttaaacgctcttcgccccgcgaaaagcgtccgatgatgtttcgagacggccctga

SEQ ID NO: 236 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctcagtgcaacttactattacggtttgnnngcaggtgcaatgcagacgaaagacccactgaagcattgcacctgcnntatgcatgcaagccttacctgtagcggtttgaagagcgtttaaacgctcttcgcccgcggtcgatgcgcaaacgacgtttcgagacggccctga

SEQ ID NO: 237 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctaccttgggactcctcggttatttagtttgnnngcaggtgcaatgctattcccctcgagtttccaccattgcacctgcnntatgcacaacttcttcgtggtatctcgtttgaagagcgtttaaacgctcttcgcccgtcctaagccggtattcgatgtttcgagacggccctga

SEQ ID NO: 238 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctacctatcagttctgagcaactcgtgtttgnnngcaggtgcaatggcctaccggagatataagacgcattgcacctgcnntatgcatcatcctgcatgctagaccagtttgaagagcgtttaaacgctcttcgccctcgagacaaattcgtcgcgtgtttcgagacggccctga

SEQ ID NO: 239 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctacctcgtcaccaagccattgtaccgtttgnnngcaggtgcaatgcccgacttcactagttagacgcattgcacctgcnntatgcacagtgcaacttactattacggtttgaagagcgtttaaacgctcttcgcccagcttcgttacggttaaacggtttcgagacggccctga

SEQ ID NO: 240 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctcaaagacctgcggcgagggcgtttgnnngcaggtgcaatggtagagaagcaacggcaaggacattgcacctgcnntatgcactaaagggcgatctcaacaagtttgaagagcgtttaaacgctcttcgccctgtcgcggcgagtatcaagagtttcgagacggccctga

SEQ ID NO: 241 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctctggaaggtgaggcctaggcgtttgnnngcaggtgcaatggactcaggacaactattccggcattgcacctgcnntatgcatgtgatggtgaatattcacagtttgaagagcgtttaaacgctcttcgccctttcgtcggcaatcgtgcgcgtttcgagacggccctga

SEQ ID NO: 242 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctgtatcctggtgggatttacagtttgnnngcaggtgcaatggcgattcaacgatcagatgcccattgcacctgcnntatgcagttacactctatctacacctgtttgaagagcgtttaaacgctcttcgcccaacacacgtagcgttacgcggtttcgagacggccctga

SEQ ID NO: 243 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctgctcaaaccattacagaagggtttgnnngcaggtgcaatgctgaaatctgcgaccaccgtacattgcacctgcnntatgcaggagagccaacccctaccaggtttgaagagcgtttaaacgctcttcgcccgcgccgaaaaacgtcggttggtttcgagacggccctga

SEQ ID NO: 244 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctgctcaaaccattacagaagggtttgnnngcaggtgcaatgccccgacatataatcttcggccattgcacctgcnntatgcatgtgatggtgaatattcacagtttgaagagcgtttaaacgctcttcgccctccgcaacgcttaacgtgtagtttcgagacggccctga

SEQ ID NO: 245 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctggactgagagctgttgacacgtttgnnngcaggtgcaatgtccgtttccacgcctaaacaccattgcacctgcnntatgcatctgggcatgtgggtccggcgtttgaagagcgtttaaacgctcttcgcccgcgcgcaataacctacccgggtttcgagacggccctga

SEQ ID NO: 246 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctctaaagggcgatctcaacaagtttgnnngcaggtgcaatgcgcaaatgcacgcttcgagatcattgcacctgcnntatgcacaatacagggatacccactagtttgaagagcgtttaaacgctcttcgccccgtatcgtatctgacgtgtggtttcgagacggccctga

SEQ ID NO: 247 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctaatcccttgccccagcacatgtttgnnngcaggtgcaatggcatcaatgcatagcacggcacattgcacctgcnntatgcactggaaggtgaggcctaggcgtttgaagagcgtttaaacgctcttcgcccaaacggtcggcccggcagtcgtttcgagacggccctga

SEQ ID NO: 248 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctaccttgtgatggtgaatattcacagtttgnnngcaggtgcaatgcggcagctagcataacaaagccattgcacctgcnntatgcactccacaaagacctgcggcggtttgaagagcgtttaaacgctcttcgccctgtcgtatgccgcttaacgcgtttcgagacggccctga

SEQ ID NO: 249 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctcgtcaccaagccattgtaccgtttgnnngcaggtgcaatgaacacacctttaacgtgcccccattgcacctgcnntatgcatgcaagccttacctgtagcggtttgaagagcgtttaaacgctcttcgcccggaacgtgtcgcgcattctagtttcgagacggccctga

SEQ ID NO: 250 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctagaagttggtgacgttctgcgtttgnnngcaggtgcaatgaccgtttaacagcccgccatccattgcacctgcnntatgcactgctagccaacaccactgagtttgaagagcgtttaaacgctcttcgccccgcacatccggcgtaggtatgtttcgagacggccctga

SEQ ID NO: 251 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctaccttgggactcctcggttatttagtttgnnngcaggtgcaatgaccacgacggaatctctcagacattgcacctgcnntatgcaacgtagatgccgttcggatagtttgaagagcgtttaaacgctcttcgccccgtcgtgctcgcacctgtaagtttcgagacggccctga

SEQ ID NO: 252 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctctggaaggtgaggcctaggcgtttgnnngcaggtgcaatgccgtagaaacatatcgcgcgtcattgcacctgcnntatgcactataccgatacggagtgacgtttgaagagcgtttaaacgctcttcgccccgatttgatattaccgcgatgtttcgagacggccctga

SEQ ID NO: 253 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctggaagggacactaacctggtgtttgnnngcaggtgcaatgtgatgtagcattagacgcccccattgcacctgcnntatgcagtaactcgtatacgcgcaaggtttgaagagcgtttaaacgctcttcgcccttaacatcgcacgtcgacgggtttcgagacggccctga

SEQ ID NO: 254 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctagctgtagtagagtccccatgtttgnnngcaggtgcaatgtccattaggcaaaacgaggcccattgcacctgcnntatgcagttgtgtcgcatcgacgagtgtttgaagagcgtttaaacgctcttcgcccacgccgagagttgttatgcggtttcgagacggccctga

SEQ ID NO: 255 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctgattccttccaataacacaagtttgnnngcaggtgcaatgccgacacccgtacattcacttcattgcacctgcnntatgcacggaacagacacgtccgaacgtttgaagagcgtttaaacgctcttcgcccataatgggcggtcgtaccgtgtttcgagacggccctga

SEQ ID NO: 256 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctcccaccaggatacgtatgatgtttgnnngcaggtgcaatgccctttcaatagcgtacccctcattgcacctgcnntatgcacgccgcacggaattcgcaaggtttgaagagcgtttaaacgctcttcgccccctcatgaccgctcgccgatgtttcgagacggccctga

SEQ ID NO: 257 - DNA, 236 bp, artificial
  oligo
  misc_feature (93)..(95): n is a, c, g, or t
  misc_feature (141)..(142): n is a, c, g, or t
gaccgcgtctcacaccgtgtgatggtgaatattcacagtttggagaccgtttaaacggtctctaccttgttgggcagctgtatgatcgtttgnnngcaggtgcaatggatatttgacccatcggcacccattgcacctgcnntatgcagcaatctacgacatcccggcgtttgaagagcgtttaaacgctcttcgccccttacgcacggatacggcgagtttcgagacggccctga

SEQ ID NO: 258 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctaccttgttgagatcgccctttagcgtttgnnngcaggtgcaatgcccagacaaagcctttggacccattgcacctgcnntatgcaagtcctacgacgcgccgcaagtttgaagagcgtttaaacgctcttcgccctagtggatccgtacatacgcgtttcgagacggccctga

SEQ ID NO: 259 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctcagcttgtccaactggtcgggtttgnnngcaggtgcaatgtactcgactaactcacggcgccattgcacctgcnntatgcagtgctataactctcgcgcgggtttgaagagcgtttaaacgctcttcgcccccggcgcttctagaccgaatgtttcgagacggccctga

SEQ ID NO: 260 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctacaggtgacccaaccttcaggtttgnnngcaggtgcaatgtgcagcatgccacatagtagccattgcacctgcnntatgcatatccggcccggaaggtcgtgtttgaagagcgtttaaacgctcttcgcccctcgtcgacttacacgctaggtttcgagacggccctga

SEQ ID NO: 261 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctggccatttatagtgttgaccgtttgnnngcaggtgcaatgcaccctcggatacatatcgcgcattgcacctgcnntatgcacgctcgtggctacgacgattgtttgaagagcgtttaaacgctcttcgcccgatctaacgcaacctacgttgtttcgagacggccctga

SEQ ID NO: 262 - DNA, 236 bp, artificial
  oligo
  misc_feature (93)..(95): n is a, c, g, or t
  misc_feature (141)..(142): n is a, c, g, or t
gaccgcgtctcacaccgcctatcatacgtatcctggtgtttggagaccgtttaaacggtctctacctcatcggcctgacccccatgcgtttgnnngcaggtgcaatgtatccaggaatcaagccagcgcattgcacctgcnntatgcatcgtccgactcaacacgatggtttgaagagcgtttaaacgctcttcgccctacagtcgccggtcgtattagtttcgagacggccctga

SEQ ID NO: 263 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctacatagacccctgttgtaaggtttgnnngcaggtgcaatgtcgaaccctgcccaccaattacattgcacctgcnntatgcaacatcttaggttatgcggcggtttgaagagcgtttaaacgctcttcgcccaaaccgatcgcgagtgcgcggtttcgagacggccctga

SEQ ID NO: 264 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctcatcggcctgacccccatgcgtttgnnngcaggtgcaatgcgttccgtacacaatccactgcattgcacctgcnntatgcactttaccgcgcgacggacaagtttgaagagcgtttaaacgctcttcgcccgcgtcgttttgcgcgattaggtttcgagacggccctga

SEQ ID NO: 265 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctctgaaattagacatcaaagcgtttgnnngcaggtgcaatgaaaatcgtcgcacgcccttctcattgcacctgcnntatgcacggcatcacaacttcgtgcggtttgaagagcgtttaaacgctcttcgccccgcatgggctcggttgcgacgtttcgagacggccctga

SEQ ID NO: 266 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctacggaccttccgaacccgacgtttgnnngcaggtgcaatgcggacaacaccttgccttcttcattgcacctgcnntatgcattacgcgtacctacgcctatgtttgaagagcgtttaaacgctcttcgcccccgacgatctaaaccgaccggtttcgagacggccctga

SEQ ID NO: 267 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctaccttaagcgacgccgatcataccgtttgnnngcaggtgcaatgccacacgagaaccacaatagccattgcacctgcnntatgcaacgagtcgtcatcgtgccgtgtttgaagagcgtttaaacgctcttcgcccgcccatagcgacggtgatcggtttcgagacggccctga

SEQ ID NO: 268 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctatcgaccggttgatcctcgagtttgnnngcaggtgcaatgaatctagcacgtgcccaacgtcattgcacctgcnntatgcatagtcgaccgcggacccgttgtttgaagagcgtttaaacgctcttcgccccagctcggtacaatcgcggcgtttcgagacggccctga

SEQ ID NO: 269 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctgttacgaccttttacccgcggtttgnnngcaggtgcaatgggaccacacaagtttaagcggcattgcacctgcnntatgcatgagtcgcacttgcggcgacgtttgaagagcgtttaaacgctcttcgccccggtaggatgtttaaccgtggtttcgagacggccctga

SEQ ID NO: 270 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctatcacggcgtaccttcatacgtttgnnngcaggtgcaatggcgaccgaatcagattgccagcattgcacctgcnntatgcacgcatttgtcgacggatgatgtttgaagagcgtttaaacgctcttcgccctttggccgcgcgctcgatacgtttcgagacggccctga

SEQ ID NO: 271 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctcgttagtgtgtcgcgcataagtttgnnngcaggtgcaatggcaacgtttgacaacgacacgcattgcacctgcnntatgcaggtcgctcgatgcacgacatgtttgaagagcgtttaaacgctcttcgcccacgaatatcacgacgtaatagtttcgagacggccctga

SEQ ID NO: 272 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctgtcacacctgagtaccactggtttgnnngcaggtgcaatgcagtagccccaattcatcgtgcattgcacctgcnntatgcagtgcatacgtacttcgtccagtttgaagagcgtttaaacgctcttcgccctcacacttggagttacagcagtttcgagacggccctga

SEQ ID NO: 273 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctcagcttcaggaccccgatccgtttgnnngcaggtgcaatggcaaacctagactgaatcgcccattgcacctgcnntatgcagcaggtccttaccttgttccgtttgaagagcgtttaaacgctcttcgcccaaccccaacatcagcaagcggtttcgagacggccctga

SEQ ID NO: 274 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctgagaagcactatgggggcatgtttgnnngcaggtgcaatggcgaccatcaagatggctgaacattgcacctgcnntatgcatacgctgcaaagtctctggcgtttgaagagcgtttaaacgctcttcgccctaaccatagtacccagttgtgtttcgagacggccctga

SEQ ID NO: 275 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctgtgtctgtatgacccacccggtttgnnngcaggtgcaatggcaacatcaccacaagttccgcattgcacctgcnntatgcatcctcagctgccatatgtgcgtttgaagagcgtttaaacgctcttcgcccatccccgtcgcagttgaactgtttcgagacggccctga

SEQ ID NO: 276 - DNA, 236 bp, artificial
  oligo
  misc_feature (93)..(95): n is a, c, g, or t
  misc_feature (141)..(142): n is a, c, g, or t
gaccgcgtctcacaccgggggcatgggccatgctgtagtttggagaccgtttaaacggtctctaccttacgctgcaaagtctctggcgtttgnnngcaggtgcaatgacgagaacgtacaccaccaagcattgcacctgcnntatgcatgctgtcgtgagtgccagccgtttgaagagcgtttaaacgctcttcgcccgcaggtccttaccttgttccgtttcgagacggccctga

SEQ ID NO: 277 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctaccttaagctgctctctagacggagtttgnnngcaggtgcaatgcggaccaacgttacccctaatcattgcacctgcnntatgcatcctgccgaggttgggtgccgtttgaagagcgtttaaacgctcttcgcccgaatctcgcgtcacgtaatcgtttcgagacggccctga

SEQ ID NO: 278 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctgcaggtccttaccttgttccgtttgnnngcaggtgcaatggcttcatttccacagtagccccattgcacctgcnntatgcagcagtgaccgtcacgacctggtttgaagagcgtttaaacgctcttcgcccttgctagttacccgtcgcgagtttcgagacggccctga

SEQ ID NO: 279 - DNA, 236 bp, artificial
  oligo
  misc_feature (93)..(95): n is a, c, g, or t
  misc_feature (141)..(142): n is a, c, g, or t
gaccgcgtctcacaccgacctgtggtggttatggggcgtttggagaccgtttaaacggtctctacctaaagcagtcgcccctgcttggtttgnnngcaggtgcaatgcccttactagcaacgtatccccattgcacctgcnntatgcatgtgcttaagaccgggaccagtttgaagagcgtttaaacgctcttcgccctcgtctgaagcgcgctgtacgtttcgagacggccctga

SEQ ID NO: 280 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctacctagccacgcttgctgatgttggtttgnnngcaggtgcaatgtgtccttaacccacctgactccattgcacctgcnntatgcattattcttgtccaggcaagggtttgaagagcgtttaaacgctcttcgcccgttagcgtccgaaaacccgggtttcgagacggccctga

SEQ ID NO: 281 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctagccacgcttgctgatgttggtttgnnngcaggtgcaatgtgcgataactcataccagccgcattgcacctgcnntatgcaaacatgactccccggaggccgtttgaagagcgtttaaacgctcttcgcccccgcaacgttcacgatccacgtttcgagacggccctga

SEQ ID NO: 282 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctatcttgcagatgctaatagcgtttgnnngcaggtgcaatgtctgtacagcgctcaacacgccattgcacctgcnntatgcatacgctgcaaagtctctggcgtttgaagagcgtttaaacgctcttcgcccaacaagcggctatgtcggacgtttcgagacggccctga

SEQ ID NO: 283 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctcagcttcaggaccccgatccgtttgnnngcaggtgcaatggagagcctaacaccacaaagccattgcacctgcnntatgcagcagtgaccgtcacgacctggtttgaagagcgtttaaacgctcttcgcccaacttgcgtgcgacgaacacgtttcgagacggccctga

SEQ ID NO: 284 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctttcctacaaccttctcgcaagtttgnnngcaggtgcaatgcgaccttcgaccgcaacatttcattgcacctgcnntatgcacagcttcaggaccccgatccgtttgaagagcgtttaaacgctcttcgcccagactactagccccgtccgcgtttcgagacggccctga

SEQ ID NO: 285 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctacctcaagggcgtgaacagcgacggtttgnnngcaggtgcaatgtgagcaataaagcgccccgaacattgcacctgcnntatgcattattcttgtccaggcaagggtttgaagagcgtttaaacgctcttcgccccgttcgagccgcgtatccgtgtttcgagacggccctga

SEQ ID NO: 286 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctgagaagcactatgggggcatgtttgnnngcaggtgcaatggtatccggacatcctagcgatcattgcacctgcnntatgcagagccgctgtgatcataccagtttgaagagcgtttaaacgctcttcgcccgtactggtgcgtccgtatctgtttcgagacggccctga

SEQ ID NO: 287 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctccaagtaccttctccagcatgtttgnnngcaggtgcaatgtacacctggaatcaagacccccattgcacctgcnntatgcacgacgttcgacaatgggatcgtttgaagagcgtttaaacgctcttcgccccgctcgatcagactaacgtagtttcgagacggccctga

SEQ ID NO: 288 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctagccacgcttgctgatgttggtttgnnngcaggtgcaatgcgcagaccacggtaatctagacattgcacctgcnntatgcagatccactcgcggaacttacgtttgaagagcgtttaaacgctcttcgccctcgacagactatctgccgtagtttcgagacggccctga

SEQ ID NO: 289 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctgccatgggcaccttctccgcgtttgnnngcaggtgcaatggccactccgatattgtccctccattgcacctgcnntatgcatgtgtcgcgagggcgattaagtttgaagagcgtttaaacgctcttcgccccttctaacgtcgtgacgagagtttcgagacggccctga

SEQ ID NO: 290 - DNA, 236 bp, artificial
  oligo
  misc_feature (93)..(95): n is a, c, g, or t
  misc_feature (141)..(142): n is a, c, g, or t
gaccgcgtctcacaccgcaagggcgtgaacagcgacggtttggagaccgtttaaacggtctctacctacctgtggtggttatggggcgtttgnnngcaggtgcaatgcccatgctagatacggacgaacattgcacctgcnntatgcacgaaattcccgacgctcccggtttgaagagcgtttaaacgctcttcgcccggtcggacctctcggcggatgtttcgagacggccctga

SEQ ID NO: 291 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgctgacatcggctttgctgcgtttgnnngcaggtgcaatgcacggaacgattcgccctgaacattgcacctgcnntatgcacgtcacggcgagcgaaccccgtttgaagagcgtttaaacgctcttcgcccccgtggatcgtttccgcactgtttcgagacggccctga

SEQ ID NO: 292 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctgtgcatacgtacttcgtccagtttgnnngcaggtgcaatgtatgcccattcacaggtccctcattgcacctgcnntatgcattaacgcgtggcatcgagatgtttgaagagcgtttaaacgctcttcgcccacgagccttcgcgccgtttcgtttcgagacggccctga

SEQ ID NO: 293 - DNA, 236 bp, artificial
  oligo
  misc_feature (93)..(95): n is a, c, g, or t
  misc_feature (141)..(142): n is a, c, g, or t
gaccgcgtctcacaccggctgacatcggctttgctgcgtttggagaccgtttaaacggtctctacctctagcaagccataacaaaacgtttgnnngcaggtgcaatgaagaacaacatcaggcgaccgcattgcacctgcnntatgcagtcgcgattcgacgtgataggtttgaagagcgtttaaacgctcttcgcccattcaacgacgaccgtgcgagtttcgagacggccctga

SEQ ID NO: 294 - DNA, 236 bp, artificial
  oligo
  misc_feature (93)..(95): n is a, c, g, or t
  misc_feature (141)..(142): n is a, c, g, or t
gaccgcgtctcacaccggtagaccaggcacccaacctgtttggagaccgtttaaacggtctctacctttattcttgtccaggcaagggtttgnnngcaggtgcaatgcctcaatcctagtcccacggacattgcacctgcnntatgcacgaattatccgagccgcaaggtttgaagagcgtttaaacgctcttcgcccacggcgaacggctagtcgccgtttcgagacggccctga

SEQ ID NO: 295 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctacctaccaggcacccaacctcggcgtttgnnngcaggtgcaatgcgatcagcatttatccggcaccattgcacctgcnntatgcacggcacgaatttacggcgcggtttgaagagcgtttaaacgctcttcgcccggcgtatccccgtacgctacgtttcgagacggccctga

SEQ ID NO: 296 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctacctgagaagcactatgggggcatgtttgnnngcaggtgcaatggcttaatcccaaggcatgtcccattgcacctgcnntatgcaccccgtcgcggtatctcgcagtttgaagagcgtttaaacgctcttcgccctcgctcgtcacgggatagtcgtttcgagacggccctga

SEQ ID NO: 297 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctaccttactcgttgcggcgataccggtttgnnngcaggtgcaatggacacacgcctataacacgagcattgcacctgcnntatgcatcacgagctatacaacgcgtgtttgaagagcgtttaaacgctcttcgcccccgcctacgccatgccgattgtttcgagacggccctga

SEQ ID NO: 298 - DNA, 236 bp, artificial
  oligo
  misc_feature (93)..(95): n is a, c, g, or t
  misc_feature (141)..(142): n is a, c, g, or t
gaccgcgtctcacaccggctgacatcggctttgctgcgtttggagaccgtttaaacggtctctacctatcgcggggtcgaaccacgagtttgnnngcaggtgcaatgtgccaacggacgaatccactccattgcacctgcnntatgcaccgacgtgtcggtacgcgccgtttgaagagcgtttaaacgctcttcgcccatcgcgtcatcgcgaaacgtgtttcgagacggccctga

SEQ ID NO: 299 - DNA, 236 bp, artificial
  oligo
  misc_feature (93)..(95): n is a, c, g, or t
  misc_feature (141)..(142): n is a, c, g, or t
gaccgcgtctcacaccgcagcttcaggaccccgatccgtttggagaccgtttaaacggtctctacctgttcgaacgcagaatgtacggtttgnnngcaggtgcaatgatctcagcatcagcacgcagtcattgcacctgcnntatgcaaccgttcgacgagtttagtcgtttgaagagcgtttaaacgctcttcgcccgtagccggcccgacgcaaacgtttcgagacggccctga

SEQ ID NO: 300 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctatcgcgcgaacttctgagtagtttgnnngcaggtgcaatgatacttggttcgacccaacggcattgcacctgcnntatgcagatacggcggtaacgtatcggtttgaagagcgtttaaacgctcttcgcccctcaatgaacgacctaccgggtttcgagacggccctga

SEQ ID NO: 301 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctacctccgcggacttaagcgcgcaagtttgnnngcaggtgcaatgctcggataagtaaaccgcacccattgcacctgcnntatgcaacgagcgatagcctaggacggtttgaagagcgtttaaacgctcttcgcccccgtcatcgatcggtagctagtttcgagacggccctga

SEQ ID NO: 302 - DNA, 236 bp, artificial
  oligo
  misc_feature (93)..(95): n is a, c, g, or t
  misc_feature (141)..(142): n is a, c, g, or t
gaccgcgtctcacaccgcttctatggcagcatctcgcgtttggagaccgtttaaacggtctctacctcattgtatacgccttcctgggtttgnnngcaggtgcaatggtacaggagaaaccagcaccacattgcacctgcnntatgcatcgggggacggttatttctggtttgaagagcgtttaaacgctcttcgcccagggcacgtcaactctacacgtttcgagacggccctga

SEQ ID NO: 303 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctacctgcgccagtgtttgcgctcccgtttgnnngcaggtgcaatgtcttcctcactgtcctagtcgcattgcacctgcnntatgcatgataagcacctggtgctccgtttgaagagcgtttaaacgctcttcgcccgtaaagtcggtccgaatcgcgtttcgagacggccctga

SEQ ID NO: 304 - DNA, 236 bp, artificial
  oligo
  misc_feature (93)..(95): n is a, c, g, or t
  misc_feature (141)..(142): n is a, c, g, or t
gaccgcgtctcacaccgaagaggcttcttatgtcggcgtttggagaccgtttaaacggtctctacctcaacggcacgtacgccatcggtttgnnngcaggtgcaatgcgatcacgacgtacagttcagcattgcacctgcnntatgcagccttgagcccggtggggctgtttgaagagcgtttaaacgctcttcgcccgtcctaccacggatatcggagtttcgagacggccctga

SEQ ID NO: 305 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctacctactgtcaggcagcaccactcgtttgnnngcaggtgcaatgcagcgttcaacctccttcctacattgcacctgcnntatgcaagggcacgtcaactctacacgtttgaagagcgtttaaacgctcttcgcccggttatccgtaagactccgggtttcgagacggccctga

SEQ ID NO: 306 - DNA, 236 bp, artificial
  oligo
  misc_feature (93)..(95): n is a, c, g, or t
  misc_feature (141)..(142): n is a, c, g, or t
gaccgcgtctcacaccgaacggcacgtacgccatcgcgtttggagaccgtttaaacggtctctacctgggccagggtctgcatatgcgtttgnnngcaggtgcaatgccacatatgcggttacacagccattgcacctgcnntatgcagtgacccgaggaaccggtgcgtttgaagagcgtttaaacgctcttcgccccagcaatacgatccgcgcgtgtttcgagacggccctga

SEQ ID NO: 307 - DNA, 236 bp, artificial
  oligo
  misc_feature (93)..(95): n is a, c, g, or t
  misc_feature (141)..(142): n is a, c, g, or t
gaccgcgtctcacaccgttgggggatactcacgggatgtttggagaccgtttaaacggtctctacctgcgccagtgtttgcgctcccgtttgnnngcaggtgcaatgccctcagtacaagacgaactccattgcacctgcnntatgcaccccgttaccggatcgtcgtgtttgaagagcgtttaaacgctcttcgcccccgcgcgtaaggtttcgtgcgtttcgagacggccctga

SEQ ID NO: 308 - DNA, 236 bp, artificial
  oligo
  misc_feature (93)..(95): n is a, c, g, or t
  misc_feature (141)..(142): n is a, c, g, or t
gaccgcgtctcacaccgtatccggttacctcatagtcgtttggagaccgtttaaacggtctctacctactcacggcagcgcacgcacgtttgnnngcaggtgcaatgtccatgcacccaaccttcctccattgcacctgcnntatgcaaaaggtccgagtatcgcgccgtttgaagagcgtttaaacgctcttcgccctgcggtcgcgggttctaatagtttcgagacggccctga

SEQ ID NO: 309 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctacctatcctccacgtgcggttcccgtttgnnngcaggtgcaatgccccaaaccattagtgttccgcattgcacctgcnntatgcaacgtccgtcggtatggacaagtttgaagagcgtttaaacgctcttcgcccggcgttagttggttacgcgcgtttcgagacggccctga

SEQ ID NO: 310 - DNA, 236 bp, artificial
  oligo
  misc_feature (93)..(95): n is a, c, g, or t
  misc_feature (141)..(142): n is a, c, g, or t
gaccgcgtctcacaccgcttctatggcagcatctcgcgtttggagaccgtttaaacggtctctacctggccagaatacaggtcccgcgtttgnnngcaggtgcaatgttcagtccgacaccgaaagaccattgcacctgcnntatgcacgaagttgccgaatgcgaatgtttgaagagcgtttaaacgctcttcgccccgatagcgagttcgtgcgacgtttcgagacggccctga

SEQ ID NO: 311 - DNA, 236 bp, artificial
  oligo
  misc_feature (93)..(95): n is a, c, g, or t
  misc_feature (141)..(142): n is a, c, g, or t
gaccgcgtctcacaccgttacattgtatacgccttccgtttggagaccgtttaaacggtctctacctgcgccagtgtttgcgctcccgtttgnnngcaggtgcaatgtcttcacagaacctcacggcacattgcacctgcnntatgcaatcgcgagcggtctacgtccgtttgaagagcgtttaaacgctcttcgcccccggtcgtcgcgttatcgtcgtttcgagacggccctga

SEQ ID NO: 312 - DNA, 236 bp, artificial
  oligo
  misc_feature (93)..(95): n is a, c, g, or t
  misc_feature (141)..(142): n is a, c, g, or t
gaccgcgtctcacaccgccgagttacctcgagctttcgtttggagaccgtttaaacggtctctacctgctcgggatccagagatggggtttgnnngcaggtgcaatgcaaacatgccccgtaccctaacattgcacctgcnntatgcacccgcgacaaatctgccgtagtttgaagagcgtttaaacgctcttcgccccgatacggtgcaccgattcagtttcgagacggccctga

SEQ ID NO: 313 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctccattaattggcgatgcgcggtttgnnngcaggtgcaatgcacacgatcatcaaccagccccattgcacctgcnntatgcactcgttacgccgctactaaggtttgaagagcgtttaaacgctcttcgccccggtacacgacttcgcccgcgtttcgagacggccctga

SEQ ID NO: 314 - DNA, 236 bp, artificial
  oligo
  misc_feature (93)..(95): n is a, c, g, or t
  misc_feature (141)..(142): n is a, c, g, or t
gaccgcgtctcacaccggaagcgagagaatctcctcggtttggagaccgtttaaacggtctctacctccggatcgtgatcctcggttgtttgnnngcaggtgcaatgctcgcctaacattgacctcggcattgcacctgcnntatgcaaccggttacgcgacttctatgtttgaagagcgtttaaacgctcttcgcccttcgtacggttggaccggttgtttcgagacggccctga

SEQ ID NO: 315 - DNA, 236 bp, artificial
  oligo
  misc_feature (93)..(95): n is a, c, g, or t
  misc_feature (141)..(142): n is a, c, g, or t
gaccgcgtctcacaccgagggcacgtcaactctacacgtttggagaccgtttaaacggtctctaccttaggtgtcggtccggattaggtttgnnngcaggtgcaatggcctctatcaaaccaacgccccattgcacctgcnntatgcatatcgattcgccccgcctgtgtttgaagagcgtttaaacgctcttcgcccgcgcacgctaccacttaggtgtttcgagacggccctga

SEQ ID NO: 316 - DNA, 236 bp, artificial
  oligo
  misc_feature (93)..(95): n is a, c, g, or t
  misc_feature (141)..(142): n is a, c, g, or t
gaccgcgtctcacaccgaagaggcttcttatgtcggcgtttggagaccgtttaaacggtctctacctgttcattgggtcggcgtcgtgtttgnnngcaggtgcaatgctccttaccagcctcatttgccattgcacctgcnntatgcacgcatacgacggaccgccgagtttgaagagcgtttaaacgctcttcgccctagccgtcgacggctattgggtttcgagacggccctga

SEQ ID NO: 317 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctactttccatgtcagccatacgtttgnnngcaggtgcaatgctcccaaaacgcgctgaaatccattgcacctgcnntatgcatgggactcctcggttatttagtttgaagagcgtttaaacgctcttcgccctcacccataggagccacagcgtttcgagacggccctga

SEQ ID NO: 318 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctttcatagacctaatgtgactgtttgnnngcaggtgcaatgcccatttcaaagcatgcgcgacattgcacctgcnntatgcatgaatgaccagggtacctgcgtttgaagagcgtttaaacgctcttcgcccgtatcctggtgggatttacagtttcgagacggccctga

SEQ ID NO: 319 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctaccttgttgggcagctgtatgatcgtttgnnngcaggtgcaatgcttgtccccaatcactggcgacattgcacctgcnntatgcacaggcacgatagatacaaaggtttgaagagcgtttaaacgctcttcgcccgattccttccaataacacaagtttcgagacggccctga

SEQ ID NO: 320 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctctgcagttgagctggcaatcgtttgnnngcaggtgcaatgcccctcgactctatctaagtgcattgcacctgcnntatgcatgggactcctcggttatttagtttgaagagcgtttaaacgctcttcgccctgttgagatcgccctttagcgtttcgagacggccctga

SEQ ID NO: 321 - DNA, 236 bp, artificial
  oligo
  misc_feature (93)..(95): n is a, c, g, or t
  misc_feature (141)..(142): n is a, c, g, or t
gaccgcgtctcacaccggctcaaaccattacagaagggtttggagaccgtttaaacggtctctacctcaacttcttcgtggtatctcgtttgnnngcaggtgcaatgacagcaagctacgtcaccctacattgcacctgcnntatgcaattcttctctgtgaatgtcggtttgaagagcgtttaaacgctcttcgcccggaagggacactaacctggtgtttcgagacggccctga

SEQ ID NO: 322 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctaccttgaatgaccagggtacctgcgtttgnnngcaggtgcaatggtcctctctgaagattgccgtcattgcacctgcnntatgcagccctttagctggtatctgcgtttgaagagcgtttaaacgctcttcgcccgattccttccaataacacaagtttcgagacggccctga

SEQ ID NO: 323 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttgtgatggtgaatattcacagtttgnnngcaggtgcaatgttccccgatacgtactcgtaccattgcacctgcnntatgcatcacatggatggttactgaagtttgaagagcgtttaaacgctcttcgccccctatcatacgtatcctggtgtttcgagacggccctga

SEQ ID NO: 324 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctacatagacccctgttgtaaggtttgnnngcaggtgcaatgctacccgtcgcctaactaggacattgcacctgcnntatgcacatcggcctgacccccatgcgtttgaagagcgtttaaacgctcttcgcccttgggactcctcggttatttgtttcgagacggccctga

SEQ ID NO: 325 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctggagagccaacccctaccaggtttgnnngcaggtgcaatgtcccagactgtttcaccatgccattgcacctgcnntatgcagtatcctggtgggatttacagtttgaagagcgtttaaacgctcttcgccccagtgcaacttactattacggtttcgagacggccctga

SEQ ID NO: 326 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctttcatagacctaatgtgactgtttgnnngcaggtgcaatgcgcaacaactataggccccatcattgcacctgcnntatgcaagcacacaagcacgttacccgtttgaagagcgtttaaacgctcttcgcccacacaacactgatgaggtccgtttcgagacggccctga

SEQ ID NO: 327 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctactttccatgtcagccatacgtttgnnngcaggtgcaatggagagaaactaatcgcacgcgcattgcacctgcnntatgcagaggcggtcaggatggctgcgtttgaagagcgtttaaacgctcttcgcccggccatttatagtgttgaccgtttcgagacggccctga

SEQ ID NO: 328 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctagaagttggtgacgttctgcgtttgnnngcaggtgcaatgttacgattccgcatcacgctccattgcacctgcnntatgcacaggcacgatagatacaaaggtttgaagagcgtttaaacgctcttcgcccgctcaaaccattacagaagggtttcgagacggccctga

SEQ ID NO: 329 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctggccatttatagtgttgaccgtttgnnngcaggtgcaatgagcgaaacagcgtaaagcgtccattgcacctgcnntatgcaaatcccttgccccagcacatgtttgaagagcgtttaaacgctcttcgcccagcacacaagcacgttacccgtttcgagacggccctga

SEQ ID NO: 330 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctccctcaccttctacccagccgtttgnnngcaggtgcaatgaacaaccgagtatatcccgcccattgcacctgcnntatgcacaaagacctgcggcgagggcgtttgaagagcgtttaaacgctcttcgcccctgaaattagacatcaaagcgtttcgagacggccctga

SEQ ID NO: 331 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctacaggtgacccaaccttcaggtttgnnngcaggtgcaatgagaagccacttccaacgttcgcattgcacctgcnntatgcacaaagacctgcggcgagggcgtttgaagagcgtttaaacgctcttcgccctgttgagatcgccctttagcgtttcgagacggccctga

SEQ ID NO: 332 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctcctatcatacgtatcctggtgtttgnnngcaggtgcaatgggcggcaaaaaaccctattcgcattgcacctgcnntatgcacaatacagggatacccactagtttgaagagcgtttaaacgctcttcgccccggctacactcgcggtcatcgtttcgagacggccctga

SEQ ID NO: 333 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctactttccatgtcagccatacgtttgnnngcaggtgcaatgtgctaactcagtgacctcacgcattgcacctgcnntatgcactgcagttgagctggcaatcgtttgaagagcgtttaaacgctcttcgcccgacttatatagcgcccgctagtttcgagacggccctga

SEQ ID NO: 334 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctgttacactctatctacacctgtttgnnngcaggtgcaatgcagcataccaccttatagccccattgcacctgcnntatgcatctttgaaccattgtgttatgtttgaagagcgtttaaacgctcttcgcccactatcacgtttagccccgcgtttcgagacggccctga

SEQ ID NO: 335 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctaccttgtcgatggcaatagccaaggtttgnnngcaggtgcaatgatgctctaccgacaggaccgtcattgcacctgcnntatgcactgcagttgagctggcaatcgtttgaagagcgtttaaacgctcttcgcccgtcgttatcacgtagcgcacgtttcgagacggccctga

SEQ ID NO: 336 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctactttccatgtcagccatacgtttgnnngcaggtgcaatgtacccatcgatatccagtccccattgcacctgcnntatgcatcacatggatggttactgaagtttgaagagcgtttaaacgctcttcgccctgaatggcggtcgatagctcgtttcgagacggccctga

SEQ ID NO: 337 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctacctatcatacgtatcctgggtttgnnngcaggtgcaatgcctccgactgtatccggtctacattgcacctgcnntatgcatctttgaaccattgtgttatgtttgaagagcgtttaaacgctcttcgccccgtcccacgcgtaacgcggtgtttcgagacggccctga

SEQ ID NO: 338 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctctggaaggtgaggcctaggcgtttgnnngcaggtgcaatgcgctaaggcaacccatatcggcattgcacctgcnntatgcacaaagacctgcggcgagggcgtttgaagagcgtttaaacgctcttcgcccatatacgcacgctagctacggtttcgagacggccctga

SEQ ID NO: 339 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctgattccttccaataacacaagtttgnnngcaggtgcaatggactattggcaacccacggaacattgcacctgcnntatgcagttacactctatctacacctgtttgaagagcgtttaaacgctcttcgccccacccggcgcgactcactaagtttcgagacggccctga

SEQ ID NO: 340 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctacctttgggactcctcggttatttgtttgnnngcaggtgcaatgcaaaactcgaccaccgtcaagcattgcacctgcnntatgcatgtcgatggcaatagccaaggtttgaagagcgtttaaacgctcttcgcccggccggacgaattacgataggtttcgagacggccctga

SEQ ID NO: 341 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctggaagggacactaacctggtgtttgnnngcaggtgcaatgtgagaaccacgagttcaagggcattgcacctgcnntatgcaagaagttggtgacgttctgcgtttgaagagcgtttaaacgctcttcgcccttcgcggcggagaagttagggtttcgagacggccctga

SEQ ID NO: 342 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctgtatcctggtgggatttacagtttgnnngcaggtgcaatgcctgtccttagcccttacatgcattgcacctgcnntatgcaactttccatgtcagccatacgtttgaagagcgtttaaacgctcttcgcccgtccccgcggtaacacgacggtttcgagacggccctga

SEQ ID NO: 343 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctaccttgcaagccttacctgtagcggtttgnnngcaggtgcaatgtgcatgaagtgacagcacgctcattgcacctgcnntatgcatgaatgaccagggtacctgcgtttgaagagcgtttaaacgctcttcgccctaatacgagcggttccggacgtttcgagacggccctga

SEQ ID NO: 344 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctcatcggcctgacccccatgcgtttgnnngcaggtgcaatgttaacggcccatcccctaacacattgcacctgcnntatgcactggaaggtgaggcctaggcgtttgaagagcgtttaaacgctcttcgcccagtcgtcgcgtgtttagtctgtttcgagacggccctga

SEQ ID NO: 345 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctaccttgttgggcagctgtatgatcgtttgnnngcaggtgcaatggaaccgacgaccaagtagaagcattgcacctgcnntatgcaagctgtagtagagtccccatgtttgaagagcgtttaaacgctcttcgccccgtgcgattttaagcgcgccgtttcgagacggccctga

SEQ ID NO: 346 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctaccttgcccacaaagtatggcggtgtttgnnngcaggtgcaatgcacactttccaaacgccggaccattgcacctgcnntatgcatgtcgatggcaatagccaaggtttgaagagcgtttaaacgctcttcgcccacgcgatatcatacaacgccgtttcgagacggccctga

SEQ ID NO: 347 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctacctcatcggcctgacccccatgcgtttgnnngcaggtgcaatgacacagcagctcaccaatcaccattgcacctgcnntatgcaagctgtagtagagtccccatgtttgaagagcgtttaaacgctcttcgccccttaaaaaccgtcattcgcggtttcgagacggccctga

SEQ ID NO: 348 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctcctatcatacgtatcctggtgtttgnnngcaggtgcaatgctcccgactataagacacgcccattgcacctgcnntatgcaacaggtgacccaaccttcaggtttgaagagcgtttaaacgctcttcgccctagtaagccgtccgatgcgcgtttcgagacggccctga

SEQ ID NO: 349 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctacatagacccctgttgtaaggtttgnnngcaggtgcaatgagatgcccgatcagctcaacccattgcacctgcnntatgcatgtcgatggcaatagccaaggtttgaagagcgtttaaacgctcttcgcccgtatcggcccgtagtacgtagtttcgagacggccctga

SEQ ID NO: 350 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctcccaccaggatacgtatgatgtttgnnngcaggtgcaatggaccttattccgattagcccgcattgcacctgcnntatgcaatcagttctgagcaactcgtgtttgaagagcgtttaaacgctcttcgcccatacgttgttagtacgtccggtttcgagacggccctga

SEQ ID NO: 351 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctacctggagagccaacccctaccaggtttgnnngcaggtgcaatgaaatgcgcccttgacacgactcattgcacctgcnntatgcagttacactctatctacacctgtttgaagagcgtttaaacgctcttcgccccttctacggagtcaacgcgcgtttcgagacggccctga

SEQ ID NO: 352 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctcagcttgtccaactggtcgggtttgnnngcaggtgcaatgcgatatgtcccctcttccctacattgcacctgcnntatgcacgcgtccactggattcataggtttgaagagcgtttaaacgctcttcgcccgttaaactaaatcggcgcgcgtttcgagacggccctga

SEQ ID NO: 353 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctaccttcacatggatggttactgaagtttgnnngcaggtgcaatgaatactcctcttccggcttcgcattgcacctgcnntatgcagttcgcgagcgtcctttagcgtttgaagagcgtttaaacgctcttcgccccgtgccggcgggtaacccctgtttcgagacggccctga

SEQ ID NO: 354 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctctgctagccaacaccactgagtttgnnngcaggtgcaatgccttgagcagcgcaccgttatcattgcacctgcnntatgcaccgctgaatccgtattgcgagtttgaagagcgtttaaacgctcttcgccctcgcgtgattagggattcgtgtttcgagacggccctga

SEQ ID NO: 355 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctaccttctttgaaccattgtgttatgtttgnnngcaggtgcaatggaaagccacacagtcatcccgcattgcacctgcnntatgcaagtaatgcacccgtcgatccgtttgaagagcgtttaaacgctcttcgcccccgcggtcacggttggtacggtttcgagacggccctga

SEQ ID NO: 356 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctaccttctttgaaccattgtgttatgtttgnnngcaggtgcaatgcagcctaagcatacgatctcccattgcacctgcnntatgcaagtacgcgcgaacaagtattgtttgaagagcgtttaaacgctcttcgcccgccgcacgaagtatcctacggtttcgagacggccctga

SEQ ID NO: 357 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctctgcagttgagctggcaatcgtttgnnngcaggtgcaatgttctacctccattcactggcgcattgcacctgcnntatgcatatgacgcgcgcccgcctcagtttgaagagcgtttaaacgctcttcgccctatagttgacggtcccgcgtgtttcgagacggccctga

SEQ ID NO: 358 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctcagcttgtccaactggtcgggtttgnnngcaggtgcaatgatcgactcgaaccagctaccacattgcacctgcnntatgcataagcggcaatcgccgtatagtttgaagagcgtttaaacgctcttcgcccgagcgttacggcacgacgccgtttcgagacggccctga

SEQ ID NO: 359 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctagctgtagtagagtccccatgtttgnnngcaggtgcaatgctcactatcacacgggactcacattgcacctgcnntatgcatactcgcgtaaacttcatacgtttgaagagcgtttaaacgctcttcgcccgtgtacgggcgtcatccgacgtttcgagacggccctga

SEQ ID NO: 360 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctcaatacagggatacccactagtttgnnngcaggtgcaatggctaaatgtaccccaacaccccattgcacctgcnntatgcactagacggcgcattagacgtgtttgaagagcgtttaaacgctcttcgccctttcatcgccgacgcgtcaggtttcgagacggccctga

SEQ ID NO: 361 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctggactgagagctgttgacacgtttgnnngcaggtgcaatgtcaatgccaacacacctctcgcattgcacctgcnntatgcatagaacccggatctcgcgaagtttgaagagcgtttaaacgctcttcgcccctttcgtcgcagcggtcatcgtttcgagacggccctga

SEQ ID NO: 362 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctggccatttatagtgttgaccgtttgnnngcaggtgcaatggcgcaccatgaataaagcaggcattgcacctgcnntatgcaacgaacttgatccgttccgtgtttgaagagcgtttaaacgctcttcgcccgacatcccgccgccgttgttgtttcgagacggccctga

SEQ ID NO: 363 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctctggaaggtgaggcctaggcgtttgnnngcaggtgcaatgcacaatccgccgtcaaatagccattgcacctgcnntatgcatcgccgtgccgtttcacgaggtttgaagagcgtttaaacgctcttcgcccgggcgcgcatgacgaccgaagtttcgagacggccctga

SEQ ID NO: 364 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctattcttctctgtgaatgtcggtttgnnngcaggtgcaatgcagaaactgaccaggcatacccattgcacctgcnntatgcattatcgcgtcgtgatcggcagtttgaagagcgtttaaacgctcttcgccctcgtacggtaacttatcgccgtttcgagacggccctga

SEQ ID NO: 365 - DNA, 236 bp, artificial
  oligo
  misc_feature (93)..(95): n is a, c, g, or t
  misc_feature (141)..(142): n is a, c, g, or t
gaccgcgtctcacaccgtgcccacaaagtatggcggtgtttggagaccgtttaaacggtctctacctcatcggcctgacccccatgcgtttgnnngcaggtgcaatgaccctgatctccaagactcgacattgcacctgcnntatgcagtcggtagacgacccggtgggtttgaagagcgtttaaacgctcttcgccctcgcgataggcggtatctgagtttcgagacggccctga

SEQ ID NO: 366 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctctgaaattagacatcaaagcgtttgnnngcaggtgcaatggctctgtaccgagtgctgctacattgcacctgcnntatgcagggcgtagttcgataacccagtttgaagagcgtttaaacgctcttcgccctatgatccctaccaagcgcggtttcgagacggccctga

SEQ ID NO: 367 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctacggggtcgaacctacgcgcgtttgnnngcaggtgcaatgcctcgagatgatcttcaccctcattgcacctgcnntatgcagccgtctagcgaaattgccggtttgaagagcgtttaaacgctcttcgcccgccgacagcgccggaaacgagtttcgagacggccctga

SEQ ID NO: 368 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctgcatcgaacgtcccgacgatgtttgnnngcaggtgcaatgcgcaaccaaaaaatagcccggcattgcacctgcnntatgcaatacgcgaatgcgttgacacgtttgaagagcgtttaaacgctcttcgcccgcactcgcgcgaatttattggtttcgagacggccctga

SEQ ID NO: 369 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctaatacgccgcgtttcgaagagtttgnnngcaggtgcaatgtacttgcccatccaagagctccattgcacctgcnntatgcatcgcggtgtggattgagtcggtttgaagagcgtttaaacgctcttcgcccatgcgaccatagtatcgccggtttcgagacggccctga

SEQ ID NO: 370 - DNA, 236 bp, artificial
  oligo
  misc_feature (93)..(95): n is a, c, g, or t
  misc_feature (141)..(142): n is a, c, g, or t
gaccgcgtctcacaccgacacaacactgatgaggtccgtttggagaccgtttaaacggtctctacctctacgacgccccgtgctatcgtttgnnngcaggtgcaatggaagcatccctcgaaaactcgcattgcacctgcnntatgcacgcgccttttcttacgataggtttgaagagcgtttaaacgctcttcgcccacgaaccggtctaccgcatagtttcgagacggccctga

SEQ ID NO: 371 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctttcttctaccgcgccgataggtttgnnngcaggtgcaatgcaaagtgcttcaacccgtccccattgcacctgcnntatgcatactcacagtacgtcgttcggtttgaagagcgtttaaacgctcttcgcccgcgtaaccgtagacccctcggtttcgagacggccctga

SEQ ID NO: 372 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctacctacctcgatacacgcgcgctagtttgnnngcaggtgcaatgatctctagcacctagcactggcattgcacctgcnntatgcagttcaagtaatggcgccgcggtttgaagagcgtttaaacgctcttcgcccgttcgtcaagtccgccgcaggtttcgagacggccctga

SEQ ID NO: 373 - DNA, 236 bp, artificial
  oligo
  misc_feature (93)..(95): n is a, c, g, or t
  misc_feature (141)..(142): n is a, c, g, or t
gaccgcgtctcacaccggagaagcactatgggggcatgtttggagaccgtttaaacggtctctacctgttcactacgagtgtattccgtttgnnngcaggtgcaatgtcctactcatgagacatcgcgcattgcacctgcnntatgcacagcttcaggaccccgatccgtttgaagagcgtttaaacgctcttcgcccgctgacatcggctttgctgcgtttcgagacggccctga

SEQ ID NO: 374 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctgcaggtccttaccttgttccgtttgnnngcaggtgcaatgctcatgtttcccaccatagcgcattgcacctgcnntatgcagtagaccaggcacccaacctgtttgaagagcgtttaaacgctcttcgccctaaccatagtacccagttgtgtttcgagacggccctga

SEQ ID NO: 375 - DNA, 236 bp, artificial
  oligo
  misc_feature (93)..(95): n is a, c, g, or t
  misc_feature (141)..(142): n is a, c, g, or t
gaccgcgtctcacaccgatcttgcagatgctaatagcgtttggagaccgtttaaacggtctctacctaccaggctgacagttatcacgtttgnnngcaggtgcaatgcttcccaaagacaaggagtcccattgcacctgcnntatgcaggggcatgggccatgctgtagtttgaagagcgtttaaacgctcttcgcccgtggaagtctgtgtcgggaagtttcgagacggccctga

SEQ ID NO: 376 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctgcttgtggtagatagcaacggtttgnnngcaggtgcaatgtccgattcgcaccaagtcaagcattgcacctgcnntatgcagccatgggcaccttctccgcgtttgaagagcgtttaaacgctcttcgcccgtgtctgtatgacccacccggtttcgagacggccctga

SEQ ID NO: 377 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctgtgcatacgtacttcgtccagtttgnnngcaggtgcaatgaccgacggataagaaaccctgcattgcacctgcnntatgcatcctgccgaggttgggtgccgtttgaagagcgtttaaacgctcttcgcccgcagtgaccgtcacgacctggtttcgagacggccctga

SEQ ID NO: 378 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctaccaggctgacagttatcacgtttgnnngcaggtgcaatgtctgattccctacaaccggaccattgcacctgcnntatgcataaccatagtacccagttgtgtttgaagagcgtttaaacgctcttcgcccgacccgaactgcgcgcgatagtttcgagacggccctga

SEQ ID NO: 379 - DNA, 236 bp, artificial
  oligo
  misc_feature (93)..(95): n is a, c, g, or t
  misc_feature (141)..(142): n is a, c, g, or t
gaccgcgtctcacaccgtaaccatagtacccagttgtgtttggagaccgtttaaacggtctctacctttattcttgtccaggcaagggtttgnnngcaggtgcaatgagacattcagcacgagccgatcattgcacctgcnntatgcaccaagtaccttctccagcatgtttgaagagcgtttaaacgctcttcgcccccccgtacgacgcaagtcgtgtttcgagacggccctga

SEQ ID NO: 380 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctgagaggtttccgaagactaagtttgnnngcaggtgcaatgccgtaaagatactagaggccccattgcacctgcnntatgcaatccccgtcgcagttgaactgtttgaagagcgtttaaacgctcttcgccccgcgcagtctaacgaagttcgtttcgagacggccctga

SEQ ID NO: 381 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctaccttcccacagttgtaccttatcgtttgnnngcaggtgcaatgcactgcactagatcaacgaggcattgcacctgcnntatgcaacctgtggtggttatggggcgtttgaagagcgtttaaacgctcttcgccccgcggtcgctagcgaaggaagtttcgagacggccctga

SEQ ID NO: 382 - DNA, 236 bp, artificial
  oligo
  misc_feature (93)..(95): n is a, c, g, or t
  misc_feature (141)..(142): n is a, c, g, or t
gaccgcgtctcacaccggttcactacgagtgtattccgtttggagaccgtttaaacggtctctacctttcttctcagttcaagtaacgtttgnnngcaggtgcaatggccgacatctcacccaaagaacattgcacctgcnntatgcaccacagtactcacttggtccgtttgaagagcgtttaaacgctcttcgcccgcgattaatacgctccgaccgtttcgagacggccctga

SEQ ID NO: 383 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctaagcactacccctgccaaccgtttgnnngcaggtgcaatgtgatcccaactcctacgccatcattgcacctgcnntatgcagttgccaggtgagcccgctcgtttgaagagcgtttaaacgctcttcgccccttaccgtattaggcacgctgtttcgagacggccctga

SEQ ID NO: 384 - DNA, 236 bp, artificial
  oligo
  misc_feature (93)..(95): n is a, c, g, or t
  misc_feature (141)..(142): n is a, c, g, or t
gaccgcgtctcacaccggagccgctgtgatcataccagtttggagaccgtttaaacggtctctaccttcctcagctgccatatgtgcgtttgnnngcaggtgcaatgagtacgagctataccgcacaccattgcacctgcnntatgcatgtgcttaagaccgggaccagtttgaagagcgtttaaacgctcttcgccctgcccgcggttcggtgttgagtttcgagacggccctga

SEQ ID NO: 385 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctctagcaagccataacaaaacgtttgnnngcaggtgcaatggcctttgaaatacctctcccccattgcacctgcnntatgcagagccgctgtgatcataccagtttgaagagcgtttaaacgctcttcgcccagttgcacccgcggttaacggtttcgagacggccctga

SEQ ID NO: 386 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctaccttcctcagctgccatatgtgcgtttgnnngcaggtgcaatgcaaatggtagtcagccacacgcattgcacctgcnntatgcagcaggtccttaccttgttccgtttgaagagcgtttaaacgctcttcgcccatcgcgttgttgcatgacgggtttcgagacggccctga

SEQ ID NO: 387 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctaaagcagtcgcccctgcttggtttgnnngcaggtgcaatgctctgaccgaatcgtacgttccattgcacctgcnntatgcatcacacttggagttacagcagtttgaagagcgtttaaacgctcttcgcccccgtcgttcccttacggatagtttcgagacggccctga

SEQ ID NO: 388 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctaccttggcattggggacctcgggtgtttgnnngcaggtgcaatgatgacgcagtaacagccatcgcattgcacctgcnntatgcaatcccgtggcgatgtatcgagtttgaagagcgtttaaacgctcttcgcccgtccggaaacgtccgctaatgtttcgagacggccctga

SEQ ID NO: 389 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctacctgagccgctgtgatcataccagtttgnnngcaggtgcaatgcagattgttatcaccgccaggcattgcacctgcnntatgcagagtaatgatgcgtcgccgagtttgaagagcgtttaaacgctcttcgccctcgatgcgtaactgcgcaccgtttcgagacggccctga

SEQ ID NO: 390 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctacctgtgtctgtatgacccacccggtttgnnngcaggtgcaatgaggaccccagacattgtttcgcattgcacctgcnntatgcacaccgtataatcggcacgttgtttgaagagcgtttaaacgctcttcgccctaggactgcacccgatcgtagtttcgagacggccctga

SEQ ID NO: 391 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctaaccccaacatcagcaagcggtttgnnngcaggtgcaatgccccggatctcaaccgtatatcattgcacctgcnntatgcatacgtcgcacgactacccatgtttgaagagcgtttaaacgctcttcgccccgcgacatatgaagagtcgagtttcgagacggccctga

SEQ ID NO: 392 - DNA, 236 bp, artificial
  oligo
  misc_feature (93)..(95): n is a, c, g, or t
  misc_feature (141)..(142): n is a, c, g, or t
gaccgcgtctcacaccgtgtgaaaaggactgcaggccgtttggagaccgtttaaacggtctctaccttcccacagttgtaccttatcgtttgnnngcaggtgcaatggcaccctgaattgaacgacaccattgcacctgcnntatgcatatgctacgctcgtcgtgcggtttgaagagcgtttaaacgctcttcgcccaatctaacggataaccgggtgtttcgagacggccctga

SEQ ID NO: 393 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctgccatgggcaccttctccgcgtttgnnngcaggtgcaatggcatgaacaaacctgcaagcccattgcacctgcnntatgcaacgactgcgttttcgactgggtttgaagagcgtttaaacgctcttcgcccgcgttacgcgaaagtccgtagtttcgagacggccctga

SEQ ID NO: 394 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctaccttgtgaaaaggactgcaggccgtttgnnngcaggtgcaatgcacattcagaacctagccgagcattgcacctgcnntatgcactaacggccgaccgccccgcgtttgaagagcgtttaaacgctcttcgccccaacgaccggtacggatcatgtttcgagacggccctga

SEQ ID NO: 395 - DNA, 236 bp, artificial
  oligo
  misc_feature (93)..(95): n is a, c, g, or t
  misc_feature (141)..(142): n is a, c, g, or t
gaccgcgtctcacaccggtagaccaggcacccaacctgtttggagaccgtttaaacggtctctacctgtcacacctgagtaccactggtttgnnngcaggtgcaatgcgccgtcatactactcacatgcattgcacctgcnntatgcatgacgtaaaagacgcgtgtcgtttgaagagcgtttaaacgctcttcgcccaagcgcctcgggtagaccgagtttcgagacggccctga

SEQ ID NO: 396 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctacctccacagtactcacttggtccgtttgnnngcaggtgcaatgtcgcatcgccactataacgtgcattgcacctgcnntatgcacgtccgggtttagcattcgagtttgaagagcgtttaaacgctcttcgcccacgtagcgcgagaactccgcgtttcgagacggccctga

SEQ ID NO: 397 - DNA, 236 bp, artificial
  oligo
  misc_feature (93)..(95): n is a, c, g, or t
  misc_feature (141)..(142): n is a, c, g, or t
gaccgcgtctcacaccgcaagggcgtgaacagcgacggtttggagaccgtttaaacggtctctacctgcagtgaccgtcacgacctggtttgnnngcaggtgcaatgcctttaaccccgatcgaaccacattgcacctgcnntatgcatttcgcgatgcgcacgtattgtttgaagagcgtttaaacgctcttcgcccttgaaatcgattggtcgcgtgtttcgagacggccctga

SEQ ID NO: 398 - DNA, 236 bp, artificial
  oligo
  misc_feature (93)..(95): n is a, c, g, or t
  misc_feature (141)..(142): n is a, c, g, or t
gaccgcgtctcacaccggtgtctgtatgacccacccggtttggagaccgtttaaacggtctctacctattgtagtgcgtccccgccggtttgnnngcaggtgcaatgaccctattctgataccagccccattgcacctgcnntatgcaggatcgcggatatgtgcggtgtttgaagagcgtttaaacgctcttcgccccgattgtcaatcattacggcgtttcgagacggccctga

SEQ ID NO: 399 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctaccttgagtcgcgatatatgccgagtttgnnngcaggtgcaatgtgcaaccgacgcagccaaacacattgcacctgcnntatgcatgatgcgcacgatcggtggcgtttgaagagcgtttaaacgctcttcgccctaacgaggacgacgctcaacgtttcgagacggccctga

SEQ ID NO: 400 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctacctcgacttcgatttgtactccggtttgnnngcaggtgcaatggacggtccatcgcctatactgcattgcacctgcnntatgcatgcgcctaacatcgtaccgcgtttgaagagcgtttaaacgctcttcgccccttgcaacctgtaccgcgtcgtttcgagacggccctga

SEQ ID NO: 401 - DNA, 236 bp, artificial
  oligo
  misc_feature (93)..(95): n is a, c, g, or t
  misc_feature (141)..(142): n is a, c, g, or t
gaccgcgtctcacaccggccatgggcaccttctccgcgtttggagaccgtttaaacggtctctacctaccaacgtcgattgaccggagtttgnnngcaggtgcaatgccccgatacgtatctacagtccattgcacctgcnntatgcataccggcctacggtcgctatgtttgaagagcgtttaaacgctcttcgcccgcgggaccccgttcggatacgtttcgagacggccctga

SEQ ID NO: 402 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctaccttttcacacggttgtcgtcgcgtttgnnngcaggtgcaatgccagtatgactttctagcccccattgcacctgcnntatgcacgacgtcgctttagctcgttgtttgaagagcgtttaaacgctcttcgcccaccaccgtgaacgtccgacggtttcgagacggccctga

SEQ ID NO: 403 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctttgggggatactcacgggatgtttgnnngcaggtgcaatgccacacaaacgcctctcgatgcattgcacctgcnntatgcatacttgtacctgaaagctcggtttgaagagcgtttaaacgctcttcgccctcgacaacccctacatcgtggtttcgagacggccctga

SEQ ID NO: 404 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctaccttatccggttacctcatagtcgtttgnnngcaggtgcaatgcaaggaatccttacgcccacacattgcacctgcnntatgcagaagcgagagaatctcctcggtttgaagagcgtttaaacgctcttcgcccttattacgtgcgccggccgggtttcgagacggccctga

SEQ ID NO: 405 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctaagaggcttcttatgtcggcgtttgnnngcaggtgcaatgcctcgctacgtcacacaacgtcattgcacctgcnntatgcagaagcgagagaatctcctcggtttgaagagcgtttaaacgctcttcgcccgctataccggtcgcgacgaggtttcgagacggccctga

SEQ ID NO: 406 - DNA, 236 bp, artificial
  oligo
  misc_feature (93)..(95): n is a, c, g, or t
  misc_feature (141)..(142): n is a, c, g, or t
gaccgcgtctcacaccgttgggggatactcacgggatgtttggagaccgtttaaacggtctctacctgtgacccgaggaaccggtgcgtttgnnngcaggtgcaatgttgagtactagcatacgcccccattgcacctgcnntatgcagaaccgcacgtggaggatgcgtttgaagagcgtttaaacgctcttcgcccgcagcacaagatcgcgcgtcgtttcgagacggccctga

SEQ ID NO: 407 - DNA, 236 bp, artificial
  oligo
  misc_feature (93)..(95): n is a, c, g, or t
  misc_feature (141)..(142): n is a, c, g, or t
gaccgcgtctcacaccggaagcgagagaatctcctcggtttggagaccgtttaaacggtctctacctagggcacgtcaactctacacgtttgnnngcaggtgcaatggagtacgccacgctaatcgagcattgcacctgcnntatgcaggggcacgatggcctttaccgtttgaagagcgtttaaacgctcttcgcccgacatatacgcgatacgacagtttcgagacggccctga

SEQ ID NO: 408 - DNA, 236 bp, artificial
  oligo
  misc_feature (93)..(95): n is a, c, g, or t
  misc_feature (141)..(142): n is a, c, g, or t
gaccgcgtctcacaccgctcgtagcttgccacactgtgtttggagaccgtttaaacggtctctaccttcgacaacccctacatcgtggtttgnnngcaggtgcaatgcccgcaaatccgcgttaaaaccattgcacctgcnntatgcacgagcgtcgaaactcgacgcgtttgaagagcgtttaaacgctcttcgccccgctgccgcgttaggacatcgtttcgagacggccctga

SEQ ID NO: 409 - DNA, 236 bp, artificial
  oligo
  misc_feature (93)..(95): n is a, c, g, or t
  misc_feature (141)..(142): n is a, c, g, or t
gaccgcgtctcacaccggcatccacattcttacaggcgtttggagaccgtttaaacggtctctacctagggcacgtcaactctacacgtttgnnngcaggtgcaatgtgcagctattgacctcatcggcattgcacctgcnntatgcatcgctaacggttggaccgtggtttgaagagcgtttaaacgctcttcgccctacgacttctacacgccgtagtttcgagacggccctga

SEQ ID NO: 410 - DNA, 236 bp, artificial
  oligo
  misc_feature (93)..(95): n is a, c, g, or t
  misc_feature (141)..(142): n is a, c, g, or t
gaccgcgtctcacaccgctcgtagcttgccacactgtgtttggagaccgtttaaacggtctctacctcattgtatacgccttcctgggtttgnnngcaggtgcaatgcctattgtaagagaacgcccccattgcacctgcnntatgcacggcgaaagcgccgattagagtttgaagagcgtttaaacgctcttcgccccggatgccgatccgttaaatgtttcgagacggccctga

SEQ ID NO: 411 - DNA, 236 bp, artificial
  oligo
  misc_feature (93)..(95): n is a, c, g, or t
  misc_feature (141)..(142): n is a, c, g, or t
gaccgcgtctcacaccggcgccagtgtttgcgctcccgtttggagaccgtttaaacggtctctacctagggcacgtcaactctacacgtttgnnngcaggtgcaatgcccatcatcggctttccacgacattgcacctgcnntatgcatcgtggcgtgcgaattcgacgtttgaagagcgtttaaacgctcttcgcccgaccggcgagtccgtaccatgtttcgagacggccctga

SEQ ID NO: 412 - DNA, 236 bp, artificial
  oligo
  misc_feature (93)..(95): n is a, c, g, or t
  misc_feature (141)..(142): n is a, c, g, or t
gaccgcgtctcacaccggctcaatctagggcgaagaggtttggagaccgtttaaacggtctctacctcaactttggctccgtgcgccgtttgnnngcaggtgcaatgccgccaaaaagccgaaagtctcattgcacctgcnntatgcatagcgcgttgtcatcccgcggtttgaagagcgtttaaacgctcttcgcccatccactcgcgacgcctaatgtttcgagacggccctga

SEQ ID NO: 413 - DNA, 236 bp, artificial
  oligo
  misc_feature (93)..(95): n is a, c, g, or t
  misc_feature (141)..(142): n is a, c, g, or t
gaccgcgtctcacaccgtacttgtacctgaaagctcggtttggagaccgtttaaacggtctctacctgaaccgcacgtggaggatgcgtttgnnngcaggtgcaatgccgacgcagattccgtactaccattgcacctgcnntatgcagggtgggtcgttactacgcggtttgaagagcgtttaaacgctcttcgcccgatcgctaggattgcgcggggtttcgagacggccctga

SEQ ID NO: 414 - DNA, 236 bp, artificial
  oligo
  misc_feature (93)..(95): n is a, c, g, or t
  misc_feature (141)..(142): n is a, c, g, or t
gaccgcgtctcacaccgctcacggcagcgcacgcacagtttggagaccgtttaaacggtctctacctctaacgtcgcgtcgagtcgggtttgnnngcaggtgcaatgcttctagaccgacctacatggcattgcacctgcnntatgcacccgtatcgccgagcaaactgtttgaagagcgtttaaacgctcttcgccccccggacggttcgtttgcgtgtttcgagacggccctga

SEQ ID NO: 415 - DNA, 236 bp, artificial
  oligo
  misc_feature (93)..(95): n is a, c, g, or t
  misc_feature (141)..(142): n is a, c, g, or t
gaccgcgtctcacaccgcaacggcacgtacgccatcggtttggagaccgtttaaacggtctctacctgacgggtaatgcgtgcgttcgtttgnnngcaggtgcaatgctcctaggtccaatcccatcccattgcacctgcnntatgcacgagtaataacgacgacttcgtttgaagagcgtttaaacgctcttcgcccactatgcggtaggtacgccggtttcgagacggccctga

SEQ ID NO: 416 - DNA, 236 bp, artificial
  oligo
  misc_feature (93)..(95): n is a, c, g, or t
  misc_feature (141)..(142): n is a, c, g, or t
gaccgcgtctcacaccgtatccggttacctcatagtcgtttggagaccgtttaaacggtctctacctagtcgtgcgatcgtggtgtggtttgnnngcaggtgcaatgcgtccccaataatataccggccattgcacctgcnntatgcaactcgcgctaatcgctaagagtttgaagagcgtttaaacgctcttcgccccggccgaacagcgtattatagtttcgagacggccctga

SEQ ID NO: 417 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctaccttatgcggcgcggttaaaagggtttgnnngcaggtgcaatgcgcagcctttacatgactcaccattgcacctgcnntatgcaccgcgttgcacttgaccggggtttgaagagcgtttaaacgctcttcgccccgtagccgcatcaaaatcgcgtttcgagacggccctga

SEQ ID NO: 418 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctaccttcacccataggagccacagcgtttgnnngcaggtgcaatgccgataaagaaccaccgacggcattgcacctgcnntatgcacagtgcaacttactattacggtttgaagagcgtttaaacgctcttcgccctctgggcatgtgggtccggcgtttcgagacggccctga

SEQ ID NO: 419 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctaccttgttgggcagctgtatgatcgtttgnnngcaggtgcaatgtgtccctatgaccgaatcgtgcattgcacctgcnntatgcactaaagggcgatctcaacaagtttgaagagcgtttaaacgctcttcgcccacctatcatacgtatcctgggtttcgagacggccctga

SEQ ID NO: 420 - DNA, 236 bp, artificial
  oligo
  misc_feature (93)..(95): n is a, c, g, or t
  misc_feature (141)..(142): n is a, c, g, or t
gaccgcgtctcacaccgggagagccaacccctaccaggtttggagaccgtttaaacggtctctaccttgttgggcagctgtatgatcgtttgnnngcaggtgcaatgcgccatcttactaccaccccacattgcacctgcnntatgcacctatcatacgtatcctggtgtttgaagagcgtttaaacgctcttcgcccttcatagacctaatgtgactgtttcgagacggccctga

SEQ ID NO: 421 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctatcagttctgagcaactcgtgtttgnnngcaggtgcaatgagcaactagtaagcaccagggcattgcacctgcnntatgcaccctcaccttctacccagccgtttgaagagcgtttaaacgctcttcgcccggactgagagctgttgacacgtttcgagacggccctga

SEQ ID NO: 422 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctctgctagccaacaccactgagtttgnnngcaggtgcaatggaaacccgaagccatcgccatcattgcacctgcnntatgcacagcttgtccaactggtcgggtttgaagagcgtttaaacgctcttcgccctctttgaaccattgtgttatgtttcgagacggccctga

SEQ ID NO: 423 - DNA, 236 bp, artificial
  oligo
  misc_feature (93)..(95): n is a, c, g, or t
  misc_feature (141)..(142): n is a, c, g, or t
gaccgcgtctcacaccggaggcggtcaggatggctgcgtttggagaccgtttaaacggtctctacctgattccttccaataacacaagtttgnnngcaggtgcaatgtggagccaacgatctagaacccattgcacctgcnntatgcacgtcaccaagccattgtaccgtttgaagagcgtttaaacgctcttcgccccagcttgtccaactggtcgggtttcgagacggccctga

SEQ ID NO: 424 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctctggaaggtgaggcctaggcgtttgnnngcaggtgcaatgtcccgtcaccatattgacagccattgcacctgcnntatgcatgttgagatcgccctttagcgtttgaagagcgtttaaacgctcttcgcccggaagggacactaacctggtgtttcgagacggccctga

SEQ ID NO: 425 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctaccttctttgaaccattgtgttatgtttgnnngcaggtgcaatgacaactcccgtcttaaacccccattgcacctgcnntatgcatcacccataggagccacagcgtttgaagagcgtttaaacgctcttcgccctgtgatggtgaatattcacagtttcgagacggccctga

SEQ ID NO: 426 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctctaaagggcgatctcaacaagtttgnnngcaggtgcaatggatcagttgcgggactaaagccattgcacctgcnntatgcactccacaaagacctgcggcggtttgaagagcgtttaaacgctcttcgcccctggaaggtgaggcctaggcgtttcgagacggccctga

SEQ ID NO: 427 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctgaggcggtcaggatggctgcgtttgnnngcaggtgcaatgcaagaagcctcaaggaagccccattgcacctgcnntatgcattcatagacctaatgtgactgtttgaagagcgtttaaacgctcttcgccctgtgatggtgaatattcacagtttcgagacggccctga

SEQ ID NO: 428 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctacaggtgacccaaccttcaggtttgnnngcaggtgcaatggcaccaagagcttcactactccattgcacctgcnntatgcagaggcggtcaggatggctgcgtttgaagagcgtttaaacgctcttcgccctctgggcatgtgggtccggcgtttcgagacggccctga

SEQ ID NO: 429 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctacctcaacttcttcgtggtatctcgtttgnnngcaggtgcaatgcgacgcctattccgaaagtgacattgcacctgcnntatgcacccaccaggatacgtatgatgtttgaagagcgtttaaacgctcttcgcccacacaacactgatgaggtccgtttcgagacggccctga

SEQ ID NO: 430 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctacctctgaaattagacatcaaagcgtttgnnngcaggtgcaatgcccgcactaatctacgctaaccattgcacctgcnntatgcatgaatgaccagggtacctgcgtttgaagagcgtttaaacgctcttcgccccgtcaccaagccattgtaccgtttcgagacggccctga

SEQ ID NO: 431 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctctgcagttgagctggcaatcgtttgnnngcaggtgcaatgccccgatctgtttcaacgtagcattgcacctgcnntatgcaagctgtagtagagtccccatgtttgaagagcgtttaaacgctcttcgcccggactgagagctgttgacacgtttcgagacggccctga

SEQ ID NO: 432 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctaatcccttgccccagcacatgtttgnnngcaggtgcaatgtgaagtgtaccacaagctcgccattgcacctgcnntatgcaactttccatgtcagccatacgtttgaagagcgtttaaacgctcttcgccccaaagacctgcggcgagggcgtttcgagacggccctga

SEQ ID NO: 433 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctacctatcatacgtatcctgggtttgnnngcaggtgcaatgtatgtgccaacagctaccgtccattgcacctgcnntatgcatgaatgaccagggtacctgcgtttgaagagcgtttaaacgctcttcgcccatagacgcgtgacaaccgaagtttcgagacggccctga

SEQ ID NO: 434 - DNA, 236 bp, artificial
  oligo
  misc_feature (93)..(95): n is a, c, g, or t
  misc_feature (141)..(142): n is a, c, g, or t
gaccgcgtctcacaccgattcttctctgtgaatgtcggtttggagaccgtttaaacggtctctaccttctgggcatgtgggtccggcgtttgnnngcaggtgcaatgggaaactccgccaatttccctcattgcacctgcnntatgcatcatcctgcatgctagaccagtttgaagagcgtttaaacgctcttcgcccccgcgagtcctggacataaggtttcgagacggccctga

SEQ ID NO: 435 - DNA, 236 bp, artificial
  oligo
  misc_feature (93)..(95): n is a, c, g, or t
  misc_feature (141)..(142): n is a, c, g, or t
gaccgcgtctcacaccggccctttagctggtatctgcgtttggagaccgtttaaacggtctctacctctgcagttgagctggcaatcgtttgnnngcaggtgcaatgccctatcccaacgtcccataacattgcacctgcnntatgcatctttgaaccattgtgttatgtttgaagagcgtttaaacgctcttcgcccgcggcgctgtcgagatacacgtttcgagacggccctga

SEQ ID NO: 436 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctaccttctgggcatgtgggtccggcgtttgnnngcaggtgcaatgtcccgacctcgatcttactcccattgcacctgcnntatgcatcacatggatggttactgaagtttgaagagcgtttaaacgctcttcgcccttcgcatcgatccgctgtaagtttcgagacggccctga

SEQ ID NO: 437 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctactttccatgtcagccatacgtttgnnngcaggtgcaatgttatccgcccagagccaaactcattgcacctgcnntatgcattcatagacctaatgtgactgtttgaagagcgtttaaacgctcttcgccccacgattcacgcgtaggcgcgtttcgagacggccctga

SEQ ID NO: 438 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctaccttgttgagatcgccctttagcgtttgnnngcaggtgcaatggagaaccagctacaccccgaacattgcacctgcnntatgcattcatagacctaatgtgactgtttgaagagcgtttaaacgctcttcgcccattacgcgtacgacgggccagtttcgagacggccctga

SEQ ID NO: 439 - DNA, 236 bp, artificial
  oligo
  misc_feature (93)..(95): n is a, c, g, or t
  misc_feature (141)..(142): n is a, c, g, or t
gaccgcgtctcacaccgtcacatggatggttactgaagtttggagaccgtttaaacggtctctacctggccatttatagtgttgaccgtttgnnngcaggtgcaatgatgcgctagactcgaattccccattgcacctgcnntatgcacaaagacctgcggcgagggcgtttgaagagcgtttaaacgctcttcgcccggggcggaccaaaattcgcggtttcgagacggccctga

SEQ ID NO: 440 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctttgggactcctcggttatttgtttgnnngcaggtgcaatgagccactacgctaaccacatgcattgcacctgcnntatgcagccctttagctggtatctgcgtttgaagagcgtttaaacgctcttcgcccgggaccgatccattaccgtagtttcgagacggccctga

SEQ ID NO: 441 - DNA, 236 bp, artificial
  oligo
  misc_feature (93)..(95): n is a, c, g, or t
  misc_feature (141)..(142): n is a, c, g, or t
gaccgcgtctcacaccgggaagggacactaacctggtgtttggagaccgtttaaacggtctctacctcgtcaccaagccattgtaccgtttgnnngcaggtgcaatgcgcctaagaactcccactgaccattgcacctgcnntatgcaacaggtgacccaaccttcaggtttgaagagcgtttaaacgctcttcgccccgaacccgtcgcccgcttacgtttcgagacggccctga

SEQ ID NO: 442 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctaccttgtcgatggcaatagccaaggtttgnnngcaggtgcaatgccgaccctaaataccttgtggcattgcacctgcnntatgcagttacactctatctacacctgtttgaagagcgtttaaacgctcttcgccccgaccgtgttgttccgtcgggtttcgagacggccctga

SEQ ID NO: 443 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctcctatcatacgtatcctggtgtttgnnngcaggtgcaatgccccgtagataaacgagcaaccattgcacctgcnntatgcactgcagttgagctggcaatcgtttgaagagcgtttaaacgctcttcgcccgtcgcgacgctaattccctagtttcgagacggccctga

SEQ ID NO: 444 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttgaatgaccagggtacctgcgtttgnnngcaggtgcaatggcccacataccaatagttccccattgcacctgcnntatgcatcacccataggagccacagcgtttgaagagcgtttaaacgctcttcgccccaagtagcgcgcttagacatgtttcgagacggccctga

SEQ ID NO: 445 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctagatcatacagctgcccaacgtttgnnngcaggtgcaatgccaatcgatcccgaatgtacgcattgcacctgcnntatgcagtatcctggtgggatttacagtttgaagagcgtttaaacgctcttcgccccgtggcccgtttagcgtgtcgtttcgagacggccctga

SEQ ID NO: 446 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctgctcaaaccattacagaagggtttgnnngcaggtgcaatggacttaccatgacctaggcgccattgcacctgcnntatgcatgcccacaaagtatggcggtgtttgaagagcgtttaaacgctcttcgcccagttgcttcgttcgaccagtgtttcgagacggccctga

SEQ ID NO: 447 - DNA, 236 bp, artificial
  oligo
  misc_feature (93)..(95): n is a, c, g, or t
  misc_feature (141)..(142): n is a, c, g, or t
gaccgcgtctcacaccgcaaagacctgcggcgagggcgtttggagaccgtttaaacggtctctacctacatagacccctgttgtaaggtttgnnngcaggtgcaatgctcgatccgcacatgcataaccattgcacctgcnntatgcacagcttgtccaactggtcgggtttgaagagcgtttaaacgctcttcgcccgggcgtatcggtcaagacgagtttcgagacggccctga

SEQ ID NO: 448 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctgaggcggtcaggatggctgcgtttgnnngcaggtgcaatgtgcatactcccactgaatccccattgcacctgcnntatgcaagatcatacagctgcccaacgtttgaagagcgtttaaacgctcttcgccctaatgctccgcggccgcttagtttcgagacggccctga

SEQ ID NO: 449 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctgtatcctggtgggatttacagtttgnnngcaggtgcaatgcgcccaaattcgacggaatcccattgcacctgcnntatgcatgtgatggtgaatattcacagtttgaagagcgtttaaacgctcttcgcccttcaccgcgcaaacgatggcgtttcgagacggccctga

SEQ ID NO: 450 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctcaacttcttcgtggtatctcgtttgnnngcaggtgcaatgccgcaacacagtataatccggcattgcacctgcnntatgcacaggcacgatagatacaaaggtttgaagagcgtttaaacgctcttcgcccgacgagtcgtcgcgctactggtttcgagacggccctga

SEQ ID NO: 451 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctatcagttctgagcaactcgtgtttgnnngcaggtgcaatgcccttgataacgaccgaagcgcattgcacctgcnntatgcacctatcatacgtatcctggtgtttgaagagcgtttaaacgctcttcgcccggccggtatacgtctacgaggtttcgagacggccctga

SEQ ID NO: 452 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctctccacaaagacctgcggcggtttgnnngcaggtgcaatggattccatcccgagcattacccattgcacctgcnntatgcattgttacagccttattacacgtttgaagagcgtttaaacgctcttcgccctcggccgtataggaatggtagtttcgagacggccctga

SEQ ID NO: 453 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctacctggaagggacactaacctggtgtttgnnngcaggtgcaatgctttcgcgccaagagacaacgcattgcacctgcnntatgcacaatcacgcgccgcgagactgtttgaagagcgtttaaacgctcttcgcccccggaatcgttaagttacgtgtttcgagacggccctga

SEQ ID NO: 454 - DNA, 236 bp, artificial
  oligo
  misc_feature (93)..(95): n is a, c, g, or t
  misc_feature (141)..(142): n is a, c, g, or t
gaccgcgtctcacaccgttgggactcctcggttatttgtttggagaccgtttaaacggtctctacctggccatttatagtgttgaccgtttgnnngcaggtgcaatgtgccccagtcagacttccacacattgcacctgcnntatgcacgtatatcccggcggcatcggtttgaagagcgtttaaacgctcttcgcccagcgttaaccgcgtggcgtggtttcgagacggccctga

SEQ ID NO: 455 - DNA, 236 bp, artificial
  oligo
  misc_feature (93)..(95): n is a, c, g, or t
  misc_feature (141)..(142): n is a, c, g, or t
gaccgcgtctcacaccggattccttccaataacacaagtttggagaccgtttaaacggtctctacctacaggtgacccaaccttcaggtttgnnngcaggtgcaatggtccagaacgactagcaagaccattgcacctgcnntatgcacgcacattagtggtgcgcgcgtttgaagagcgtttaaacgctcttcgcccaccggcggaactattcgacggtttcgagacggccctga

SEQ ID NO: 456 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttgggactcctcggttatttagtttgnnngcaggtgcaatggcgcctaaagcagcacaatcccattgcacctgcnntatgcaactgcgcgtataacgatagggtttgaagagcgtttaaacgctcttcgcccttacagcttacgtcggcacggtttcgagacggccctga

SEQ ID NO: 457 - DNA, 236 bp, artificial
  oligo
  misc_feature (93)..(95): n is a, c, g, or t
  misc_feature (141)..(142): n is a, c, g, or t
gaccgcgtctcacaccgcagtgcaacttactattacggtttggagaccgtttaaacggtctctacctagaagttggtgacgttctgcgtttgnnngcaggtgcaatgtcccagatgcgcaaaaagcaccattgcacctgcnntatgcaaccgaccaaattccccggcggtttgaagagcgtttaaacgctcttcgcccactgattcgaactcgccgtggtttcgagacggccctga

SEQ ID NO: 458 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctaccttctgggcatgtgggtccggcgtttgnnngcaggtgcaatgatccaccgtactccttagacccattgcacctgcnntatgcaaagggtagcgtaattcgtcggtttgaagagcgtttaaacgctcttcgcccataggggcggcgattccgacgtttcgagacggccctga

SEQ ID NO: 459 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctaccttgcccacaaagtatggcggtgtttgnnngcaggtgcaatgtagcagaccatccggccgaaacattgcacctgcnntatgcacggcgaaacgtaagcacgccgtttgaagagcgtttaaacgctcttcgccccggattgcgaaactatccgggtttcgagacggccctga

SEQ ID NO: 460 - DNA, 236 bp, artificial
  oligo
  misc_feature (93)..(95): n is a, c, g, or t
  misc_feature (141)..(142): n is a, c, g, or t
gaccgcgtctcacaccgcaatacagggatacccactagtttggagaccgtttaaacggtctctacctctgaaattagacatcaaagcgtttgnnngcaggtgcaatgagctgaacttcaccgacgacgcattgcacctgcnntatgcagccgaattgcgacacgaggtgtttgaagagcgtttaaacgctcttcgcccaacggcggacgacctacaatgtttcgagacggccctga

SEQ ID NO: 461 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctctccacaaagacctgcggcggtttgnnngcaggtgcaatgcctatatggacactcagctcgcattgcacctgcnntatgcatacactacgggaactcgccggtttgaagagcgtttaaacgctcttcgcccgaacgtctaccgtcggcggagtttcgagacggccctga

SEQ ID NO: 462 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctctgctagccaacaccactgagtttgnnngcaggtgcaatgcttagtacctccaacaaccggcattgcacctgcnntatgcatctacggccgcggttacgttgtttgaagagcgtttaaacgctcttcgccccacatctcgtcggacgctcggtttcgagacggccctga

SEQ ID NO: 463 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctatcagttctgagcaactcgtgtttgnnngcaggtgcaatgtcttctctccacggtgtgctacattgcacctgcnntatgcaatatgctagcgtcgcattcggtttgaagagcgtttaaacgctcttcgccctccgcaagttgcgtacgacggtttcgagacggccctga

SEQ ID NO: 464 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctcaggcacgatagatacaaaggtttgnnngcaggtgcaatgcaccacacaggtcgtttacagcattgcacctgcnntatgcacgacgttgagcgttagactggtttgaagagcgtttaaacgctcttcgccccagtatcgggtgcataacgcgtttcgagacggccctga

SEQ ID NO: 465 - DNA, 236 bp, artificial
  oligo
  misc_feature (93)..(95): n is a, c, g, or t
  misc_feature (141)..(142): n is a, c, g, or t
gaccgcgtctcacaccgggactgagagctgttgacacgtttggagaccgtttaaacggtctctacctttgttacagccttattacacgtttgnnngcaggtgcaatgcccgtacctatagcactaggccattgcacctgcnntatgcatatagcgagcgacggacgccgtttgaagagcgtttaaacgctcttcgccccgtctgctcgaagtcgagttgtttcgagacggccctga

SEQ ID NO: 466 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctggactgagagctgttgacacgtttgnnngcaggtgcaatgctcatgcaattagacacgcggcattgcacctgcnntatgcatcgctctaatccggatgccggtttgaagagcgtttaaacgctcttcgcccaatcgtatttggtcgcgcgagtttcgagacggccctga

SEQ ID NO: 467 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctacctcgtcaccaagccattgtaccgtttgnnngcaggtgcaatgccccatatgctcgacagaggacattgcacctgcnntatgcactcgatatcaaccccgtaacgtttgaagagcgtttaaacgctcttcgcccactcgattgcgagcgtacccgtttcgagacggccctga

SEQ ID NO: 468 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctaccttcgtttggccagacgcgtcagtttgnnngcaggtgcaatgggaagagtgcgtcatactccacattgcacctgcnntatgcacttacgatattagcgtcgaagtttgaagagcgtttaaacgctcttcgcccgcctcatcgcgacatcgtaggtttcgagacggccctga

SEQ ID NO: 469 - DNA, 236 bp, artificial
  oligo
  misc_feature (93)..(95): n is a, c, g, or t
  misc_feature (141)..(142): n is a, c, g, or t
gaccgcgtctcacaccgcagcttgtccaactggtcgggtttggagaccgtttaaacggtctctacctaattattagattcgcgcgtcgtttgnnngcaggtgcaatgaactcagcgccgcaacataagcattgcacctgcnntatgcatcgccacctaattcgacggtgtttgaagagcgtttaaacgctcttcgcccgtgttcgacgcctctccggagtttcgagacggccctga

SEQ ID NO: 470 - DNA, 236 bp, artificial
  oligo
  misc_feature (93)..(95): n is a, c, g, or t
  misc_feature (141)..(142): n is a, c, g, or t
gaccgcgtctcacaccgcaggcacgatagatacaaaggtttggagaccgtttaaacggtctctacctcgcgctagtccgacaccaaagtttgnnngcaggtgcaatgaccacatccgcttacactctgcattgcacctgcnntatgcactcgatcgtgcggtatacgtgtttgaagagcgtttaaacgctcttcgccccgcgcctatatgcggattccgtttcgagacggccctga

SEQ ID NO: 471 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctgatcacgaatttatcgccgtgtttgnnngcaggtgcaatgagccaccgacctagtcttagccattgcacctgcnntatgcaaatccgctcgtgtgcgctatgtttgaagagcgtttaaacgctcttcgccccgttacgcctccgccgttccgtttcgagacggccctga

SEQ ID NO: 472 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctaccttcgcacccgttattgaagcggtttgnnngcaggtgcaatgttcccacttacacgagatcggcattgcacctgcnntatgcagcgatacccggtgttcgattgtttgaagagcgtttaaacgctcttcgccctcgcgcatcgaaaagtaactgtttcgagacggccctga

SEQ ID NO: 473 - DNA, 236 bp, artificial
  oligo
  misc_feature (93)..(95): n is a, c, g, or t
  misc_feature (141)..(142): n is a, c, g, or t
gaccgcgtctcacaccgcaacttcttcgtggtatctcgtttggagaccgtttaaacggtctctacctattcgcgttttagccactcggtttgnnngcaggtgcaatggctctacgaatacactctgcgcattgcacctgcnntatgcatgttgcgacctttcgccgtagtttgaagagcgtttaaacgctcttcgccccgcccgaagtcggttagtctgtttcgagacggccctga

SEQ ID NO: 474 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctacctgccatgggcaccttctccgcgtttgnnngcaggtgcaatgaaaccgccctaacttatgccccattgcacctgcnntatgcagttgccaggtgagcccgctcgtttgaagagcgtttaaacgctcttcgcccgctgacatcggctttgctgcgtttcgagacggccctga

SEQ ID NO: 475 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctacctgagccgctgtgatcataccagtttgnnngcaggtgcaatggtaagccaagcagacagatggcattgcacctgcnntatgcagttgccaggtgagcccgctcgtttgaagagcgtttaaacgctcttcgcccgcaaagccgatgtcagcgccgtttcgagacggccctga

SEQ ID NO: 476 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctgttgccaggtgagcccgctcgtttgnnngcaggtgcaatggtcagagctcacgcatttacccattgcacctgcnntatgcagtgcatacgtacttcgtccagtttgaagagcgtttaaacgctcttcgcccaacatgactccccggaggccgtttcgagacggccctga

SEQ ID NO: 477 - DNA, 236 bp, artificial
  oligo
  misc_feature (93)..(95): n is a, c, g, or t
  misc_feature (141)..(142): n is a, c, g, or t
gaccgcgtctcacaccgtgctgtcgtgagtgccagccgtttggagaccgtttaaacggtctctaccttggcattggggacctcgggtgtttgnnngcaggtgcaatgacgcagctacttcacacatcccattgcacctgcnntatgcagtggaagtctgtgtcgggaagtttgaagagcgtttaaacgctcttcgccctcctcagctgccatatgtgcgtttcgagacggccctga

SEQ ID NO: 478 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctcaagggcgtgaacagcgacggtttgnnngcaggtgcaatggtccgaagccttcaacagatccattgcacctgcnntatgcaaccaggcacccaacctcggcgtttgaagagcgtttaaacgctcttcgcccgagaagcactatgggggcatgtttcgagacggccctga

SEQ ID NO: 479 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctgtgtctgtatgacccacccggtttgnnngcaggtgcaatgcctagttcgtcagatacccagcattgcacctgcnntatgcatgtgcttaagaccgggaccagtttgaagagcgtttaaacgctcttcgccctcgtggcgggtcgaattcgcgtttcgagacggccctga

SEQ ID NO: 480 - DNA, 236 bp, artificial
  oligo
  misc_feature (93)..(95): n is a, c, g, or t
  misc_feature (141)..(142): n is a, c, g, or t
gaccgcgtctcacaccggagaagcactatgggggcatgtttggagaccgtttaaacggtctctacctgttgccaggtgagcccgctcgtttgnnngcaggtgcaatgtgccattccggtccagtatagcattgcacctgcnntatgcagcaggtccttaccttgttccgtttgaagagcgtttaaacgctcttcgcccgtgcgcgcgtagcgatcgacgtttcgagacggccctga

SEQ ID NO: 481 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctaccaggctgacagttatcacgtttgnnngcaggtgcaatgcaacccgaactcgaaatgacccattgcacctgcnntatgcatggcattggggacctcgggtgtttgaagagcgtttaaacgctcttcgccctcgaaatcgacacgtcgcgagtttcgagacggccctga

SEQ ID NO: 482 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctgttgccaggtgagcccgctcgtttgnnngcaggtgcaatggccctcttcaggaagaatgagcattgcacctgcnntatgcagtagaccaggcacccaacctgtttgaagagcgtttaaacgctcttcgcccgttcgccgaataggtcgctagtttcgagacggccctga

SEQ ID NO: 483 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctaccttgctgtcgtgagtgccagccgtttgnnngcaggtgcaatgccaccaacgtaggtagaaacccattgcacctgcnntatgcaagccacgcttgctgatgttggtttgaagagcgtttaaacgctcttcgcccgtgtcgtagtacgtcgacaagtttcgagacggccctga

SEQ ID NO: 484 - DNA, 236 bp, artificial
  oligo
  misc_feature (93)..(95): n is a, c, g, or t
  misc_feature (141)..(142): n is a, c, g, or t
gaccgcgtctcacaccgtcctcagctgccatatgtgcgtttggagaccgtttaaacggtctctacctgagaggtttccgaagactaagtttgnnngcaggtgcaatgccacggttacgcacaatcctgcattgcacctgcnntatgcagcttgtggtagatagcaacggtttgaagagcgtttaaacgctcttcgcccagcgtgttgcgtccgccgtagtttcgagacggccctga

SEQ ID NO: 485 - DNA, 236 bp, artificial
  oligo
  misc_feature (93)..(95): n is a, c, g, or t
  misc_feature (141)..(142): n is a, c, g, or t
gaccgcgtctcacaccgaagcactacccctgccaaccgtttggagaccgtttaaacggtctctacctccaagtaccttctccagcatgtttgnnngcaggtgcaatgagacttgcactttagccccaccattgcacctgcnntatgcagtagaccaggcacccaacctgtttgaagagcgtttaaacgctcttcgccctgttacgccacgctatttcggtttcgagacggccctga

SEQ ID NO: 486 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctctagcaagccataacaaaacgtttgnnngcaggtgcaatgctaccacgaccgaagcttagacattgcacctgcnntatgcagtagaccaggcacccaacctgtttgaagagcgtttaaacgctcttcgccccatcgtgaatcgatccgaaggtttcgagacggccctga

SEQ ID NO: 487 - DNA, 236 bp, artificial
  oligo
  misc_feature (93)..(95): n is a, c, g, or t
  misc_feature (141)..(142): n is a, c, g, or t
gaccgcgtctcacaccgatccccgtcgcagttgaactgtttggagaccgtttaaacggtctctacctaagcactacccctgccaaccgtttgnnngcaggtgcaatgtcacgcaaccctactggcaaacattgcacctgcnntatgcagtgcatacgtacttcgtccagtttgaagagcgtttaaacgctcttcgcccagataatatgcgccgctcgggtttcgagacggccctga

SEQ ID NO: 488 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctttcttctcagttcaagtaacgtttgnnngcaggtgcaatgttccgactaccggcatacactcattgcacctgcnntatgcatcctgccgaggttgggtgccgtttgaagagcgtttaaacgctcttcgccctacggtcacacgttgcgcctgtttcgagacggccctga

SEQ ID NO: 489 - DNA, 236 bp, artificial
  oligo
  misc_feature (93)..(95): n is a, c, g, or t
  misc_feature (141)..(142): n is a, c, g, or t
gaccgcgtctcacaccgaaccccaacatcagcaagcggtttggagaccgtttaaacggtctctacctgtgcatacgtacttcgtccagtttgnnngcaggtgcaatgccgcactacacgcgtttaatgcattgcacctgcnntatgcacgcgacatgcatcgctttcggtttgaagagcgtttaaacgctcttcgccccccccgtcggacgcgtatgagtttcgagacggccctga

SEQ ID NO: 490 - DNA, 236 bp, artificial
  oligo
  misc_feature (93)..(95): n is a, c, g, or t
  misc_feature (141)..(142): n is a, c, g, or t
gaccgcgtctcacaccgatcttgcagatgctaatagcgtttggagaccgtttaaacggtctctaccttcccacagttgtaccttatcgtttgnnngcaggtgcaatgtttcaatccttcatccgccgccattgcacctgcnntatgcacaatcgcacgcgtaaccgtagtttgaagagcgtttaaacgctcttcgcccgcgagtcgagcaatttggcggtttcgagacggccctga

SEQ ID NO: 491 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctaccttggcattggggacctcgggtgtttgnnngcaggtgcaatgcgcaactaccctcatcctgaccattgcacctgcnntatgcatgtgcgccgtcgcaatagtagtttgaagagcgtttaaacgctcttcgccctcgtgaaagcgtaccgcactgtttcgagacggccctga

SEQ ID NO: 492 - DNA, 236 bp, artificial
  oligo
  misc_feature (93)..(95): n is a, c, g, or t
  misc_feature (141)..(142): n is a, c, g, or t
gaccgcgtctcacaccgacctgtggtggttatggggcgtttggagaccgtttaaacggtctctacctgtggaagtctgtgtcgggaagtttgnnngcaggtgcaatgtaccatccgtgaacaaccagccattgcacctgcnntatgcaacgttagtgtcgcaccgacggtttgaagagcgtttaaacgctcttcgcccctaatcggcgtcttcgcggcgtttcgagacggccctga

SEQ ID NO: 493 - DNA, 236 bp, artificial
  oligo
  misc_feature (93)..(95): n is a, c, g, or t
  misc_feature (141)..(142): n is a, c, g, or t
gaccgcgtctcacaccggcaggtccttaccttgttccgtttggagaccgtttaaacggtctctaccttcacacttggagttacagcagtttgnnngcaggtgcaatgcccctttattccacgttctgccattgcacctgcnntatgcaccgcgaccggacgtttatccgtttgaagagcgtttaaacgctcttcgcccgcgtcacttcgtcgaatcccgtttcgagacggccctga

SEQ ID NO: 494 - DNA, 236 bp, artificial
  oligo
  misc_feature (93)..(95): n is a, c, g, or t
  misc_feature (141)..(142): n is a, c, g, or t
gaccgcgtctcacaccggtcacacctgagtaccactggtttggagaccgtttaaacggtctctacctgcaaagccgatgtcagcgccgtttgnnngcaggtgcaatggaaacgacgatcgtcaaaggccattgcacctgcnntatgcatatgggctcgtcatcgttcggtttgaagagcgtttaaacgctcttcgcccaagcgttgttaatcgccgtagtttcgagacggccctga

SEQ ID NO: 495 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctgtggaagtctgtgtcgggaagtttgnnngcaggtgcaatgcagacctcgagacacacacaacattgcacctgcnntatgcagtaacaacgaaccggcccgggtttgaagagcgtttaaacgctcttcgcccgtcgacacgttagtagggcggtttcgagacggccctga

SEQ ID NO: 496 - DNA, 236 bp, artificial
  oligo
  misc_feature (93)..(95): n is a, c, g, or t
  misc_feature (141)..(142): n is a, c, g, or t
gaccgcgtctcacaccggcagtgaccgtcacgacctggtttggagaccgtttaaacggtctctaccttcccacagttgtaccttatcgtttgnnngcaggtgcaatgctctgcttccacactgatcctcattgcacctgcnntatgcacgtgttaggtcgccgagcttgtttgaagagcgtttaaacgctcttcgcccacgtctattcgggcgtcgacgtttcgagacggccctga

SEQ ID NO: 497 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctacctaccaggcacccaacctcggcgtttgnnngcaggtgcaatggagatcggcaagaccaaacgacattgcacctgcnntatgcagtcccgtgacaaaccgtcgtgtttgaagagcgtttaaacgctcttcgccccgcgtattaccggtcttatggtttcgagacggccctga

SEQ ID NO: 498 - DNA, 236 bp, artificial
  oligo
  misc_feature (93)..(95): n is a, c, g, or t
  misc_feature (141)..(142): n is a, c, g, or t
gaccgcgtctcacaccgaaagcagtcgcccctgcttggtttggagaccgtttaaacggtctctacctgtcacacctgagtaccactggtttgnnngcaggtgcaatgcccaacgctcgataagctcaccattgcacctgcnntatgcaacatcgcgtcggtctagtgagtttgaagagcgtttaaacgctcttcgccctgattcgacgggtagtcgcagtttcgagacggccctga

SEQ ID NO: 499 - DNA, 236 bp, artificial
  oligo
  misc_feature (93)..(95): n is a, c, g, or t
  misc_feature (141)..(142): n is a, c, g, or t
gaccgcgtctcacaccgtaaccatagtacccagttgtgtttggagaccgtttaaacggtctctacctcggtagttgcgtagtagacggtttgnnngcaggtgcaatgggaattaactgcatcctcgcccattgcacctgcnntatgcatacggcgttttgaccccgtagtttgaagagcgtttaaacgctcttcgccccgttagttatgcgaacgcatgtttcgagacggccctga

SEQ ID NO: 500 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctcgggaagcctattcgccgcggtttgnnngcaggtgcaatggtactccactatgcacagaggcattgcacctgcnntatgcactaatcgtacgtatcgagtcgtttgaagagcgtttaaacgctcttcgccccactcgtcgctcgacaagatgtttcgagacggccctga

SEQ ID NO: 501 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctacctcgctaaggacgctcccgcatgtttgnnngcaggtgcaatgcacgatctagccctgaacaagcattgcacctgcnntatgcagcgagtcgctccgtcatagggtttgaagagcgtttaaacgctcttcgcccggtcgaggggcgcgcatttcgtttcgagacggccctga

SEQ ID NO: 502 - DNA, 236 bp, artificial
  oligo
  misc_feature (93)..(95): n is a, c, g, or t
  misc_feature (141)..(142): n is a, c, g, or t
gaccgcgtctcacaccggagaagcactatgggggcatgtttggagaccgtttaaacggtctctacctccgttgacctttcggcgtcggtttgnnngcaggtgcaatgctcgtattccgaccctcgtttcattgcacctgcnntatgcaacgagtctggacgctcgacggtttgaagagcgtttaaacgctcttcgcccttgccgaagcgcggtagtatgtttcgagacggccctga

SEQ ID NO: 503 - DNA, 236 bp, artificial
  oligo
  misc_feature (93)..(95): n is a, c, g, or t
  misc_feature (141)..(142): n is a, c, g, or t
gaccgcgtctcacaccggcttgtggtagatagcaacggtttggagaccgtttaaacggtctctacctcggggtacgcggtcgcgaatgtttgnnngcaggtgcaatggaacgcaccactagctttctgcattgcacctgcnntatgcagacccgcgagattccgtattgtttgaagagcgtttaaacgctcttcgccccgcgcgccgagggctgtattgtttcgagacggccctga

SEQ ID NO: 504 - DNA, 236 bp, artificial
  oligo
  misc_feature (93)..(95): n is a, c, g, or t
  misc_feature (141)..(142): n is a, c, g, or t
gaccgcgtctcacaccggtgacccgaggaaccggtgcgtttggagaccgtttaaacggtctctacctctcctgggaaccgcacgtgggtttgnnngcaggtgcaatgtctaggtaacccacgcatcgtcattgcacctgcnntatgcatcgggggacggttatttctggtttgaagagcgtttaaacgctcttcgcccgcgccagtgtttgcgctcccgtttcgagacggccctga

SEQ ID NO: 505 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctaccttatccggttacctcatagtcgtttgnnngcaggtgcaatgagcaaaagcgaaaccccggtgcattgcacctgcnntatgcacggccatgcctgccagcttcgtttgaagagcgtttaaacgctcttcgcccccggctgtcgacgccagtacgtttcgagacggccctga

SEQ ID NO: 506 - DNA, 236 bp, artificial
  oligo
  misc_feature (93)..(95): n is a, c, g, or t
  misc_feature (141)..(142): n is a, c, g, or t
gaccgcgtctcacaccgcaactttggctccgtgcgccgtttggagaccgtttaaacggtctctacctctcctgggaaccgcacgtgggtttgnnngcaggtgcaatgcccaatcttcgtcaaacgggtcattgcacctgcnntatgcagtgcgctgccgtgagttgccgtttgaagagcgtttaaacgctcttcgccctagcgaaatcgtacccgagcgtttcgagacggccctga

SEQ ID NO: 507 - DNA, 236 bp, artificial
  oligo
  misc_feature (93)..(95): n is a, c, g, or t
  misc_feature (141)..(142): n is a, c, g, or t
gaccgcgtctcacaccggctcgggatccagagatggggtttggagaccgtttaaacggtctctacctagggcacgtcaactctacacgtttgnnngcaggtgcaatgcgagcttctccctaagatcagcattgcacctgcnntatgcagccttgagcccggtggggctgtttgaagagcgtttaaacgctcttcgcccccttacgctgcgtccgcgttgtttcgagacggccctga

SEQ ID NO: 508 - DNA, 236 bp, artificial
  oligo
  misc_feature (93)..(95): n is a, c, g, or t
  misc_feature (141)..(142): n is a, c, g, or t
gaccgcgtctcacaccggaagcgagagaatctcctcggtttggagaccgtttaaacggtctctacctatcctccacgtgcggttcccgtttgnnngcaggtgcaatgtgcgcagacaagaggtatgcacattgcacctgcnntatgcattctcggaagtcgaggacaggtttgaagagcgtttaaacgctcttcgccccgtataagtcctaagcgacggtttcgagacggccctga

SEQ ID NO: 509 - DNA, 236 bp, artificial
  oligo
  misc_feature (93)..(95): n is a, c, g, or t
  misc_feature (141)..(142): n is a, c, g, or t
gaccgcgtctcacaccggcgccagtgtttgcgctcccgtttggagaccgtttaaacggtctctacctctcgtagcttgccacactgtgtttgnnngcaggtgcaatgcccattgaacgactcatgcgtcattgcacctgcnntatgcaccggatccgttatctccggagtttgaagagcgtttaaacgctcttcgcccactccttcgctcgaattgcggtttcgagacggccctga

SEQ ID NO: 510 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctacctttctcggaagtcgaggacaggtttgnnngcaggtgcaatgtgtcccgaagatacacccctgcattgcacctgcnntatgcatcaacgtagcgctattatcggtttgaagagcgtttaaacgctcttcgccccttggcgcgattcgggttgcgtttcgagacggccctga

SEQ ID NO: 511 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctacctgaaccgcacgtggaggatgcgtttgnnngcaggtgcaatgcctccaggtttaacacagcctcattgcacctgcnntatgcacccgaaagggcgaatcgcgggtttgaagagcgtttaaacgctcttcgccctgcgtcgcgacccccatatcgtttcgagacggccctga

SEQ ID NO: 512 - DNA, 236 bp, artificial
  oligo
  misc_feature (93)..(95): n is a, c, g, or t
  misc_feature (141)..(142): n is a, c, g, or t
gaccgcgtctcacaccggctaccagcaccggttcctcgtttggagaccgtttaaacggtctctacctcgcccagggagcgcaaacacgtttgnnngcaggtgcaatggcaaccgccaacctgattcatcattgcacctgcnntatgcacgtcggacgttacgtaacttgtttgaagagcgtttaaacgctcttcgccctcgtatgacaatcgcgaaacgtttcgagacggccctga

SEQ ID NO: 513 - DNA, 236 bp, artificial
  oligo
  misc_feature (93)..(95): n is a, c, g, or t
  misc_feature (141)..(142): n is a, c, g, or t
gaccgcgtctcacaccgcggccatgcctgccagcttcgtttggagaccgtttaaacggtctctacctgggccagggtctgcatatgcgtttgnnngcaggtgcaatgccactaggcctcatcaattcgcattgcacctgcnntatgcaagggtatcgaccgtcgccctgtttgaagagcgtttaaacgctcttcgcccactaacgacgtcgactagccgtttcgagacggccctga

SEQ ID NO: 514 - DNA, 236 bp, artificial
  oligo
  misc_feature (93)..(95): n is a, c, g, or t
  misc_feature (141)..(142): n is a, c, g, or t
gaccgcgtctcacaccgatcctccacgtgcggttcccgtttggagaccgtttaaacggtctctacctggccagaatacaggtcccgcgtttgnnngcaggtgcaatgagggactcagcaacgacaccacattgcacctgcnntatgcacgaccgaatctcgtgaacgggtttgaagagcgtttaaacgctcttcgccctcttcgacgctcggctaaacgtttcgagacggccctga

SEQ ID NO: 515 - DNA, 236 bp, artificial
  oligo
  misc_feature (93)..(95): n is a, c, g, or t
  misc_feature (141)..(142): n is a, c, g, or t
gaccgcgtctcacaccggtgcgctgccgtgagttgccgtttggagaccgtttaaacggtctctacctacttatcgttcgcgacgtaagtttgnnngcaggtgcaatgtgttcttatatcccagcgccccattgcacctgcnntatgcagtcacccggcggacttaagagtttgaagagcgtttaaacgctcttcgccccgccgatgacgtatggttgcgtttcgagacggccctga

SEQ ID NO: 516 - DNA, 236 bp, artificial
  oligo
  misc_feature (93)..(95): n is a, c, g, or t
  misc_feature (141)..(142): n is a, c, g, or t
gaccgcgtctcacaccgcggccatgcctgccagcttcgtttggagaccgtttaaacggtctctacctgcagcagtccgccgatttcggtttgnnngcaggtgcaatggcacatcacgcgaatacacctcattgcacctgcnntatgcatagatttcgcgcaccgtgtcgtttgaagagcgtttaaacgctcttcgcccctagcgcgccttccgagtatgtttcgagacggccctga

SEQ ID NO: 517 - DNA, 236 bp, artificial
  oligo
  misc_feature (93)..(95): n is a, c, g, or t
  misc_feature (141)..(142): n is a, c, g, or t
gaccgcgtctcacaccgccgagttacctcgagctttcgtttggagaccgtttaaacggtctctacctcgattccccgttacggtgatgtttgnnngcaggtgcaatgtcgcacgtacgatttactccccattgcacctgcnntatgcaggtacgtgtcatatacgtcggtttgaagagcgtttaaacgctcttcgcccgttgccggattacgtgtaatgtttcgagacggccctga

SEQ ID NO: 518 - DNA, 236 bp, artificial
  oligo
  misc_feature (93)..(95): n is a, c, g, or t
  misc_feature (141)..(142): n is a, c, g, or t
gaccgcgtctcacaccgttacattgtatacgccttccgtttggagaccgtttaaacggtctctacctcgcatttcgcgctcgtatcggtttgnnngcaggtgcaatgccaggaagaacaacgtagacccattgcacctgcnntatgcacgttattccgagactacgtcgtttgaagagcgtttaaacgctcttcgcccttatacgcgccgaagtggatgtttcgagacggccctga

SEQ ID NO: 519 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctaccttgaatgaccagggtacctgcgtttgnnngcaggtgcaatgggcaccttctcacctatgaaccattgcacctgcnntatgcaattcttctctgtgaatgtcggtttgaagagcgtttaaacgctcttcgccccctatcatacgtatcctggtgtttcgagacggccctga

SEQ ID NO: 520 - DNA, 236 bp, artificial
  oligo
  misc_feature (93)..(95): n is a, c, g, or t
  misc_feature (141)..(142): n is a, c, g, or t
gaccgcgtctcacaccgggccatttatagtgttgaccgtttggagaccgtttaaacggtctctaccttgggactcctcggttatttagtttgnnngcaggtgcaatgagcctctaatccagctctacccattgcacctgcnntatgcactaaagggcgatctcaacaagtttgaagagcgtttaaacgctcttcgccctctgggcatgtgggtccggcgtttcgagacggccctga

SEQ ID NO: 521 - DNA, 236 bp, artificial
  oligo
  misc_feature (93)..(95): n is a, c, g, or t
  misc_feature (141)..(142): n is a, c, g, or t
gaccgcgtctcacaccgtgggactcctcggttatttagtttggagaccgtttaaacggtctctacctacatagacccctgttgtaaggtttgnnngcaggtgcaatgcctcagcgacaaatcctaacccattgcacctgcnntatgcagctcaaaccattacagaagggtttgaagagcgtttaaacgctcttcgccccgtcaccaagccattgtaccgtttcgagacggccctga

SEQ ID NO: 522 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctacctcatcggcctgacccccatgcgtttgnnngcaggtgcaatgttacgcccaaaagcgacagagcattgcacctgcnntatgcacccaccaggatacgtatgatgtttgaagagcgtttaaacgctcttcgccctcatcctgcatgctagaccagtttcgagacggccctga

SEQ ID NO: 523 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctgctcaaaccattacagaagggtttgnnngcaggtgcaatgcccagactgaaagtactagcccattgcacctgcnntatgcagattccttccaataacacaagtttgaagagcgtttaaacgctcttcgcccctaaagggcgatctcaacaagtttcgagacggccctga

SEQ ID NO: 524 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctctgaaattagacatcaaagcgtttgnnngcaggtgcaatgcacatgcccgaattacctctccattgcacctgcnntatgcacaggcacgatagatacaaaggtttgaagagcgtttaaacgctcttcgccctgcccacaaagtatggcggtgtttcgagacggccctga

SEQ ID NO: 525 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctaccttgggactcctcggttatttagtttgnnngcaggtgcaatgccgaactgttacacaagcacgcattgcacctgcnntatgcagttacactctatctacacctgtttgaagagcgtttaaacgctcttcgccccctatcatacgtatcctggtgtttcgagacggccctga

SEQ ID NO: 526 - DNA, 236 bp, artificial
  oligo
  misc_feature (93)..(95): n is a, c, g, or t
  misc_feature (141)..(142): n is a, c, g, or t
gaccgcgtctcacaccgactttccatgtcagccatacgtttggagaccgtttaaacggtctctacctgattccttccaataacacaagtttgnnngcaggtgcaatgcgcgataccacgactagtacacattgcacctgcnntatgcatctgggcatgtgggtccggcgtttgaagagcgtttaaacgctcttcgcccagctgtagtagagtccccatgtttcgagacggccctga

SEQ ID NO: 527 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctaccttcacatggatggttactgaagtttgnnngcaggtgcaatgattcagtagaccgttcgtcggcattgcacctgcnntatgcaagcacacaagcacgttacccgtttgaagagcgtttaaacgctcttcgcccatcagttctgagcaactcgtgtttcgagacggccctga

SEQ ID NO: 528 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctaccttgtgatggtgaatattcacagtttgnnngcaggtgcaatggatcacgttgaaacccgactgcattgcacctgcnntatgcaccctcaccttctacccagccgtttgaagagcgtttaaacgctcttcgccctgcaagccttacctgtagcggtttcgagacggccctga

SEQ ID NO: 529 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctcagcttgtccaactggtcgggtttgnnngcaggtgcaatgtgtagtacccaggcagcagctcattgcacctgcnntatgcatgttgagatcgccctttagcgtttgaagagcgtttaaacgctcttcgccccaggcacgatagatacaaaggtttcgagacggccctga

SEQ ID NO: 530 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctttgggactcctcggttatttgtttgnnngcaggtgcaatggacttaacggaagttaccgcgcattgcacctgcnntatgcacgtcaccaagccattgtaccgtttgaagagcgtttaaacgctcttcgcccacacaacactgatgaggtccgtttcgagacggccctga

SEQ ID NO: 531 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctagcacacaagcacgttacccgtttgnnngcaggtgcaatgccagtctcaccaaacgaagggcattgcacctgcnntatgcaattcttctctgtgaatgtcggtttgaagagcgtttaaacgctcttcgccccagcttgtccaactggtcgggtttcgagacggccctga

SEQ ID NO: 532 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctagaagttggtgacgttctgcgtttgnnngcaggtgcaatgactaaggcccacagcgagaaacattgcacctgcnntatgcatgcccacaaagtatggcggtgtttgaagagcgtttaaacgctcttcgcccggccatttatagtgttgaccgtttcgagacggccctga

SEQ ID NO: 533 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctacctacaggtgacccaaccttcaggtttgnnngcaggtgcaatgtcggatactccaagctagctccattgcacctgcnntatgcactgcagttgagctggcaatcgtttgaagagcgtttaaacgctcttcgcccttgttacagccttattacacgtttcgagacggccctga

SEQ ID NO: 534 - DNA, 236 bp, artificial
  oligo
  misc_feature (93)..(95): n is a, c, g, or t
  misc_feature (141)..(142): n is a, c, g, or t
gaccgcgtctcacaccgctgcagttgagctggcaatcgtttggagaccgtttaaacggtctctacctttgggactcctcggttatttgtttgnnngcaggtgcaatgtcagccgccaggaaaaagaaccattgcacctgcnntatgcacccaccaggatacgtatgatgtttgaagagcgtttaaacgctcttcgcccgtccgttgggtataacaacggtttcgagacggccctga

SEQ ID NO: 535 - DNA, 236 bp, artificial
  oligo
  misc_feature (93)..(95): n is a, c, g, or t
  misc_feature (141)..(142): n is a, c, g, or t
gaccgcgtctcacaccgtgaatgaccagggtacctgcgtttggagaccgtttaaacggtctctacctttgggactcctcggttatttgtttgnnngcaggtgcaatgccgtccaccagactatacgcacattgcacctgcnntatgcaggactgagagctgttgacacgtttgaagagcgtttaaacgctcttcgcccttgatcgggttcgtcggagcgtttcgagacggccctga

SEQ ID NO: 536 - DNA, 236 bp, artificial
  oligo
  misc_feature (93)..(95): n is a, c, g, or t
  misc_feature (141)..(142): n is a, c, g, or t
gaccgcgtctcacaccgccctcaccttctacccagccgtttggagaccgtttaaacggtctctaccttcatcctgcatgctagaccagtttgnnngcaggtgcaatgcgactcatacatcgacagctgcattgcacctgcnntatgcagccctttagctggtatctgcgtttgaagagcgtttaaacgctcttcgcccagcgtaaacgtcgtccgcgagtttcgagacggccctga

SEQ ID NO: 537 - DNA, 236 bp, artificial
  oligo
  misc_feature (93)..(95): n is a, c, g, or t
  misc_feature (141)..(142): n is a, c, g, or t
gaccgcgtctcacaccgtctgggcatgtgggtccggcgtttggagaccgtttaaacggtctctaccttctttgaaccattgtgttatgtttgnnngcaggtgcaatggagcaatcacgatgaccacaccattgcacctgcnntatgcaagaagttggtgacgttctgcgtttgaagagcgtttaaacgctcttcgcccacgggccgttccggaccgttgtttcgagacggccctga

SEQ ID NO: 538 - DNA, 236 bp, artificial
  oligo
  misc_feature (93)..(95): n is a, c, g, or t
  misc_feature (141)..(142): n is a, c, g, or t
gaccgcgtctcacaccgtcacatggatggttactgaagtttggagaccgtttaaacggtctctaccttgcaagccttacctgtagcggtttgnnngcaggtgcaatgtcacttccagccccaggaaatcattgcacctgcnntatgcaacacaacactgatgaggtccgtttgaagagcgtttaaacgctcttcgccctcgtaagcggtcgaaaacgagtttcgagacggccctga

SEQ ID NO: 539 - DNA, 236 bp, artificial
  oligo
  misc_feature (93)..(95): n is a, c, g, or t
  misc_feature (141)..(142): n is a, c, g, or t
gaccgcgtctcacaccggtgacttggtgtaagcacaagtttggagaccgtttaaacggtctctacctatcagttctgagcaactcgtgtttgnnngcaggtgcaatgccgcaccttgttactaacccacattgcacctgcnntatgcactggaaggtgaggcctaggcgtttgaagagcgtttaaacgctcttcgccctagcgaccgtaccgccgattgtttcgagacggccctga

SEQ ID NO: 540 - DNA, 236 bp, artificial
  oligo
  misc_feature (93)..(95): n is a, c, g, or t
  misc_feature (141)..(142): n is a, c, g, or t
gaccgcgtctcacaccgttcatagacctaatgtgactgtttggagaccgtttaaacggtctctacctcaggcacgatagatacaaaggtttgnnngcaggtgcaatgctcacgagctaattgcctacccattgcacctgcnntatgcacgtcaccaagccattgtaccgtttgaagagcgtttaaacgctcttcgccccgcggtcgctagcgaaggaagtttcgagacggccctga

SEQ ID NO: 541 - DNA, 236 bp, artificial
  oligo
  misc_feature (93)..(95): n is a, c, g, or t
  misc_feature (141)..(142): n is a, c, g, or t
gaccgcgtctcacaccgtgttgagatcgccctttagcgtttggagaccgtttaaacggtctctacctgtgacttggtgtaagcacaagtttgnnngcaggtgcaatgcaaaccaccggatcgcactaccattgcacctgcnntatgcaacacaacactgatgaggtccgtttgaagagcgtttaaacgctcttcgccccgacgagtttgtctcgataggtttcgagacggccctga

SEQ ID NO: 542 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctacatagacccctgttgtaaggtttgnnngcaggtgcaatgccatacgttagaccctaaccgcattgcacctgcnntatgcagtgacttggtgtaagcacaagtttgaagagcgtttaaacgctcttcgccccggatgttacctcggtagacgtttcgagacggccctga

SEQ ID NO: 543 - DNA, 236 bp, artificial
  oligo
  misc_feature (93)..(95): n is a, c, g, or t
  misc_feature (141)..(142): n is a, c, g, or t
gaccgcgtctcacaccggttacactctatctacacctgtttggagaccgtttaaacggtctctacctcaacttcttcgtggtatctcgtttgnnngcaggtgcaatgtactatgacgaccaccgcctccattgcacctgcnntatgcatcacatggatggttactgaagtttgaagagcgtttaaacgctcttcgccctcgtatcgggcgaattcctagtttcgagacggccctga

SEQ ID NO: 544 - DNA, 236 bp, artificial
  oligo
  misc_feature (93)..(95): n is a, c, g, or t
  misc_feature (141)..(142): n is a, c, g, or t
gaccgcgtctcacaccgctggaaggtgaggcctaggcgtttggagaccgtttaaacggtctctacctacatagacccctgttgtaaggtttgnnngcaggtgcaatggatcagcacgaaaactagcggcattgcacctgcnntatgcatgaatgaccagggtacctgcgtttgaagagcgtttaaacgctcttcgccccccgtatcgccgagcaaactgtttcgagacggccctga

SEQ ID NO: 545 - DNA, 236 bp, artificial
  oligo
  misc_feature (93)..(95): n is a, c, g, or t
  misc_feature (141)..(142): n is a, c, g, or t
gaccgcgtctcacaccgcccaccaggatacgtatgatgtttggagaccgtttaaacggtctctacctcagcttgtccaactggtcgggtttgnnngcaggtgcaatgctgacgctacagacactagaccattgcacctgcnntatgcattgttacagccttattacacgtttgaagagcgtttaaacgctcttcgccccgggattatcgatggacggtgtttcgagacggccctga

SEQ ID NO: 546 - DNA, 236 bp, artificial
  oligo
  misc_feature (93)..(95): n is a, c, g, or t
  misc_feature (141)..(142): n is a, c, g, or t
gaccgcgtctcacaccgagaagttggtgacgttctgcgtttggagaccgtttaaacggtctctaccttgttgggcagctgtatgatcgtttgnnngcaggtgcaatggccccatctctgaaatccactcattgcacctgcnntatgcatgcaagccttacctgtagcggtttgaagagcgtttaaacgctcttcgcccgacgatcgcgcgcatatatcgtttcgagacggccctga

SEQ ID NO: 547 - DNA, 236 bp, artificial
  oligo
  misc_feature (93)..(95): n is a, c, g, or t
  misc_feature (141)..(142): n is a, c, g, or t
gaccgcgtctcacaccgttgttacagccttattacacgtttggagaccgtttaaacggtctctacctattcttctctgtgaatgtcggtttgnnngcaggtgcaatgcgcattgtcaacgcaacttcccattgcacctgcnntatgcacaatacagggatacccactagtttgaagagcgtttaaacgctcttcgcccaccgattccccgttcggactgtttcgagacggccctga

SEQ ID NO: 548 - DNA, 236 bp, artificial
  oligo
  misc_feature (93)..(95): n is a, c, g, or t
  misc_feature (141)..(142): n is a, c, g, or t
gaccgcgtctcacaccgtgttgggcagctgtatgatcgtttggagaccgtttaaacggtctctaccttgtcgatggcaatagccaaggtttgnnngcaggtgcaatgggatttaacacgctacgagcgcattgcacctgcnntatgcaggactgagagctgttgacacgtttgaagagcgtttaaacgctcttcgcccgagcgtcgtcgcacactagtgtttcgagacggccctga

SEQ ID NO: 549 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctctgaaattagacatcaaagcgtttgnnngcaggtgcaatgcgctctcctacagactatagccattgcacctgcnntatgcatgttgggcagctgtatgatcgtttgaagagcgtttaaacgctcttcgcccttgtacgcttcgggcgcgatgtttcgagacggccctga

SEQ ID NO: 550 - DNA, 236 bp, artificial
  oligo
  misc_feature (93)..(95): n is a, c, g, or t
  misc_feature (141)..(142): n is a, c, g, or t
gaccgcgtctcacaccgtgcaagccttacctgtagcggtttggagaccgtttaaacggtctctacctctaaagggcgatctcaacaagtttgnnngcaggtgcaatgactttcctatgtccgccactgcattgcacctgcnntatgcatgcccacaaagtatggcggtgtttgaagagcgtttaaacgctcttcgccccctgctacgcgtctacacgtgtttcgagacggccctga

SEQ ID NO: 551 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctacctacaggtgacccaaccttcaggtttgnnngcaggtgcaatgtacgcacatcccatcctagagcattgcacctgcnntatgcaacctatcatacgtatcctgggtttgaagagcgtttaaacgctcttcgcccaccgactgcgatctaaccgcgtttcgagacggccctga

SEQ ID NO: 552 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctaccttgtcgatggcaatagccaaggtttgnnngcaggtgcaatgcgtgagacaaactcaaccggacattgcacctgcnntatgcattgttacagccttattacacgtttgaagagcgtttaaacgctcttcgccctcaacgcgcgtagggcggatgtttcgagacggccctga

SEQ ID NO: 553 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctcgtcaccaagccattgtaccgtttgnnngcaggtgcaatggagctactcgcctttagatgccattgcacctgcnntatgcaactttccatgtcagccatacgtttgaagagcgtttaaacgctcttcgcccagtacgtcccgatggtatcggtttcgagacggccctga

SEQ ID NO: 554 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctaccttgaatgaccagggtacctgcgtttgnnngcaggtgcaatgtggcttgtcgtgacgaaacaccattgcacctgcnntatgcaggccgcggataatttcgccagtttgaagagcgtttaaacgctcttcgccctccccgtcggagtcgtacgcgtttcgagacggccctga

SEQ ID NO: 555 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctggccatttatagtgttgaccgtttgnnngcaggtgcaatggagatccaccgcattaagtgccattgcacctgcnntatgcagacttcgacaccggccgtttgtttgaagagcgtttaaacgctcttcgccctatgctacgctcgtcgtgcggtttcgagacggccctga

SEQ ID NO: 556 - DNA, 236 bp, artificial
  oligo
  misc_feature (93)..(95): n is a, c, g, or t
  misc_feature (141)..(142): n is a, c, g, or t
gaccgcgtctcacaccgctgctagccaacaccactgagtttggagaccgtttaaacggtctctacctgattccttccaataacacaagtttgnnngcaggtgcaatgctccactagagccacttccgacattgcacctgcnntatgcaactatgcggtaggtacgccggtttgaagagcgtttaaacgctcttcgcccgtgcggacgttacacccgatgtttcgagacggccctga

SEQ ID NO: 557 - DNA, 236 bp, artificial
  oligo
  misc_feature (93)..(95): n is a, c, g, or t
  misc_feature (141)..(142): n is a, c, g, or t
gaccgcgtctcacaccgatcagttctgagcaactcgtgtttggagaccgtttaaacggtctctacctggaagggacactaacctggtgtttgnnngcaggtgcaatggacgcagcacataggtaagtgcattgcacctgcnntatgcaagcggcgtaagacgattatcgtttgaagagcgtttaaacgctcttcgccccctcatgaccgctcgccgatgtttcgagacggccctga

SEQ ID NO: 558 - DNA, 236 bp, artificial
  oligo
  misc_feature (93)..(95): n is a, c, g, or t
  misc_feature (141)..(142): n is a, c, g, or t
gaccgcgtctcacaccgtctttgaaccattgtgttatgtttggagaccgtttaaacggtctctacctcatcggcctgacccccatgcgtttgnnngcaggtgcaatggaccaacacaatctcgtgacgcattgcacctgcnntatgcatacgaatcgtcagagcggatgtttgaagagcgtttaaacgctcttcgcccgctacggtagacgtcgacaagtttcgagacggccctga

SEQ ID NO: 559 - DNA, 236 bp, artificial
  oligo
  misc_feature (93)..(95): n is a, c, g, or t
  misc_feature (141)..(142): n is a, c, g, or t
gaccgcgtctcacaccggtatcctggtgggatttacagtttggagaccgtttaaacggtctctacctcagcttgtccaactggtcgggtttgnnngcaggtgcaatgcactagaattgcagcaacgcccattgcacctgcnntatgcagtcccttactacgtacgcgggtttgaagagcgtttaaacgctcttcgcccaaggctcgatagttcgcgacgtttcgagacggccctga

SEQ ID NO: 560 - DNA, 236 bp, artificial
  oligo
  misc_feature (93)..(95): n is a, c, g, or t
  misc_feature (141)..(142): n is a, c, g, or t
gaccgcgtctcacaccgacaggtgacccaaccttcaggtttggagaccgtttaaacggtctctacctcaatacagggatacccactagtttgnnngcaggtgcaatgaccttccagcctaacctcaagcattgcacctgcnntatgcacgaacgatatccgaatgcacgtttgaagagcgtttaaacgctcttcgccctcggtactttgaaaccgcgcgtttcgagacggccctga

SEQ ID NO: 561 - DNA, 236 bp, artificial
  oligo
  misc_feature (93)..(95): n is a, c, g, or t
  misc_feature (141)..(142): n is a, c, g, or t
gaccgcgtctcacaccgctgaaattagacatcaaagcgtttggagaccgtttaaacggtctctacctctgcagttgagctggcaatcgtttgnnngcaggtgcaatgtaatatcgtcccctacctcggcattgcacctgcnntatgcatgtaacgtcttaagccgcgggtttgaagagcgtttaaacgctcttcgcccggtttgcgctaacgtgtggcgtttcgagacggccctga

SEQ ID NO: 562 - DNA, 236 bp, artificial
  oligo
  misc_feature (93)..(95): n is a, c, g, or t
  misc_feature (141)..(142): n is a, c, g, or t
gaccgcgtctcacaccgagcacacaagcacgttacccgtttggagaccgtttaaacggtctctacctccctcaccttctacccagccgtttgnnngcaggtgcaatggctgggaaagctattacctcgcattgcacctgcnntatgcacgaacgacgaggtactagcggtttgaagagcgtttaaacgctcttcgcccaactcgcttcgtcggatatagtttcgagacggccctga

SEQ ID NO: 563 - DNA, 236 bp, artificial
  oligo
  misc_feature (93)..(95): n is a, c, g, or t
  misc_feature (141)..(142): n is a, c, g, or t
gaccgcgtctcacaccgacctatcatacgtatcctgggtttggagaccgtttaaacggtctctacctctgctagccaacaccactgagtttgnnngcaggtgcaatgacaggaaatcagccaagaccgcattgcacctgcnntatgcagttcgtcaagtccgccgcaggtttgaagagcgtttaaacgctcttcgcccccgcccacgatattggcggagtttcgagacggccctga

SEQ ID NO: 564 - DNA, 236 bp, artificial
  oligo
  misc_feature (93)..(95): n is a, c, g, or t
  misc_feature (141)..(142): n is a, c, g, or t
gaccgcgtctcacaccgaatcccttgccccagcacatgtttggagaccgtttaaacggtctctaccttcacccataggagccacagcgtttgnnngcaggtgcaatgtagcagggtaccgtactgacacattgcacctgcnntatgcaaacgatagcgcgcttaagcggtttgaagagcgtttaaacgctcttcgcccggttgaccgcgaccgttcgggtttcgagacggccctga

SEQ ID NO: 565 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctagcacacaagcacgttacccgtttgnnngcaggtgcaatgtctttcctctaagcccccagacattgcacctgcnntatgcactcgacgcaaccgcagatcggtttgaagagcgtttaaacgctcttcgccccgtttaagttggtaccccgcgtttcgagacggccctga

SEQ ID NO: 566 - DNA, 236 bp, artificial
  oligo
  misc_feature (93)..(95): n is a, c, g, or t
  misc_feature (141)..(142): n is a, c, g, or t
gaccgcgtctcacaccgctaaagggcgatctcaacaagtttggagaccgtttaaacggtctctacctacatagacccctgttgtaaggtttgnnngcaggtgcaatgtaggaactcacccgtaagccgcattgcacctgcnntatgcaatattgccggcacgatagatgtttgaagagcgtttaaacgctcttcgccctgacaacgcggatttggtcggtttcgagacggccctga

SEQ ID NO: 567 - DNA, 236 bp, artificial
  oligo
  misc_feature (93)..(95): n is a, c, g, or t
  misc_feature (141)..(142): n is a, c, g, or t
gaccgcgtctcacaccgtgtcgatggcaatagccaaggtttggagaccgtttaaacggtctctacctacacaacactgatgaggtccgtttgnnngcaggtgcaatgtgactacacctacagcgcacccattgcacctgcnntatgcaccgccagttgcaatcgtacagtttgaagagcgtttaaacgctcttcgccccgccatacgaggcgaaagcagtttcgagacggccctga

SEQ ID NO: 568 - DNA, 236 bp, artificial
  oligo
  misc_feature (93)..(95): n is a, c, g, or t
  misc_feature (141)..(142): n is a, c, g, or t
gaccgcgtctcacaccgctccacaaagacctgcggcggtttggagaccgtttaaacggtctctacctagctgtagtagagtccccatgtttgnnngcaggtgcaatgagcccgaaataacctgaagggcattgcacctgcnntatgcattgtgaccgttcgatgttcggtttgaagagcgtttaaacgctcttcgccctgacgcccctcgacggcatagtttcgagacggccctga

SEQ ID NO: 569 - DNA, 236 bp, artificial
  oligo
  misc_feature (93)..(95): n is a, c, g, or t
  misc_feature (141)..(142): n is a, c, g, or t
gaccgcgtctcacaccgtcatcctgcatgctagaccagtttggagaccgtttaaacggtctctacctgatccactcgcggaacttacgtttgnnngcaggtgcaatgcaccgcagcttacatacaggacattgcacctgcnntatgcatacgatccgcgcgcgcgggagtttgaagagcgtttaaacgctcttcgcccagtcgttcggcccgtctagtgtttcgagacggccctga

SEQ ID NO: 570 - DNA, 236 bp, artificial
  oligo
  misc_feature (93)..(95): n is a, c, g, or t
  misc_feature (141)..(142): n is a, c, g, or t
gaccgcgtctcacaccgagatcatacagctgcccaacgtttggagaccgtttaaacggtctctacctcggacgtcccgtatcgggatgtttgnnngcaggtgcaatgcaggcaactcagtcacctatccattgcacctgcnntatgcaatcccgtggcgatgtatcgagtttgaagagcgtttaaacgctcttcgccctcgcgaacgctcggagatgtgtttcgagacggccctga

SEQ ID NO: 571 - DNA, 236 bp, artificial
  oligo
  misc_feature (93)..(95): n is a, c, g, or t
  misc_feature (141)..(142): n is a, c, g, or t
gaccgcgtctcacaccgtcacccataggagccacagcgtttggagaccgtttaaacggtctctacctcgttgtagtccgcgcgcgacgtttgnnngcaggtgcaatggaaacgcacctcaagaaacggcattgcacctgcnntatgcaccgaatgtgcgctaaatcgagtttgaagagcgtttaaacgctcttcgcccgttaaactaaatcggcgcgcgtttcgagacggccctga

SEQ ID NO: 572 - DNA, 236 bp, artificial
  oligo
  misc_feature (93)..(95): n is a, c, g, or t
  misc_feature (141)..(142): n is a, c, g, or t
gaccgcgtctcacaccgacatagacccctgttgtaaggtttggagaccgtttaaacggtctctaccttgcgtcaatcgcagcgtctagtttgnnngcaggtgcaatgccgttccaaacacaaacacggcattgcacctgcnntatgcacttaaattcccgcgccgcgagtttgaagagcgtttaaacgctcttcgccccaggtacggccgacgagctagtttcgagacggccctga

SEQ ID NO: 573 - DNA, 236 bp, artificial
  oligo
  misc_feature (93)..(95): n is a, c, g, or t
  misc_feature (141)..(142): n is a, c, g, or t
gaccgcgtctcacaccgagctgtagtagagtccccatgtttggagaccgtttaaacggtctctacctgcgcgacacatgggcggttcgtttgnnngcaggtgcaatgcccatagcccctaagattctccattgcacctgcnntatgcatagacggacgttactgcacggtttgaagagcgtttaaacgctcttcgccctcgacgacgataaaagcgctgtttcgagacggccctga

SEQ ID NO: 574 - DNA, 236 bp, artificial
  oligo
  misc_feature (93)..(95): n is a, c, g, or t
  misc_feature (141)..(142): n is a, c, g, or t
gaccgcgtctcacaccgcatcggcctgacccccatgcgtttggagaccgtttaaacggtctctaccttccgcatttcgcgcgtgcaagtttgnnngcaggtgcaatggcatactctgctactcgaccacattgcacctgcnntatgcaccgaataccgagtcgttacggtttgaagagcgtttaaacgctcttcgcccatgcgcgcgctggcgctgtagtttcgagacggccctga

SEQ ID NO: 575 - DNA, 236 bp, artificial
  oligo
  misc_feature (93)..(95): n is a, c, g, or t
  misc_feature (141)..(142): n is a, c, g, or t
gaccgcgtctcacaccgccaagtaccttctccagcatgtttggagaccgtttaaacggtctctacctccacagtactcacttggtccgtttgnnngcaggtgcaatgccgaccggaaaacccttactgcattgcacctgcnntatgcagagaagcactatgggggcatgtttgaagagcgtttaaacgctcttcgccctggcattggggacctcgggtgtttcgagacggccctga

SEQ ID NO: 576 - DNA, 236 bp, artificial
  oligo
  misc_feature (93)..(95): n is a, c, g, or t
  misc_feature (141)..(142): n is a, c, g, or t
gaccgcgtctcacaccgttcttctcagttcaagtaacgtttggagaccgtttaaacggtctctaccttcacacttggagttacagcagtttgnnngcaggtgcaatgagatgttgcacacccaactcccattgcacctgcnntatgcaaccaggctgacagttatcacgtttgaagagcgtttaaacgctcttcgcccgtgtctgtatgacccacccggtttcgagacggccctga

SEQ ID NO: 577 - DNA, 236 bp, artificial
  oligo
  misc_feature (93)..(95): n is a, c, g, or t
  misc_feature (141)..(142): n is a, c, g, or t
gaccgcgtctcacaccgacctgtggtggttatggggcgtttggagaccgtttaaacggtctctacctatccccgtcgcagttgaactgtttgnnngcaggtgcaatgcctgattaagcacttagccgccattgcacctgcnntatgcattattcttgtccaggcaagggtttgaagagcgtttaaacgctcttcgccctaagctgctctctagacggagtttcgagacggccctga

SEQ ID NO: 578 - DNA, 236 bp, artificial
  oligo
  misc_feature (93)..(95): n is a, c, g, or t
  misc_feature (141)..(142): n is a, c, g, or t
gaccgcgtctcacaccgccacagtactcacttggtccgtttggagaccgtttaaacggtctctaccttaaccatagtacccagttgtgtttgnnngcaggtgcaatgccccagctcagaaagacttagcattgcacctgcnntatgcagtcacacctgagtaccactggtttgaagagcgtttaaacgctcttcgcccgtggaagtctgtgtcgggaagtttcgagacggccctga

SEQ ID NO: 579 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctaccttcacacttggagttacagcagtttgnnngcaggtgcaatgctcgaataatagacgctgccgcattgcacctgcnntatgcataagctgctctctagacggagtttgaagagcgtttaaacgctcttcgcccggggcatgggccatgctgtagtttcgagacggccctga

SEQ ID NO: 580 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctgtgcatacgtacttcgtccagtttgnnngcaggtgcaatgctgacacccaacgaagtgactcattgcacctgcnntatgcagttcactacgagtgtattccgtttgaagagcgtttaaacgctcttcgcccgtatatgccgctacgcctttgtttcgagacggccctga

SEQ ID NO: 581 - DNA, 236 bp, artificial
  oligo
  misc_feature (93)..(95): n is a, c, g, or t
  misc_feature (141)..(142): n is a, c, g, or t
gaccgcgtctcacaccgtaaccatagtacccagttgtgtttggagaccgtttaaacggtctctacctgcaaagccgatgtcagcgccgtttgnnngcaggtgcaatgctcctgttccatctataccggcattgcacctgcnntatgcatcctcagctgccatatgtgcgtttgaagagcgtttaaacgctcttcgcccgcgaccgcacttccgttctagtttcgagacggccctga

SEQ ID NO: 582 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctagccacgcttgctgatgttggtttgnnngcaggtgcaatgacggacgaatgtaaaagcccccattgcacctgcnntatgcaatccccgtcgcagttgaactgtttgaagagcgtttaaacgctcttcgccctaccgtacgtggaactccgcgtttcgagacggccctga

SEQ ID NO: 583 - DNA, 236 bp, artificial
  oligo
  misc_feature (93)..(95): n is a, c, g, or t
  misc_feature (141)..(142): n is a, c, g, or t
gaccgcgtctcacaccgtggcattggggacctcgggtgtttggagaccgtttaaacggtctctaccttaccttgagactccaccctcgtttgnnngcaggtgcaatgacaacaccatattgagcggcgcattgcacctgcnntatgcagtagaccaggcacccaacctgtttgaagagcgtttaaacgctcttcgcccattcgcgttatcgaacacgtgtttcgagacggccctga

SEQ ID NO: 584 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctaccttgctgtcgtgagtgccagccgtttgnnngcaggtgcaatgcacatgtccttacgccaatcccattgcacctgcnntatgcagagaggtttccgaagactaagtttgaagagcgtttaaacgctcttcgccccggacgtagcaaccgttgaagtttcgagacggccctga

SEQ ID NO: 585 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctacctctagcaagccataacaaaacgtttgnnngcaggtgcaatggcgcgacaactcaagttatgccattgcacctgcnntatgcaaaccccaacatcagcaagcggtttgaagagcgtttaaacgctcttcgccccgattccccgttacggtgatgtttcgagacggccctga

SEQ ID NO: 586 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctaagcactacccctgccaaccgtttgnnngcaggtgcaatgcctaagaccgcacccattgaccattgcacctgcnntatgcacagcttcaggaccccgatccgtttgaagagcgtttaaacgctcttcgcccttcaactaacgcgcccgctagtttcgagacggccctga

SEQ ID NO: 587 - DNA, 236 bp, artificial
  oligo
  misc_feature (93)..(95): n is a, c, g, or t
  misc_feature (141)..(142): n is a, c, g, or t
gaccgcgtctcacaccggtgcatacgtacttcgtccagtttggagaccgtttaaacggtctctacctttcttctcagttcaagtaacgtttgnnngcaggtgcaatgagccaggaccacaattcccatcattgcacctgcnntatgcatcccacagttgtaccttatcgtttgaagagcgtttaaacgctcttcgccctttggccgcgcgctcgatacgtttcgagacggccctga

SEQ ID NO: 588 - DNA, 236 bp, artificial
  oligo
  misc_feature (93)..(95): n is a, c, g, or t
  misc_feature (141)..(142): n is a, c, g, or t
gaccgcgtctcacaccggtggaagtctgtgtcgggaagtttggagaccgtttaaacggtctctacctgccatgggcaccttctccgcgtttgnnngcaggtgcaatgcgaacaacttcctccacttcccattgcacctgcnntatgcatgtgaaaaggactgcaggccgtttgaagagcgtttaaacgctcttcgcccttatcgcgtcgtgatcggcagtttcgagacggccctga

SEQ ID NO: 589 - DNA, 236 bp, artificial
  oligo
  misc_feature (93)..(95): n is a, c, g, or t
  misc_feature (141)..(142): n is a, c, g, or t
gaccgcgtctcacaccgctagcaagccataacaaaacgtttggagaccgtttaaacggtctctaccttcctgccgaggttgggtgccgtttgnnngcaggtgcaatgcgataataccacccacggtcgcattgcacctgcnntatgcagtcacacctgagtaccactggtttgaagagcgtttaaacgctcttcgccccgaagttgccgaatgcgaatgtttcgagacggccctga

SEQ ID NO: 590 - DNA, 236 bp, artificial
  oligo
  misc_feature (93)..(95): n is a, c, g, or t
  misc_feature (141)..(142): n is a, c, g, or t
gaccgcgtctcacaccggcaaagccgatgtcagcgccgtttggagaccgtttaaacggtctctacctatcttgcagatgctaatagcgtttgnnngcaggtgcaatgcgaacccgctacagcttagtccattgcacctgcnntatgcaccgtcgttcccttacggatagtttgaagagcgtttaaacgctcttcgcccgggaccgatccattaccgtagtttcgagacggccctga

SEQ ID NO: 591 - DNA, 236 bp, artificial
  oligo
  misc_feature (93)..(95): n is a, c, g, or t
  misc_feature (141)..(142): n is a, c, g, or t
gaccgcgtctcacaccgtcctgccgaggttgggtgccgtttggagaccgtttaaacggtctctaccttaaccatagtacccagttgtgtttgnnngcaggtgcaatggcgcaacacggtgaaaagatgcattgcacctgcnntatgcacgccgaatattagcccgaacgtttgaagagcgtttaaacgctcttcgccctgcgattgcggcgactaaacgtttcgagacggccctga

SEQ ID NO: 592 - DNA, 236 bp, artificial
  oligo
  misc_feature (93)..(95): n is a, c, g, or t
  misc_feature (141)..(142): n is a, c, g, or t
gaccgcgtctcacaccgagccacgcttgctgatgttggtttggagaccgtttaaacggtctctacctgagaagcactatgggggcatgtttgnnngcaggtgcaatgcgaacccttgtgccaacataccattgcacctgcnntatgcattaagcgaccgcgatactccgtttgaagagcgtttaaacgctcttcgcccaccgaccaaattccccggcggtttcgagacggccctga

SEQ ID NO: 593 - DNA, 236 bp, artificial
  oligo
  misc_feature (93)..(95): n is a, c, g, or t
  misc_feature (141)..(142): n is a, c, g, or t
gaccgcgtctcacaccgaacatgactccccggaggccgtttggagaccgtttaaacggtctctaccttggcattggggacctcgggtgtttgnnngcaggtgcaatgcctatatggtccctatccccacattgcacctgcnntatgcacggctatacggtgtagcaacgtttgaagagcgtttaaacgctcttcgcccgataccgaaacgatgtcgtcgtttcgagacggccctga

SEQ ID NO: 594 - DNA, 236 bp, artificial
  oligo
  misc_feature (93)..(95): n is a, c, g, or t
  misc_feature (141)..(142): n is a, c, g, or t
gaccgcgtctcacaccgtaagctgctctctagacggagtttggagaccgtttaaacggtctctacctgagccgctgtgatcataccagtttgnnngcaggtgcaatgcccctagcaatatggacaacgcattgcacctgcnntatgcaatcgcgttgttgcatgacgggtttgaagagcgtttaaacgctcttcgcccacgcgcgtaacctgtcgcacgtttcgagacggccctga

SEQ ID NO: 595 - DNA, 236 bp, artificial
  oligo
  misc_feature (93)..(95): n is a, c, g, or t
  misc_feature (141)..(142): n is a, c, g, or t
gaccgcgtctcacaccgaccaggctgacagttatcacgtttggagaccgtttaaacggtctctacctgccatgggcaccttctccgcgtttgnnngcaggtgcaatgacacctgttcacaactccaggcattgcacctgcnntatgcagggccgtgcacgcgttttaggtttgaagagcgtttaaacgctcttcgccctagcccgatccgagcgcatagtttcgagacggccctga

SEQ ID NO: 596 - DNA, 236 bp, artificial
  oligo
  misc_feature (93)..(95): n is a, c, g, or t
  misc_feature (141)..(142): n is a, c, g, or t
gaccgcgtctcacaccggcaggtccttaccttgttccgtttggagaccgtttaaacggtctctacctgtggaagtctgtgtcgggaagtttgnnngcaggtgcaatgcgaatccgcgacattctaccgcattgcacctgcnntatgcagatcgacaaatataacgcgcgtttgaagagcgtttaaacgctcttcgcccggacgcccggcgcgtctaacgtttcgagacggccctga

SEQ ID NO: 597 - DNA, 236 bp, artificial
  oligo
  misc_feature (93)..(95): n is a, c, g, or t
  misc_feature (141)..(142): n is a, c, g, or t
gaccgcgtctcacaccgtaccttgagactccaccctcgtttggagaccgtttaaacggtctctaccttcacacttggagttacagcagtttgnnngcaggtgcaatgctcgacaccttaaagcggcaacattgcacctgcnntatgcactagcggcgcaatacgtgctgtttgaagagcgtttaaacgctcttcgcccgtcggcgataagatggacgcgtttcgagacggccctga

SEQ ID NO: 598 - DNA, 236 bp, artificial
  oligo
  misc_feature (93)..(95): n is a, c, g, or t
  misc_feature (141)..(142): n is a, c, g, or t
gaccgcgtctcacaccgtcccacagttgtaccttatcgtttggagaccgtttaaacggtctctacctttcctacaaccttctcgcaagtttgnnngcaggtgcaatggccccgaactcaatcattccacattgcacctgcnntatgcacgaccggccacatcgactaggtttgaagagcgtttaaacgctcttcgccccgttacgcctccgccgttccgtttcgagacggccctga

SEQ ID NO: 599 - DNA, 236 bp, artificial
  oligo
  misc_feature (93)..(95): n is a, c, g, or t
  misc_feature (141)..(142): n is a, c, g, or t
gaccgcgtctcacaccgttattcttgtccaggcaagggtttggagaccgtttaaacggtctctaccttacgctgcaaagtctctggcgtttgnnngcaggtgcaatgcccccaactaaagtagtccgacattgcacctgcnntatgcaacgacggcattcatctcgatgtttgaagagcgtttaaacgctcttcgccccgctcgtggctacgacgattgtttcgagacggccctga

SEQ ID NO: 600 - DNA, 236 bp, artificial
  oligo
  misc_feature (93)..(95): n is a, c, g, or t
  misc_feature (141)..(142): n is a, c, g, or t
gaccgcgtctcacaccggttcactacgagtgtattccgtttggagaccgtttaaacggtctctacctctccggcgaccggaaatatagtttgnnngcaggtgcaatggcacaccaagcacgatatcagcattgcacctgcnntatgcatgcgcgatgatctaacgcacgtttgaagagcgtttaaacgctcttcgccctagatttcgcgcaccgtgtcgtttcgagacggccctga

SEQ ID NO: 601 - DNA, 236 bp, artificial
  oligo
  misc_feature (93)..(95): n is a, c, g, or t
  misc_feature (141)..(142): n is a, c, g, or t
gaccgcgtctcacaccgtgtgcttaagaccgggaccagtttggagaccgtttaaacggtctctacctccgaagtgtatcccgatccggtttgnnngcaggtgcaatgcgagaaaacccgtatcatcgccattgcacctgcnntatgcataagcgacgccgatcataccgtttgaagagcgtttaaacgctcttcgccctatggcgcgctcgattcagggtttcgagacggccctga

SEQ ID NO: 602 - DNA, 236 bp, artificial
  oligo
  misc_feature (93)..(95): n is a, c, g, or t
  misc_feature (141)..(142): n is a, c, g, or t
gaccgcgtctcacaccgaccaggcacccaacctcggcgtttggagaccgtttaaacggtctctacctacggttcattcgataaggatgtttgnnngcaggtgcaatgaacacgcacctttatgaccggcattgcacctgcnntatgcatctacggccgcggttacgttgtttgaagagcgtttaaacgctcttcgccctctctatccgttcgcgcgcggtttcgagacggccctga

SEQ ID NO: 603 - DNA, 236 bp, artificial
  oligo
  misc_feature (93)..(95): n is a, c, g, or t
  misc_feature (141)..(142): n is a, c, g, or t
gaccgcgtctcacaccgtaccttgagactccaccctcgtttggagaccgtttaaacggtctctacctgatagctcaagcgcgacgaagtttgnnngcaggtgcaatggagcacatcgaatccgtttcccattgcacctgcnntatgcacggaccttgtagccgcgtacgtttgaagagcgtttaaacgctcttcgccccgagcgtcgaaactcgacgcgtttcgagacggccctga

SEQ ID NO: 604 - DNA, 236 bp, artificial
  oligo
  misc_feature (93)..(95): n is a, c, g, or t
  misc_feature (141)..(142): n is a, c, g, or t
gaccgcgtctcacaccgttcctacaaccttctcgcaagtttggagaccgtttaaacggtctctacctcgcgcgccgagggctgtattgtttgnnngcaggtgcaatggcttgtccaaatacagtcccccattgcacctgcnntatgcaagttgcttcgttcgaccagtgtttgaagagcgtttaaacgctcttcgcccgaccgcgtagcaattcgtatgtttcgagacggccctga

SEQ ID NO: 605 - DNA, 236 bp, artificial
  oligo
  misc_feature (93)..(95): n is a, c, g, or t
  misc_feature (141)..(142): n is a, c, g, or t
gaccgcgtctcacaccgcgcccagggagcgcaaacacgtttggagaccgtttaaacggtctctacctatcctccacgtgcggttcccgtttgnnngcaggtgcaatggctcgatacgatttgaacccgcattgcacctgcnntatgcaccgagttacctcgagctttcgtttgaagagcgtttaaacgctcttcgcccctcgtagcttgccacactgtgtttcgagacggccctga

SEQ ID NO: 606 - DNA, 236 bp, artificial
  oligo
  misc_feature (93)..(95): n is a, c, g, or t
  misc_feature (141)..(142): n is a, c, g, or t
gaccgcgtctcacaccgtcgggggacggttatttctggtttggagaccgtttaaacggtctctaccttcagaagcatgataagcaccgtttgnnngcaggtgcaatgacagactagcactaacaccgccattgcacctgcnntatgcacggccatgcctgccagcttcgtttgaagagcgtttaaacgctcttcgcccgacttcactagagcgccgtagtttcgagacggccctga

SEQ ID NO: 607 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctctcctgggaaccgcacgtgggtttgnnngcaggtgcaatgaccgtccctattgaaacgccccattgcacctgcnntatgcacgcgcaccatagcatcacgcgtttgaagagcgtttaaacgctcttcgcccttatcgacaatccgctcactgtttcgagacggccctga

SEQ ID NO: 608 - DNA, 236 bp, artificial
  oligo
  misc_feature (93)..(95): n is a, c, g, or t
  misc_feature (141)..(142): n is a, c, g, or t
gaccgcgtctcacaccgctcctgggaaccgcacgtgggtttggagaccgtttaaacggtctctacctttgggggatactcacgggatgtttgnnngcaggtgcaatgcgtgcacaccaagttccgatacattgcacctgcnntatgcaccgagttacctcgagctttcgtttgaagagcgtttaaacgctcttcgcccatccgtcgcgctaacgacgggtttcgagacggccctga

SEQ ID NO: 609 - DNA, 236 bp, artificial
  oligo
  misc_feature (93)..(95): n is a, c, g, or t
  misc_feature (141)..(142): n is a, c, g, or t
gaccgcgtctcacaccggaaccgcacgtggaggatgcgtttggagaccgtttaaacggtctctaccttgataagcacctggtgctccgtttgnnngcaggtgcaatgccacgtacgaataaactgccgcattgcacctgcnntatgcatcgacaacccctacatcgtggtttgaagagcgtttaaacgctcttcgccccgttcggctaggccaatctggtttcgagacggccctga

SEQ ID NO: 610 - DNA, 236 bp, artificial
  oligo
  misc_feature (93)..(95): n is a, c, g, or t
  misc_feature (141)..(142): n is a, c, g, or t
gaccgcgtctcacaccgtgtggcaagctacgagaaccgtttggagaccgtttaaacggtctctaccttcgacaacccctacatcgtggtttgnnngcaggtgcaatgcacgacgcacatctttggcttcattgcacctgcnntatgcaaacactacgtccctcgtactgtttgaagagcgtttaaacgctcttcgcccaatatccgttagcggggtcggtttcgagacggccctga

SEQ ID NO: 611 - DNA, 236 bp, artificial
  oligo
  misc_feature (93)..(95): n is a, c, g, or t
  misc_feature (141)..(142): n is a, c, g, or t
gaccgcgtctcacaccgactcacggcagcgcacgcacgtttggagaccgtttaaacggtctctacctttctcggaagtcgaggacaggtttgnnngcaggtgcaatgacccgccacatctaggtaatgcattgcacctgcnntatgcacggaccaatcatcatcggatgtttgaagagcgtttaaacgctcttcgccctgagtcgcgatatatgccgagtttcgagacggccctga

SEQ ID NO: 612 - DNA, 236 bp, artificial
  oligo
  misc_feature (93)..(95): n is a, c, g, or t
  misc_feature (141)..(142): n is a, c, g, or t
gaccgcgtctcacaccgcattgtatacgccttcctgggtttggagaccgtttaaacggtctctacctactcacggcagcgcacgcacgtttgnnngcaggtgcaatgccccatgcgacacgtatttcgcattgcacctgcnntatgcagccgccgtagtaaaaccgtcgtttgaagagcgtttaaacgctcttcgcccgctcgcgctatcatgcgttggtttcgagacggccctga

SEQ ID NO: 613 - DNA, 236 bp, artificial
  oligo
  misc_feature (93)..(95): n is a, c, g, or t
  misc_feature (141)..(142): n is a, c, g, or t
gaccgcgtctcacaccgggccagaatacaggtcccgcgtttggagaccgtttaaacggtctctacctcgcgcaccatagcatcacgcgtttgnnngcaggtgcaatgaatcagagccatccgactacgcattgcacctgcnntatgcagctatcgcttgccggagccggtttgaagagcgtttaaacgctcttcgcccatctgtattaacgcggtacagtttcgagacggccctga

SEQ ID NO: 614 - DNA, 236 bp, artificial
  oligo
  misc_feature (93)..(95): n is a, c, g, or t
  misc_feature (141)..(142): n is a, c, g, or t
gaccgcgtctcacaccgcgcgcaccatagcatcacgcgtttggagaccgtttaaacggtctctacctggggcacgatggcctttaccgtttgnnngcaggtgcaatgatcagacacgacgatctccaccattgcacctgcnntatgcagggcgcgcatgacgaccgaagtttgaagagcgtttaaacgctcttcgcccgaatctcgcgtcacgtaatcgtttcgagacggccctga

SEQ ID NO: 615 - DNA, 236 bp, artificial
  oligo
  misc_feature (93)..(95): n is a, c, g, or t
  misc_feature (141)..(142): n is a, c, g, or t
gaccgcgtctcacaccgaagaggcttcttatgtcggcgtttggagaccgtttaaacggtctctacctgtgacccgaggaaccggtgcgtttgnnngcaggtgcaatgagaacattcctctccgactggcattgcacctgcnntatgcacgtatcgtatctgacgtgtggtttgaagagcgtttaaacgctcttcgccccggtcgactggtcgatcattgtttcgagacggccctga

SEQ ID NO: 616 - DNA, 236 bp, artificial
  oligo
  misc_feature (93)..(95): n is a, c, g, or t
  misc_feature (141)..(142): n is a, c, g, or t
gaccgcgtctcacaccggccttgagcccggtggggctgtttggagaccgtttaaacggtctctacctctcgtagttcaaacgacgtcgtttgnnngcaggtgcaatggcacgcagaaacccattgatccattgcacctgcnntatgcagattttcgcgcattacgcaggtttgaagagcgtttaaacgctcttcgccctaaaacgccgcgtcggaatggtttcgagacggccctga

SEQ ID NO: 617 - DNA, 236 bp, artificial
  oligo
  misc_feature (93)..(95): n is a, c, g, or t
  misc_feature (141)..(142): n is a, c, g, or t
gaccgcgtctcacaccgtcgacaacccctacatcgtggtttggagaccgtttaaacggtctctaccttcagcgggccgacatcgacggtttgnnngcaggtgcaatgcgcaagtacccagatgatccacattgcacctgcnntatgcagatcgctaggattgcgcggggtttgaagagcgtttaaacgctcttcgcccaaacggtcgccggccggagtgtttcgagacggccctga

SEQ ID NO: 618 - DNA, 236 bp, artificial
  oligo
  misc_feature (93)..(95): n is a, c, g, or t
  misc_feature (141)..(142): n is a, c, g, or t
gaccgcgtctcacaccggtgacccgaggaaccggtgcgtttggagaccgtttaaacggtctctacctgtgcgctaatagatgcgcgggtttgnnngcaggtgcaatgagcacccgcacttgactttaccattgcacctgcnntatgcaaatcgtatttggtcgcgcgagtttgaagagcgtttaaacgctcttcgcccgctctcgataccgccgtgtcgtttcgagacggccctga

SEQ ID NO: 619 - DNA, 236 bp, artificial
  oligo
  misc_feature (93)..(95): n is a, c, g, or t
  misc_feature (141)..(142): n is a, c, g, or t
gaccgcgtctcacaccgatcctccacgtgcggttcccgtttggagaccgtttaaacggtctctacctcgtgacgaggctataccggtgtttgnnngcaggtgcaatgccccaagaacgaggctaaatccattgcacctgcnntatgcacgcgtcgctagtgtgtccatgtttgaagagcgtttaaacgctcttcgcccccacgattcgcgcgaatcctgtttcgagacggccctga

SEQ ID NO: 620 - DNA, 236 bp, artificial
  oligo
  misc_feature (93)..(95): n is a, c, g, or t
  misc_feature (141)..(142): n is a, c, g, or t
gaccgcgtctcacaccgtcgatcggatgacggtatgtgtttggagaccgtttaaacggtctctacctctgtccgttcgcgtagattagtttgnnngcaggtgcaatggaccagttcaactgcactcgacattgcacctgcnntatgcagacccgaactgcgcgcgatagtttgaagagcgtttaaacgctcttcgcccaacactacgtccctcgtactgtttcgagacggccctga

SEQ ID NO: 621 - DNA, 236 bp, artificial
  oligo
  misc_feature (93)..(95): n is a, c, g, or t
  misc_feature (141)..(142): n is a, c, g, or t
gaccgcgtctcacaccgaccgccgaatcgagcctgcggtttggagaccgtttaaacggtctctaccttcgtggcgggtcgaattcgcgtttgnnngcaggtgcaatgtgacccgatttaacccgccatcattgcacctgcnntatgcacaacgaccggtacggatcatgtttgaagagcgtttaaacgctcttcgcccagataatatgcgccgctcgggtttcgagacggccctga

SEQ ID NO: 622 - DNA, 236 bp, artificial
  oligo
  misc_feature (93)..(95): n is a, c, g, or t
  misc_feature (141)..(142): n is a, c, g, or t
gaccgcgtctcacaccggggcgtatcggtcaagacgagtttggagaccgtttaaacggtctctacctatcactaatgaatcgcggtagtttgnnngcaggtgcaatggcgaccaccgtaattgccaaacattgcacctgcnntatgcaaggacgcgcgtaacctccacgtttgaagagcgtttaaacgctcttcgcccaacaagcggctatgtcggacgtttcgagacggccctga

SEQ ID NO: 623 - DNA, 236 bp, artificial
  oligo
  misc_feature (93)..(95): n is a, c, g, or t
  misc_feature (141)..(142): n is a, c, g, or t
gaccgcgtctcacaccgcaagcgcgttagcggtcgtcgtttggagaccgtttaaacggtctctacctaaaccgatcgcgagtgcgcggtttgnnngcaggtgcaatgtagcgcaacctacgtttcacccattgcacctgcnntatgcagtcggcgataacgcatagctgtttgaagagcgtttaaacgctcttcgcccccgcggcggataacgtgtttgtttcgagacggccctga

SEQ ID NO: 624 - DNA, 236 bp, artificial
  oligo
  misc_feature (93)..(95): n is a, c, g, or t
  misc_feature (141)..(142): n is a, c, g, or t
gaccgcgtctcacaccgataacgccgcgaccaacgtagtttggagaccgtttaaacggtctctacctatagccttgcgtcgagtatggtttgnnngcaggtgcaatggctaacctattggacgactcccattgcacctgcnntatgcacgaagcgggatttacgtctagtttgaagagcgtttaaacgctcttcgcccgcttacggacccccccgatagtttcgagacggccctga

SEQ ID NO: 625 - DNA, 236 bp, artificial
  oligo
  misc_feature (93)..(95): n is a, c, g, or t
  misc_feature (141)..(142): n is a, c, g, or t
gaccgcgtctcacaccggtcggcgataagatggacgcgtttggagaccgtttaaacggtctctacctgcgcgaagcgtatttggcgggtttgnnngcaggtgcaatggtatccctatagcccaatcggcattgcacctgcnntatgcatagacctcgacgaacgctttgtttgaagagcgtttaaacgctcttcgcccctgcgctcgcttaaggttaggtttcgagacggccctga

SEQ ID NO: 626 - DNA, 236 bp, artificial
  oligo
  misc_feature (93)..(95): n is a, c, g, or t
  misc_feature (141)..(142): n is a, c, g, or t
gaccgcgtctcacaccgggttctacgcccgcggctcagtttggagaccgtttaaacggtctctacctgggaccgatccattaccgtagtttgnnngcaggtgcaatgcaaaggagcacacgcctcaatcattgcacctgcnntatgcagataccgacctctcgccgcggtttgaagagcgtttaaacgctcttcgccccaagtagcgcgcttagacatgtttcgagacggccctga

SEQ ID NO: 627 - DNA, 236 bp, artificial
  oligo
  misc_feature (93)..(95): n is a, c, g, or t
  misc_feature (141)..(142): n is a, c, g, or t
gaccgcgtctcacaccgtcgaacgcgaagggtcgcgtgtttggagaccgtttaaacggtctctacctactaacgacgtcgactagccgtttgnnngcaggtgcaatgggtagacaccagacctaacagcattgcacctgcnntatgcagcgagtcgagcaatttggcggtttgaagagcgtttaaacgctcttcgcccctattacctttcgacgcgatgtttcgagacggccctga

SEQ ID NO: 628 - DNA, 236 bp, artificial
  oligo
  misc_feature (93)..(95): n is a, c, g, or t
  misc_feature (141)..(142): n is a, c, g, or t
gaccgcgtctcacaccgacgaaccggtctaccgcatagtttggagaccgtttaaacggtctctacctcgagcgtcgaaactcgacgcgtttgnnngcaggtgcaatgccccagataagtcctcactcacattgcacctgcnntatgcatcgcctaccgacgcgagattgtttgaagagcgtttaaacgctcttcgcccggtcgctcgatgcacgacatgtttcgagacggccctga

SEQ ID NO: 629 - DNA, 236 bp, artificial
  oligo
  misc_feature (93)..(95): n is a, c, g, or t
  misc_feature (141)..(142): n is a, c, g, or t
gaccgcgtctcacaccggagcgttacggcacgacgccgtttggagaccgtttaaacggtctctacctacccgtattcggacgtaccggtttgnnngcaggtgcaatgccgcactcatctcgttctctacattgcacctgcnntatgcaccggtcgaggcgactactccgtttgaagagcgtttaaacgctcttcgcccaataacggcgcgttaagcgtgtttcgagacggccctga

SEQ ID NO: 630 - DNA, 236 bp, artificial
  oligo
  misc_feature (93)..(95): n is a, c, g, or t
  misc_feature (141)..(142): n is a, c, g, or t
gaccgcgtctcacaccgaaccggcggtacgcgcgtttgtttggagaccgtttaaacggtctctacctatagacgcgtgacaaccgaagtttgnnngcaggtgcaatggaccatcagacaacccgtcaacattgcacctgcnntatgcacgtcttatagtaatcggcgggtttgaagagcgtttaaacgctcttcgcccttcgcggcggagaagttagggtttcgagacggccctga

SEQ ID NO: 631 - DNA, 236 bp, artificial
  oligo
  misc_feature (93)..(95): n is a, c, g, or t
  misc_feature (141)..(142): n is a, c, g, or t
gaccgcgtctcacaccggacgccttaatcatcgagatgtttggagaccgtttaaacggtctctacctacgcaatcagcccaagcgttgtttgnnngcaggtgcaatgcagtaatccatcgacgctccccattgcacctgcnntatgcacgcattgaccgacccgtcgagtttgaagagcgtttaaacgctcttcgccctagtcgcaccgtaagccgaggtttcgagacggccctga

SEQ ID NO: 632 - DNA, 236 bp, artificial
  oligo
  misc_feature (93)..(95): n is a, c, g, or t
  misc_feature (141)..(142): n is a, c, g, or t
gaccgcgtctcacaccgagtacgtcccgatggtatcggtttggagaccgtttaaacggtctctacctcgaaactatggccggcgaatgtttgnnngcaggtgcaatggtcctaacaccttcgtacgtccattgcacctgcnntatgcacgcgaagcgttgaacgacaggtttgaagagcgtttaaacgctcttcgcccgacatcccgccgccgttgttgtttcgagacggccctga

SEQ ID NO: 633 - DNA, 236 bp, artificial
  oligo
  misc_feature (93)..(95): n is a, c, g, or t
  misc_feature (141)..(142): n is a, c, g, or t
gaccgcgtctcacaccgacggttcattcgataaggatgtttggagaccgtttaaacggtctctacctcccggcggttcgtcttcgccgtttgnnngcaggtgcaatggtgcagcagtgtgttctaggacattgcacctgcnntatgcagcgatttgcgtgacgcacgggtttgaagagcgtttaaacgctcttcgcccgctaaaccggctcgggctatgtttcgagacggccctga

SEQ ID NO: 634 - DNA, 236 bp, artificial
  oligo
  misc_feature (93)..(95): n is a, c, g, or t
  misc_feature (141)..(142): n is a, c, g, or t
gaccgcgtctcacaccgagcgcgctagcgcagagtacgtttggagaccgtttaaacggtctctacctccccgactcgagtaacgatcgtttgnnngcaggtgcaatggcctgcacctacctccagtaacattgcacctgcnntatgcaccgtacgactatctaaagctgtttgaagagcgtttaaacgctcttcgccctaggcggcttatgtccggttgtttcgagacggccctga

SEQ ID NO: 635 - DNA, 236 bp, artificial
  oligo
  misc_feature (93)..(95): n is a, c, g, or t
  misc_feature (141)..(142): n is a, c, g, or t
gaccgcgtctcacaccgatacgagactcttgcgcgacgtttggagaccgtttaaacggtctctacctagtaatgcacccgtcgatccgtttgnnngcaggtgcaatgctctacgacgaagagaacccccattgcacctgcnntatgcaatacgcgaatgcgttgacacgtttgaagagcgtttaaacgctcttcgccccgattcgcgtactgagccgagtttcgagacggccctga

SEQ ID NO: 636 - DNA, 236 bp, artificial
  oligo
  misc_feature (93)..(95): n is a, c, g, or t
  misc_feature (141)..(142): n is a, c, g, or t
gaccgcgtctcacaccgtattcgttcgccgcgcgactgtttggagaccgtttaaacggtctctacctacgcgcccggttacctcggagtttgnnngcaggtgcaatgcgcgcctataaattacacggccattgcacctgcnntatgcaaaacggcggatctaggaacggtttgaagagcgtttaaacgctcttcgccctatcgcgaagtgccgtgagtgtttcgagacggccctga

SEQ ID NO: 637 - DNA, 236 bp, artificial
  oligo
  misc_feature (93)..(95): n is a, c, g, or t
  misc_feature (141)..(142): n is a, c, g, or t
gaccgcgtctcacaccgtcgagacaaattcgtcgcgtgtttggagaccgtttaaacggtctctacctctctaaacgtcgaaccgtccgtttgnnngcaggtgcaatgagtgtggacgaaccccttcaccattgcacctgcnntatgcaactgcgcgtataacgatagggtttgaagagcgtttaaacgctcttcgccccgcacgccgatatcattcgggtttcgagacggccctga

SEQ ID NO: 638 - DNA, 236 bp, artificial
  oligo
  misc_feature (93)..(95): n is a, c, g, or t
  misc_feature (141)..(142): n is a, c, g, or t
gaccgcgtctcacaccggtataatagacgccgacgcagtttggagaccgtttaaacggtctctaccttgcgtcaatcgcagcgtctagtttgnnngcaggtgcaatgcaccccaagacatcgatcgtgcattgcacctgcnntatgcagcgaggttcgccgtcgtgatgtttgaagagcgtttaaacgctcttcgcccgcgagaaccggcattggattgtttcgagacggccctga

SEQ ID NO: 639 - DNA, 236 bp, artificial
  oligo
  misc_feature (93)..(95): n is a, c, g, or t
  misc_feature (141)..(142): n is a, c, g, or t
gaccgcgtctcacaccgagttgcttcgttcgaccagtgtttggagaccgtttaaacggtctctaccttatccgctcgtccgaacacggtttgnnngcaggtgcaatgccatctctagacggcaacctacattgcacctgcnntatgcactgtgggttaacgtcgcggtgtttgaagagcgtttaaacgctcttcgccctaccccgcctaattaccggtgtttcgagacggccctga

SEQ ID NO: 640 - DNA, 236 bp, artificial
  oligo
  misc_feature (93)..(95): n is a, c, g, or t
  misc_feature (141)..(142): n is a, c, g, or t
gaccgcgtctcacaccggcgcgccagatcgtaattaagtttggagaccgtttaaacggtctctacctctcgatatcaaccccgtaacgtttgnnngcaggtgcaatggtccatgtatagacccactgccattgcacctgcnntatgcatcgtttggccagacgcgtcagtttgaagagcgtttaaacgctcttcgccccgccgaatgaaaacgccggcgtttcgagacggccctga

SEQ ID NO: 641 - DNA, 236 bp, artificial
  oligo
  misc_feature (93)..(95): n is a, c, g, or t
  misc_feature (141)..(142): n is a, c, g, or t
gaccgcgtctcacaccgcacgcaactgtcgtaacacggtttggagaccgtttaaacggtctctacctcggaccggaagcgtctttcggtttgnnngcaggtgcaatgtcacttgacgtacagaccgaccattgcacctgcnntatgcataagcgacgccgatcataccgtttgaagagcgtttaaacgctcttcgccccgcactcgacctacgataccgtttcgagacggccctga

SEQ ID NO: 642 - DNA, 236 bp, artificial
  oligo
  misc_feature (93)..(95): n is a, c, g, or t
  misc_feature (141)..(142): n is a, c, g, or t
gaccgcgtctcacaccggttgtgtcgcatcgacgagtgtttggagaccgtttaaacggtctctacctacctcgatacacgcgcgctagtttgnnngcaggtgcaatgcgcacggtgacaccactctatcattgcacctgcnntatgcatgcgcgttgtcgattaccgggtttgaagagcgtttaaacgctcttcgcccctacgacgccccgtgctatcgtttcgagacggccctga

SEQ ID NO: 643 - DNA, 236 bp, artificial
  oligo
  misc_feature (93)..(95): n is a, c, g, or t
  misc_feature (141)..(142): n is a, c, g, or t
gaccgcgtctcacaccgacatatccgcggcgactcgggtttggagaccgtttaaacggtctctacctctcgtagttcaaacgacgtcgtttgnnngcaggtgcaatgctgccccttactgaatgacaccattgcacctgcnntatgcagataccgaaacgatgtcgtcgtttgaagagcgtttaaacgctcttcgccccgagattacggatcgcgagagtttcgagacggccctga

SEQ ID NO: 644 - DNA, 236 bp, artificial
  oligo
  misc_feature (93)..(95): n is a, c, g, or t
  misc_feature (141)..(142): n is a, c, g, or t
gaccgcgtctcacaccgcggcgaaagcgccgattagagtttggagaccgtttaaacggtctctacctcgggtgcgtttactcgtctagtttgnnngcaggtgcaatgctctgtctaccgctgcggtatcattgcacctgcnntatgcaactcgcgctaatcgctaagagtttgaagagcgtttaaacgctcttcgcccgacatatacgcgatacgacagtttcgagacggccctga

SEQ ID NO: 645 - DNA, 236 bp, artificial
  oligo
  misc_feature (93)..(95): n is a, c, g, or t
  misc_feature (141)..(142): n is a, c, g, or t
gaccgcgtctcacaccggaatttacggcggtcaccgggtttggagaccgtttaaacggtctctacctggcgtatccccgtacgctacgtttgnnngcaggtgcaatgtgcagtaccctaaccagtgcacattgcacctgcnntatgcagtcatgatcggaccgttcgagtttgaagagcgtttaaacgctcttcgccccgggtaagttgcgctaccgtgtttcgagacggccctga

SEQ ID NO: 646 - DNA, 236 bp, artificial
  oligo
  misc_feature (93)..(95): n is a, c, g, or t
  misc_feature (141)..(142): n is a, c, g, or t
gaccgcgtctcacaccgacgtcaaaccggtcgcttaggtttggagaccgtttaaacggtctctacctaacgatcgagcggtactctcgtttgnnngcaggtgcaatggccagaaggacattacatcgccattgcacctgcnntatgcaatatgctagcgtcgcattcggtttgaagagcgtttaaacgctcttcgcccaaggctcgatagttcgcgacgtttcgagacggccctga

SEQ ID NO: 647 - DNA, 236 bp, artificial
  oligo
  misc_feature (93)..(95): n is a, c, g, or t
  misc_feature (141)..(142): n is a, c, g, or t
gaccgcgtctcacaccgtaatgtctaacgctcgcgtcgtttggagaccgtttaaacggtctctacctcgatcgtgtggctcgtcgaggtttgnnngcaggtgcaatgtaatctgacacacccgcgagtcattgcacctgcnntatgcatagcatgcgctttgcggcgagtttgaagagcgtttaaacgctcttcgcccgtacgtttgtaactcggtaggtttcgagacggccctga

SEQ ID NO: 648 - DNA, 236 bp, artificial
  oligo
  misc_feature (93)..(95): n is a, c, g, or t
  misc_feature (141)..(142): n is a, c, g, or t
gaccgcgtctcacaccgactatgcggtaggtacgccggtttggagaccgtttaaacggtctctacctcagcgtaggtaatcgttgacgtttgnnngcaggtgcaatgggcgcacttggttcggctaatcattgcacctgcnntatgcataaactatgatcgcggacgtgtttgaagagcgtttaaacgctcttcgcccccgttggtgcgccaatgccggtttcgagacggccctga

SEQ ID NO: 649 - DNA, 236 bp, artificial
  oligo
  misc_feature (93)..(95): n is a, c, g, or t
  misc_feature (141)..(142): n is a, c, g, or t
gaccgcgtctcacaccgtcgattggccactaacggttgtttggagaccgtttaaacggtctctacctctcggccggatcacgacaaagtttgnnngcaggtgcaatgcgaccagcatgctatcaatgccattgcacctgcnntatgcacgttattccgagactacgtcgtttgaagagcgtttaaacgctcttcgcccttagagtacgcgataggctcgtttcgagacggccctga

SEQ ID NO: 650 - DNA, 236 bp, artificial
  oligo
  misc_feature (93)..(95): n is a, c, g, or t
  misc_feature (141)..(142): n is a, c, g, or t
gaccgcgtctcacaccgggccggacgaattacgataggtttggagaccgtttaaacggtctctaccttcgcctaaccgtattcgttcgtttgnnngcaggtgcaatgcagcacggacaagatcaactgcattgcacctgcnntatgcattacgcagcgacgtcttaatgtttgaagagcgtttaaacgctcttcgccctagttcgttcgacggagcgtgtttcgagacggccctga

SEQ ID NO: 651 - DNA, 236 bp, artificial
  oligo
  misc_feature (93)..(95): n is a, c, g, or t
  misc_feature (141)..(142): n is a, c, g, or t
gaccgcgtctcacaccggctactaccgcgtactacgcgtttggagaccgtttaaacggtctctacctacatcttaggttatgcggcggtttgnnngcaggtgcaatgccaagaatggatatccccagccattgcacctgcnntatgcagtacatcgccccggtcgattgtttgaagagcgtttaaacgctcttcgccccggtcgactggtcgatcattgtttcgagacggccctga

SEQ ID NO: 652 - DNA, 236 bp, artificial
  oligo
  misc_feature (93)..(95): n is a, c, g, or t
  misc_feature (141)..(142): n is a, c, g, or t
gaccgcgtctcacaccgcgcccgaagtcggttagtctgtttggagaccgtttaaacggtctctacctccgtcgtcgtgtgcagttgcgtttgnnngcaggtgcaatgcgaccgatctaccggaaatgtcattgcacctgcnntatgcaatacgaaagcgacgcgtatcgtttgaagagcgtttaaacgctcttcgcccgacttcactagagcgccgtagtttcgagacggccctga

SEQ ID NO: 653 - DNA, 236 bp, artificial
  oligo
  misc_feature (93)..(95): n is a, c, g, or t
  misc_feature (141)..(142): n is a, c, g, or t
gaccgcgtctcacaccggtatgtccgatccgcggtatgtttggagaccgtttaaacggtctctacctagcgcgacccgacgagaaacgtttgnnngcaggtgcaatggacctctacaatacagtgccccattgcacctgcnntatgcaaaaccgcgacgcactcctaggtttgaagagcgtttaaacgctcttcgcccaccgaccaaattccccggcggtttcgagacggccctga

SEQ ID NO: 654 - DNA, 236 bp, artificial
  oligo
  misc_feature (93)..(95): n is a, c, g, or t
  misc_feature (141)..(142): n is a, c, g, or t
gaccgcgtctcacaccggtgcgggcgttaaccagattgtttggagaccgtttaaacggtctctacctgcccgcagcaatcgtacgcggtttgnnngcaggtgcaatgattgcaccgacgtacacgtgacattgcacctgcnntatgcaccgtaatataccgtcctgtcgtttgaagagcgtttaaacgctcttcgccccgtgttaggtcgccgagcttgtttcgagacggccctga

SEQ ID NO: 655 - DNA, 236 bp, artificial
  oligo
  misc_feature (93)..(95): n is a, c, g, or t
  misc_feature (141)..(142): n is a, c, g, or t
gaccgcgtctcacaccggcgcatctaggcgttaccccgtttggagaccgtttaaacggtctctacctagaacgtcgtgtccgtaccagtttgnnngcaggtgcaatggtccatgaagcagcaattcggcattgcacctgcnntatgcaacgtccgtcggtatggacaagtttgaagagcgtttaaacgctcttcgccccgggaagcctattcgccgcggtttcgagacggccctga

SEQ ID NO: 656 - DNA, 236 bp, artificial
  oligo
  misc_feature (93)..(95): n is a, c, g, or t
  misc_feature (141)..(142): n is a, c, g, or t
gaccgcgtctcacaccgcgttcagtcgttagggtacggtttggagaccgtttaaacggtctctacctgagcgacaacgggcgataccgtttgnnngcaggtgcaatgtccgcacaaccctaaggtaagcattgcacctgcnntatgcagcgggcggtaacaagcgcgagtttgaagagcgtttaaacgctcttcgcccacgactgcgttttcgactgggtttcgagacggccctga

SEQ ID NO: 657 - DNA, 236 bp, artificial
  oligo
  misc_feature (93)..(95): n is a, c, g, or t
  misc_feature (141)..(142): n is a, c, g, or t
gaccgcgtctcacaccgccgcgcgtaaggtttcgtgcgtttggagaccgtttaaacggtctctacctcacgctatcccgtgaatgatgtttgnnngcaggtgcaatgcacactcggaactcactgcctcattgcacctgcnntatgcacgacttcgatttgtactccggtttgaagagcgtttaaacgctcttcgcccgccgattatcggtaattccggtttcgagacggccctga

SEQ ID NO: 658 - DNA, 236 bp, artificial
  oligo
  misc_feature (93)..(95): n is a, c, g, or t
  misc_feature (141)..(142): n is a, c, g, or t
gaccgcgtctcacaccgggtacgtgtcatatacgtcggtttggagaccgtttaaacggtctctacctagcggcgtaagacgattatcgtttgnnngcaggtgcaatgaccgatgtaaagctcaagccgcattgcacctgcnntatgcatatcgattcgccccgcctgtgtttgaagagcgtttaaacgctcttcgcccccggacgcggcgtatgatacgtttcgagacggccctga

SEQ ID NO: 659 - DNA, 236 bp, artificial
  oligo
  misc_feature (93)..(95): n is a, c, g, or t
  misc_feature (141)..(142): n is a, c, g, or t
gaccgcgtctcacaccgttcgcgcaacttcgtcgcaggtttggagaccgtttaaacggtctctaccttcgatcacgcgacggccccggtttgnnngcaggtgcaatggcgtaaaccgctacagccatacattgcacctgcnntatgcaccgcatcgtaggtgtcgaaagtttgaagagcgtttaaacgctcttcgcccttgatcgggttcgtcggagcgtttcgagacggccctga

SEQ ID NO: 660 - DNA, 236 bp, artificial
  oligo
  misc_feature (93)..(95): n is a, c, g, or t
  misc_feature (141)..(142): n is a, c, g, or t
gaccgcgtctcacaccgacgagcgatagcctaggacggtttggagaccgtttaaacggtctctacctccccgcgtattacgagatctgtttgnnngcaggtgcaatgcttacaggcaaaggctactcccattgcacctgcnntatgcacgtccgggtttagcattcgagtttgaagagcgtttaaacgctcttcgcccctcgacgcaaccgcagatcggtttcgagacggccctga

SEQ ID NO: 661 - DNA, 236 bp, artificial
  oligo
  misc_feature (93)..(95): n is a, c, g, or t
  misc_feature (141)..(142): n is a, c, g, or t
gaccgcgtctcacaccgtacactacgggaactcgccggtttggagaccgtttaaacggtctctacctgcggaaccatcgtcagtacggtttgnnngcaggtgcaatgccacggaagaaactgacgttccattgcacctgcnntatgcaccgttccgagtagccgtgtagtttgaagagcgtttaaacgctcttcgcccacgagtctggacgctcgacggtttcgagacggccctga

SEQ ID NO: 662 - DNA, 236 bp, artificial
  oligo
  misc_feature (93)..(95): n is a, c, g, or t
  misc_feature (141)..(142): n is a, c, g, or t
gaccgcgtctcacaccgtttcgcgatgcgcacgtattgtttggagaccgtttaaacggtctctacctactcgttcgaccgcgtcgcggtttgnnngcaggtgcaatgtcaacaaccagtacccggcagcattgcacctgcnntatgcatcaacgtagcgctattatcggtttgaagagcgtttaaacgctcttcgcccggtttgcgctaacgtgtggcgtttcgagacggccctga

SEQ ID NO: 663 - DNA, 236 bp, artificial
  oligo
  misc_feature (93)..(95): n is a, c, g, or t
  misc_feature (141)..(142): n is a, c, g, or t
gaccgcgtctcacaccgcccccgtcggacgcgtatgagtttggagaccgtttaaacggtctctacctagcgcacgttagacaccgcggtttgnnngcaggtgcaatgagcttccccactacatcagtgcattgcacctgcnntatgcaaccgttcgtacgaccaatccgtttgaagagcgtttaaacgctcttcgcccagtcgatcgcactcgacctagtttcgagacggccctga

SEQ ID NO: 664 - DNA, 236 bp, artificial
  oligo
  misc_feature (93)..(95): n is a, c, g, or t
  misc_feature (141)..(142): n is a, c, g, or t
gaccgcgtctcacaccgcggtagttgcgtagtagacggtttggagaccgtttaaacggtctctacctaattattagattcgcgcgtcgtttgnnngcaggtgcaatgcataaacgagcagacctcacccattgcacctgcnntatgcacggggcatagctcgtcggacgtttgaagagcgtttaaacgctcttcgcccaagcacgtaatccgtcctatgtttcgagacggccctga

SEQ ID NO: 665 - DNA, 236 bp, artificial
  oligo
  misc_feature (93)..(95): n is a, c, g, or t
  misc_feature (141)..(142): n is a, c, g, or t
gaccgcgtctcacaccggctgtacgaccgcgataagcgtttggagaccgtttaaacggtctctacctattcaacgacgaccgtgcgagtttgnnngcaggtgcaatgtgaagatagccacatcagcgccattgcacctgcnntatgcattaccgcatccgcttcgagcgtttgaagagcgtttaaacgctcttcgccccgcgcgccgagggctgtattgtttcgagacggccctga

SEQ ID NO: 666 - DNA, 236 bp, artificial
  oligo
  misc_feature (93)..(95): n is a, c, g, or t
  misc_feature (141)..(142): n is a, c, g, or t
gaccgcgtctcacaccgacggcgaacggctagtcgccgtttggagaccgtttaaacggtctctaccttacgggaccgatccggcaaagtttgnnngcaggtgcaatgcagtgcaccaatcagagttggcattgcacctgcnntatgcaccacgccgaacgccgtcagtgtttgaagagcgtttaaacgctcttcgcccccggctgtcgacgccagtacgtttcgagacggccctga

SEQ ID NO: 667 - DNA, 236 bp, artificial
  oligo
  misc_feature (93)..(95): n is a, c, g, or t
  misc_feature (141)..(142): n is a, c, g, or t
gaccgcgtctcacaccgaaccgggttacgggtctacggtttggagaccgtttaaacggtctctacctggcgtcgtacaatctcgagcgtttgnnngcaggtgcaatgcaaagcaccctgttagtcacccattgcacctgcnntatgcagcccatagcgacggtgatcggtttgaagagcgtttaaacgctcttcgccctgatgtaagctcgtatcgaagtttcgagacggccctga

SEQ ID NO: 668 - DNA, 236 bp, artificial
  oligo
  misc_feature (93)..(95): n is a, c, g, or t
  misc_feature (141)..(142): n is a, c, g, or t
gaccgcgtctcacaccggtagccggcccgacgcaaacgtttggagaccgtttaaacggtctctacctctcgatcgtgcggtatacgtgtttgnnngcaggtgcaatgctactagacagactacagcggcattgcacctgcnntatgcaggttcgacagcgttaggcccgtttgaagagcgtttaaacgctcttcgccctaccctcggcattcgaccgagtttcgagacggccctga

SEQ ID NO: 669 - DNA, 236 bp, artificial
  oligo
  misc_feature (93)..(95): n is a, c, g, or t
  misc_feature (141)..(142): n is a, c, g, or t
gaccgcgtctcacaccggttcgaacgcagaatgtacggtttggagaccgtttaaacggtctctacctttggggcgtagtcgtccgctgtttgnnngcaggtgcaatggccattcagtaactcccgatgcattgcacctgcnntatgcaagtacgcgcgaacaagtattgtttgaagagcgtttaaacgctcttcgccctcgcgtcgatggtcgagatagtttcgagacggccctga

SEQ ID NO: 670 - DNA, 236 bp, artificial
  oligo
  misc_feature (93)..(95): n is a, c, g, or t
  misc_feature (141)..(142): n is a, c, g, or t
gaccgcgtctcacaccgcacacgttatgttcgcgcgtgtttggagaccgtttaaacggtctctacctcgaccggccacatcgactaggtttgnnngcaggtgcaatggttcgccaaaccatccctttgcattgcacctgcnntatgcagattcatttccgcgccgtatgtttgaagagcgtttaaacgctcttcgccccgtgccggctcatcgttcgagtttcgagacggccctga

SEQ ID NO: 671 - DNA, 236 bp, artificial
  oligo
  misc_feature (93)..(95): n is a, c, g, or t
  misc_feature (141)..(142): n is a, c, g, or t
gaccgcgtctcacaccgcccgcgacaaatctgccgtagtttggagaccgtttaaacggtctctacctcgtggcccgtttagcgtgtcgtttgnnngcaggtgcaatgcactttccccttacctgcctgcattgcacctgcnntatgcactcgtcgcatagcgggtgaggtttgaagagcgtttaaacgctcttcgcccccgcgttgcacttgaccggggtttcgagacggccctga

SEQ ID NO: 672 - DNA, 236 bp, artificial
  oligo
  misc_feature (93)..(95): n is a, c, g, or t
  misc_feature (141)..(142): n is a, c, g, or t
gaccgcgtctcacaccgcgatcaaacgctgagtggacgtttggagaccgtttaaacggtctctacctcgttagtgtgtcgcgcataagtttgnnngcaggtgcaatgccacctttcgaatatggcacccattgcacctgcnntatgcaatcgcaccgcgaataacgcagtttgaagagcgtttaaacgctcttcgcccagtcctacgacgcgccgcaagtttcgagacggccctga

SEQ ID NO: 673 - DNA, 236 bp, artificial
  oligo
  misc_feature (93)..(95): n is a, c, g, or t
  misc_feature (141)..(142): n is a, c, g, or t
gaccgcgtctcacaccgagttgcacccgcggttaacggtttggagaccgtttaaacggtctctacctctttaccgcgcgacggacaagtttgnnngcaggtgcaatggaatcccccaattagcaagcgcattgcacctgcnntatgcacacctaactacgcccgcgacgtttgaagagcgtttaaacgctcttcgcccatcatcgttccgcgtcaaccgtttcgagacggccctga

SEQ ID NO: 674 - DNA, 236 bp, artificial
  oligo
  misc_feature (93)..(95): n is a, c, g, or t
  misc_feature (141)..(142): n is a, c, g, or t
gaccgcgtctcacaccgtcgctaacggttggaccgtggtttggagaccgtttaaacggtctctacctcgtcggtcacgagtacatgcgtttgnnngcaggtgcaatgctagacgctcataccgacgaacattgcacctgcnntatgcacttaccgtattaggcacgctgtttgaagagcgtttaaacgctcttcgccccgcgacatgatggtacgcgtgtttcgagacggccctga

SEQ ID NO: 675 - DNA, 236 bp, artificial
  oligo
  misc_feature (93)..(95): n is a, c, g, or t
  misc_feature (141)..(142): n is a, c, g, or t
gaccgcgtctcacaccgccttcgcgctatcttatgcggtttggagaccgtttaaacggtctctacctatctacgtaccgttagcggagtttgnnngcaggtgcaatgtcccctaaggcacacccgaatcattgcacctgcnntatgcactcgacgcgaaatttatctagtttgaagagcgtttaaacgctcttcgcccggtttaataccggcgcgtcagtttcgagacggccctga

SEQ ID NO: 676 - DNA, 236 bp, artificial
  oligo
  misc_feature (93)..(95): n is a, c, g, or t
  misc_feature (141)..(142): n is a, c, g, or t
gaccgcgtctcacaccgtcgcgataggcggtatctgagtttggagaccgtttaaacggtctctacctacttgcgctgtatcgcgccggtttgnnngcaggtgcaatgcacagcgcacgatctttggaacattgcacctgcnntatgcacgtggcgaatcgcggctaaagtttgaagagcgtttaaacgctcttcgcccccgtggatcgtttccgcactgtttcgagacggccctga

SEQ ID NO: 677 - DNA, 236 bp, artificial
  oligo
  misc_feature (93)..(95): n is a, c, g, or t
  misc_feature (141)..(142): n is a, c, g, or t
gaccgcgtctcacaccgttaatcctcggacgcggcgagtttggagaccgtttaaacggtctctacctaactcgcttcgtcggatatagtttgnnngcaggtgcaatgcgatccctccataccagtttgcattgcacctgcnntatgcaacgtatctcgtccaatcgcagtttgaagagcgtttaaacgctcttcgccctggaacggtcgacttcgggcgtttcgagacggccctga

SEQ ID NO: 678 - DNA, 236 bp, artificial
  oligo
  misc_feature (93)..(95): n is a, c, g, or t
  misc_feature (141)..(142): n is a, c, g, or t
gaccgcgtctcacaccgtgatgattcggcttgggcgcgtttggagaccgtttaaacggtctctacctgagcgtcgtcgcacactagtgtttgnnngcaggtgcaatgacgaccagtgcaactcaaagccattgcacctgcnntatgcagatagcgggtcgaactgctcgtttgaagagcgtttaaacgctcttcgcccccgaacctagtaccgcgttagtttcgagacggccctga

SEQ ID NO: 679 - DNA, 236 bp, artificial
  oligo
  misc_feature (93)..(95): n is a, c, g, or t
  misc_feature (141)..(142): n is a, c, g, or t
gaccgcgtctcacaccggctcgcgcattcttacgtcggtttggagaccgtttaaacggtctctacctagtctcgaccggttatccgtgtttgnnngcaggtgcaatgcaggaacacatcggtcaactgcattgcacctgcnntatgcatagacgtaggtacgccggtcgtttgaagagcgtttaaacgctcttcgcccatccactcgcgacgcctaatgtttcgagacggccctga

SEQ ID NO: 680 - DNA, 236 bp, artificial
  oligo
  misc_feature (93)..(95): n is a, c, g, or t
  misc_feature (141)..(142): n is a, c, g, or t
gaccgcgtctcacaccggattttcgaccccgaagcgtgtttggagaccgtttaaacggtctctacctcgttcgagccgcgtatccgtgtttgnnngcaggtgcaatggacaccagactatgatgcacccattgcacctgcnntatgcaattacgtttaatggcgcgtcgtttgaagagcgtttaaacgctcttcgccccatatcggcgcggcgcatttgtttcgagacggccctga

SEQ ID NO: 681 - DNA, 236 bp, artificial
  oligo
  misc_feature (93)..(95): n is a, c, g, or t
  misc_feature (141)..(142): n is a, c, g, or t
gaccgcgtctcacaccgtgcgggtatccgtaggacgcgtttggagaccgtttaaacggtctctaccttccgtaggatctcgacgccagtttgnnngcaggtgcaatgccagcgtacttagccgatagacattgcacctgcnntatgcagattacgtaatgtcgcgggcgtttgaagagcgtttaaacgctcttcgcccaattcgcgcgaattggcggggtttcgagacggccctga

SEQ ID NO: 682 - DNA, 236 bp, artificial
  oligo
  misc_feature (93)..(95): n is a, c, g, or t
  misc_feature (141)..(142): n is a, c, g, or t
gaccgcgtctcacaccgcggctatacggtgtagcaacgtttggagaccgtttaaacggtctctacctgtacaggccgacttcgcgacgtttgnnngcaggtgcaatgcacgtcccgtctaaaaagccacattgcacctgcnntatgcacggtccgatatccgccgggggtttgaagagcgtttaaacgctcttcgcccgtcacccggcggacttaagagtttcgagacggccctga

SEQ ID NO: 683 - DNA, 236 bp, artificial
  oligo
  misc_feature (93)..(95): n is a, c, g, or t
  misc_feature (141)..(142): n is a, c, g, or t
gaccgcgtctcacaccgatacggctaatcgaggcggagtttggagaccgtttaaacggtctctacctgcgccgaaactacaccgagggtttgnnngcaggtgcaatgcgcacgatcagaacacttaggcattgcacctgcnntatgcatctatcatatcgcacgtggtgtttgaagagcgtttaaacgctcttcgccctgcgcggacgctggtagcgagtttcgagacggccctga

SEQ ID NO: 684 - DNA, 236 bp, artificial
  oligo
  misc_feature (93)..(95): n is a, c, g, or t
  misc_feature (141)..(142): n is a, c, g, or t
gaccgcgtctcacaccgaacggcggacgacctacaatgtttggagaccgtttaaacggtctctacctgtacgtctagatgtcgatcggtttgnnngcaggtgcaatggcgaacaagagcacacgggaacattgcacctgcnntatgcatcgacagactatctgccgtagtttgaagagcgtttaaacgctcttcgccccactacgcgggttactagcggtttcgagacggccctga

SEQ ID NO: 685 - DNA, 236 bp, artificial
  oligo
  misc_feature (93)..(95): n is a, c, g, or t
  misc_feature (141)..(142): n is a, c, g, or t
gaccgcgtctcacaccgcgtttaagttggtaccccgcgtttggagaccgtttaaacggtctctaccttatggccgatgcaacgcaacgtttgnnngcaggtgcaatgccgcgaaccgaacagatgatccattgcacctgcnntatgcagttcattgggtcggcgtcgtgtttgaagagcgtttaaacgctcttcgcccccgggtattcgtgtggcgtcgtttcgagacggccctga

SEQ ID NO: 686 - DNA, 236 bp, artificial
  oligo
  misc_feature (93)..(95): n is a, c, g, or t
  misc_feature (141)..(142): n is a, c, g, or t
gaccgcgtctcacaccggcgtcaacggggatgaccgcgtttggagaccgtttaaacggtctctaccttgcgcgacccaacgatctcagtttgnnngcaggtgcaatgctaccactcgtccagctctatcattgcacctgcnntatgcatatgatccctaccaagcgcggtttgaagagcgtttaaacgctcttcgccccgcggaaatcgagcatcacggtttcgagacggccctga

SEQ ID NO: 687 - DNA, 236 bp, artificial
  oligo
  misc_feature (93)..(95): n is a, c, g, or t
  misc_feature (141)..(142): n is a, c, g, or t
gaccgcgtctcacaccggtgcgtggtcggatcgatatgtttggagaccgtttaaacggtctctacctgcccgacgattcgtcgcttggtttgnnngcaggtgcaatgcccttaccacgaaagtaaccccattgcacctgcnntatgcagtccccgcggtaacacgacggtttgaagagcgtttaaacgctcttcgcccagcggatgcgagcacggtatgtttcgagacggccctga

SEQ ID NO: 688 - DNA, 236 bp, artificial
  oligo
  misc_feature (93)..(95): n is a, c, g, or t
  misc_feature (141)..(142): n is a, c, g, or t
gaccgcgtctcacaccggcgccgaaaaacgtcggttggtttggagaccgtttaaacggtctctacctctataccgatacggagtgacgtttgnnngcaggtgcaatggcccctttatatcaaagcccgcattgcacctgcnntatgcatcgaaatcgacacgtcgcgagtttgaagagcgtttaaacgctcttcgcccccgcgagtcctggacataaggtttcgagacggccctga

SEQ ID NO: 689 - DNA, 236 bp, artificial
  oligo
  misc_feature (93)..(95): n is a, c, g, or t
  misc_feature (141)..(142): n is a, c, g, or t
gaccgcgtctcacaccgcgtacaaacgcgggagatccgtttggagaccgtttaaacggtctctacctatcccgtggcgatgtatcgagtttgnnngcaggtgcaatggccaccaagttcttcccaaggcattgcacctgcnntatgcacccctcgccatacgcgtaaggtttgaagagcgtttaaacgctcttcgccctgaacacgatacgcgcacgcgtttcgagacggccctga

SEQ ID NO: 690 - DNA, 236 bp, artificial
  oligo
  misc_feature (93)..(95): n is a, c, g, or t
  misc_feature (141)..(142): n is a, c, g, or t
gaccgcgtctcacaccgcggtattggacgaacgcgctgtttggagaccgtttaaacggtctctaccttgagtcgcgatatatgccgagtttgnnngcaggtgcaatgtgacacacgcgacctcctatccattgcacctgcnntatgcacgacgacctacaatggcggtgtttgaagagcgtttaaacgctcttcgccccgtccggtctatcgactaccgtttcgagacggccctga

SEQ ID NO: 691 - DNA, 236 bp, artificial
  oligo
  misc_feature (93)..(95): n is a, c, g, or t
  misc_feature (141)..(142): n is a, c, g, or t
gaccgcgtctcacaccgcgcgaataggtttgcgaatggtttggagaccgtttaaacggtctctacctttggtcccgcgaatcgcgttgtttgnnngcaggtgcaatggatgctcttagccacttacgccattgcacctgcnntatgcatcgtaagcggtcgaaaacgagtttgaagagcgtttaaacgctcttcgccccgaaattcccgacgctcccggtttcgagacggccctga

SEQ ID NO: 692 - DNA, 236 bp, artificial
  oligo
  misc_feature (93)..(95): n is a, c, g, or t
  misc_feature (141)..(142): n is a, c, g, or t
gaccgcgtctcacaccgacggcttgccaatccgtccggtttggagaccgtttaaacggtctctacctataagcgcggtatacgtaccgtttgnnngcaggtgcaatgtaccaagatctgtatcgccgccattgcacctgcnntatgcatcgcgaccccgatattcacggtttgaagagcgtttaaacgctcttcgccctagattggcgttcggtcgtcgtttcgagacggccctga

SEQ ID NO: 693 - DNA, 236 bp, artificial
  oligo
  misc_feature (93)..(95): n is a, c, g, or t
  misc_feature (141)..(142): n is a, c, g, or t
gaccgcgtctcacaccgtacgtcccgaagcgtccctcgtttggagaccgtttaaacggtctctaccttcgagtccgtaatttaacgtgtttgnnngcaggtgcaatgcacctgaccaaacgtcaccctcattgcacctgcnntatgcagtaaaccggcgccgacccgtgtttgaagagcgtttaaacgctcttcgcccgtatatgccgctacgcctttgtttcgagacggccctga

SEQ ID NO: 694 - DNA, 236 bp, artificial
  oligo
  misc_feature (93)..(95): n is a, c, g, or t
  misc_feature (141)..(142): n is a, c, g, or t
gaccgcgtctcacaccgcaccgcggctatcaaattcggtttggagaccgtttaaacggtctctacctgtctggtaaccgcgcgaacggtttgnnngcaggtgcaatgatcgcatattgtccacgaccgcattgcacctgcnntatgcagaatgatgcgccgcggtacagtttgaagagcgtttaaacgctcttcgccccgatttgatattaccgcgatgtttcgagacggccctga

SEQ ID NO: 695 - DNA, 236 bp, artificial
  oligo
  misc_feature (93)..(95): n is a, c, g, or t
  misc_feature (141)..(142): n is a, c, g, or t
gaccgcgtctcacaccgtcgtccgactcaacacgatggtttggagaccgtttaaacggtctctacctgagtcgtattaccacgctcggtttgnnngcaggtgcaatgctcatgagcgattactaccggcattgcacctgcnntatgcagtggacgatacatacaacgcgtttgaagagcgtttaaacgctcttcgcccttgatacgatctatccgggagtttcgagacggccctga

SEQ ID NO: 696 - DNA, 236 bp, artificial
  oligo
  misc_feature (93)..(95): n is a, c, g, or t
  misc_feature (141)..(142): n is a, c, g, or t
gaccgcgtctcacaccgcgtatcgtatctgacgtgtggtttggagaccgtttaaacggtctctacctagtgaacggttgggtacgccgtttgnnngcaggtgcaatgcgaagcaatcctagccgtatccattgcacctgcnntatgcaacgagtcgtcatcgtgccgtgtttgaagagcgtttaaacgctcttcgcccgaccgcaaccgtcgttcaaggtttcgagacggccctga

SEQ ID NO: 697 - DNA, 236 bp, artificial
  oligo
  misc_feature (93)..(95): n is a, c, g, or t
  misc_feature (141)..(142): n is a, c, g, or t
gaccgcgtctcacaccgccgatataatatcggggaaggtttggagaccgtttaaacggtctctacctattcgccgctaatcacggatgtttgnnngcaggtgcaatgggctatttgatccacccgatgcattgcacctgcnntatgcagcgtactactactaagcggtgtttgaagagcgtttaaacgctcttcgcccgttagcgtccgaaaacccgggtttcgagacggccctga

SEQ ID NO: 698 - DNA, 236 bp, artificial
  oligo
  misc_feature (93)..(95): n is a, c, g, or t
  misc_feature (141)..(142): n is a, c, g, or t
gaccgcgtctcacaccgtgtcgttcgatgactcggatgtttggagaccgtttaaacggtctctacctgcagcagtccgccgatttcggtttgnnngcaggtgcaatgcctattgaacccggaaatccgcattgcacctgcnntatgcaacgtatagtacgcttcgcacgtttgaagagcgtttaaacgctcttcgccctccggccgcattcccgtaccgtttcgagacggccctga

SEQ ID NO: 699 - DNA, 236 bp, artificial
  oligo
  misc_feature (93)..(95): n is a, c, g, or t
  misc_feature (141)..(142): n is a, c, g, or t
gaccgcgtctcacaccgccggatcgtgatcctcggttgtttggagaccgtttaaacggtctctaccttatcctcgcaatatacgcgagtttgnnngcaggtgcaatgacttcagtagatccccaggcccattgcacctgcnntatgcatcgtgccaagcactagtacggtttgaagagcgtttaaacgctcttcgcccgatcgcgttgataaggcgtcgtttcgagacggccctga

SEQ ID NO: 700 - DNA, 236 bp, artificial
  oligo
  misc_feature (93)..(95): n is a, c, g, or t
  misc_feature (141)..(142): n is a, c, g, or t
gaccgcgtctcacaccgtagcgaaatcgtacccgagcgtttggagaccgtttaaacggtctctacctgcgcgacacatgggcggttcgtttgnnngcaggtgcaatggcactttacgtccgcttactgcattgcacctgcnntatgcacatatttacgtggtcgtcgtgtttgaagagcgtttaaacgctcttcgcccgaccggcgagtccgtaccatgtttcgagacggccctga

SEQ ID NO: 701 - DNA, 236 bp, artificial
  oligo
  misc_feature (93)..(95): n is a, c, g, or t
  misc_feature (141)..(142): n is a, c, g, or t
gaccgcgtctcacaccgttatagcgtaataccgccgggtttggagaccgtttaaacggtctctacctgttaaactaaatcggcgcgcgtttgnnngcaggtgcaatgtcaaccaccacatgacggaagcattgcacctgcnntatgcagtaactacggcgcatcgtctgtttgaagagcgtttaaacgctcttcgcccgcgaacatgatcgctacgaggtttcgagacggccctga

SEQ ID NO: 702 - DNA, 236 bp, artificial
  oligo
  misc_feature (93)..(95): n is a, c, g, or t
  misc_feature (141)..(142): n is a, c, g, or t
gaccgcgtctcacaccgcgcgacatgcatcgctttcggtttggagaccgtttaaacggtctctacctagtcgaacccggcgcgattggtttgnnngcaggtgcaatggcacaccatcgacttccgtatcattgcacctgcnntatgcacgcggcgtgaacagaccgatgtttgaagagcgtttaaacgctcttcgcccagcgaatacgtacccgtgtagtttcgagacggccctga

SEQ ID NO: 703 - DNA, 236 bp, artificial
  oligo
  misc_feature (93)..(95): n is a, c, g, or t
  misc_feature (141)..(142): n is a, c, g, or t
gaccgcgtctcacaccgcgtcacggcgagcgaaccccgtttggagaccgtttaaacggtctctacctttattacgtgcgccggccgggtttgnnngcaggtgcaatgctaacagccgaaaagcccaagcattgcacctgcnntatgcacggtacacgacttcgcccgcgtttgaagagcgtttaaacgctcttcgccctaccgcggtcgtttagatcggtttcgagacggccctga

SEQ ID NO: 704 - DNA, 9550 bp, artificial
  PAC Vector
  misc_feature (7488)..(7488): n is a, c, g, or t
  misc_feature (7490)..(7491): n is a, c, g, or t
  misc_feature (7493)..(7493): n is a, c, g, or t
ttaagcttgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatcgcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgtccctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggatagctagagccagacatgataagatacattgatgagtttggacaaaccacaactagaatgcagtgaaaaaaatgctttatttgtgaaatttgtgatgctattgctttatttgtaaccattataagctgcaataaacaagttcctctcactctctgatattcatttctttgcaagttataaatactgaataataagatgacatgaactactatacgtactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagagccagtacaagcaaaaagcagatcttgtcatcgttgggagtgaattagcccttccagtccccccttttcttttaaaaagtggctaagatctacagctgccttgtaagtcattggtcttaaagtcgacgcggggaggcggcccaaagggagatccgactcgtctgagggcgaaggcgaacacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgttgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattgtcgacttaacgcgtggatccttatctgtgccccagtttgctagggaggtcgcagtatctggccacagccacctcgtgctgctcgacgtaggtttctttgtcggcctccttgattctttccagccttctgtccacatagtagacgccgggcatcttgaggttcttagcgggtttcttggatctgtatgtggttttcaagttgcagatcaggtggcccccgcccacgagcttcagggccatgtcggctctgccttccaggccgccgtcagcggggtacagggtttcggtggaggcctcccagccgagtgttttcttctgcatcacagggccgttggatgggaagttcacccctctgatcttgacgttgtagatgaggcagccgtcctggaggctggtgtcctgggtagcggtcagcacgcccccgtcctcgtatgtggtgactctctcccatgtgaagccctcggggaaggactgcttaaagaagtcggggatgccctgggtgtggttgatgaaggttttgctgccgtacatgaagctggtagccaggatgtcgaaggcgaaggggagagggccgccctcgaccgccttgattctcatggtctgggtgccctcgtagggcttgccttcgccctcggatgtgcacttgaagtggtggttgttcacggtgccctccatgtacagcttcatgtgcatgttctccttaatcagctcgctcacggggccgggattttcctccacgtccccgcatgttagcagacttcccctgccctcggcaccgggcttgcgggtcatgcaccaggtgcgcggtccttcgggcacctcgacgtcggcggtgacggtgaagccgagccgctcgtagaaggggaggttgcggggcgcggaggtttccaggaaggcgggcaccccggcgcgctcggccgcctccactccggggagcacgacggcgctgcccagacccttgccctggtggtcgggcgagactccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccacggcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtggccgtacgtcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccaacgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggatcaattgccgacccctccccccaacttctcggggactgtgggccatgtgcgctctgcccactgacgggcaccggagccaattcccactcctttcaagacctagaaggtccattagctgcaaagattcctctctgtttaaaactttatccatctttgcacccgggccccctcgagccggcgccaaagtggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctcaagatctagttacgccaagcttggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcagcttgtccaactggtcgggtttaagagctatgctggaaacagcatagcaagtttaaataaggctagtccgttatcaacttgaaaaagtggcaccgagtcggtgctggttccatggtgtaatggttagcactctggactctgaatccagcgatccgagttcaaatctcggtggaacctagcacacaagcacgttacccgtttgagagctatgccgggansnnsnaaccggcatagcaagttcaaataaggctagtccgttatcaacttggcccggctttaaggccggtcctagcaacgggccaagtggcaccgagtcggtgctgcatgggtggttcagtggtagaattctcgcctgccacgcgggaggcccgggttcgattcccggcccatgcaggactgagagctgttgacacgtttgagagctatgctggagcccgaacagcatagcaagttcaaataaggctagtccgttatcaacttggcccgaaaaaaaagaaaaaaagaaaaaaagaaaaacgggccaagtggcaccgagtcggtgctttttttcaccgtggaatgcgagaattctgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttgctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccaacgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagagccgtttatgtatcgagctaggcacttaaatacaatatctctgcaatgcggcaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactaca

SEQ ID NO: 705 - DNA, 72 bp, Artificial Sequence
  tRNA1
ggttccatggtgtaatggttagcactctggactctgaatccagcgatccgagttcaaatctcggtggaacct

SEQ ID NO: 706 - DNA, 71 bp, Artificial Sequence
  tRNA2
gcatgggtggttcagtggtagaattctcgcctgccacgcgggaggcccgggttcgattcccggcccatgca

SEQ ID NO: 707 - DNA, 72 bp, Artificial Sequence
  tRNA3
ggctcgttggtctaggggtatgattctcgcttagggtgcgagaggtcccgggttcaaatcccggacgagccc

SEQ ID NO: 708 - DNA, 4943 bp, Artificial Sequence
  scaffold1-tRNA1 plasmid
  misc_feature (667)..(667): n is a, c, g, or t
  misc_feature (669)..(670): n is a, c, g, or t
  misc_feature (672)..(672): n is a, c, g, or t
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcggagcatcgtcttccacctgcctcggtttgagagctatgccgggansnnsnaaccggcatagcaagttcaaataaggctagtccgttatcaacttggcccggctttaaggccggtcctagcaacgggccaagtggcaccgagtcggtgctgcatgggtggttcagtggtagaattctcgcctgccacgcgggaggcccgggttcgattcccggcccatgcaaacggcaggtggcggaagacatcgagcggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagagcccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 709 - DNA, 4939 bp, Artificial Sequence
  scaffold2-tRNA2 plasmid
  misc_feature (665)..(665): n is a, c, g, or t
  misc_feature (667)..(668): n is a, c, g, or t
  misc_feature (670)..(670): n is a, c, g, or t
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcggagcatgtcttctcgctcttcggtttcagagctatgccgggansnnsnaaccggcatagcaagttgaaataaggctagtccgttatcaacttggcccggctcacctattagcggctaaggcgggccaagtggcaccgagtcggtgctggctcgttggtctaggggtatgattctcgcttagggtgcgagaggtcccgggttcaaatcccggacgagccccgaagagcgcgggaagactcgagcggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaggagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagagcccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 710 - DNA, 4898 bp, Artificial Sequence
  scaffold3-tRNA3 plasmid
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcggagcatgtggcggatccttcacgggtctcggttttagagctagaaatagcaagttaaaataaggctagtccgttatcaacttgaaaaagtggcaccgagtcggtgcggttccatggtgtaatggttagcactctggactctgaatccagcgatccgagttcaaatctcggtggaacctcgagaccctggcggaattcggcgcggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaggagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagagcccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 711 - DNA, 232 bp, Artificial Sequence
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgaggtggctatggcaggtcttgtttggagaccttnnntgtggtctctacctttaggaaccccatggtgaccgtttgnnngcaggtgcaatgtcatgctaggaaacccacagccattgcacctgcnntatgcagtaccatgttgggctcccaggtttaagtcttctgnnatcgaagacatgcccaggctataagctaacattccgtttcgagacggccc

SEQ ID NO: 712 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgcatccttatactcataccgggtttggagaccttnnntgtggtctctacctggcgacctttaccgatggaggtttgnnngcaggtgcaatggcacgcacgtcattaatgagccattgcacctgcnntatgcattacatattatagcaatcgtgtttaagtcttctgnnatcgaagacatgcccggactcggatgacgtgcccagtttcgagacggccc

SEQ ID NO: 713 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgacagtatacgtggtgcacgagtttggagaccttnnntgtggtctctacctccaggataagggcatcgtcggtttgnnngcaggtgcaatgacgaaaataaggacctcgccgcattgcacctgcnntatgcacatggattacaaccgcagcggtttaagtcttctgnnatcgaagacatgcccgctgcaccgagtcgtagtcggtttcgagacggccc

SEQ ID NO: 714 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgtagagcgggtcatgaatcacgtttggagaccttnnntgtggtctctacctctggaagctggcgggaccacgtttgnnngcaggtgcaatgggaagagagcaccgactaacacattgcacctgcnntatgcaaagcatactccagtcgaacagtttaagtcttctgnnatcgaagacatgcccaggagaactcgagtttgacggtttcgagacggccc

SEQ ID NO: 715 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgactacacacggatgaaggacgtttggagaccttnnntgtggtctctacctcgtacctttggcaagggctagtttgnnngcaggtgcaatgagtcctcgttcaagaacgacccattgcacctgcnntatgcagcaggggcggcgggctgtacgtttaagtcttctgnnatcgaagacatgcccgtagagttcagcattcaaccgtttcgagacggccc

SEQ ID NO: 716 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgctacggacgctccttcacacgtttggagaccttnnntgtggtctctacctacggtcggtagctctgcaccgtttgnnngcaggtgcaatgccgctaaataaacgtaccccgcattgcacctgcnntatgcaagtccaccatagcgtcgtccgtttaagtcttctgnnatcgaagacatgccccccatcgtcaccgcggggccgtttcgagacggccc

SEQ ID NO: 717 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgactggctccacaatcactgcgtttggagaccttnnntgtggtctctacctcttctccgggcgccggatgcgtttgnnngcaggtgcaatgccacctttcgaatatggcacccattgcacctgcnntatgcagagagcaaggagatcgtgcggtttaagtcttctgnnatcgaagacatgcccgcaagttcaagtctctgatcgtttcgagacggccc

SEQ ID NO: 718 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccgaggaaccgagaagctactaagtttggagaccttnnntgtggtctctacctgtttggcaaccagttccaaagtttgnnngcaggtgcaatgtacaccaacacgaaacgccagcattgcacctgcnntatgcatgagttcaaacccatgacacgtttaagtcttctgnnatcgaagacatgccctgtgatgatggtcaacaaccgtttcgagacggccc

SEQ ID NO: 719 - DNA, 232 bp, artificial
  oligo
  misc_feature (49)..(51): n is a, c, g, or t
  misc_feature (91)..(93): n is a, c, g, or t
  misc_feature (139)..(140): n is a, c, g, or t
  misc_feature (181)..(182): n is a, c, g, or t
ccgcgtctcacaccggcagctggaacaaggcatccgtttggagaccttnnntgtggtctctacctgatgtcgaattctaacccaggtttgnnngcaggtgcaatgggaccgtacaagccaagacaccattgcacctgcnntatgcacagcagcactatagcggcacgtttaagtcttctgnnatcgaagacatgcccggagaaaggctcgtgcaggagtttcgagacggccc

(19) United States
(12) Patent Application Publication
(10) Pub. No.: US 2024/0287506 A1
(43) Pub. Date: Aug. 29, 2024

(54) LIBRARY CONSTRUCTION METHOD BASED ON LONG OVERHANG SEQUENCE LIGATION

(71) Applicant: WESTLAKE UNIVERSITY, Hangzhou (CN)

(72) Inventors: Zhike LU, Hangzhou (CN); Lijia MA, Hangzhou (CN)

(21) Appl. No.: 18/572,473

(22) PCT Filed: May 31, 2022
(86) PCT No.: PCT/CN2022/096250

§ 371 Date: Dec. 20, 2023

(30) Foreign Application Priority Data
Jun. 21, 2021 (CN) 202110686751.3

(51) Int. Cl. C12N 15/10; C12N 9/22; C12N 15/11; C12N 15/86; C12Q 1/686

(52) U.S. Cl. CPC C12N 15/1068; C12N 9/22; C12N 15/1096; C12N 15/11; C12N 15/86; C12Q 1/686; C12N 2310/20; C12N 2740/15043

(57) ABSTRACT

Provided is a library construction method based on long overhang sequence ligation, a library constructed by the library construction method, for example, a CRISPR library of pair-specific multiplexed guide RNA (gRNA) combinations, and a use of the library. The CRISPR library of pair-specific multiplexed gRNA combinations can be used to simultaneously perturb multiple (e.g., 4) pre-designed targets in CRISPR/Cas9 screening. The CRISPR library of pair-specific multiplexed gRNA combinations can be a powerful tool for studying the combinatorial outcomes from coordinated gene behaviors.

### INFORMATION OF PRIORITY

The present application claims the benefit of Chinese application for invention No. CN202110686751.3 filed on Jun. 21, 2021, the content of which is incorporated herein in its entirety.

### TECHNICAL FIELD

The present invention pertains to the field of genetic engineering. Specifically, the present invention relates to a library construction method based on long overhang sequence ligation, a library constructed by the library construction method, for example, a CRISPR library of pair-specific multiplexed gRNA (guide RNA) combinations, and a use of the library.

### BACKGROUND ART

With the development of systemic biology, various high-throughput biotechnology methods have emerged. In the field of molecular biology, various library screening techniques can identify genotype-phenotype relationships without prior knowledge. The core steps of library screening technology include: (1) high-complexity library construction, and (2) phenotype screening. Among them, the library construction mainly involves the design and synthesis of high-complexity oligonucleotide fragment (oligo) library, and the commonly used synthesis techniques that meet the requirements are mainly chip-based oligonucleotide pool (oligo pool) synthesis. The limitations of price, quality and length of oligonucleotide pool synthesis have currently become the main bottlenecks in the field.

There are 20,000 to 30,000 genes in the human body, and their functions vary in different biological processes and environments. In order to study the function of each gene in specific biological processes and environments, a variety of high-throughput gene function screening tools have been developed. Genetic screening based on CRISPR (clustered regularly interspaced short palindromic repeats) editing technology is an important tool for such studies. In CRISPR technology, a guide RNA (guide RNA, gRNA) binding a gene is designed to guide a corresponding enzyme to intervene the expression of a target gene. At present, genetic screening based on CRISPR editing technology can intervene a gene in a single cell and intervene different genes in different cells, thereby achieving the effect of screening all functional genes in a cell population.

However, from a biological perspective, combinatorial behaviors of genes widely exist in cells, which is a common characteristic of complex organisms. In other words, most biological states and functions of cells are not determined by molecules expressed by a single gene, but are jointly regulated by groups of molecules expressed by multiple genes. These molecules are successively expressed by genes and activated to transmit biological signals and regulate various biological states and behaviors of cells. In cells, the combined effects of various regulatory genes are complex and diverse, and there are compensatory mechanisms. The intervention of a combined effect of multiple genes cannot be effectively achieved by targeting a single gene, which hinders the application of CRISPR editing technology to the screening and research of the biological state and behavior of cells regulated by multiple genes. The lack of high throughput methodology hinders the combinatorial mapping from genotypes to phenotype. In many cases, disturbing a single gene is insufficient to direct to a phenotype of interest. For example, in cancer progression, sets of transcription factors crosstalk with each other to orchestrate the invasion-metastasis cascade. Therefore, a screening method for high-order combinatorial genetic perturbation is urgently needed to accelerate the research and discovery of the complex gene coordination.

From a technical view, a library with smaller complexity is favored in many genetic screenings, as large-sized library typically requires more efforts to construct and needs large amount of host cells to achieve decent coverage. In applications where cells are difficult to obtain, or subjected to inject into animals, optimized small libraries have intrinsic advantages.

So far, it is still challenging to multiplex more than two pre-designed genes in CRISPR/Cas9 screens. The challenge stems from the length limitation of oligo pool synthesis, which is typically around 150-nt and can fit 2 to 3 sgRNAs in maximum. Although longer oligo synthesis is theoretically possible, the error rate and cost quickly increase along with the length, so that the oligo pool synthesized in regular length is still more preferred and practical. The crRNA for the Cpf1 editing has its advantage of shorter unit length, and may fit in up to three units into oligo sequence with proper design, but the limited options on the PAM sequences, especially around promoter regions with high GC content, restricted it from being a universal solution for high-order combinatorial genetic screens.

Therefore, there is an urgent need in the art for a library construction method based on long overhang sequence ligation with cloning accuracy and efficiency reaching the level of library construction, and a library constructed by the library construction method.

### SUMMARY OF INVENTION

In the present application, the present inventors have developed as exemplary embodiments a high-complexity library construction method that realizes long fragment ligation at the library level, specifically a library construction method based on long overhang sequence ligation, as well as constructed and obtained a pair-specific multiplexed gRNA combination library for CRISPR editing and screening by the library construction method, and realized the purpose of performing CRISPR library screening for combinations of 4 gRNAs at the same time.

For example, in order to solve at least one of the problems existing in the prior art and enable massively parallel characterization of multiplexed and pre-designed gRNA combinations, the present inventors developed an in-library ligation method that enables the ligation of thousands of sequences to their specific counterparts, which generated a 4gRNA-comb library with high accuracy. Compared to the previous two-gRNA screening, the exemplary 4gRNA-comb library of the present invention facilitated the discovery of high-order gene coordination with higher efficiency, which cannot be achieved by the existing gRNA screening techniques. Besides, the 4gRNA-comb library could significantly reduce the required library complexity for the discovery of candidates, as a 4-gRNA combination contains multiple subsets, for example, including four single-gRNA subset, six double gRNA pairs and four three-gRNA subsets. Moreover, candidate 4gRNA combination from screening could be dissected for further investigation, e.g., to further analyze and probe the screened subset, or to identify synergistic effects among genes, and so on.

One object of the present invention is to provide a CRISPR library of pair-specific multiplexed gRNA combinations, so as to perform high-throughput screening for effect of specific multi-gene combinations in regulating the biological state and behavior of cells.

Another object of the present invention is to provide a library construction method based on long overhang sequence ligation, in which the library construction method can optimize the cloning method of pair-specific multiplexed gRNA combinations, so that the cloning efficiency can reach the level for library construction.

In a first aspect, the present invention provides a CRISPR library of pair-specific multi-gene combinations, in which the CRISPR library comprises a plurality of vectors each carrying more than two kinds of gRNA sequences, for example, each vector in the CRISPR library carries 3 to 6 kinds of gRNA sequences, for example, each vector in the CRISPR library carries 4 kinds of gRNA sequences.

The more than two kinds of gRNA sequences carried on each vector in the CRISPR library are capable of performing co-editing of more than two kinds of important molecules (e.g., genes). For example, when each vector in the CRISPR library carries 4 kinds of gRNA sequences, the 4 kinds of gRNA sequences carried on each vector are capable of performing the co-editing of four kinds of important molecules (e.g., genes). The more than two kinds of important molecules may be molecules in one or more signaling pathways, or one or more gene families, for example, may be molecules in the same signaling pathway or the same gene family, or may be molecules in different signaling pathways or different gene families.

In one embodiment, the more than two kinds of gRNA sequences may be a combination of 3 kinds of gRNA sequences, a combination of 4 kinds of gRNA sequences, a combination of 5 kinds of gRNA sequences, a combination of 6 kinds of gRNA sequences, etc.; however, considering factors such as construction cost and error rate, a combination of 4 kinds of gRNA sequences is preferred. In addition, the more than two kinds of gRNA sequences are separated between each other by tRNA. When more than two kinds of tRNAs are present, the sequences of the tRNAs can be the same or different.

In one embodiment, the present invention provides a CRISPR library of pair-specific multi-gene combinations, the CRISPR library comprises a plurality of vectors each carrying 4 kinds of gRNA sequences, and the 4 kinds of gRNA sequences carried on each vector are capable of targeting and co-editing any 4 kinds of important molecules.

Those skilled in the art can understand that the gRNA sequences can be selected and designed according to the gene sequences and gene number in the pathways and gene families to be screened to determine whether synergistic genes exist, and the number of recombinant vectors in the CRISPR library can be determined according to the desired coverage rate after the gRNA sequences are combined.

For example, in one embodiment, the CRISPR library of pair-specific multi-gene combinations comprises: approximately more than 6000 vectors each carrying 4 kinds of gRNA sequences, in which the 4 kinds of gRNA sequences carried on each vector are directed to co-editing of 4 kinds of important molecules.

In one embodiment, in the CRISPR library of pair-specific multi-gene combinations, each vector comprises an insert fragment as shown by gRNA1-tRNA1-gRNA2-tRNA2-gRNA3-tRNA3-gRNA4, wherein gRNA1, gRNA2, gRNA3 and gRNA4 are directed to editing of 4 different genes, respectively.

In one embodiment, the insert fragment shown by gRNA1-tRNA1-gRNA2-tRNA2-gRNA3-tRNA3-gRNA4 further comprises a U6 promoter, preferably a human U6 promoter, at the N-terminus.

In one embodiment, the sequences of tRNA1, tRNA2 and tRNA3 may all be the same, or two of them may be the same and one is different, or all three of them may be different. In addition, the tRNA1, tRNA2 and tRNA3 can be derived from different species, which can be appropriately selected according to the research purpose.

In a preferred embodiment, the sequences of tRNA1, tRNA2 and tRNA3 can be represented by SEQ ID NOs: 705, 706 or 707, respectively.

tRNA1
(SEQ ID NO: 705)
GGTTCCATGGTGTAATGGTTAGCACTCTGGACTCTGAATCCAGCGATCCG
AGTTCAAATCTCGGTGGAACCT
tRNA2
(SEQ ID NO: 706)
GCATGGGTGGTTCAGTGGTAGAATTCTCGCCTGCCACGCGGGAGGCCCGG
GTTCGATTCCCGGCCCATGCA
tRNA3
(SEQ ID NO: 707)
GGCTCGTTGGTCTAGGGGTATGATTCTCGCTTAGGGTGCGAGAGGTCCCG
GGTTCAAATCCCGGACGAGCCC

In a second aspect, the present invention provides a method for constructing a CRISPR library of pair-specific multiplexed gRNA combinations based on long overhang sequence ligation, the method comprising the following steps:
(1) designing a sequence library of pair-specific multiplexed gRNA combinations according to the pathway or gene family to be screened, and synthesizing a mixture of two or more oligonucleotide chain pools according to the sequences of the library, wherein each oligonucleotide sequence in each oligonucleotide chain pool comprises one or more kinds of gRNAs, wherein for 3′-end of each sequence in one oligonucleotide chain pool, there is only one kind of 5′-end sequence completely complementary thereto in another oligonucleotide chain pool, and the complementary portion has a sequence length of 2-100 nucleotides, such as 4-50, 10-40, 15-35, 20-30, preferably 21 nucleotides (21 nt);
(2) performing PCR amplification with the mixture of two or more oligonucleotide chain pools as templates, respectively, to obtain two or more corresponding library chain pools, respectively;
(3) using a nicking endonuclease to digest the two or more corresponding library chain pools obtained by PCR amplification in step (2) respectively to generate products each having one or two complementary long overhangs of 2-100 nucleotides, such as 4-50, 10-40, 15-35, 20-30, preferably 21 nt, and mixing the digested products from each of the library chain pools and performing ligation of the digested products from the library chain pools by annealing to generate linear gRNA library sequences, each of which comprises the pair-specific multiplexed gRNA combinations;
(4) inserting the linear gRNA library sequence obtained in step (3) into a vector to form a primary library vector; and
(5) sequentially inserting a tRNA sequence between two adjacent gRNAs in the primary library vector to form a complete library vector, wherein the complete library vector comprises pair-specific multiplexed gRNA combinations which comprise a tRNA sequence between any two adjacent gRNAs.

In one embodiment, those skilled in the art will appreciate that in step (1), a mixture of two or more oligonucleotide chain pools, such as a mixture of oligonucleotide chain pools 1, 2 and 3, a mixture of oligonucleotide chain pools 1, 2, 3 and 4, and the like, may be designed and synthesized, according to the number of gRNAs to be constructed in one vector of the CRISPR library and the requirements in practice, and each oligonucleotide sequence in each oligonucleotide chain pool may comprise a suitable number of gRNAs, for example, one or more kinds of gRNAs. There is no limitation to the number of the oligonucleotide chain pools in the mixture, provided that, no matter how many oligonucleotide chain pools are designed in the mixture, they will be ligated by their complementary long overhangs into linear library sequences, after PCR amplification and nicking endonuclease digestion. For example, if the mixture of two or more oligonucleotide chain pools comprises three oligonucleotide chain pools, i.e., oligonucleotide chain pools 1, 2 and 3, for 3′-end of each sequence in oligonucleotide chain pool 1, there is only one kind of 5′-end sequence completely complementary thereto in oligonucleotide chain pool 2, and for 3′-end of each sequence in oligonucleotide chain pool 2, there is only one kind of 5′-end sequence completely complementary thereto in oligonucleotide chain pool 3, wherein the complementary portion has a sequence length of 2-100 nucleotides, such as 4-50, 10-40, 15-35, 20-30, preferably 21 nucleotides (21 nt). In this way, after PCR amplification and nicking endonuclease digestion, the sequence derived from oligonucleotide chain pool 1, the sequence derived from oligonucleotide chain pool 2, and the sequence derived from oligonucleotide chain pool 3, may be ligated into a linear sequence (i.e., by their complementary long overhangs), which comprises pair-specific multiplexed gRNA combinations.

For example, for constructing a CRISPR library of pair-specific 4-gRNA combinations, a mixture of two oligonucleotide chain pools may be designed, in which each oligonucleotide sequence in either oligonucleotide chain pool comprises 2 kinds of gRNAs, or each oligonucleotide sequence in one oligonucleotide chain pool comprises 1 gRNA and each oligonucleotide sequence in the other oligonucleotide chain pool comprises 3 kinds of gRNAs; or a mixture of three oligonucleotide chain pools may be designed, in which each oligonucleotide sequence in one oligonucleotide chain pool comprises 2 kinds of gRNAs, and each oligonucleotide sequence in the remaining two oligonucleotide chain pools comprises 1 gRNA.

In another embodiment, for constructing a CRISPR library of pair-specific 5-gRNA combinations, a mixture of two oligonucleotide chain pools may be designed, in which each oligonucleotide sequence in one oligonucleotide chain pool comprises 2 kinds of gRNAs, and each oligonucleotide sequence in the other oligonucleotide chain pool comprises 3 kinds of gRNAs; or a mixture of three oligonucleotide chain pools may be designed, in which each oligonucleotide sequence in one oligonucleotide chain pool comprises 1 gRNA, and each oligonucleotide sequence in two oligonucleotide chain pools comprises 2 kinds of gRNAs, and the like.

In one embodiment, the present invention provides a method for constructing a CRISPR library of pair-specific multiplexed gRNA combinations based on long overhang sequence ligation, the method comprising the following steps:
(1) designing a sequence library of pair-specific multiplexed gRNA combinations according to the pathway or gene family to be screened, and synthesizing a mixture of oligonucleotide chain pools 1 and 2 (referred to as oligo pool 1 and oligo pool 2 hereafter) according to the sequences of the library, wherein each oligonucleotide sequence in the oligonucleotide chain pool 1 comprises gRNA1 and gRNA2, and each oligonucleotide sequence in the oligonucleotide chain pool 2 comprises gRNA3 and gRNA4, wherein for the 3′ end of each sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt);
(2) performing PCR amplification with the mixture of oligonucleotide chain pools 1 and 2 as templates, respectively, to obtain corresponding library chain pools 1 and 2, respectively;
(3) using a nicking endonuclease to digest the library chain pools 1 and 2 obtained by PCR amplification in step (2) respectively to generate products each having a complementary long overhang of 2-100 nucleotides, such as 4-50, 10-40, 15-35, 20-30, preferably 21 nt, and performing ligation of the digested products from the library chain pools 1 and 2 by annealing to generate gRNA library sequences each of which is shown by gRNA1-gRNA2-gRNA3-gRNA4;
(4) inserting the gRNA library sequence obtained in step (3) into a vector to form a primary library vector; and
(5) sequentially inserting tRNA1, tRNA2 and tRNA3 sequences into the primary library vector to form a complete library vector, wherein the complete library vector comprises the insert fragment shown by gRNA1-tRNA1-gRNA2-tRNA2-gRNA3-tRNA3-gRNA4.

In one embodiment, for the 3′ end of each kind of sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt) in the present invention. The sequence of the complementary portion is capable of forming a complementary overhang after it is subjected to PCR amplification and nicking endonuclease digestion. That is, the term “long overhang” or “long overhang sequence” as used herein corresponds to the complementary portion between the oligonucleotide chain pool 1 and the oligonucleotide chain pool 2. Generally, the long overhang sequence may have a length of 2-100 nucleotides, such as 4-50, 10-40, 15-35, 20-30, preferably 21 nt.

It will be understood by those skilled in the art that the length of the complementary portion sequence (i.e., the long overhang sequence to be generated therefrom) may be longer or shorter in specific instances. The selection of the length should be considered in two aspects: (1) it is long enough to ensure that enough one-to-one correspondence combinations between the single-chain pool 1 (i.e., the oligonucleotide chain pool 1) and the single-chain pool 2 (i.e., the oligonucleotide chain pool 2) can be generated; (2) the total length of the designed sequence does not exceed an upper limit of the current general synthesis of oligonucleotide single-chain pools.

In one embodiment, in step (2), the reverse primer in the primer pair used for the amplification of the oligonucleotide chain pool 1 is biotinylated, and the forward primer in the primer pair used for the amplification of the oligonucleotide chain pool 2 is biotinylated, so that a double-stranded amplification product with biotin in one amplified chain is obtained, and in step (3), a biotin-carrying small fragment generated during the nicking endonuclease digestion can be removed by using a streptavidin magnetic bead, thereby more thoroughly exposing the 3′- and 5′-overhang products of the oligonucleotide chain pools.

In one embodiment, in step (3), the library chain pools 1 and 2 obtained by the PCR amplification are digested by nicking endonuclease, respectively, wherein the sequence in library chain pool 1 forms 3′-end long overhang of 21 nt, and the sequence in library chain pool 2 forms 5′-end long overhang of 21 nt; for the pair-specific gRNA combinations, the two overhang sequences are complementary, and the corresponding two DNA chains can be specifically ligated under the action of T4 ligase, thereby obtaining a DNA sequence shown by gRNA1-gRNA2-gRNA3-gRNA4.

In one embodiment, the nicking endonuclease used in step (3) may be selected from, but not limited to, Nb.BsrDI or Nt.BspQI nicking endonucleases.

In one embodiment, the double-stranded library fragments obtained by annealing and ligation in step (3) may have double-stranded fragments that are poorly matched (for example, mismatched), therefore, before step (4), T7 endonuclease I (T7E1) may be used to perform a digestion reaction to remove these poorly matched double-stranded fragments.

In one embodiment, the vector used in step (4) may be a viral vector, for example, a lentiviral vector, a retroviral vector, an adenoviral vector, an adeno-associated virus vector, but not limited thereto, and those skilled in the art can select an appropriate vector according to actual requirements.

In one embodiment, the sequences of tRNA1, tRNA2 and tRNA3 used in step (5) are all the same, or two of them are the same and one is different, or all three of them are different. In addition, tRNA1, tRNA2 and tRNA3 may be derived from different species, which may be appropriately selected according to the research purpose.

In one embodiment, in step (5), tRNA1, tRNA2 and tRNA3 are sequentially introduced through golden gate assembly to form a vector comprising the insert fragment shown by gRNA1-tRNA1-gRNA2-tRNA2-gRNA3-tRNA3-gRNA4. In the introduction of tRNA1, tRNA2 and tRNA3, different endonucleases are used respectively to ensure that each insertion is at a pre-designed position.

In a preferred embodiment, the TypeIIS endonucleases used in the three-step reaction for the introduction of tRNA1, tRNA2 and tRNA3 may be AarI, BbsI and BsaI, respectively. Those skilled in the art can design desired restriction sites and select corresponding endonucleases according to actual needs. The method of sequentially introducing tRNA1, tRNA2 and tRNA3 is also within the capacity of those skilled in the art.

In one embodiment, the insert fragment shown by gRNA1-tRNA1-gRNA2-tRNA2-gRNA3-tRNA3-gRNA4 is under the control of a U6 promoter, preferably a human U6 promoter.

In a specific embodiment, the complete library vectors obtained in step (5) are about 6000 recombinant vectors, and each vector comprises different 4-gRNA combinations, which are capable of performing co-editing of 4 kinds of important molecules in a signaling pathway or a gene family.

In a specific embodiment, the vector used in step (4) is a lentiviral vector, therefore, after obtaining the complete library vector in step (5), the following step is also included: (6) a step of packaging lentivirus with the constructed library vector and detecting lentivirus titer.

In a specific embodiment, the method for constructing a CRISPR library of pair-specific multiplexed gRNA combinations based on long overhang sequence ligation of the present invention comprises the following steps:
(1) designing a sequence library of pair-specific multiplexed gRNA combinations according to the pathway or gene family to be screened, and synthesizing a mixture of oligonucleotide chain pools 1 and 2 according to the sequences of the library, wherein each oligonucleotide sequence in the oligonucleotide chain pool 1 comprises gRNA1 and gRNA2, and each oligonucleotide sequence in the oligonucleotide chain pool 2 comprises gRNA3 and gRNA4, wherein for the 3′ end of each sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt);
(2) performing PCR amplification with the mixture of oligonucleotide chain pools 1 and 2 as templates, respectively, to obtain library chain pools 1 and 2, respectively, wherein the reverse primer in the primer pair used for amplification of the oligonucleotide chain pool 1 is biotinylated, and the forward primer in the primer pair used for amplification of the oligonucleotide chain pool 2 is biotinylated;
(3) using a nicking endonuclease to digest the library chain pools 1 and 2 obtained by PCR amplification in step (2) respectively to generate products each having a complementary long overhang of 2-100 nucleotides, such as 4-50, 10-40, 15-35, 20-30, preferably 21 nt, performing ligation of the digested products from the library chain pools 1 and 2 by annealing after biotin-carrying small fragments are removed by purification with streptavidin magnetic beads, performing digestion of the ligation products with T7 endonuclease I (T7E1) to remove poorly matched double-stranded fragments and finally generate gRNA library sequences, each of which is shown by gRNA1-gRNA2-gRNA3-gRNA4;
(4) inserting the gRNA library sequence obtained in step (3) into a vector to form a primary library vector; and
(5) sequentially inserting tRNA1, tRNA2 and tRNA3 sequences into the primary library vector to form a complete library vector, wherein the complete library vector comprises the insert fragment shown by gRNA1-tRNA1-gRNA2-tRNA2-gRNA3-tRNA3-gRNA4.

In one embodiment, the vector used in step (4) may be a viral vector, for example, a lentiviral vector, a retroviral vector, an adenoviral vector, an adeno-associated virus vector, but not limited thereto, and those skilled in the art may select an appropriate vector according to actual needs.

In one embodiment, the vector used in step (4) is a lentiviral vector, and the method further comprises the following step after obtaining the complete library vector in step (5): (6) a step of packaging lentivirus with the constructed library vector and detecting lentivirus titer.

In an alternative embodiment, the present application provides a method for constructing a CRISPR library of pair-specific multiplexed gRNA combinations based on long overhang sequence ligation, comprising:
(1) designing a sequence library of pair-specific multiplexed gRNA combinations according to the pathway or gene family to be screened, and synthesizing a mixture A of two oligonucleotide chain pools A1 and A2 and a mixture B of two oligonucleotide chain pools B1 and B2, according to the sequences of the library, wherein each oligonucleotide sequence in each oligonucleotide chain pool comprises one or more kinds of gRNAs,
wherein for 3′-end of each sequence in oligonucleotide chain pool A1, there is only one kind of 5′-end sequence completely complementary thereto in oligonucleotide chain pool A2, and the complementary portion has a sequence length of 2-100 nucleotides, such as 4-50, 10-40, 15-35, 20-30, preferably 21 nucleotides (21 nt);
wherein for 3′-end of each sequence in oligonucleotide chain pool B1, there is only one kind of 5′-end sequence completely complementary thereto in oligonucleotide chain pool B2, and the complementary portion has a sequence length of 2-100 nucleotides, such as 4-50, 10-40, 15-35, 20-30, preferably 21 nucleotides (21 nt);
wherein for 3′-end of each sequence in oligonucleotide chain pool A2, there is only one kind of 5′-end sequence completely complementary thereto in oligonucleotide chain pool B1, and the complementary portion has a sequence length of 2-100 nucleotides, such as 4-50, 10-40, 15-35, 20-30, preferably 21 nucleotides (21 nt);
(2) performing PCR amplification with the mixture A as templates, respectively, to obtain corresponding library chain pools A1 and A2, respectively; and performing PCR amplification with the mixture B as templates, respectively, to obtain corresponding library chain pools B1 and B2, respectively;
(3) using a nicking endonuclease to digest the library chain pools A1 and A2 obtained by PCR amplification in step (2) respectively to generate products each having one or two complementary long overhangs of 2-100 nucleotides, such as 4-50, 10-40, 15-35, 20-30, preferably 21 nt; using a nicking endonuclease to digest the library chain pools B1 and B2 obtained by PCR amplification in step (2) respectively to generate products each having one or two complementary long overhangs of 2-100 nucleotides, such as 4-50, 10-40, 15-35, 20-30, preferably 21 nt; and mixing the digested products from the library chain pools A1, A2, B1 and B2 and performing ligation of the digested products from the library chain pools A1, A2, B1 and B2 by annealing to generate linear gRNA library sequences A1-A2-B1-B2, each of which comprises the pair-specific multiplexed gRNA combinations;
(4) inserting the gRNA library sequence A1-A2-B1-B2 obtained in step (3) into a vector to form a primary library vector; and
(5) sequentially inserting a tRNA sequence between two adjacent gRNAs in the primary library vector to form a complete library vector, wherein the complete library vector comprises pair-specific multiplexed gRNA combinations which comprise a tRNA sequence between any two adjacent gRNAs.

Those skilled in the art may appreciate that in step (1), two or more additional mixtures of two oligonucleotide chain pools may be further synthesized according to the designed sequences of the library and the requirements in practice, wherein each oligonucleotide sequence in each oligonucleotide chain pool comprises one or more kinds of gRNAs. Each oligonucleotide sequence in each oligonucleotide chain pool follows the same principle of complementary, so that after PCR amplification and nicking endonuclease digestion, the oligonucleotide sequences derived from each oligonucleotide chain pool will be ligated into a linear sequence (i.e., by their complementary long overhangs), which comprises pair-specific multiplexed gRNA combinations.

In one embodiment, step (3) may be performed as follows:
using a nicking endonuclease to digest the library chain pools A1 and A2 obtained by PCR amplification in step (2) respectively to generate products each having one or two complementary long overhangs of 2-100 nucleotides, such as 4-50, 10-40, 15-35, 20-30, preferably 21 nt, and mixing the digested products from the library chain pools A1 and A2 and performing ligation of the digested products from the library chain pools A1 and A2 by annealing to generate gRNA library sequences A1-A2;
using a nicking endonuclease to digest the library chain pools B1 and B2 obtained by PCR amplification in step (2) respectively to generate products each having one or two complementary long overhangs of 2-100 nucleotides, such as 4-50, 10-40, 15-35, 20-30, preferably 21 nt, and mixing the digested products from the library chain pools B1 and B2 and performing ligation of the digested products from the library chain pools B1 and B2 by annealing to generate gRNA library sequences B1-B2; and
mixing the gRNA library sequences A1-A2 and gRNA library sequences B1-B2 and performing ligation of them by annealing to generate gRNA library sequences A1-A2-B1-B2, each of which comprises the pair-specific multiplexed gRNA combinations.

In a third aspect, the present invention provides a library construction method based on long overhang sequence ligation, the method comprising the following steps:
(1) designing and synthesizing a mixture of oligonucleotide chain pools 1 and 2, wherein each oligonucleotide in the oligonucleotide chain pool 1 has a length of 141-165 bp, and each oligonucleotide in the oligonucleotide chain pool 2 has a length of 150 bp; and, for the 3′ end of each kind of sequence in the oligonucleotide chain pool 1, there is only one kind of 5′-end sequence completely complementary thereto in the oligonucleotide chain pool 2, and the complementary portion has a sequence length of 15-35 nucleotides, preferably 20-30 nucleotides, more preferably 21 nucleotides (21 nt);
(2) performing PCR amplification with the mixture of oligonucleotide chain pools 1 and 2 as templates, respectively, to obtain library chain pools 1 and 2, respectively, wherein the reverse primer in the primer pair used for amplification of the oligonucleotide chain pool 1 is biotinylated, and the forward primer in the primer pair used for amplification of the oligonucleotide chain pool 2 is biotinylated;
(3) using a nicking endonuclease to digest the library chain pools 1 and 2 obtained by PCR amplification in step (2) respectively to generate products each having a complementary long overhang of 15-35 nucleotides, preferably 20-30 nucleotides, more preferably 21 nt, performing ligation of the digested products from the library chain pools 1 and 2 by annealing after biotin-carrying small fragments are removed by purification with streptavidin magnetic beads, performing digestion of the ligation product with T7 endonuclease I (T7E1) to remove poorly matched double-stranded fragments and finally generate insert sequences;
(4) inserting the insert sequence obtained in step (3) into a vector to form a primary library vector.

Wherein, in step (1), “for the 3′ end of each kind of sequence in the oligonucleotide chain pool 1, there is only one kind of 5′-end sequence completely complementary thereto in the oligonucleotide chain pool 2”, means that the 5′ end of each sequence in the oligonucleotide chain pool 2 can be completely complementary to the 3′ end of one or more sequences in the oligonucleotide chain pool 1; for example, in one embodiment, the 5′ end of each sequence in the oligonucleotide chain pool 2 is completely complementary to the 3′ end of one sequence in the oligonucleotide chain pool 1; in another embodiment, the 5′ end of each sequence in the oligonucleotide chain pool 2 is completely complementary to the 3′ end of a plurality of sequences in the oligonucleotide chain pool 1, so that one target sequence can test a variety of other additional sequences that can be paired with it, which can reduce the cost of library construction, and many such target sequences can be tested simultaneously during a test performed in the library.

Those skilled in the art will appreciate that in step (1), a mixture of more than two oligonucleotide chain pools, such as a mixture of oligonucleotide chain pools 1, 2 and 3, a mixture of oligonucleotide chain pools 1, 2, 3 and 4, and the like, can be designed and synthesized, according to the requirements in practice. There is no limitation to the number of the oligonucleotide chain pools in the mixture. No matter how many oligonucleotide chain pools are designed in the mixture, they will be ligated by their complementary long overhangs into a linear sequence, after PCR amplification and nicking endonuclease digestion. For example, if the mixture of more than two oligonucleotide chain pools comprises three oligonucleotide chain pools, i.e., oligonucleotide chain pools 1, 2 and 3, for 3′-end of each sequence in oligonucleotide chain pool 1, there is only one kind of 5′-end sequence completely complementary thereto in oligonucleotide chain pool 2, and for 3′-end of each sequence in oligonucleotide chain pool 2, there is only one kind of 5′-end sequence completely complementary thereto in oligonucleotide chain pool 3, wherein the complementary portion has a sequence length of 15-35 nucleotides, preferably 20-30 nucleotides, more preferably 21 nucleotides (21 nt). In this way, after PCR amplification and nicking endonuclease digestion, the sequence derived from oligonucleotide chain pool 1, the sequence derived from oligonucleotide chain pool 2, and the sequence derived from oligonucleotide chain pool 3, may be ligated into a linear sequence by their complementary long overhangs.

In one embodiment, the nicking endonuclease used in step (3) may be selected from, but not limited to, Nb.BsrDI or Nt.BspQI nicking endonuclease.

In one embodiment, the vector used in step (4) can be a viral vector, for example, a lentiviral vector, a retroviral vector, an adenoviral vector, an adeno-associated virus vector, but not limited thereto, and those skilled in the art can select appropriate vectors according to actual needs.

In a specific embodiment, the vector used in step (4) is a lentiviral vector, therefore, after obtaining the primary library vector in step (4), the method further comprises the following steps: (5) a step of packaging lentivirus with the constructed library vector and detecting lentivirus titer.

In one embodiment, the primary library vector obtained in step (4) can be further processed, for example, by introducing another required insert fragment to form a complete library vector.

According to the disclosure in the second aspect and the third aspect, those skilled in the art can understand that the term “in-library ligation” refers to a high-throughput and specific fragment ligation in a library, which is a nucleic acid sample (library) composed of a large number of mixed sequences, to achieve the purpose of extending sequence length and/or increasing sequence diversity. By using nicking endonuclease (e.g., Nb.BsrDI), this method can generate on nucleic acid fragments long overhangs that have one-to-one correspondence and can realize pairwise complementary ligation between nucleic acid sequences. Thus, in a high-throughput library, the ligation reaction occurs only between pre-designed sequences. The key difference between this ligation reaction and the general restriction endonuclease-mediated digestion reaction lies in that: the in-library ligation reaction can generate overhang sequences with variable lengths on nucleic acid sequences through pre-designed complementary sequences and nicking restriction enzymes. Since the length of the overhang sequence determines the number of fragment combinations that can achieve pairwise complementarity (the theoretical value is 4ⁿ, wherein n is the number of nucleotides in the overhang sequence, for example, the theoretical value for an overhang sequence with a length of 10 nucleotides is 4¹⁰). The in-library ligation reaction can theoretically realize the one-to-one corresponding ligation among the internal sequences of a mixed nucleic acid sample with extremely high complexity. However, the common restriction enzymes (e.g., EcoRI, BamHI, etc.), firstly, have very limited types, and secondly, can generate overhang sequences that are generally 4 to 6 nucleotides with a specific sequence, so that the library constructed by using common restriction enzymes does not comprise a high-throughput one-to-one corresponding ligation among specified sequences.

In the in-library ligation design, a nicking endonuclease, for example, Nb. BsrDI, is used to generate nicks on the double strand DNA sequences. The Nb. BsrDI is one example of so-called “nicking endonuclease”, which has different cutting pattern from the commonly used restriction enzymes (e.g., EcoRI). To apply the Nb. BsrDI digestion, two recognition sites were designed to ligate two sub-pools. One recognition site located on the top strand of DNA, generated one nick at the top strand. Another recognition site located on the bottom strand of DNA, generated one nick at the bottom strand. And the two recognition sites were apart away from each other, resulted two nicks that were also apart from each other. Importantly, the distance between the two nicks is flexible. When we design the oligo, we could adjust the distance between the two recognition sites to determine the distance between the two nicks. This is why long overhangs of 21-nt (or shorter, or longer, e.g., 2-100 nucleotides, such as 4-50, 10-40, 15-35, 20-30, preferably 21 nt) can be generated by using nicking endonuclease.

In a fourth aspect, the present invention provides a CRISPR library constructed by the method of the second or third aspect.

In a fifth aspect, the present invention provides a host cell transformed with the CRISPR library of the first or fourth aspect.

In one embodiment, the host cell used to transform the CRISPR library can be a prokaryotic cell, such as a bacterial cell, such as, but not limited to, an E. coli cell, etc., or a eukaryotic cell such as a fungal cell (e.g., yeast cell), or a mammalian cell, such as, but not limited to, a murine cell or a human cell, and the like.

In a preferred embodiment, the host cell used to transform the CRISPR library is a mammalian cell, such as, but not limited to, a murine cell or a human cell, and the like.

In a sixth aspect, the present invention provides a use of the CRISPR library of pair-specific multi-gene combinations. Specifically, the CRISPR library can be used for a high-throughput screening of a signaling pathway or gene family that determines a specific cell biological event, so as to obtain a plurality of kinds of interacting genes corresponding to a specific phenotype.

In a seventh aspect, the present invention provides a high-throughput method for combined screening of a plurality of kinds of interacting genes, the method is performed by using the CRISPR library of pair-specific multi-gene combinations described in the first aspect of the present invention.

In summary, the present application provides a fragment to realize high-throughput pair-specific ligation in library by using a long overhang sequence that is generated with nicking restriction enzyme, can achieve pairwise complementarity and has one-to-one correspondence. By optimizing the cloning method of pair-specific multiplexed gRNA combinations so that the cloning efficiency can reach the level of library construction, an innovative library construction scheme of multi-gene editing system is provided. The establishment of this library construction scheme makes it possible to study the role of multi-gene combined functions in cell regulation. That is, the present invention has achieved at least one of the following beneficial technical effects:
(1) The method for constructing a library of multiplexed gRNA combinations of the present invention overcomes the length limitation of oligonucleotide chain pool synthesis and allows the expression of a plurality of kinds of pre-designed gRNAs from one vector. The cloning method developed based on the method of the present invention can efficiently construct a CRISPR library of pair-specific multi-gene combinations, and provides a new powerful tool for the application of CRISPR/Cas9 screening to promote large-scale, programmable complex cell perturbations; and
(2) Using this library, high-throughput screening can be performed for the role of specific multi-gene combinations in regulating the biological state and behavior of cells, and the role of multi-gene combined functions in cell regulation can be studied.

The above is an overview and therefore contains simplifications, generalizations and omissions of details where necessary. Accordingly, those skilled in the art will recognize that this overview is illustrative only and is not intended to limit the present invention in any way. Other aspects, features and advantages of the method, CRISPR library and/or other subject matter described herein will be apparent from the teachings presented herein. An overview is provided to briefly introduce some selected concepts that are further described in the detailed description below. This overview is not intended to identify key features or essential features of the claimed subject matter, nor is it intended to be used as an aid in determining the scope of the claimed subject matter. Furthermore, the contents of all references, patents, and published patent applications cited throughout this application are incorporated herein by reference in their entirety.

### BRIEF DESCRIPTION OF FIGURES

FIG. 1 schematically shows the working principle of the 4-gRNA expression cassette and the editing efficiency test, wherein:
(A) shows that 4 different gRNAs are spaced by three tRNAs, and the tRNAs are cleaved and removed after transcription to release single gRNAs that bind to Cas9 endonuclease, respectively;
(B) shows the editing efficiency of gRNAs expressed as single transcripts or multiple transcripts. The gRNAs targeting EMX1, DYRK1A, DMD and VEGFA were cloned into 4 individual vectors, respectively, or cloned into one vector as a multiplexed expression cassette; and editing efficiencies were compared for cells transfected with each single gRNA (1× single gRNA, blue bars), 4-gRNA combinations (i.e., 4× gRNA multiplexed, red bars), and 4 individual gRNAs (4× single gRNA, green bars); i.e., the three comparative experimental results for each group were represented by three bars, from left to right: editing efficiencies of cells transfected with each single gRNA, 4-gRNA combinations, and 4 individual gRNAs. At all 4 targeted sites, the editing efficiency of the 4-gRNA combinations was essentially at the same level as the editing efficiency of the 4 individual gRNAs, and the reduction in the editing efficiency of the 4-gRNA combinations and the 4 individual gRNAs as compared to the editing efficiency of each single gRNA could be due to the competition of multiplex gRNAs for Cas9. The experiments were performed in triplicate.

FIG. 2 schematically shows the construction scheme of 4 kinds of gRNA combinations according to an example of the present invention:
(1) In-library ligation: An oligonucleotide library with two oligonucleotide chain pools (i.e., a mixture of oligonucleotide pools 1 and 2) was synthesized. The mixture of oligonucleotide pools was used as temperate, so that each of the oligonucleotide chain pools was subjected to PCR amplification in two separate reactions by specific PCR primer binding sites located at the 5′ and 3′ ends of oligonucleotide to generate sub-pool-1 and sub-pool-2, respectively. The reverse PCR primer for the oligonucleotide chain pool 1 (grey line in sub-pool-1) and the forward PCR primer for the oligonucleotide chain pool 2 (grey line in sub-pool-2) were modified with 5′-biotin. These PCR products had two Nb.BsrDI recognition sites near the complementary regions, one on the upper chain and the other on the lower chain. After Nb.BsrDI digestion, staggered nicks were generated, and small fragments were released from the 3′ end of the PCR product of the oligonucleotide chain pool 1 and the 5′ end of the PCR product of the oligonucleotide chain pool 2. These small fragments comprised biotin and were then captured and removed by streptavidin beads via the biotin modification thereon. The oligonucleotide chain pool with 3′-overhangs and the oligonucleotide chain pool with 5′-overhangs were then combined and ligated by sequence complementarity via annealing. Each product derived from the oligonucleotide chain pool 1 had one and only one counterpart derived from the oligonucleotide chain pool 2, and undesired ligation products carrying mismatches would be removed by T7E1 digestion (i.e., “mismatch cleavage”). The grey lines containing grey rectangles schematically represent the sequences derived from the oligonucleotide chain pool 1 or 2, and the grey rectangles in sub-pool-1 and sub-pool-2 schematically represent the spacer sequences of gRNAs;
(2) Vector library construction: The correct ligation product was cloned between the human U6 promoter and scaffold 4 of the lentiviral backbone by Golden Gate Assembly. Three more sequential Golden Gate assemblies were then performed, and the following elements were cloned in sequence: scaffold 1 and human Gln-tRNA (i.e., tRNA1), scaffold 2 and human Gly-tRNA (i.e., tRNA2), scaffold 3 and human Pro-tRNA (i.e., tRNA3). The final resulting vector consisted of a multiplexed gRNA expression cassette (spaced with 3 tRNAs) driven by the U6 promoter.

FIG. 3 shows the design principle of complementary sequences for in-library ligation: Random 21 nt oligonucleotide sequences were generated, and the 21 nt overhang sequences for in-library ligation must meet all of the following criteria: (1) GC content was 45% to 60%, Tm was 60° C. to 65° C.; (2) oligonucleotide secondary structure energy predicted by RNAfold must be less than −3 kcal/mol; (3) there was no restriction enzyme recognition site used in downstream cloning steps; (4) compared with any other sequences in the oligonucleotide chain pools, they could handle more than 5 mismatches, and at least one mismatch was located within 4 oligonucleotides at the 5′ end or at the 3′ end; (5) the secondary structure energy of the duplex with any other sequence in the oligonucleotide chain pools must be less than −15 kcal/mol as predicted by RNAfold. The sequences meeting all of the above criteria would be included in the oligonucleotide library. The search stopped when a certain number of sequences were generated. In this example, the number was 6236.

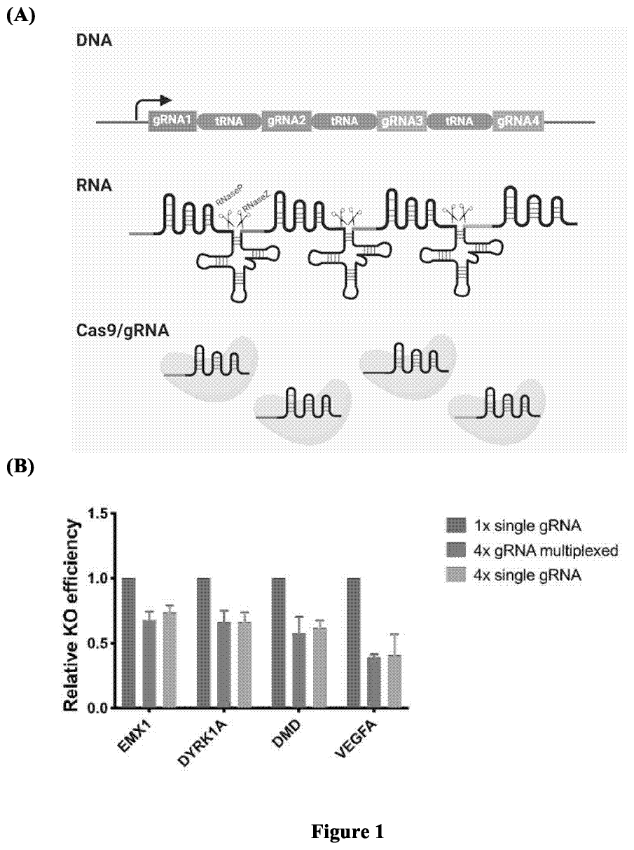

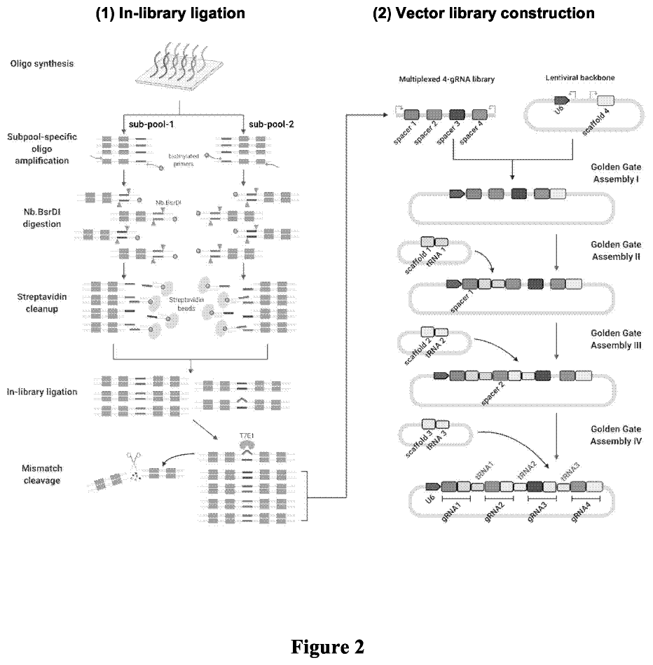

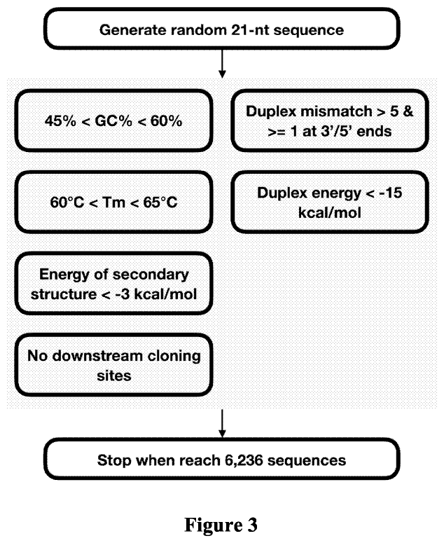

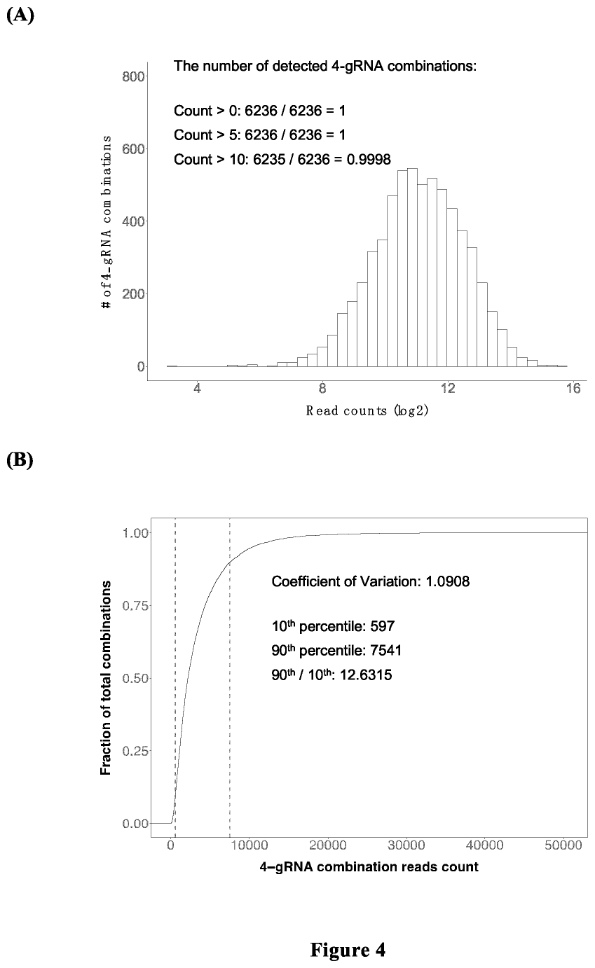

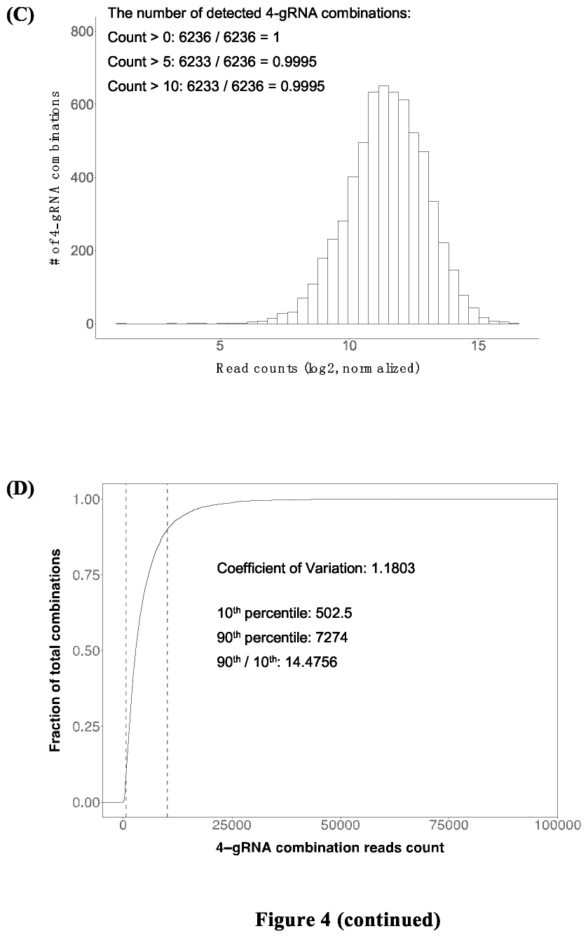

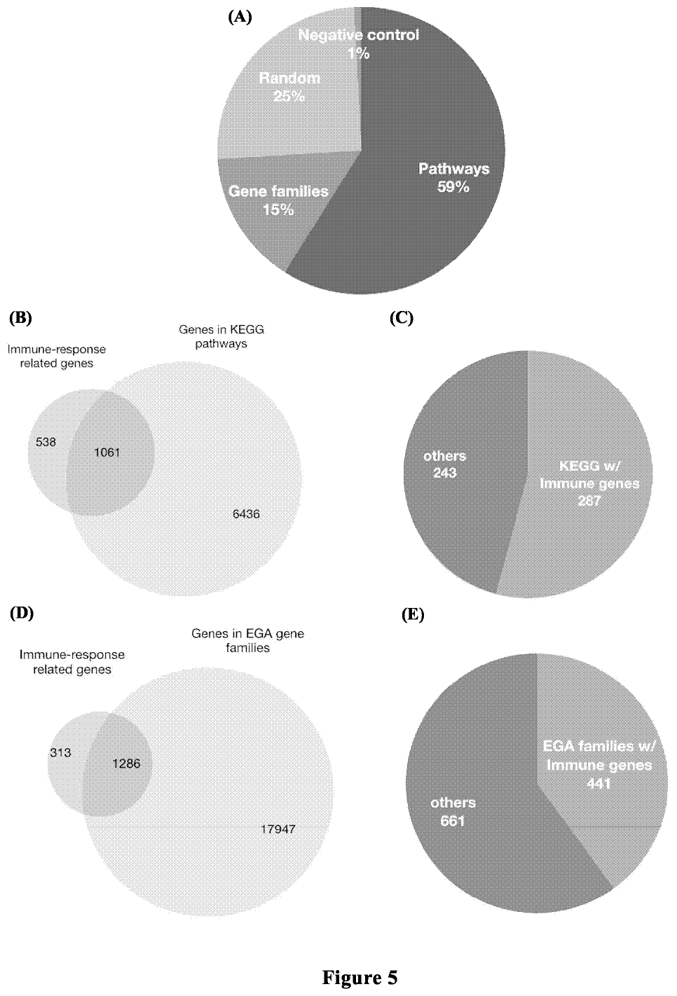

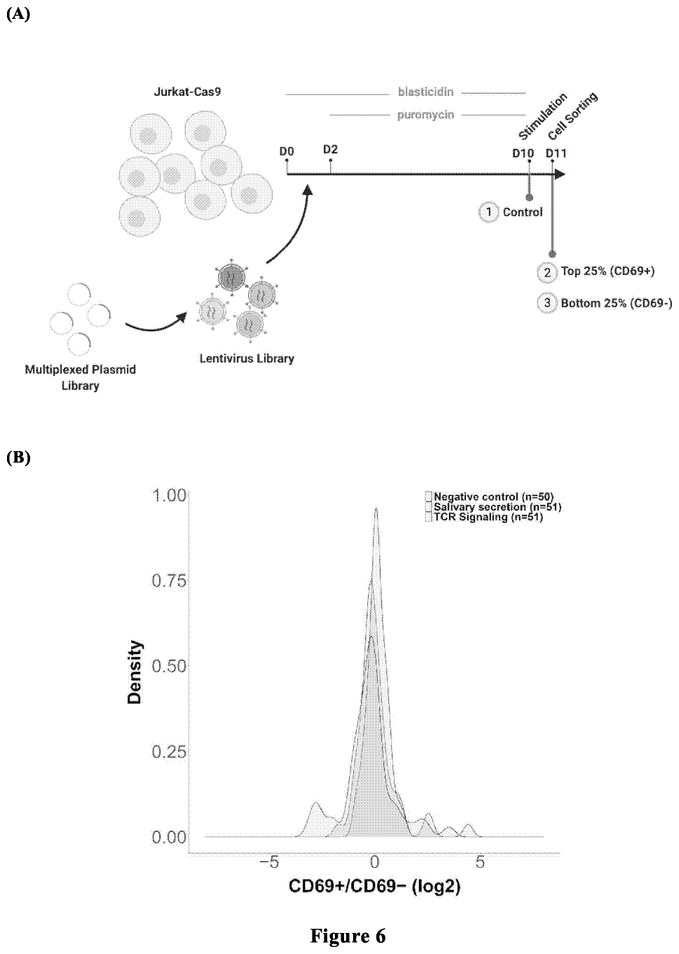

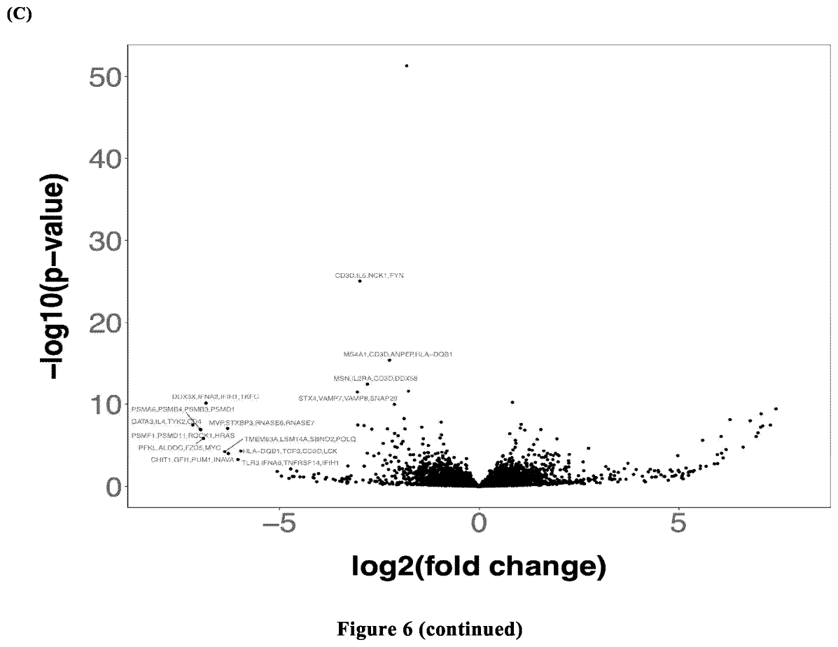

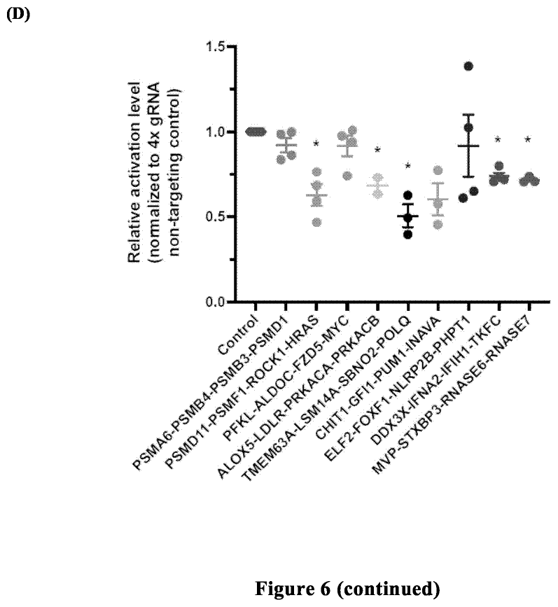

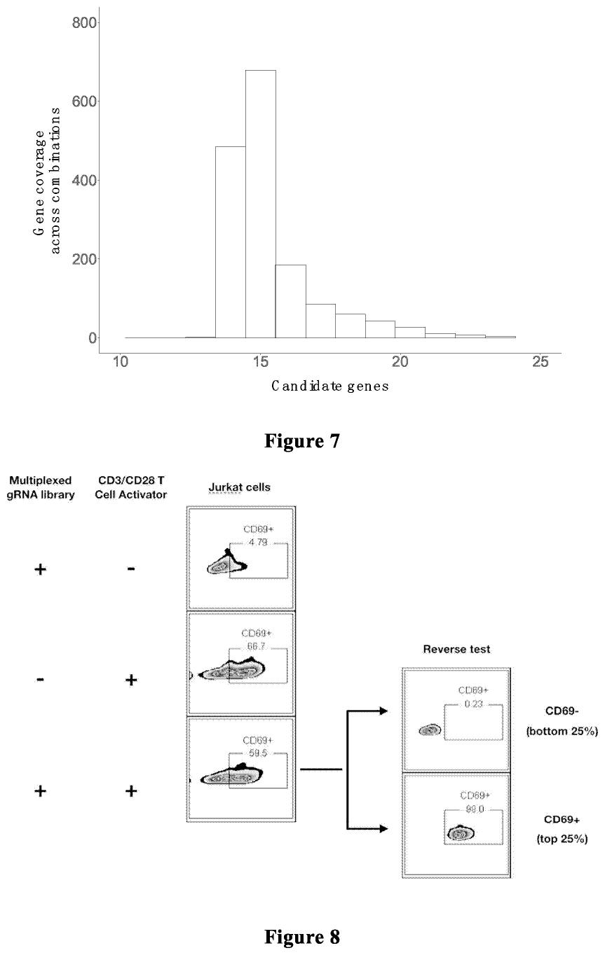

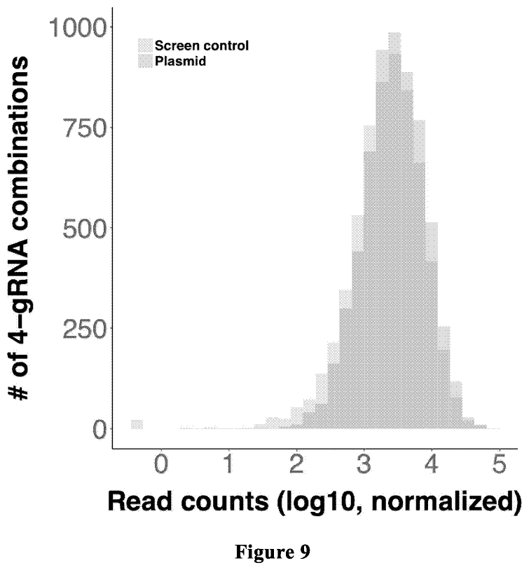

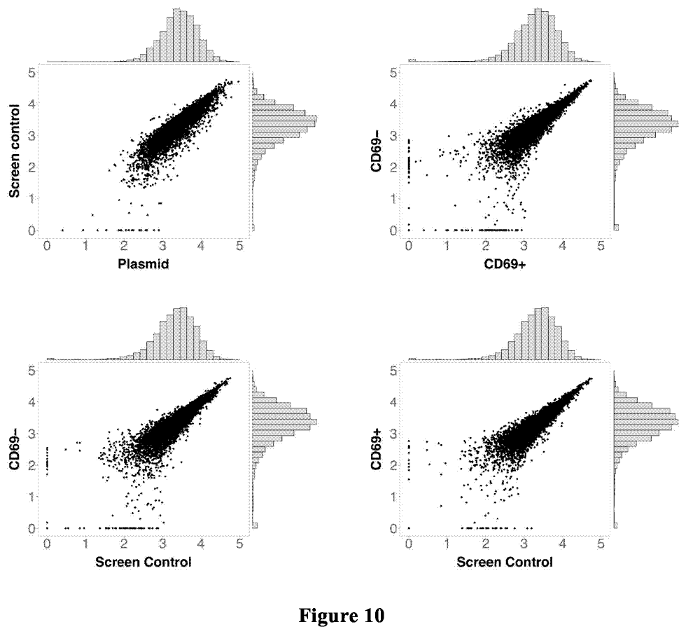

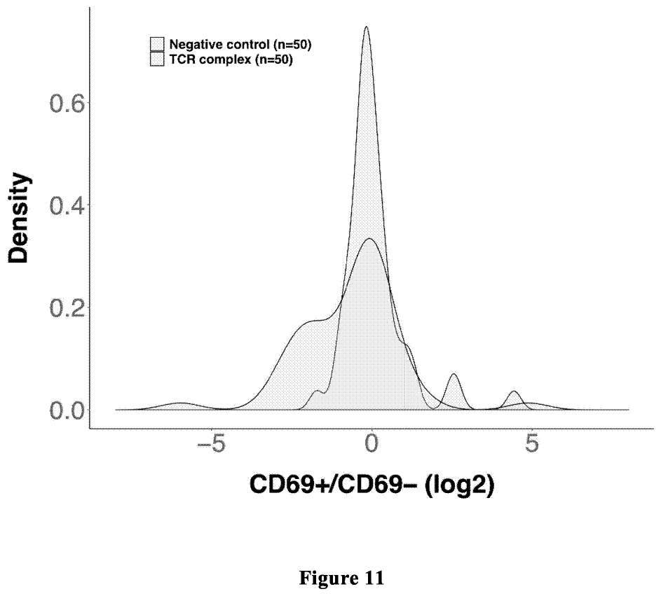

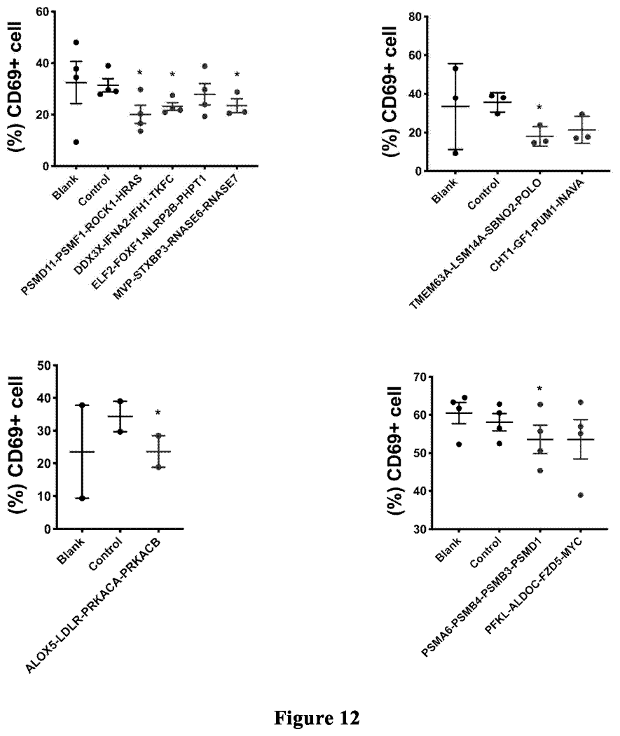

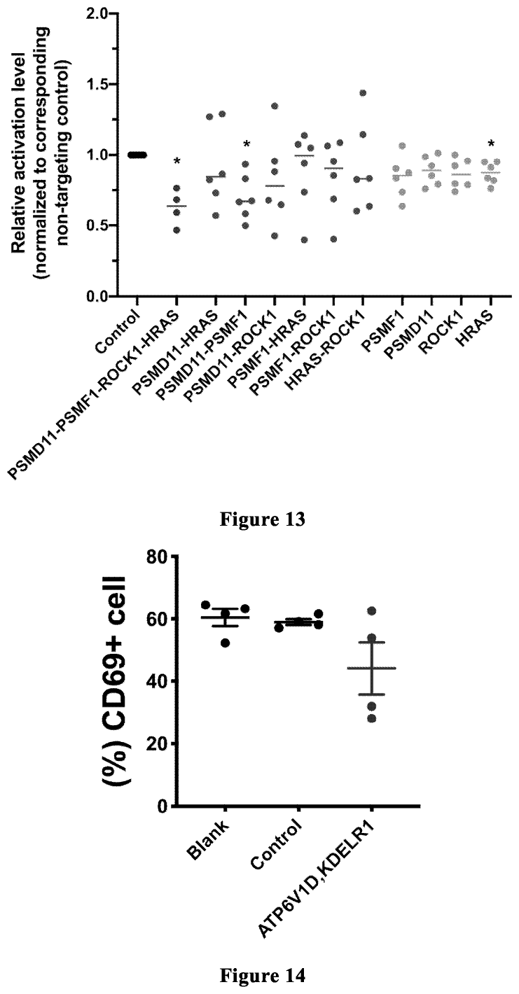

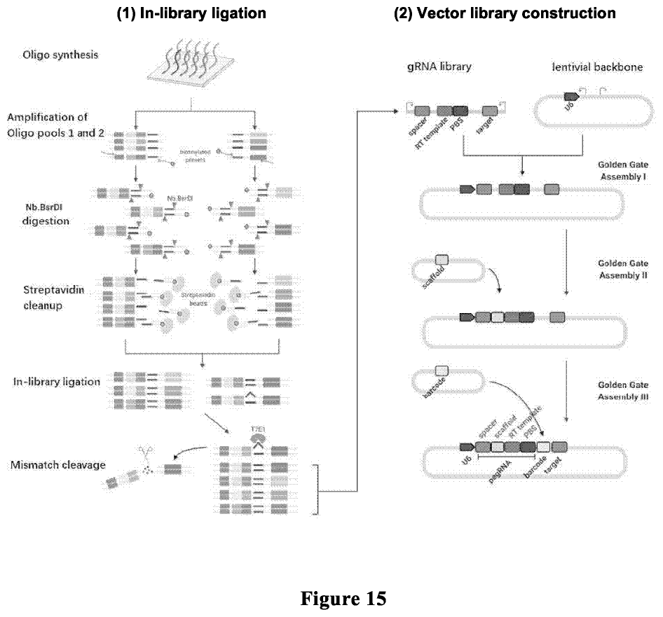

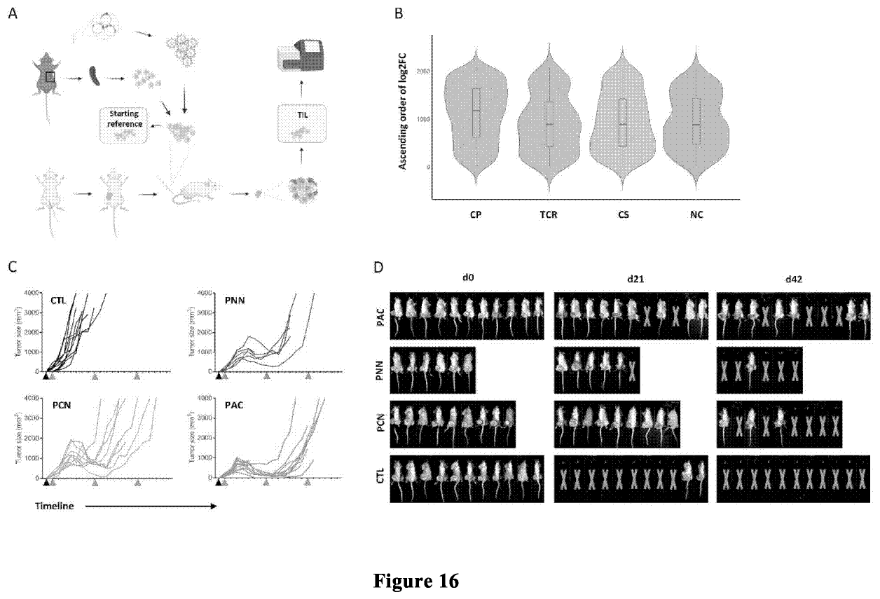

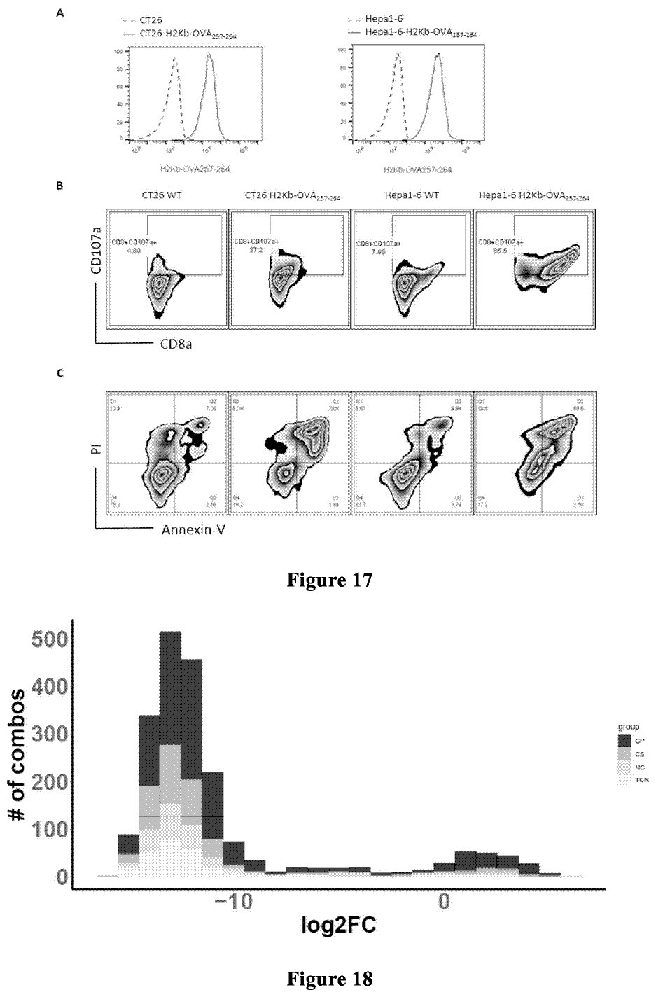

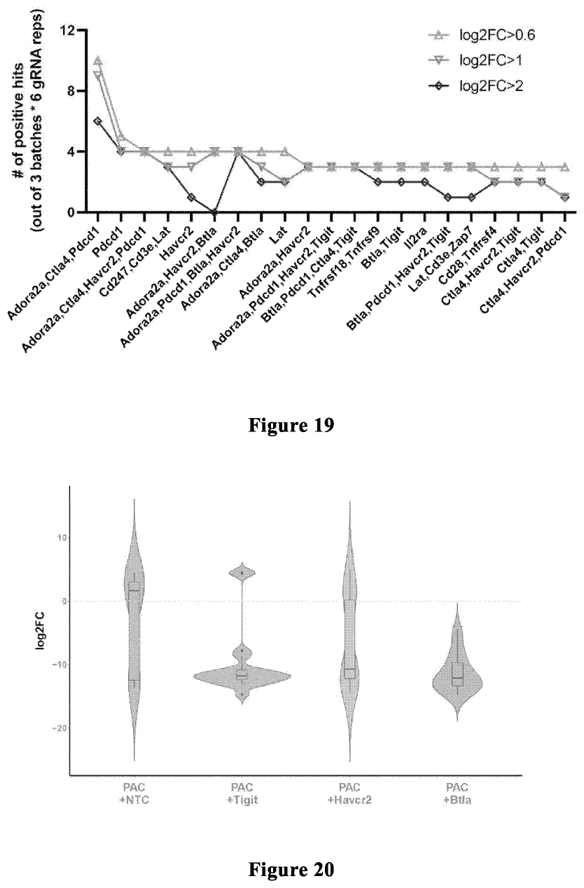

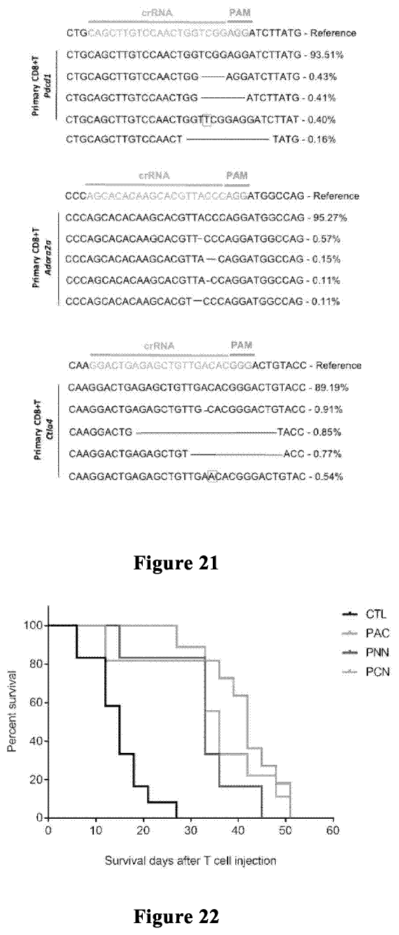

FIG. 4 shows the evaluation of the construction results of 4-gRNA combination library according to one example of the present invention.
(A) Histogram of the read counts of combinations: All pre-designed 4-gRNA combinations were covered by the first-round golden gate assembly products and were evenly distributed. The text in the graph indicated the number of combinations covered under different numbers of sequencing reads;
(B) The pre-designed 4-gRNA combinations were rapidly covered by sequencing reads and reached saturation in the first-round golden gate assembly products. When all 4-gRNA combinations were sorted from low to high sequencing reads, the dotted lines that were vertical to the horizontal axis from left to right in the figure represented the sequencing reads of the 4-gRNA combinations ranked in the 10^{th} percentile and the sequencing reads of the 4-gRNA combination ranked in the 90^{th} percentile, respectively, and the ratio (90^{th}/10^{th}=12.6315) indicated that the library was well homogeneous.
(C) Histogram of the read counts of combination: All pre-designed 4-gRNA combinations were covered by the fourth-round golden gate assembly products and were evenly distributed. The text in the graph indicated the number of combinations covered under different numbers of sequencing reads;
(D) The pre-designed 4-gRNA combinations were rapidly covered by sequencing reads and reached saturation in the fourth round of Golden Gate assembly products. When all 4-gRNA combinations were sorted from low to high sequencing reads, the dotted lines that were vertical to the horizontal axis from left to right in the figure represented the sequencing reads of the 4-gRNA combinations ranked in the 10^{th} percentile and the sequencing reads of the 4-gRNA combinations ranked in the 90^{th} percentile, respectively, and the ratio (90^{th}/10^{th}=14.4756) indicated that the library was well homogeneous.

FIG. 5 shows the design principle and selection of gRNA combinations from pathways and gene families.
(A) According to the examples of the present invention, the present inventors designed 6236 4-gRNA combinations, each 4-gRNA combination targeted 4 different genes. Among them, 3672 4-gRNA combinations (59%) were designed according to the KEGG pathway, 945 4-gRNA combinations (15%) were designed according to the EGA gene family, and 1569 4-gRNA combinations (25%) were designed by randomly selecting candidate genes in order to balance the gene coverage across combinations, and additional 50 4-gRNA combinations were used as negative controls;
(B) Immune response-related genes (IRRGs) were overlapped with KEGG genes. From the Gene Ontology database, 1601 immune response-related genes (IRRGs) were selected and targeted by the multiplexed CRISPR library, of which 1061 (66.3%) were also genes in the KEGG pathways;
(C) 287 (54.2%) of the 530 KEGG pathway genes contained immune response-related genes (IRRGs);
(D) 1286 (80.3%) also belonged to the EGA gene family;
(E) 441 (40.1%) of 1102 EGA family genes contained immune response-related genes (IRRGs).

FIG. 6 shows the implementation of the CRISPR library screening strategy and results.
(A) Jurkat activation screening protocol: The multiplexed vector library was packaged into lentiviruses for infection of Jurkat cells with stable Cas9 expression. 48 hours after infection, antibiotics were applied to select for successfully infected cells. On day 10, one group of cells was retained as a screening control, and the remaining cells were stimulated with CD3/CD28 T cell activator. After 24 hours, the cells were sorted according to cell surface CD69, and the cell populations ranked in the top 25% and the bottom 25% were selected;
(B) Distribution of the 4-gRNA combinations between CD69⁺ and CD69⁻ cell populations: Cell distribution between CD69⁺ and CD69⁻ populations was plotted. For each 4-gRNA combination, the proportions of the normalized read counts within the two populations were calculated and converted into log 2 values. Plotting and comparison were performed for the three combination subsets. As predicted, the distribution of the 50 combinations designed to target the TCR signaling pathway shifted toward the CD69⁻ cell population, the 51 combinations from the salivary secretion pathway distributed centrally, and overlapped primarily with the 50 combinations from the negative control subset;
(C) Volcano plot of Jurkat screening results: The screening results for each combination were quantified and plotted as a volcano plot. The combinations marked in blue satisfied the cut-off value of p-value<0.01; the combinations marked in grey (4 types in total) satisfied the cut-off value of p-value<10⁻¹⁰ and −5<log₂FC<−2 (FC meant fold change), 3 of these 4-gRNA combinations included CD3D, the CD3D encoded the transmembrane chain of TCR complex, the CD3D protein was directly related to T cell activation and was a positive control that should be screened. This result showed that this CRISPR library screening was effective;
(D) Candidate validation: A total of 9 combinations (sequences are shown in Table 0) were selected, and cloned, packaged and infected Jurkat cells with stable Cas9 expression (Jurkat-Cas9 cells), respectively. After CD3/CD28 stimulation, the percentage of CD69⁺ Jurkat-Cas9 cells was analyzed by flow cytometry. This experiment was repeated on two independent Jurkat-Cas9 monoclones. These 9 combinations exhibited reduction at certain level in the percentage of CD69 positive cells compared to the controls, and in the paired one-way ANOVA test, 5 of them showed statistical significance. A vector containing 4 non-target gRNAs was used as the control. All data were shown as mean±SEM (*P<0.05, compared with the control in one-way ANOVA test followed by multiple comparisons).

FIG. 7 shows a histogram of total counts of candidate genes in all 4-gRNA combinations according to the example of the present invention. The gene coverage rates of all 1599 immune response-related genes screened by the 4-gRNA combinations were calculated and plotted as a histogram. The gene coverage rate was defined as the number of times that a gene was picked in the combinations.

FIG. 8 shows anti-CD69 cell sorting. At the end of antibiotic selection, the cells were stimulated with CD3/CD28 T cell activators. After 24 hours of stimulation, the CD69⁺ cells sorted in the top 25% and the CD69⁻ cells sorted in the bottom 25% were used for downstream library preparation. The cells that were transduced with the multiplexed gRNA library but not activated with CD3/CD28 were used as the negative control for stimulation. The cells that were not transfected with the library but activated with CD3/CD28 served as the blank control for stimulation. To verify the surface CD69 expression of CD69⁻ and CD69⁺ cell populations, a reverse test was performed. 0.23% of the CD69⁻ cells showed CD69 signal, while 99.0% of the CD69⁺ showed CD69 signal.

FIG. 9 shows a histogram of read counts for the 4-gRNA combinations in vector and control samples. The normalized read counts for all 4-gRNA combinations were plotted as a histogram. After lentiviral transduction, a small number of 4-gRNA combinations were eliminated after puromycin selection. These combinations were removed from the analysis of the post-stimulation samples.

FIG. 10 shows the distribution and correlation of read counts (log 10) for the 4-gRNA combinations. The read counts for all 4-gRNA combinations were visualized as scatter plot between samples. The read counts distributions were visualized as histogram at the side.

FIG. 11 shows the distribution of the 4-gRNA combinations between the CD69⁺ and CD69⁻ cell populations: The cell distribution between the CD69⁺ and CD69⁻ populations was plotted. For each 4-gRNA combination, the proportion of normalized read counts within the two populations was calculated and converted into log 2 value. Plotting and comparison of the two combination subsets were performed. As predicted, compared with the distribution of the 50 negative control combinations (i.e., highest peak in the figure), the distribution of the 50 combinations consisting of TCR complex genes shifted towards the CD69⁻ cell population.

FIG. 12 shows the reduced CD69⁺ levels of candidate 4-gRNA combinations. The 4 candidate 4-gRNA combinations were cloned into lentiviral vectors, respectively. TCR activation experiments were performed to verify the activation reduction effect in the screening. Different knockout targets were listed on individual panels. Because the experiments were performed in different batches, the groupings represented the percent reduction of the original.

FIG. 13 shows the inhibition of T cell activation by gRNA combinations and subsets. The multiplexed PSMF1-PSMD11-ROCK1-HRAS vector, all 6 2-gRNA combinations (i.e., combinations of any two gRNAs, as shown in the figure), and 4 individual gRNA vectors were cloned, packaged, and transfected into Jurkat-Cas9 cells. After CD3/CD28 stimulation, the percentage of CD69⁺ Jurkat-Cas9 cells was analyzed by flow cytometry. In the experiments, normalization was performed against the matched control, and the relative activation levels were plotted. Vectors with 4, 2 or 1 kind of non-target gRNA were used as controls. (*P<0.05, compared with the control in one-way ANOVA test followed by multiple comparisons).

FIG. 14 shows the inhibitory effect of the combination subset candidates on T cell activation. The multiplexed ATP6V1D-KDELR1 vector was cloned, packaged and transfected into Jurkat-Cas9 cells. After CD3/CD28 stimulation, the percentage of CD69⁺ Jurkat-Cas9 cells was analyzed by flow cytometry. The experiments were performed on two independent Jurkat-Cas9 monoclones, each in duplicate. All data were expressed as mean±SEM.

FIG. 15 shows the construction method of the combinatorial screening library of spacer and PBS+ reverse transcription template designed for the Prime Editor system in one example of the present invention. As in FIG. 2, the whole process comprises two main steps: (1) in-library ligation, and (2) vector library construction.

FIG. 16 (A to D) illustrates in vivo screening and validation for combinatorial checkpoint blockades to boost T cells.

FIG. 16A is a schema of an in vivo screening for check point blockade. CD8+ T cells were collected from OT-1 mice (dark grey), which were infected by a screening library and further injected into recipient mice (grey) inoculated with Hepa1-6 cells with stable H2Kb-OVA257-264 expression. (TIL: tumor-infiltrating lymphocytes.)

FIG. 16B show ranks of the log 2FC values of the engineered T cells across the CP, TCR, CS, and NC groups. More T cells from the CP group were enriched into the tumor samples compared to T cells from the groups of TCR, CS, and NC. (CP: Checkpoint group; TCR: T cell receptor group; CS: Co-stimulatory molecule group; NC: Negative control group.)

FIG. 16C shows validation of the PAC combinatory gene perturbation. Tumor size curves were plotted for the mice receiving OT-1 CD8+T cells with a combined Adora2a, Ctla4 and Pdcd1 disruption (PAC), a combined Ctla4 and Pdcd1 disruption (PCN), or only a Pdcd1 disruption (PNN), and the mice that did not receive a CD8+T injection (CTL). The black arrow (timeline) indicated the day of tumor cell line inoculation; the triangles indicated d0, d21, and d42 after T cell injection. Tumor sizes were recorded every 3 days. The number of mice in each group was: 11 in PAC, 6 in PNN, 9 in PCN, and 12 in CTL.

FIG. 16D shows in vivo imaging of mice. In vivo imaging for mice receiving OT-1 CD8+T cells with the PAC, PCN, or PNN disruption, and the mice that did not receive CD8+T injection (CTL). The crosses (X) indicated dead mice or mice sacrificed because of tumor size limitation (≤4000 mm3).

FIG. 17 (A to C) shows establishment of H2Kb-OVA257-264 expression tumor cells and cytotoxicity of T cell against H2Kb-OVA257-264+ tumor cells in vitro.

FIG. 17A shows expression of H2Kb-OVA257-264 on tumor cell lines.

FIG. 17B shows CD107a and CD8a expression in OT-1 CD8+ T cells co-cultured with tumor cell lines with or without H2Kb-OVA257-264 expression.

FIG. 17C shows PI and Annexin V staining in tumor cell lines co-cultured with OT-1 CD8+ T cells.

FIG. 18 shows a histogram distribution of the log 2FC values of engineered T cells. Only a small number of gRNA combinations showed positive log 2FC, which indicated that most T cells were not capable of infiltrating into tumors.

FIG. 19 shows ranks of gRNA-combinations. The gRNA-combinations were ranked according to their enrichment in three batches of screen and six gRNA replicates in a designed multiplexed CRISPR library. The PAC combination (Adora2a, Ctla4, and Pdcd1) showed the most positive hits under different log 2FC cutoffs. Only the gRNA combinations that showed at least 3 positive hits were plotted in FIG. 19.

FIG. 20 shows log 2FC values of gRNA-combinations with PAC gene disruption. The distribution of log 2FC of four different PAC-containing combinations were plotted as violin plots.

FIG. 21 illustrates knockout efficiencies of the gRNAs of the PAC-combination. FIG. 21 shows representative amplicon sequencing of the sgRNA target sites in Cas9+ OT1 CD8+ T cells at day 3 post lentivirus transduction.

FIG. 22 shows survival curves of mice in a validation experiment. Survival rate for the mice that received OT-1 CD8+T cells with a combined Adora2a, Ctla4, and Pdcd1 disruption (PAC), a combined Ctla4 and Pdcd1 disruption (PCN), or only a Pdcd1 disruption (PNN), and the mice that did not receive CD8+T injection (CTL). The number of mice in each group was: 11 in PAC, 6 in PNN, 9 in PCN, and 12 in CTL.

### DETAILED DESCRIPTION OF INVENTION

Although the present invention may be embodied in many different forms, disclosed herein are specific illustrative embodiments thereof that demonstrate the principles of the present invention. It should be emphasized that the present invention is not limited to the specific embodiments illustrated herein. Furthermore, any section headings used herein are for organizational purposes only and are not to be construed as limiting the subject matter described.

Unless otherwise defined herein, scientific and technical terms used in conjunction with the present invention have the meanings commonly understood by one of ordinary skill in the art. Furthermore, unless the context otherwise requires, terms in the singular forms shall include the plural forms thereof, and terms in the plural forms shall include the singular forms thereof. More specifically, as used in the description and the appended claims, the singular forms “a,” “an” and “the” include plural referents unless the context clearly dictates otherwise. In this application, the use of “or” means “and/or” unless stated otherwise. Furthermore, the use of the term “comprising” and other forms such as “including” and “containing” is not restrictive. Furthermore, the ranges provided in the description and the appended claims include all values between the endpoints and the breakpoints.

### Definition

For better understanding the present invention, definitions and explanations of related terms are provided below.

The term CRISPR (Clustered regularly interspaced short palindromic repeats) refers to a repetitive sequence in the genome of prokaryotes, it is an immune weapon produced by bacteria and viruses in the history of life evolution. Briefly, during the infection with viruses, viruses can integrate their genes into the bacterial genome, and use the bacterial cell tools to serve their own gene replication; however, in order to remove the foreign invasion genes of viruses, the bacteria have evolved a CRISPR-Cas9 system, by using this system, the bacteria can silently excise the integrated viral genes from their own chromosomes, and this is the bacteria's unique immune system. CRISPR technology was discovered in the early 1990s, and as the research progressed, it quickly became the most popular gene-editing tool in fields such as human biology, agriculture, and microbiology.

In general, “CRISPR system” is collectively referred to as transcripts and other elements involved in the expression or directing activity of CRISPR-associated (“Cas”) gene, including sequence encoding Cas gene, tracr (transactivating CRISPR) sequence (e.g., tracrRNA or active part of tracrRNA), tracr pairing sequence (covering “direct repeat” and partial direct repeat of tracrRNA processing in the context of endogenous CRISPR system), guide sequence (also known as “spacer” in the context of endogenous CRISPR system”), or other sequence and transcript from CRISPR locus. In some embodiments, one or more elements of CRISPR system are derived from Type I, Type II, or Type III CRISPR system. In some embodiments, one or more elements of the CRISPR system are derived from a particular organism comprising an endogenous CRISPR system, such as Streptococcus pyogenes. In general, CRISPR system is characterized by elements that facilitate the formation of a CRISPR complex (also referred to as a protospacer in the context of an endogenous CRISPR system) at the site of a target sequence. In the context of CRISPR complex formation, “target sequence” refers to a sequence for which a guide sequence is designed to be complementary thereto, in which the hybridization between the target sequence and guide sequence promotes the formation of the CRISPR complex. Perfect complementarity is not required, provided that sufficient complementarity is present to cause hybridization and facilitate the formation of a CRISPR complex. A target sequence can comprise any polynucleotide, such as DNA or RNA polynucleotide. In some embodiments, the target sequence is located in the nucleus or cytoplasm of the cell. In some embodiments, the target sequence may be located in an organelle such as mitochondria or chloroplast of a eukaryotic cell. A sequence or template that can be used for recombination into a target locus that includes the target sequence is referred to as an “editing template” or “editing polynucleotide” or “editing sequence.” In the present invention, the exogenous template polynucleotide may be referred to as an editing template. In one aspect of the present invention, the recombination is homologous recombination.

In various aspects of the present invention, the terms “chimeric RNA”, “chimeric guide RNA”, “guide RNA”, “single guide RNA” and “synthetic guide RNA” are used interchangeably and refer to a polynucleotide sequence comprising guide sequence, tracr sequence, and tracr-pairing sequence. The term “guide sequence” refers to a sequence of approximately 20 bp within the guide RNA of a designated target site, and is used interchangeably with the term “guide” or “spacer.” The term “tracr-pairing sequence” is also used interchangeably with the term “direct repeat(s)”. The 20-nucleotide sequence at the 5′ end of guide gRNA is designated as spacer sequence (i.e., spacer) and is used to identify and bind to the complementary target sequence in the genome. The spacer sequence represents the specificity of gRNA. In a gRNA library, usually only the spacer sequence representing the specificity of gRNA is different between each sequence in the library. The spacer sequence of approximately 20 nucleotides, together with the downstream dozens of nucleotides, forms some special structures on the secondary structure, and binds a nuclease (e.g., Cas9) to direct the Cas nuclease to the target sequence for gene edition.

The terms “polynucleotide”, “nucleotide”, “nucleotide sequence”, “nucleic acid” and “oligonucleotide” are used interchangeably. They refer to a polymeric form of nucleotides in any length, either deoxyribonucleotides or ribonucleotides, or analogs thereof. A polynucleotide can have any three-dimensional structure and can perform any function, known or unknown. The following are non-limiting examples of polynucleotide: coding or non-coding region of a gene or gene fragment, multiple loci (one locus) defined by ligation analysis, exon, intron, messenger RNA (mRNA), transfer RNA, ribosomal RNA, short hairpin RNA (shRNA), micro-RNA (miRNA), ribozyme, cDNA, recombinant polynucleotide, branched polynucleotide, plasmid, vector, isolated DNA of any sequence, isolated RNA of any sequence, nucleic acid probe, and primer. A polynucleotide may contain one or more modified nucleotides, such as methylated nucleotides and nucleotide analogs. If present, modification of the nucleotide structure can be performed before or after polymer assembly. The sequence of nucleotide can be interrupted by a non-nucleotide component. Polynucleotide can be further modified after polymerization, such as by conjugation to a labeled component.

“Complementarity” refers to the ability of a nucleic acid sequence to form one or more hydrogen bonds with another nucleic acid sequence by means of classical Watson-Crick or other non-classical types of interaction. The percentage of complementarity represents the percentage of residues in a nucleic acid molecule that can form hydrogen bonds (e.g., Watson-Crick base pairing) with a second nucleic acid sequence (e.g., 5, 6, 7, 8, 9, 10 out of 10 mean 50%, 60%, 70%, 80%, 90%, and 100% complementary). “Completely complementary” means that all contiguous residues of a nucleic acid sequence form hydrogen bonds with the same number of contiguous residues in a second nucleic acid sequence. “Substantially complementary” as used herein means being at least 60%, 65%, 70%, 75%, 80%, 85%, 90%, 95%, 97%, 98%, 99% or 100% complementary over a region of 8, 9, 10, 11, 12, 13, 14, 15, 16, 17, 18, 19, 20, 21, 22, 23, 24, 25, 30, 35, 40, 45, 50 or more nucleotides; alternatively, refers to the case where two nucleic acids are capable of hybridizing under stringent conditions.

“Expression” as used herein refers to a process by which a polynucleotide is transcribed (e.g., into mRNA or other RNA transcripts) from a DNA template and/or a process by which the transcribed mRNA is subsequently translated into a peptide, polypeptide or protein. Transcript and encoded polypeptide may be collectively referred to as “gene products.” If the polynucleotide is derived from a genomic DNA, the expression may comprise splicing of mRNA in a eukaryotic cell.

Generally, and throughout this description, the term “vector” refers to a nucleic acid molecule capable of transporting another nucleic acid molecule to which it is linked. Vectors include, but are not limited to, single-stranded, double-stranded, or partially double-stranded nucleic acid molecules; nucleic acid molecules comprising one or more free ends, no free end (e.g., circular); nucleic acid molecules comprising DNA, RNA or both; and a wide variety of other polynucleotides known in the art. One type of vector is a “plasmid,” which refers to a circular double-stranded DNA loop into which an additional DNA fragment can be inserted, for example, by standard molecular cloning techniques. Another type of vector is a viral vector, in which a virus-derived DNA or RNA sequence is present in a vector for packaging a virus (e.g., retrovirus, replication-defective retrovirus, adenovirus, replication-defective adenovirus, and adeno-associated virus). Viral vectors also include a polynucleotide carried by a virus used for transfection into a host cell. Certain vectors (e.g., bacterial vectors with bacterial replication origin, and episomal mammalian vectors) are capable of autonomous replication in a host cell into which they are introduced. Other vectors (e.g., non-episomal mammalian vectors) integrate into a host cell's genome upon introduction into the host cell, and thus replicate together with the host genome. Furthermore, certain vectors are capable of directing the expression of a gene to which they are operably linked. Such vectors are referred to herein as “expression vectors”. Common expression vectors used in recombinant DNA technology are usually in the form of plasmids.

Recombinant expression vectors may comprise the nucleic acid of the present invention in a form suitable for nucleic acid expression in host cells, which means that these recombinant expression vectors contain one or more regulatory elements selected based on the host cell to be used for expression, and the regulatory elements are operably linked to the nucleic acid sequence to be expressed. Within the recombinant expression vector, “operably linked” is intended to mean that the nucleotide sequence of interest is linked to the one or more regulatory elements in a manner that allows for expression of the nucleotide sequence (e.g., in an in vitro transcription/translation system or in a host cell when the vector is introduced into the host cell).

The term “regulatory elements” are intended to include promoter, enhancer, internal ribosome entry site (IRES), and other expression control elements (e.g., transcription termination signal such as polyadenylation signal and polyU sequence). Such regulatory sequences are described, for example, in Goeddel, GENE EXPRESSION TECHNOLOGY: METHODS IN ENZYMOLOGY, 185, Academic Press, San Diego, California, 1990. Regulatory elements include those sequences that direct constitutive expression of a nucleotide sequence in many types of host cells as well as those sequences that direct expression of the nucleotide sequence only in certain host cells (e.g., tissue-specific regulatory sequences). Tissue-specific promoters can primarily direct expression in the desired tissue of interest, such as muscle, neuron, bone, skin, blood, specific organ (e.g., liver, pancreas), or specific cell type (e.g., lymphocyte). Regulatory elements may also direct expression in a time-dependent manner (e.g., in a cell cycle-dependent or developmental stage-dependent manner), which may or may not be tissue- or cell type-specific.

Those skilled in the art will appreciate that the design of expression vector may depend on factors such as the choice of host cell to be transformed, the desired level of expression, and the like. A vector can be introduced into a host cell to produce a transcript, protein, or peptide, including fusion protein or peptide encoded by the nucleic acid as described herein (e.g., clustered regularly interspaced short palindromic repeat (CRISPR) transcript, protein, enzyme, mutant form thereof, fusion protein thereof, etc.).

Favorable vectors include lentiviruses and adeno-associated viruses, and the vectors of this type can also be selected to target specific types of cells.

The term “in-library ligation” refers to a high-throughput, specific fragment ligation of a nucleic acid sample (library) composed of a large number of mixed sequences in a library, so as to achieve the purposes of extending sequence length and/or improving sequence diversity, etc. Using nicking endonuclease (e.g., Nb.BsrDI), this method can generate on a nucleic acid fragment a long overhang that has one-to-one correspondence and can realize pairwise complementary ligation between nucleic acid sequences. Thus, in a high-throughput library, the ligation reaction occurs only between pre-designed sequences. The key difference between this ligation reaction and the general restriction endonuclease-mediated digestion reaction lies in that the in-library ligation reaction can generate on a nucleic acid sequence a long overhang with variable length through pre-designed complementary sequences and nicking restriction enzymes. Since the overhang sequence length determines the number of fragment combinations that can achieve pairwise complementarity (the theoretical value is 4ⁿ, wherein n is the number of nucleotides in the overhang sequence, for example, the theoretical value of the number of overhang sequence with a length of 10 nucleotides is 4¹⁰). The in-library ligation reaction can theoretically realize the one-to-one corresponding ligation between the internal sequences of a mixed nucleic acid sample with extremely high complexity. However, the common restriction enzymes (e.g., EcoRI, BamHI, etc.), firstly, have very limited types, and secondly, can generate overhang sequences that are generally 4 to 6 nucleotides with a specific sequence, so that the library constructed by using common restriction enzymes does not comprise a high-throughput one-to-one corresponding ligation between specified sequences.

In the in-library ligation design, a nicking endonuclease, for example, Nb. BsrDI, is used to generate nicks on the double strand DNA sequences. The Nb. BsrDI is one example of so-called “nicking endonuclease”, which has different cutting pattern from the commonly used restriction enzymes (e.g., EcoRI). To apply the Nb. BsrDI digestion, two recognition sites were designed to ligate two sub-pools. One recognition site located on the top strand of DNA, generated one nick at the top strand. Another recognition site located on the bottom strand of DNA, generated one nick at the bottom strand. And the two recognition sites were apart away from each other, resulted two nicks that were also apart from each other. Importantly, the distance between the two nicks is flexible. When we design the oligo, we could adjust the distance between the two recognition sites to determine the distance between the two nicks. This is why long overhangs of 21-nt (or a little shorter, or longer) can be generated by using nicking endonuclease.

The present invention provides the following exemplary embodiments:
1. A CRISPR library of pair-specific multi-gene combinations, wherein the CRISPR library comprises a plurality of vectors each carrying more than two kinds of gRNA sequences, and the more than two kinds of gRNA sequences carried on each vector are capable of performing co-editing of more than two kinds of important molecules, for example, each vector in the CRISPR library carries 3 to 6 kinds of gRNA sequences.
2. The CRISPR library of pair-specific multi-gene combinations according to Embodiment 1, wherein the CRISPR library comprises a plurality of vectors each carrying 4 kinds of gRNA sequences, and the 4 kinds of gRNA sequences carried on each vector are capable of performing co-editing of 4 kinds of important molecules.
3. The CRISPR library of pair-specific multi-gene combinations according to Embodiment 2, wherein each vector in the CRISPR library comprises an insert fragment shown in gRNA1-tRNA1-gRNA2-tRNA2-gRNA3-tRNA3-gRNA4, in which gRNA1, gRNA2, gRNA3 and gRNA4 are respectively directed to 4 different genes of any known sequences.
4. The CRISPR library of pair-specific multi-gene combinations according to Embodiment 3, wherein the insert fragment shown in gRNA1-tRNA1-gRNA2-tRNA2-gRNA3-tRNA3-gRNA4 further comprises a U6 promoter, preferably a human U6 promoter, at the N-terminus.
5. The CRISPR library of pair-specific multi-gene combinations according to Embodiment 3, wherein the sequences of tRNA1, tRNA2 and tRNA3 are all the same, two are the same and one is different, or three are different.
6. A method for constructing a CRISPR library of pair-specific multiplexed gRNA combinations based on long overhang sequence ligation, the method comprising the following steps:
(1) designing a sequence library of pair-specific multiplexed gRNA combinations according to the pathway or gene family to be screened, and synthesizing a mixture of two or more oligonucleotide chain pools according to the sequences of the library, wherein each oligonucleotide sequence in each oligonucleotide chain pool comprises one or more kinds of gRNAs, wherein for 3′-end of each sequence in one oligonucleotide chain pool, there is only one kind of 5′-end sequence completely complementary thereto in another oligonucleotide chain pool, and the complementary portion has a sequence length of 2-100 nucleotides, such as 4-50, 10-40, 15-35, 20-30, preferably 21 nucleotides (21 nt);
(2) performing PCR amplification with the mixture of two or more oligonucleotide chain pools as templates, respectively, to obtain two or more corresponding library chain pools, respectively;
(3) using a nicking endonuclease to digest the two or more corresponding library chain pools obtained by PCR amplification in step (2) respectively to generate products each having one or two complementary long overhangs of 2-100 nucleotides, such as 4-50, 10-40, 15-35, 20-30, preferably 21 nt, and mixing the digested products from each of the library chain pools and performing ligation of the digested products from the library chain pools by annealing to generate linear gRNA library sequences, each of which comprises the pair-specific multiplexed gRNA combinations;
(4) inserting the linear gRNA library sequence obtained in step (3) into a vector to form a primary library vector; and
(5) sequentially inserting a tRNA sequence between two adjacent gRNAs in the primary library vector to form a complete library vector, wherein the complete library vector comprises pair-specific multiplexed gRNA combinations which comprise a tRNA sequence between any two adjacent gRNAs.
7. A method for constructing a CRISPR library of pair-specific multiplexed gRNA combinations based on long overhang sequence ligation, the method comprising the following steps:
(1) designing a sequence library of pair-specific multiplexed gRNA combinations according to the pathway or gene family to be screened, and synthesizing a mixture of oligonucleotide chain pools 1 and 2 according to the sequences of the library, wherein each oligonucleotide sequence in the oligonucleotide chain pool 1 comprises gRNA1 and gRNA2, and each oligonucleotide sequence in the oligonucleotide chain pool 2 comprises gRNA3 and gRNA4, wherein for the 3′ end of each sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt);
(2) performing PCR amplification with the mixture of oligonucleotide chain pools 1 and 2 as templates, respectively, to obtain corresponding library chain pools 1 and 2, respectively;
(3) using a nicking endonuclease to digest the library chain pools 1 and 2 obtained by PCR amplification in step (2) respectively to generate products each having a complementary long overhang of 2-100 nucleotides, such as 4-50, 10-40, 15-35, 20-30, preferably 21 nt, and performing ligation of the digested products from the library chain pools 1 and 2 by annealing to generate gRNA library sequences each of which is shown by gRNA1-gRNA2-gRNA3-gRNA4;
(4) inserting the gRNA library sequence obtained in step (3) into a vector to form a primary library vector; and
(5) sequentially inserting tRNA1, tRNA2 and tRNA3 sequences into the primary library vector to form a complete library vector, wherein the complete library vector comprises the insert fragment shown by gRNA1-tRNA1-gRNA2-tRNA2-gRNA3-tRNA3-gRNA4.
8. The method according to Embodiment 7, wherein the reverse primer in the primer pair used for the amplification of the oligonucleotide chain pool 1 in step (2) is biotinylated, and the forward primer in the primer pair used for the amplification of the oligonucleotide chain pool 2 is biotinylated.
9. The method according to Embodiment 7, wherein the nicking endonuclease used in step (3) is selected from Nb.BsrDI or Nt.BspQI nicking endonuclease.
10. The method according to Embodiment 8, wherein in step (3), after nicking endonuclease cleavage and before annealing ligation, streptavidin magnetic beads are used to purify and remove biotin-carrying small fragments.
11. The method according to Embodiment 7, wherein after the library chain pools 1 and 2 are digested with the nicking endonuclease and annealed in step (3), and before step (4), the method further comprises using T7 endonuclease I to digest poorly matched ligation products.
12. A method for constructing a CRISPR library of pair-specific multiplexed gRNA combinations based on long overhang sequence ligation, comprising the following steps:
(1) designing a sequence library of pair-specific multiplexed gRNA combinations according to the pathway or gene family to be screened, and synthesizing a mixture of oligonucleotide chain pools 1 and 2 according to the sequences of the library, wherein each oligonucleotide sequence in the oligonucleotide chain pool 1 comprises gRNA1 and gRNA2, and each oligonucleotide sequence in the oligonucleotide chain pool 2 comprises gRNA3 and gRNA4, wherein for the 3′ end of each sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt);
(2) performing PCR amplification with the mixture of oligonucleotide chain pools 1 and 2 as templates, respectively, to obtain library chain pools 1 and 2, respectively, wherein the reverse primer in the primer pair used for amplification of the oligonucleotide chain pool 1 is biotinylated, and the forward primer in the primer pair used for amplification of the oligonucleotide chain pool 2 is biotinylated;
(3) using a nicking endonuclease to digest the library chain pools 1 and 2 obtained by PCR amplification in step (2) respectively to generate products each having a complementary long overhang of 2-100 nucleotides, such as 4-50, 10-40, 15-35, 20-30, preferably 21 nt, performing ligation of the digested products from the library chain pools 1 and 2 by annealing after biotin-carrying small fragments are removed by purification with streptavidin magnetic beads, performing digestion of the ligation product with T7 endonuclease I (T7E1) to remove poorly matched double-stranded fragments and finally generate gRNA library sequences, each of which is shown by gRNA1-gRNA2-gRNA3-gRNA4;
(4) inserting the gRNA library sequence obtained in step (3) into a vector to form a primary library vector; and
(5) sequentially inserting tRNA1, tRNA2 and tRNA3 sequences into the primary library vector to form a complete library vector, wherein the complete library vector comprises the insert fragment shown by gRNA1-tRNA1-gRNA2-tRNA2-gRNA3-tRNA3-gRNA4.
13. A method for constructing a CRISPR library of pair-specific multiplexed gRNA combinations based on long overhang sequence ligation, comprising:
(1) designing a sequence library of pair-specific multiplexed gRNA combinations according to the pathway or gene family to be screened, and synthesizing a mixture A of two oligonucleotide chain pools A1 and A2 and a mixture B of two oligonucleotide chain pools B1 and B2, according to the sequences of the library, wherein each oligonucleotide sequence in each oligonucleotide chain pool comprises one or more kinds of gRNAs,
wherein for 3′-end of each sequence in oligonucleotide chain pool A1, there is only one kind of 5′-end sequence completely complementary thereto in oligonucleotide chain pool A2, and the complementary portion has a sequence length of 2-100 nucleotides, such as 4-50, 10-40, 15-35, 20-30, preferably 21 nucleotides (21 nt);
wherein for 3′-end of each sequence in oligonucleotide chain pool B1, there is only one kind of 5′-end sequence completely complementary thereto in oligonucleotide chain pool B2, and the complementary portion has a sequence length of 2-100 nucleotides, such as 4-50, 10-40, 15-35, 20-30, preferably 21 nucleotides (21 nt);
wherein for 3′-end of each sequence in oligonucleotide chain pool A2, there is only one kind of 5′-end sequence completely complementary thereto in oligonucleotide chain pool B1, and the complementary portion has a sequence length of 2-100 nucleotides, such as 4-50, 10-40, 15-35, 20-30, preferably 21 nucleotides (21 nt);
(2) performing PCR amplification with the mixture A as templates, respectively, to obtain corresponding library chain pools A1 and A2, respectively; and performing PCR amplification with the mixture B as templates, respectively, to obtain corresponding library chain pools B1 and B2, respectively;
(3) using a nicking endonuclease to digest the library chain pools A1 and A2 obtained by PCR amplification in step (2) respectively to generate products each having one or two complementary long overhangs of 2-100 nucleotides, such as 4-50, 10-40, 15-35, 20-30, preferably 21 nt; using a nicking endonuclease to digest the library chain pools B1 and B2 obtained by PCR amplification in step (2) respectively to generate products each having one or two complementary long overhangs of 2-100 nucleotides, such as 4-50, 10-40, 15-35, 20-30, preferably 21 nt; and mixing the digested products from the library chain pools A1, A2, B1 and B2 and performing ligation of the digested products from the library chain pools A1, A2, B1 and B2 by annealing to generate linear gRNA library sequences A1-A2-B1-B2, each of which comprises the pair-specific multiplexed gRNA combinations;
(4) inserting the gRNA library sequence A1-A2-B1-B2 obtained in step (3) into a vector to form a primary library vector; and
(5) sequentially inserting a tRNA sequence between two adjacent gRNAs in the primary library vector to form a complete library vector, wherein the complete library vector comprises pair-specific multiplexed gRNA combinations which comprise a tRNA sequence between any two adjacent gRNAs.
14. The method according to any one of Embodiments 7 and 12-13, wherein the vector used in step (4) is a viral vector, for example, a lentiviral vector, a retroviral vector, an adenoviral vector, an adeno-associated viral vector.
15. The method according to any one of Embodiments 7 and 12-13, wherein the vector used in step (4) is a lentiviral vector, and the method further comprises the following step after obtaining the complete library vector in step (5): (6) a step of packaging a lentivirus with the constructed library vector and detecting a lentivirus titer.
16. The method according to Embodiment 7 or 12, wherein in step (5), tRNA1, tRNA2 and tRNA3 are sequentially introduced through golden gate assembly, wherein the sequences of tRNA1, tRNA2 and tRNA3 are all the same, two are the same and one is different, or all three are different.
17. The method according to Embodiment 7 or 12, wherein the insert fragment shown by gRNA1-tRNA1-gRNA2-tRNA2-gRNA3-tRNA3gRNA4 is under the control of a U6 promoter, preferably under the control of a human U6 promoter.
18. The method according to Embodiment 7 or 12, wherein in step (5), tRNA1, tRNA2 and tRNA3 are sequentially introduced by golden gate assembly, and different endonucleases are respectively used in the reactions for introducing tRNA1, tRNA2 and tRNA3.
19. A library construction method based on long overhang sequence ligation, the method comprising the steps of:
(1) designing and synthesizing a mixture of oligonucleotide chain pools 1 and 2, wherein each oligonucleotide in the oligonucleotide chain pool 1 has a length of 141-165 bp, and each oligonucleotide in the oligonucleotide chain pool 2 has a length of 150 bp; and, for the 3′ end of each kind of sequence in the oligonucleotide chain pool 1, there is only one kind of 5′-end sequence completely complementary thereto in the oligonucleotide chain pool 2, and the complementary portion has a sequence length of 15-35 nucleotides, preferably 20-30 nucleotides, more preferably 21 nucleotides (21 nt);
(2) performing PCR amplification with the mixture of oligonucleotide chain pools 1 and 2 as templates, respectively, to obtain library chain pools 1 and 2, respectively, wherein the reverse primer in the primer pair used for amplification of the oligonucleotide chain pool 1 is biotinylated, and the forward primer in the primer pair used for amplification of the oligonucleotide chain pool 2 is biotinylated;
(3) using a nicking endonuclease to digest the library chain pools 1 and 2 obtained by PCR amplification in step (2) respectively to generate products each having a complementary long overhang of 15-35 nucleotides, preferably 20-30 nucleotides, more preferably 21 nt, performing ligation of the digested products from the library chain pools 1 and 2 by annealing after biotin-carrying small fragments are removed by purification with streptavidin magnetic beads, performing digestion of the ligation product with T7 endonuclease I (T7E1) to remove poorly matched double-stranded fragments and finally generate insert sequences; and
(4) inserting the insert sequence obtained in step (3) into a vector to form a primary library vector.
20. The method according to Embodiment 19, wherein the 5′ end of each sequence in the oligonucleotide chain pool 2 in step (1) is completely complementary to the 3′ end of one or more sequences in the oligonucleotide chain pool 1.
21. The method according to Embodiment 19, wherein the nicking endonuclease used in step (3) is selected from Nb.BsrDI or Nt.BspQI nicking endonuclease.
22. The method according to Embodiment 19, wherein the vector used in step (4) is a viral vector, for example, a lentiviral vector, a retroviral vector, an adenoviral vector or an adeno-associated viral vector.
23. The method according to Embodiment 22, wherein the vector used in step (4) is a lentiviral vector, and, after obtaining the primary library vector in step (4), the method further comprises the following step: (5) a step of packaging a lentivirus with the constructed library vector and detecting a lentivirus titer.
24. A host cell transformed with the CRISPR library according to any one of Embodiments 1 to 5, wherein the host cell is a prokaryotic cell or a eukaryotic cell, preferably a bacterial cell, a fungal cell or a mammalian cell, more preferably a murine cell or a human cell.
25. A high-throughput method for combined screening of incorporated multiple genes, the method comprising using the CRISPR library according to any one of Embodiments 1 to 5.

### Example

The present invention, as generally described herein, will be more readily understood by reference to the following examples, which are provided by way of illustration and are not intended to limit the present invention. These examples do not imply that the experiments below are all or only experiments performed.

### Example 1: Designing a Sequence Library of Pair-Specific Multiplexed gRNA Combinations, and Synthesizing Oligonucleotide Chain Pools 1 and 2 According to the Sequences of the Library

Oligonucleotide chain pools 1 and 2 are designed for each signaling pathway of a cell to form a CRISPR library of pair-specific multiplexed gRNA combinations (see the schematic diagram in FIG. 2). The specific steps were as follows:

1.1 Selecting Sequence of Original gRNA Library^{[25]}

TABLE 0
Oligo (i.e., gRNA1-gRNA2-gRNA3-gRNA4)

| gene | |
|---|---|
| pair | oligo |
| PSMA6, | ccgcgtctcacaccgAGGTGGCTATGGCAGGTCTTgttt |
| PSMB4, | ggagaccttnnntgtggtctctacctTTAGGAACCCCAT |
| PSMB3, | GGTGACCgtttgnnngcaggtgcaatgTCATGCTAGGAA |
| PSMD1 | ACCCACAGCcattgcacctgcnntatgcaGTACCATGTT |
| | GGGCTCCCAGgtttaagtcttctgnnatcgaagacatgc |
| | ccAGGCTATAAGCTAACATTCCgtttcgagacggccc |
| | (SEQ ID NO: 711) |
| PSMF1, | ccgcgtctcacaccgCATCCTTATACTCATACCGGgttt |
| PSMD11, | ggagaccttnnntgtggtctctacctGGCGACCTTTACC |
| ROCK1, | GATGGAGgtttgnnngcaggtgcaatgGCACGCACGTCA |
| HRAS | TTAATGAGCcattgcacctgcnntatgcaTTACATATTA |
| | TAGCAATCGTgtttaagtcttctgnnatcgaagacatgc |
| | ccGGACTCGGATGACGTGCCCAgtttcgagacggccc |
| | (SEQ ID NO: 712) |
| PFKL, | ccgcgtctcacaccgACAGTATACGTGGTGCACGAgttt |
| ALDOC, | ggagaccttnnntgtggtctctacctCCAGGATAAGGGC |
| FZD5, | ATCGTCGgtttgnnngcaggtgcaatgACGAAAATAAGG |
| MYC | ACCTCGCCGcattgcacctgcnntatgcaCATGGATTAC |
| | AACCGCAGCGgtttaagtcttctgnnatcgaagacatgc |
| | ccGCTGCACCGAGTCGTAGTCGgtttcgagacggccc |
| | (SEQ ID NO: 713) |
| ALOX5, | ccgcgtctcacaccgTAGAGCGGGTCATGAATCACgttt |
| LDLR, | ggagaccttnnntgtggtctctacctCTGGAAGCTGGCG |
| PRKACB, | GGACCACgtttgnnngcaggtgcaatgGGAAGAGAGCAC |
| PRKACA | CGACTAACAcattgcacctgcnntatgcaAAGCATACTC |
| | CAGTCGAACAgtttaagtcttctgnnatcgaagacatgc |
| | ccAGGAGAACTCGAGTTTGACGgtttcgagacggccc |
| | (SEQ ID NO: 714) |
| TMEM63A, | ccgcgtctcacaccgACTACACACGGATGAAGGACgttt |
| LSM14A, | ggagaccttnnntgtggtctctacctCGTACCTTTGGCA |
| SBN02, | AGGGCTAgtttgnnngcaggtgcaatgAGTCCTCGTTCA |
| POLQ | AGAACGACCcattgcacctgcnntatgcaGCAGGGGCGG |
| | CGGGCTGTACgtttaagtcttctgnnatcgaagacatgc |
| | ccGTAGAGTTCAGCATTCAACCgtttcgagacggccc |
| | (SEQ ID NO: 715) |
| CHIT1, | ccgcgtctcacaccgCTACGGACGCTCCTTCACACgttt |
| GFI1, | ggagaccttnnntgtggtctctacctACGGTCGGTAGCT |
| PUM1, | CTGCACCgtttgnnngcaggtgcaatgCCGCTAAATAAA |
| INAVA | CGTACCCCGcattgcacctgcnntatgcaAGTCCACCAT |
| | AGCGTCGTCCgtttaagtcttctgnnatcgaagacatgc |
| | ccCCCATCGTCACCGCGGGGCCgtttcgagacggccc |
| | (SEQ ID NO: 716) |
| ELF2, | ccgcgtctcacaccgACTGGCTCCACAATCACTGCgttt |
| FOXF1, | ggagaccttnnntgtggtctctacctCTTCTCCGGGCGC |
| PHPT1, | CGGATGCgtttgnnngcaggtgcaatgCCACCTTTCGAA |
| NLRP2B | TATGGCACCcattgcacctgcnntatgcaGAGAGCAAGG |
| | AGATCGTGCGgtttaagtcttctgnnatcgaagacatgc |
| | ccGCAAGTTCAAGTCTCTGATCgtttcgagacggccc |
| | (SEQ ID NO: 717) |
| DDX3X, | ccgcgtctcacaccgAGGAACCGAGAAGCTACTAAgttt |
| IFNA2, | ggagaccttnnntgtggtctctacctGTTTGGCAACCAG |
| IFIH1, | TTCCAAAgtttgnnngcaggtgcaatgTACACCAACACG |
| TKFC | AAACGCCAGcattgcacctgcnntatgcaTGAGTTCAAA |
| | CCCATGACACgtttaagtcttctgnnatcgaagacatgc |
| | ccTGTGATGATGGTCAACAACCgtttcgagacggccc |
| | (SEQ ID NO: 718) |
| MVP, | ccgcgtctcacaccgGCAGCTGGAACAAGGCATCCgttt |
| STXBP3, | ggagaccttnnntgtggtctctacctGATGTCGAATTCT |
| RNASE6, | AACCCAGgtttgnnngcaggtgcaatgGGACCGTACAAG |
| RNASE7 | CCAAGACACcattgcacctgcnntatgcaCAGCAGCACT |
| | ATAGCGGCACgtttaagtcttctgnnatcgaagacatgc |
| | ccGGAGAAAGGCTCGTGCAGGAgtttcgagacggccc |
| | (SEQ ID NO: 719) |

1.1.1 Selecting gene sequences containing downstream NGG PAM sites in human genome;
Protospacer adjacent motif, also known as PAM, generally referred to a 3-base DNA sequence downstream of the DNA sequence bound to Cas9, and generally existed in the form of NGG (N: represented any one of A, T, C, G). The presence of PAM sequence was a necessary condition for the successful binding of Cas9 to the DNA sequence to exert cleavage function. If there was a PAM sequence downstream of a gene sequence, it could be used as a target gene sequence of gRNA.
1.1.2 Calculating off-target rate of the selected sequence according to a mismatching probability of each base of gRNA;
1.1.3 Retaining among these sequences a sequence as potential gRNA sequence, which is capable of locating a coding region of a gene (CDS) or a region of 100 base pairs (bps) upstream and downstream of the CDS, and has an off-target rate of less than 0.05;
1.1.4 Combining the above sequences with the Brunello gRNA library^{[25]}, eliminating gRNA sequences containing 4 consecutive thymines or BsmBI and AarI restriction sites, and sorting the gRNAs by using target genes thereof as units from low to high according to off-target rates thereof,
1.1.5 After sorting, from gRNAs of each gene, selecting top 10 gRNA sequences with low off-target rate that were sorted from low to high, and collecting them into the original gRNA library.
   1.2 Determining gRNA Combinations
1.2.1 Selecting genes related to immune response as candidate genes according to the annotation of the GO database (http://geneontology.org/);
1.2.2 Respectively performing gene pairing of the candidate genes according to the signaling pathways and gene families annotated in the KEGG database (https://www.genome.jp/kegg/) and EGA database (https://www.ebi.ac.uk/ega/home), and selecting targeted gRNAs for each gene from the original gRNA library for combination;

In this experiment, the pairing principle was as follows:
(1) All gene combinations appeared only once regardless of order;
(2) Four gRNA sequences were combined in pairs to ensure that the long sequence after combination had a free energy of not less than −48 kcal/mol;
(3) The number of occurrences of each gene was limited (e.g., limit specification: limit times were 15 times for the initial condition; in the pairing order (as shown in (i) to (vi) below), the steps (ii), (iv) and (v) were limited, the steps (i) and (iii) were not limited; after step (vi), for all genes, limit times increased 1 per cycle. The initial condition was obtained by: 12472 oligonucleotide chains in the library (chain pool 1+chain pool 2)/the number of genes, so as to ensure uniformity;
(4) Pairing was performed in cycle way according to signaling pathways and gene families, respectively;

The pairing sequences were as follows:
(i) a signaling pathway contained only 4 or 5 candidate genes, all possible candidate gene combinations were paired within the pathway, and only the gRNA sequence with the lowest off-target rate (i.e., off-target rate rank Top1) was selected;
(ii) a signaling pathway contained more than 5 candidate genes, random pairing was performed within the pathway to ensure that at least 4 groups of genes were paired, and the gRNA sequences were randomly selected from the off-target rate Top10 ranked from low to high;
(iii) a gene family contained only 4 or 5 candidate genes, all possible candidate gene combinations were paired within the family, and only the gRNA sequence with the lowest off-target rate (i.e., off-target rate rank Top1) was selected;
(iv) a gene family contained more than 5 candidate genes, random pairing was performed within the family to ensure that at least 4 groups of genes were paired, and the gRNA sequences were randomly selected from the off-target rate Top10 ranked from low to high;
(v) genes of different signaling pathways that were randomly combined, their candidate genes were selected for pairing, and the gRNA sequences were randomly selected from the off-target rate Top10 ranked from low to high;
(vi) genes that were completely randomly paired, the gRNA sequences were randomly selected from the off-target rate Top10 ranked from low to high.
   1.3 Strategy for Pair-Specific gRNA Combinations

The 3′ end of each oligonucleotide in the oligonucleotide chain pool 1 and the 5′ end of each oligonucleotide in the oligonucleotide chain pool 2 were respectively added with a sequence, and the two sequences were specifically complementary in the two pair-specific oligonucleotides so as to ensure the specific pairing of the four gRNAs.

### Results

Referring to the schematic diagram of FIG. 2, according to the above-mentioned design and combination strategy, oligonucleotide chain pools 1 and 2 of pair-specific multiplexed gRNA combinations were obtained for subsequent experiments.

### Example 2: PCR Amplification with Oligonucleotide Chain Pools as Templates

As described in Example 1, the oligonucleotide chain pools 1 and 2 synthesized by the biotechnology company did not reach the amount for library construction and storage, and thus PCR amplification was required.

### 2.1 Amplification of Oligonucleotide Chain Pools 1 and 2

Objective: PCR amplification of oligonucleotide chain pools 1 and 2 was performed to achieve a sufficient amount to construct a CRISPR library of pair-specific multiplexed gRNA combinations.

### Materials:

(1) Reaction substrates: diluted oligonucleotide chain pools 1 and 2 (named TF oligo sub pool 1/2, designed and synthesized according to the method of Example 1).
(2) Primers (see: Table 1). Primers were designed by the present inventors and synthesized by a biotechnology company.

TABLE 1
Primers used for amplification of oligonucleotide
chain pools 1 and 2

| Primer name | Sequence (5′→3′) |
|---|---|
| Fwd-sub-pool | GACCGCGTCTCACACCG |
| 1 primer | (SEQ ID NO: 4) |
| Biotinylated | biotin-CTGCGCTCCA |
| Rev-sub-pool | CGAGCCCGACGCAATG |
| 1 primer | (SEQ ID NO: 5) |
| Biotinylated | biotin-CTGGCGTGGT |
| Fwd-sub-pool | CGCGTGCTCGGCAATG |
| 2 primer | (SEQ ID NO: 6) |
| Rev-sub-pool | GATCAGGGCCGTCTCGAAAC |
| 2 primer | (SEQ ID NO: 7) |

It can be seen from Table 1 that one primer of the primer pairs used in each of the amplification of oligonucleotide chain pools 1 and 2 was biotinylated, so that a double-stranded amplification product with biotin in one amplification chain was obtained. The biotin-bearing small fragments (i.e., small fragments without gRNA combination, see FIG. 2(1)) produced in the nicking endonuclease digestion step could be removed using streptavidin magnetic beads, so that the 3′- and 5′-overhang products of the oligonucleotide chain pools were more thoroughly exposed.

### 2.1.1 PCR Amplification of Oligonucleotide Chain Pool 1 (TF Oligo Sub Pool 1) (See FIG. 2).

Usually, a 50 μl PCR reaction system was used as a single system, and 1 μl of 20 ng/l oligonucleotide chain pool was used as a PCR template in the single system, and a total of 24 single systems were made.

The PCR reaction system was as follows:

| | | | Total amount of | |
|---|---|---|---|---|
| Contents | Amount | | 24 reactions | |
| NEBNext ® Ultra ™ II Q5 ® | 25 | μl | 600 | μl |
| Master Mix (NEB #M0544S) | | | | |
| oligo pool 1 | 1 μl (20 ng) | | 60 | μl |
| 10 μM Fwd-sub-pool 1 primer | 2.5 | μl | 60 | μl |
| 10 μM Biotinylated-Rev-sub-pool | 2.5 | μl | 24 | μl |
| 1 primer | | | | |
| ddH₂O | Added up to 50 μl | | 456 | μl |
| Total | 50 | μl | 1200 | μl |

The PCR reaction was as follows:

| Step | Temperature | Time | | Cycles |
|---|---|---|---|---|
| Initiate denaturation | 98° C. | 30 | seconds | 1 |
| Denaturation | 98° C. | 10 | seconds | 4 |
| Annealing | 67° C. | 20 | seconds | |
| Extension | 72° C. | 10 | seconds | |
| Denaturation | 98° C. | 10 | seconds | 9 |
| Annealing | 70° C. | 20 | seconds | |
| Extension | 72° C. | 10 | seconds | |
| Last extension | 72° C. | 2 | minutes | 1 |

Results: The oligonucleotide chain pool 1 was successfully amplified into the library chain pool 1 through the above PCR system and reaction conditions.

### 2.1.2 PCR Amplification of Oligonucleotide Chain Pool 2 (TF Oligo Sub Pool 2) (See FIG. 2).

The PCR reaction system was as follows:

| | | | Total amount of | |
|---|---|---|---|---|
| Contents | Amount | | 24 reactions | |
| NEBNext ® Ultra ™ II Q5 ® | 25 | μl | 600 | μl |
| Master Mix (NEB #M0544S) | | | | |
| oligo pool 2 | 1 μl (20 ng) | | 60 | μl |
| 10 μM Biotinylated-Fwd-sub-pool | 2.5 | μl | 60 | μl |
| 2 primer | | | | |
| 10 μM Rev-sub-pool 2 primer | 2.5 | μl | 24 | μl |
| ddH₂O | Added up to 50 μl | | 456 | μl |
| Total | 50 | μl | 1200 | μl |

The PCR reaction was as follows:

| Step | Temperature | Time | | Cycles |
|---|---|---|---|---|
| Initiate denaturation | 98° C. | 30 | seconds | 1 |
| Denaturation | 98° C. | 10 | seconds | 4 |
| Annealing | 67° C. | 20 | seconds | |
| Extension | 72° C. | 10 | seconds | |
| Denaturation | 98° C. | 10 | seconds | 9 |
| Annealing | 70° C. | 20 | seconds | |
| Extension | 72° C. | 10 | seconds | |
| Last extension | 72° C. | 2 | minutes | 1 |

Results: The oligonucleotide chain pool 2 was successfully amplified into the library chain pool 2 through the above PCR system and reaction conditions.

### 2.2 Concentration and Purification of Amplified Library Chain Pools 1 and 2

Since the residues in the PCR amplification reaction system would affect the digestion efficiency of the library chain pools 1 and 2, it was necessary to concentrate and purify the PCR amplification products to remove the residues.

The steps of concentration and purification were as follows:
(1) Using concentration kit: Amicon® Mltra-0.5 3K (purchased from Sigma Aldrich, #UFC500324); molecular mass limit: 3K; corresponding nucleotide length range: 137-1159 bp;
(2) Centrifugation: The PCR amplification product to be concentrated was dissolved in 450 μl of ddH₂O, and centrifuged at 14,000 g at room temperature; the amount of concentration sample per column: 500 μl for 10 minutes+500 μl for 10 minutes+200 μl for 2 minutes;
(3) Electrophoresis: The concentrated product was loaded onto a 2.5% agarose gel, processed with an electrophoresis apparatus at 120V for 10 minutes, and then at 80V for 60 minutes;
(4) Gel recovery: The target gel band was cut off, and QIAgen Gel Extraction Kit (purchased from QIAgen, #28606) was used to recover the target nucleotide;
wherein the conditions for dissolving gel were: 50° C. metal bath, shaking, 550 rpm, until the gel was completely melt; the recovered library was dissolved in 100 ul ddH₂O, and the detection was performed to determine the quality of the recovered oligonucleotide;
(5) The notary was extracted and purified with phenol/chloroform, the recovered notary was dissolved in 20 ul ddH₂O, and the detection was performed to determine the quality of the recovered library.

### Results:

The success of purification was confirmed according to the detected quality of the library. The purified PCR product could be used in subsequent reactions.

### Example 3: Digestion of PCR Product with Nicking Endonuclease and Ligation by Annealing to Generate gRNA Library Sequences to be Inserted into Vector

### 3.1 Digestion of PCR Products of Oligonucleotide Chain Pools by Nicking Endonuclease Digestion and Purification

Each sequence in the library was digested by nicking endonuclease to generate specific sticky ends, which were used to complete the ligation of library chain pools 1 and 2 between each other so as to form a double-stranded ligation library (see FIG. 2). The nicking endonuclease selected in this example was Nb.BsrDI (NEB #R0648L).

### 3.1.1 Nb.BsrDI Enzyme Digestion:

The reaction system and conditions of Nb.BsrDI enzyme digestion of oligonucleotide chain pool 1 were as follows:

| | | | Final |
|---|---|---|---|
| | 50 μl Reaction | | concen- |
| Contents | system | | tration |
| Oligonucleotide chain pool 1 (TF | Up to 1 μg | | |
| oligo sub pool 1) PCR product | | | |
| Nb.BsrDI (NEB#R0648L, 10 U/μl) | 1 | μl | 0.2 U/μl |
| 10X NEBuffer ™ 3.1 (NEB#B7203S) | 5 | μl | |
| ddH₂O | Added up to 50 μl | | |
| Incubation | 60° | C. | |

Digestion time: 4 hours.

The reaction system and conditions of Nb.BsrDI enzyme digestion of oligonucleotide chain pool 2 were as follows:

| | | | Final |
|---|---|---|---|
| | 50 μl Reaction | | concen- |
| Contents | system | | tration |
| Oligonucleotide chain pool 1 (TF | Up to 1 μg | | |
| oligo sub pool 1) PCR product | | | |
| Nb.BsrDI (NEB#R0648L, 10 U/μl) | 1 | μl | 0.2 U/μl |
| 10X NEBuffer ™ 3.1 (NEB#B7203S) | 5 | μl | |
| ddH₂O | Added up to 50 μl | | |
| Incubation | 60° | C. | |

Digestion time: 4 hours.

Oligonucleotide chain pools 1 and 2 were separately digested with Nb.BsrDI enzyme to generate products with 21 nt overhangs. In this example, the oligonucleotide chain pool 1 generated 3′-overhang products, and the oligonucleotide chain pool 2 generated 5′-overhang products. For pair-specific gRNA combinations, these two overhangs were complementary to each other.

### 3.1.2 Purification and Quantification

The products of the digestion in 3.1.1 were purified with streptavidin magnetic beads. This allowed the removal of biotin-carrying small fragments of digestion products, thereby more thoroughly exposing the 3′- and 5′-overhang products of the oligonucleotide pools.

The kits used were as follows:
a) Dynabeads™ MyOne™ Streptavidin C1 (purchased from Thermo, #Catalog Nos. 65001, 65002, 10 mg/ml); and
b) QIAquick Nucleotide Removal Kit (purchased from Qiagen, Cat. No. 28306).

Purify the digested nucleotide library according to the kit instructions for subsequent reactions.

### 3.2 Annealing of Library Chain Pools 1 and 2 to Generate Double-Stranded Ligation Library

A schematic diagram of the annealing of library chain pools 1 and 2 to generate a double-stranded ligation library was shown in FIG. 2. The digested products with 21 nt overhangs were annealed to their pre-designed counterparts in the presence of HiFi Taq DNA ligase (NEB, M0647S). Then, the ligation products with mismatches were digested with T7 endonuclease I (T7EI, NEB, M0302L).

### (1) Annealing to Ligate Library Chain Pools 1 and 2

The reaction system was as follows:

| | | | Final |
|---|---|---|---|
| | 50 μl reaction | | concen- |
| Contents | system | | tration |
| Library chain pool 1 | 350 | ng | 350 ng/50 μl |
| Library chain pool 2 | 350 | ng | 350 ng/50 μl |
| 10X HiFi Taq ligase reaction buffer | 5 | μl | |
| HiFi Taq DNA ligase (NEB#M0647S) | 2 | μl | 5 U/μl |
| (40 U/μl) | | | |
| ddH₂O | Added up to 50 μl | | |
| Total | 50 | μl | |

The reaction conditions were as follows:

| Step | Temperature | Time | |
|---|---|---|---|
| 1 | 62° C. | 5 | hours |
| 2 | 55° C., decreased by 0.1° C. per second | 1.5 | hours |
| 3 | 50° C., decreased by 0.1° C. per second | 1.5 | hours |
| 4 | 45° C., decreased by 0.1° C. per second | 1.5 | hours |

| | | |
|---|---|---|
| Last extension | 4° C. | permanent |

(2) Digestion and Removal of Poorly Matched Double-Stranded Ligation Library Fragments with T7E1 Enzyme

| Contents | 55 μl reaction system | | Final concentration |
|---|---|---|---|
| Ligation product | 50 | μl | |
| T7 Endonuclease I (T7E1, | 5 | μl | 0.5 U/μl |
| #M0302L, 10 U/μl) | | | |
| Incubation time | 30 | minutes | |
| Incubation temperature | 37° | C. | |

After 30 minutes of digestion, 4 μl of 0.5M EDTA was added to the reaction system to stop the reaction.

### Results:

Through the above-mentioned annealing and ligation procedure and the T7E1 enzyme digestion procedure on the poorly matched double-stranded ligated library fragments, the library chain pools 1 and 2 were successfully ligated to generate a double-stranded library.
3.3 Purification and quality inspection of double-stranded ligation library fragments

Objective: To remove the residues in the reaction to improve the quality of the reaction products for subsequent reactions.

1.2× Ampure NXP beads (purchased from Beckman, A63882) were used to purify the library fragments, the purified library fragments were dissolved in 20 μl ddH₂O, and detected with Qubit to determine the quality of the samples.

### Results:

The purified high-quality double-stranded ligation library (i.e., spacer1-spacer2-spacer3-spacer4 shown in FIG. 2(2), which was also referred to as gRNA1-gRNA2-gRNA3-gRNA4) was obtained for subsequent reactions.

### Example 4: Inserting gRNA Library Sequences into Vectors to Form Primary Library Vectors

4.1 Inserting Double-Stranded Ligation Library into lentiGuide-Puro Vector

The double-stranded ligation library prepared in Example 3 (i.e., gRNA1-gRNA2-gRNA3⁻ gRNA4) was cloned into a modified lentiGuide-Puro backbone with mKate2 (Addgene, 52963) by Golden gate reaction.

The golden gate reaction (the reaction included two groups: sample and control) conditions were as follows:

The molar ratio of vector to insert was 1:3.5, and the amount of insert was 145 fmol.

| Contents | Sample | | Control | |
|---|---|---|---|---|
| lentiGuide-Puro vector (purchased from Addgene) | 1 | μl | 1 | μl |
| T4 DNA ligase (Thermo#EL0014 5Weiss U/μl) | 0.5 | μl | 0.5 | μl |
| Double-stranded ligation library prepared in Example 3 | 12 | μl | 0 | μl |
| 10X T4 DNA ligase buffer (Thermo#B69) | 5 | μl | 5 | μl |
| Esp3I (Bsmbl) (10 U/μl) (Thermo #ER0451) | 2.5 | μl | 2.5 | μl |
| ddH₂O | 29 | μl | 41 | μl |
| Total | 50 | μl | 50 | μl |

The reaction conditions were as follows:

| | | |
|---|---|---|
| Temperature | Time | Cycles |

| | | | |
|---|---|---|---|
| 37° C. | 5 | minutes | 90 |
| 22° C. | 5 | minutes | |
| 65° C. | 30 | minutes | 1 |

The library fragments were purified using 0.7× Ampure XP beads (Beckman, A63882), and dissolved in 10 μl ddH₂O, and the sample quality was checked by Qubit.

### Results:

Through the above procedures, the present inventors successfully inserted the double-stranded ligation library prepared in Example 3 into the lentiGuide-Puro vector.

### 4.2 Large-Scale Library Electroporation

Objective: To electroporate the lentiGuide-Puro vector carrying the double-stranded ligation library into competent cells for amplification for subsequent reactions; and to perform sequencing on random clones to confirm successful insertion of the library.

Preparation before electroporation: A LB dish and a LB medium containing ampicillin were preheated at 37° C. for 30 minutes; E. coli Endura electroporation competent cells (Lucigen, 60242-2) were thawed on ice; sample vial for electroporation and EP tube containing 2 μl of goldengate reaction product was cooled on ice.
4.2.1 When the competent cells were completely thawed, the competent cells were gently mixed, and 25 μl of the cells were aliquoted into EP tubes containing 2 μl of the reaction product;
4.2.2 27 μl of the mixture of the competent cells and the reaction product was gently transferred into a cooled electroporation vial, and the mixture was quickly shaken into the bottom of the vial, taking care to avoid air bubbles;
4.2.3 The electroporation vial was subjected to 1700V electroshock (Eppendorf 2510, 1700V), then quickly added with 1 ml of recovery medium, and all the mixture was transferred to a new tube, 1 ml of recovery medium was added into the tube, and the tube containing the mixture was shaken in a 37° C. shaker at 200 rpm for 1 hour;
4.2.4 Calculation of transformation efficiency: The shaken bacterial solution was diluted by 2000 folds, 20000 folds and 200000 folds, respectively, 100 μl was taken from each dilution and spread on a 10 cm petri dish, and incubated in a 30° C. incubator for 20 hours;
4.2.5 Calculation of transformation efficiency;
4.2.6 The scale of electroporation was expanded according to the above-measured appropriate scheme, the mixture was spread on a 24.5 cm² culture dish and incubated at 30° C. for 20 hours;
4.2.7 2 ml of LB was added to the 24.5 cm² culture dish to wet the surface of medium, and all bacterial colony clones were collected. The library contained in the colony clones was designated as Golden Gate Assembly I library;
4.2.8 Additional 20 colony clones were collected for sequencing.

Results: The Endura bacterial solution containing the library Golden Gate Assembly I was obtained, and the sequencing of random bacterial colony clone showed the insertion of the library sequence.

### 4.3 Vector Extraction, Vector Purification and Vector Measurement

Objective: To extract the vector, purify and remove the residues in the reaction to improve the quality of the reaction product for subsequent reactions.
4.3.1 QIAGEN Plasmid Plus Midi Kit (QIAGEN, 12945) was used to extract the vector, and the vector was dissolved in 200 μl ddH₂O;
4.3.2 The library was extracted and purified with phenol/chloroform, the recovered library was dissolved in 20 ul ddH₂O, and the quality of the recovered library was checked.

### 4.4 Using NGS (Next Generation Sequencing) to Check the Quality of the First Round of Library Construction

Objective: To construct an NGS sequencing library, and to determine the quality including homogeneity and diversity of the library carried in the vector by sequencing.

TABLE 2
Primers used for library sequencing:

| Primer | |
|---|---|
| name | Sequence (5′→3′) |
| Fwd- | AATGATACGGCGACCACCGAGATCTACACTCTTT |
| oligo- | CCCTACACGACGCTCTTCCGATCTCCGCGTCTCA |
| lib5seq1 | CACCG |
| Rev-oligo- | CAAGCAGAAGACGGCATACGAGAT-index |
| lib5seq86 | (tctcttcc)-GTGACTGGAGTTCAGACGT |
| | GTGCTCTTCCGATCTACGCCACGCTCTTCG |
| Fwd- | AATGATACGGCGACCACCGAGATCTACACTCTTT |
| oligo- | CCCTACACGACGCTCTTCCGATCTCCGTGCAGCT |
| lib5seq2 | CTTCC |
| Rev-oligo- | CAAGCAGAAGACGGCATACGAGAT-index |
| lib5seq85 | (ctggagta)-GTGACTGGAGTTCAGACGT |
| | GTGCTCTTCCGATCTTGGCCGTCTCGAAAC |
| Fwd-lib1 | AATGATACGGCGACCACCGAGATCTACACTCTTT |
| | CCCTACACGACGCTCTTCCGATCTcttgtggaaa |
| | ggacgaaaCAC |
| Rev- | CAAGCAGAAGACGGCATACGAGAT-index |
| libseq84 | (tggctatc)-GTGACTGGAGTTCAGACGT |
| | GTGCTCTTCCGATCTCTTATTTGAACTTGC |
| | TATGCTGTTTCC |

Samples for constructing the sequencing library:

| | Tm in first | Tm in second | Size |
|---|---|---|---|
| Sample | step | step | (bp) |
| Oligonucleotide chain pool 1 - | 67° C. | 72° C. | 267 |
| before amplification | | | |
| Library chain pool 1 - after | 67° C. | 72° C. | 267 |
| amplification | | | |
| Oligonucleotide chain pool 2 - | 63° C. | 72° C. | 267 |
| before amplification | | | |
| Library chain pool 2 - after | 63° C. | 72° C. | 267 |
| amplification | | | |
| 1st lentiGuide-Puro library (first | 65° C. | 72° C. | 396 |
| round library construction | | | |
| product) | | | |

### 4.4.1 PCR Amplification of Sequencing Library

The reaction system was as follows:

| | | | Final |
|---|---|---|---|
| Contents | 200 μl reaction | | concentration |
| NEBNext ® Mltra ™ II Q5 ® | 100 | μl | 1X |
| Master Mix (NEB #M0544S) | | | |
| TF-oligo pool | 4X | μl | |
| 10 μM forward primer | 10 | μl | 0.25 μM |
| 10 μM reverse primer | 10 | μl | 0.25 μM |
| ddH₂O | up to 200 μl | | |
| Total | 200 | μl | |

The reaction conditions were as follows:

| Step | Temperature | Time | | Cycles |
|---|---|---|---|---|
| Initial denaturation | 98° C. | 30 | seconds | 1 |
| Denaturation | 98° C. | 10 | seconds | 6 |
| Annealing | 67° C. | 30 | seconds | |
| Extension | 72° C. | 30 | seconds | |
| | 98° C. | 10 | seconds | 15~35 |
| | 72° C. | 1 | minute | |
| Last extension | 72° C. | 2 | minutes | 1 |

Results: The NGS sequencing library was successfully constructed.

### 4.4.2 Purification and Measurement of Reaction Product

0.7× Ampure NXP beads were used to purify the sequencing library fragments, the purified library fragments were dissolved in 10 μl ddH2O, and the sample quality was checked with Qubit. Qualified sample was subjected to Illumina MiSeq or NextSeq sequencing.

Results: Sequencing results indicated that the insert library in the first-round reaction had good diversity and homogeneity (FIG. 4).

### Example 5: Insertion of tRNA Sequences into Primary Library Vectors to Form Complete Library Vectors

For the primary library prepared in Example 4 (i.e., the recombinant lentiGuide-Puro vector containing gRNA1-gRNA2-gRNA3-gRNA4), scaffold1-tRNA1, scaffold2-tRNA2 and scaffold3-tRNA3 sequences were inserted sequentially between two adjacent gRNAs, that was, a total of 3 insertions were performed.

TABLE 3
Sequences of inserted tRNAs

| | |
|---|---|
| tRNA1 | GGTTCCATGGTGTAATGGTTAGCACTCTGGACTCTGA |
| | ATCCAGCGATCCGAGTTCAAATCTCGGTGGAACCT |
| | (SEQ ID NO: 705) |
| tRNA2 | GCATGGGTGGTTCAGTGGTAGAATTCTCGCCTGCCAC |
| | GCGGGAGGCCCGGGTTCGATTCCCGGCCCATGCA |
| | (SEQ ID NO: 706) |
| tRNA3 | GGCTCGTTGGTCTAGGGGTATGATTCTCGCTTAGGGT |
| | GCGAGAGGTCCCGGGTTCAAATCCCGGACGAGCCC |
| | (SEQ ID NO: 707) |

### 5.1 First-Round Reaction

Objective: In this round of reaction, one tRNA sequence (hereafter referred to as tRNA) in an exogenous plasmid (e.g., a plasmid containing scaffold1-tRNA1, which had a sequence shown in SEQ ID NO: 708, and was synthesized by China General Biosystems (Anhui) Co., Ltd., http://www.generalbiol.com) was inserted into the library vector in the first-round reaction (see FIG. 2).

5.1.1 Insertion of First tRNA Sequence

### Materials:

Golden Gate Assembly I library (generated from the first-round reaction); plasmid containing scaffold1-tRNA1 (purchased from Addgene).
Golden gate assembly reaction (the reaction comprised two groups: sample and control)

The molar ratio of the Golden Gate Assembly I library to the plasmid containing scaffold1-tRNA1 was 1:4.9, and 31 fmol of the Golden Gate Assembly I library was used.

| Contents | Sample | | Control | |
|---|---|---|---|---|
| 1^{st} lentiGuide-Puro library | 1 | μl | 1 | μl |
| T4 DNA ligase (Thermo#EL0014 5Weiss U/μl) | 0.5 | μl | 0.5 | μl |
| scaffold1-tRNA1 plasmid | 1 | μl | 0 | μl |
| 10X T4 DNA ligase buffer (Thermo#B69) | 5 | μl | 5 | μl |
| AarI (2 U/μl) (Thermo #ER1582) | 1 | μl | 1 | μl |
| ddH₂O | 40.5 | μl | 41.5 | μl |
| 50X oligonucleotide (0.025 mM) | 1 | μl | 1 | μl |
| Total | 50 | μl | 50 | μl |

The reaction conditions were as follows:

| | | |
|---|---|---|
| Temperature | Time | Cycles |

| | | | |
|---|---|---|---|
| 37° C. | 5 | minutes | 90 |
| 22° C. | 5 | minutes | |
| 65° C. | 30 | minutes | 1 |
| 37° C. | 3 | hours | |
| 37° C. | | | permanent |

### Results:

The first tRNA sequence (i.e., tRNA1) was successfully inserted into the vector carrying the library to form the Golden Gate Assembly II library.

### 5.1.2 Purification and Measurement of Reaction Product

0.7× Ampure beads were used to purify the library fragments, the purified library fragments were dissolved in 10 μl ddH₂O, and the sample quality was checked with Qubit.

Results: A purified vector carrying one tRNA sequence (i.e., tRNA1) and the library was obtained.

5.1.3 Electroporation of the First-Round Reaction Product Obtained after Purification

Objective: To electroporate a vector carrying one tRNA sequence and a double-stranded ligation library into competent cells for amplification for subsequent reactions.

Preparation before electroporation: A LB dish containing ampicillin and a recovery medium were preheated at 37° C. for 30 minutes; Endura electroporation competent cells were thawed on ice; sample vial for electroporation and EP tube containing 4 μl of goldengate reaction product were cooled on ice.
(1) When the competent cells were completely thawed, the competent cells were gently mixed, and 50 μl of the cells was aliquoted into EP tube containing 4 μl of reaction product;
(2) 54 μl of the mixture of the competent cells and the reaction product was gently transferred into a cooled electroporation vial, and the mixture was quickly shaken into the bottom of the vial, taking care to avoid air bubbles;
(3) The electroporation vial was subjected to 1700V electroshock, then quickly added with 1 ml of recovery medium, all the mixture was transferred to a new tube, then 3 ml of recovery medium was added into the tube, the tube containing the mixture was shaken in a 37° C. shaker at a rate of 200 rpm for 1 hour;
(4) The mixture solution was spread on a 24.5 cm² culture dish and incubated for 20 hours;
(5) 15 to 25 ml of LB was added to the 24.5 cm² culture dish to wet the surface of medium, and all the colony clones were collected. The library contained in the colony clones was designated as Golden Gate Assembly II library.

Results: The Golden Gate Assembly II library was amplified using competent cells.

### 5.1.4 Extraction of Vector Containing Second-Round Reaction Product, Purification of Vector and Measurement of Vector

Objective: To extract the vector carrying Golden Gate Assembly II library from competent cells, and purify the vector for subsequent reactions.
(1) QIAGEN Plasmid Plus Midi Kit was used to extract the vector, and the vector was dissolved in 200 μl of ddH₂O;
(2) The library was extracted and purified with phenol/chloroform, the recovered library was dissolved in 20 μl of ddH₂O, and the quality of the recovered library was checked.

Results: The purified Golden Gate Assembly II library was obtained

### 5.2 Second-Round Reaction

Objective: In this round of reaction, one tRNA sequence (hereafter referred to as tRNA2) in an exogenous plasmid (for example, a plasmid containing scaffold2-tRNA2, which had a sequence shown in SEQ ID NO: 709, and was synthesized by China General Biosystems (Anhui) Co., Ltd., http://www.generalbiol.com) was inserted into the library vector in the second-round reaction (see FIG. 2).

5.2.1 Insertion Reaction of tRNA Sequence

Materials: Golden Gate Assembly II library; plasmid containing scaffold2-tRNA2.

### (1) Goldengate Reaction (the Reaction Included Two Groups: Sample and Control)

The molar ratio of the Golden Gate Assembly II library to the plasmid containing scaffold2-tRNA2 was 1:3, and 35 fmol of the Golden Gate Assembly II library was used.

| Contents | Sample | | Control | |
|---|---|---|---|---|
| 2^{nd} lentiGuide-Puro library | 1 | μl | 1 | μl |
| T4 DNA ligase (Thermo#EL0014, 5Weiss U/μl) | 0.5 | μl | 0.5 | μl |
| scaffold2-tRNA2 plasmid | 1.2 | μl | 0 | μl |
| 10X Cutsmart buffer (NEB, B72045) | 5 | μl | 5 | μl |
| BbsI-HF (20 U/μl, NEB#R3539L) | 2.5 | μl | 2.5 | μl |
| ATP (Thermo, 100 mM, R0441) | 0.5 | μl | 0.5 | μl |
| DTT (Invitrogen, 100 mM, Y00147) | 0.5 | μl | 0.5 | μl |
| ddH₂O | 38.8 | μl | 40 | μl |
| Total | 50 | μl | 50 | μl |

The reaction conditions were as follows:

| | | |
|---|---|---|
| Temperature | Time | Cycles |

| | | | |
|---|---|---|---|
| 37° C. | 5 | minutes | 90 |
| 22° C. | 5 | minutes | |
| 65° C. | 30 | minutes | 1 |
| 37° C. | 3 | hours | |
| 37° C. | | | permanent |

Results: The second tRNA sequence (i.e., tRNA2) was successfully inserted into the vector carrying the 4-gRNA combinations.

### 5.2.2 Purification and Measurement of Reaction Product

0.7× Ampure beads were used to purify the library fragments, the purified library fragments were dissolved in 15 μl of ddH₂O, and the sample quality was checked with Qubit.

Results: The purified vector carrying two tRNA sequences (i.e., tRNA1 and tRNA2) and the library was obtained.

5.2.3 Electroporation of Third-Round Reaction Product Obtained after Purification

Objective: To electroporate the vector carrying two tRNA sequences and the library into competent cells for amplification for subsequent reactions.

Preparation before electroporation: A LB dish containing ampicillin and a recovery medium were preheated at 37° C. for 30 minutes; Endura electroporation competent cells were thawed on ice; sample vial for electroporation and EP tube containing 4 μl goldengate reaction product were cooled on ice.
(1) When the competent cells were completely thawed, the competent cells were gently mixed, and 50 μl of the cells were aliquoted into EP tube containing 4 μl of reaction product;
(2) 54 μl of the mixture of the competent cells and the reaction product was gently transferred into a cold electroporation vial, and the mixture was quickly shaken into the bottom of the vial, taking care to avoid air bubbles;
(3) The electroporation vial was subjected to 1700V electroshock, then quickly added with 1 ml of recovery medium, all the mixture was transferred to a new tube, then 3 ml of recovery medium was added into the tube, the tube containing the mixture was shaken in a shaker at 37° C. at a rate of 200 rpm for 1 hour;
(4) The mixture was spread to a 24.5 cm² culture dish and incubated for 20 hours;
(5) 15 to 25 ml of LB was added to the 24.5 cm² culture dish to wet the surface of medium, and all the bacterial colony clones were collected and extracted to obtain the vector. The library contained in the vector was designated as 3rd lentiGuide-Puro library.

Results: The Golden Gate Assembly III library was amplified by competent cells, and the purified Golden Gate Assembly III library was obtained.

### 5.3 Third-Round Reaction

Objective: In this round of reaction, one tRNA sequence (hereafter referred to as tRNA3) in an exogenous plasmid (for example, a plasmid containing scaffold3-tRNA3, which had a sequence shown in SEQ ID NO: 710, and was synthesized by China General Biosystems (Anhui) Co., Ltd., http://www.generalbiol.com) was inserted into the library vector in the third-round reaction (see FIG. 2).

5.3.1 Insertion Reaction of tRNA Sequence

Materials: Golden Gate Assembly III library; plasmid containing scaffold3-tRNA3.

### (1) Goldengate Reaction (the Reaction Included Two Groups: Sample and Control)

The molar ratio of the Golden Gate Assembly III library to the plasmid containing BsaI-tRNA was 1:3.5, and 30 fmol of the Golden Gate Assembly III library was used.

| Contents | Sample | | Control | |
|---|---|---|---|---|
| 3^{rd} lentiGuide-Puro library | 1 | μl | 1 | μl |
| T4 DNA ligase (Thermo#EL0014 5Weiss U/μl) | 0.5 | μl | 0.5 | μl |
| scaffold3-tRNA3 plasmid | 3 | μl | 0 | μl |
| 10X Cutsmart buffer (NEB, B72045) | 5 | μl | 5 | μl |
| BsaI-HF v2 (20 U/μl, NEB #R3733) | 2 | μl | 2 | μl |
| ddH₂O | 38.5 | μl | 41.5 | μl |
| Total | 50 | μl | 50 | μl |

The reaction conditions were as follows:

| | | |
|---|---|---|
| Temperature | Time | Cycles |

| | | | |
|---|---|---|---|
| 37° C. | 5 | minutes | 90 |
| 22° C. | 5 | minutes | |
| 65° C. | 30 | minutes | 1 |
| 37° C. | 3 | hours | |
| 37° C. | | | permanent |

Results: The third tRNA sequence (i.e., tRNA3) was successfully inserted into the vector carrying the library, and a vector carrying pair-specific multiplexed 4-gRNA combinations and 3 tRNAs was obtained.

### 5.3.2 Purification and Measurement of Reaction Product

0.7× Ampure beads were used to purify the library fragments, the purified library fragments were dissolved in 15 μl and ddH2O, and the sample quality was checked with Qubit.

Results: The purified vector carrying three tRNA sequences (i.e., tRNA1, tRNA2 and tRNA3) and the library was obtained.

5.3.3 Electroporation of Third-Round Reaction Product Obtained after Purification

Objective: To electroporate lentiGuide-The Puro vector carrying three tRNA sequences and the double-stranded ligation library into competent cells for amplification and for subsequent reactions.

Preparation before electroporation: A LB dish containing ampicillin and a recovery medium were preheated at 37° C. for 30 minutes; Endura electroporation competent cells were thawed on ice; sample vial for electroporation and EP tube containing 4 μl of goldengate reaction product were cooled on ice.
(1) When the competent cells were completely thawed, the competent cells were gently mixed, and 50 μl of the cells were aliquoted into EP tubes containing 4 μl of reaction product;
(2) 54 μl of the mixture of the competent cells and the reaction product was gently transferred into a cooled electroporation vial, and the mixture was quickly shaken into the bottom of the vial, taking care to avoid air bubbles;
(3) The electroporation vial was subjected to 1700V electroshock, then quickly added with 1 ml of recovery medium, all the mixture was transferred to a new tube, then 3 ml of recovery medium was added into the tube, the tube containing the mixture was shaken in a shaker at 37° C. at 200 rpm for 1 hour;
(4) The mixture was spread to a 24.5 cm² culture dish and incubated for 30 hours;
(5) 15 to 25 ml of LB was added to the 24.5 cm² culture dish to wet the surface of medium, all bacterial colony clones were collected for the collection of vector, and the library contained in the vector was designated as 4th lentiGuide-Puro library.

Results: The Golden Gate Assembly IV library was amplified with competent cells, and the purified Golden Gate Assembly IV library was obtained.

### 5.4 Construction of Golden Gate Assembly IV Sequencing Library by PCR

Materials: Golden Gate Assembly IV library and primers (Table 4)

TABLE 4
Primer (5′→′3)

Rev-libseq-TCATCTCC:
CAAGCAGAAGACGGCATACGAGATGGAGATGAGTGACTGGAGTTCA
GACGTGTGCTCTTCCGATCTTGCTGTTTCCAGCATAGCTC
Fwd-libseq-U6:
AATGATACGGCGACCACCGAGATCTACACTCTTTCCCTACACGACG
CTCTTCCGATCTGGACTATCATATGCTTACCGTAAC
Fwd-libseq-gly:
AATGATACGGCGACCACCGAGATCTACACTCTTTCCCTACACGACG
CCTCTTCCGATTGGTCTAGTGGTAGAATAGTACCC
Fwd-libseq-gln:
AATGATACGGCGACCACCGAGATCTACACTCTTTCCCTACACGACG
CTCTTCCGATCTGGTTAGCACTCTGGACTCTG

The reaction system was as follows:

| | | | Final |
|---|---|---|---|
| Contents | 10 μl reaction | | concentration |
| NEBNext ® Ultra ™ II Q5 ® | 25 | μl | 1X |
| Master Mix(NEB# M0544S) | | | |
| TF lib4 | 1.25 | μl | |
| Fwd-libseq-U6 | 0.2 | μl | 0.25 μM |
| Rev-libseq-Gly-GCC-TCATCTCC | 0.2 | μl | 0.25 μM |
| ddH₂O | 38.35 | μl | |
| Total | 50 | μl | |

The reaction conditions were as follows:

| Step | Temperature | Time | | Cycles |
|---|---|---|---|---|
| Initial denaturation | 98° C. | 30 | seconds | 1 |
| Denaturation | 98° C. | 10 | seconds | 16 |
| Annealing | 63° C. | 30 | seconds | |
| Extension | 72° C. | 20 | seconds | |
| Last extension | 72° C. | 2 | minutes | 1 |

Results: The NGS sequencing library was successfully constructed.

### 5.3.4 Purification and Measurement of Reaction Product

0.7× Ampure NXP beads were used to purify the sequencing library fragments, the purified library fragments were dissolved in 10 μl of ddH₂O, and the sample quality was checked with Qubit. The qualified sample was subjected to Illumina MiSeq or NextSeq sequencing.

Results: The sequencing results indicated that the insert library in the first-round reaction had good diversity and homogeneity (FIG. 4).

### Example 6: Lentiviral Packaging of Golden Gate Assembly IV Library and Titer Detection of Lentivirus Carrying Golden Gate Assembly IV Library

(1) Lentiviral packaging was performed using three-plasmid system of pvMD2G (purchased from Addgene), pSPAX2 (purchased from Addgene) and Golden Gate Assembly IV library vector;
(2) Lentiviruses were concentrated by ultracentrifugation;
(3) The lentiviruses were diluted by gradient;
(4) The lentiviruses of each dilution concentration were used to infect Jurkat cells expressing Cas9 (presented by other laboratory);
(5) The Jurkat cells infected with lentivirus were cultured for 2 days, and the proportion of cells that reported fluorescence positive for lentivirus was detected by flow cytometry.

Results: According to the results of the flow cytometry, the titer of the concentrated lentiviruses was concluded to reach a level of 108, which could be used to infect human cell lines to deliver the CRISPR library of pair-specific multiplexed gRNA combinations for subsequent high-throughput screening.

### Example 7: Quality Detection Using the CRISPR Library of Multiplexed gRNA Combinations

A total of 21,938,825 sequence reads were obtained by NGS sequencing via PE150 for the CRISPR library constructed by the method of the present invention. Using cutadapt (v2.6), low-quality sequence reads and sequencing adapter sequences were removed, and the sequence reads after filtration were aligned with the designed oligonucleotide chain pools using Bowtie2 (v 2.3.5.1), the alignment rate of proper pair reads was 96.74%, with a total of 21,223,537 reads, indicating that 96.74% of the clones were correct. Among them, 9,916,616 gRNA sequences were completely correct, and the other 11,307,121 sequences contained errors generated during DNA synthesis, and this correct rate met the requirements for library construction.

FIG. 4A as a control chart of the CRISPR library showed that the distribution of insert sequencing reads in the library was relatively concentrated, and the distribution trend conformed to the Poisson distribution. A total of 6,236 oligonucleotides were designed in the library, and the library coverage was almost 100%. FIG. 4B showed the cumulative distribution of sequencing reads, in which the number of oligonucleotide chains distributed in 90% reads was 12 times that in 10%, and the variation coefficient of the sequencing reads in the library was 1.0908, indicating that the library had good homogeneity.

### Example 8: Validation Experiment of the Use of CRISPR Library of Multiplexed gRNA Combinations to Screen Cell Signaling Pathway

The present inventors used Jurkat T cell receptor (TCR) signaling pathway activation model to verify the effectiveness of the CRISPR library of multiplexed gRNA combinations constructed in the present invention for screening signaling pathway.

### Cultivation of Cells:

Cas9 encoding gene was inserted into Jurkat cells using lentiCas9-Blast lentivirus (Addgene, 52962). The Jurkat-Cas9 cell line was selected using 2 ug/mL blasticidin as determined by blasticidin killing curve. Following the blasticidin selection, viable cells were collected, and the cells were sorted by BD FACS fusion flow cytometer (BD Bioscience). Then, Cas9-expressing Jurkat cell monoclones were established in the presence of 2 ug/mL blasticidin. The Cas9 expression of each monoclone was verified by Western blotting (Cell Signaling, Mouse anti-Cas9, 7A9-3A3).

### Generation and Transduction of Viral Library:

The vector library of 4-gRNA combinations, pMD2.G (Addgene, 12259) envelope plasmid and psPAX2 packaging plasmid (Addgene, 12260) were mixed in a mass ratio of 5:2:3, and incubated with 250 uM calcium chloride. An equal volume of 2×HeBS (280 mM NaCl, 1.5 mM Na₂HPO₄, 50 mM HEPES, pH 7.05) was added to the above DNA-CaCl₂ and incubated for 15 minutes at room temperature. This mixture was added dropwise to 80% confluent HEK293T cells for transfection. Lentiviral supernatants were collected at 48 and 72 hours after transfection, filtered through 0.45 m filters (Millipore, SLHV033RB), and concentrated by ultracentrifugation at 70,000 g for 2 hours at 4° C. A total of 20×10⁶ Jurkat-Cas9 cells were infected with the concentrated viral library at MOI ≤0.3 in RPMI-1640 medium containing 8 ug/ml polybrene. Spinfection was performed by centrifuging the culture plate at 700 g for 2 hours at 32° C. The cells were verified for mKate2 expression by flow cytometry (Cytoflex, Beckman) 48 hours after the transduction, and this expression indicated successful transduction. The proportion of mKate2-positive cells was typically about 30%. During the next 6 to 10 days, the cells were grown under antibiotic selection of 2 ug/mL puromycin and 2 ug/mL blasticidin, and the cell concentration was maintained at 5×10⁵ cells/mL. During antibiotic selection, the cells were monitored for mKate2 expression by flow cytometry until 95% of the cells were mKate2-positive.

### Activation Experiment:

6×10⁶ successfully infected Jurkat-Cas9 cells were collected as a starting reference (the control in FIG. 6A), and an additional 30×10⁶ Jurkat-Cas9 cells were stimulated with 25 μL/mL ImmunoCult™ Human CD3/CD28 T Cell Activator (STEMCELL, 10971) (in RPMI containing 10% FBS and 1× Pen-Strep, the cell density was 5×10⁶ cells/mL). 24 hours after stimulation, the cells were stained with anti-CD69 (Biolegend, FN50), and CD69⁺ (top 25%) and CD69⁻ (bottom 25%) cell populations were sorted and collected using FACS (Fusion, BD). A total of 5×10⁶ cells for each population were collected (the “CD69⁺” and “CD69⁻” in FIG. 6A).

### Validation of Candidate:

To validate the inhibitory effect of a single candidate gRNA vector or a gRNA combination vector, validation experiments were performed following the same procedure for large-scale library transduction and activation. The difference was that the starting number of Jurkat-Cas9 cells per viral transduction experiment was 5×10⁵. 24 hours after stimulation, the percentage of CD69⁺ cells was examined using flow cytometry (Cytoflex, Beckman). All flow cytometry data were analyzed by Flowjo v10.

### Results

In the experiments, the cells with highly activated and inactivated TCR signaling pathway were collected separately and their genomic DNAs were extracted, followed by NGS sequencing after amplification and insertion of gRNA sequences. According to the sequencing results, the present inventors performed the sorting from high to low according to the amount of enriched multiplexed gRNA combinations in the inactivated cells, which confirmed that various gRNA combinations targeting the TCR signaling pathway were significantly enriched in the inactivated cells (Log 2 FC<−1 or >1; −log 10(P-value)>1) (FIG. 6C).

(1) T Cell Activation Screening Through Library of 4-gRNA Combinations

The present inventors reasoned that multiple genes involved in the same pathway or the same gene family might exhibit more functional relevance and lead to genetic compensation when only one of them was functionally disrupted. Therefore, the present inventors hypothesized that disturbing multiple genes in the same pathway or gene family might help with identifying new candidates that shared coordinated behavior, comparing to disturbing one single target. To facilitate this goal, the present inventors designed most of the 4-gRNA combinations either from the same pathway (3,672) or from the same gene family (945). To balance the coverage among targeting genes, the present inventors generated 1,569 random combinations by picking genes according to their occurrences across the established combinations in descending order. 50 negative controls were also included. Finally, in the designed library, each of the 1,599 candidate genes were covered by 15 combinations in average (minimum 13 combinations) (FIG. 7).

To demonstrate the performance of the 4-gRNA combinations (4gRNA-comb) in CRISPR screening, the present inventors applied the library into a canonical T cell activation model by interrogating genes in combinations. The activation of T cell receptor (TCR) promoted signal transduction cascades that ultimately activated transcription factors such as NF-κB, NFAT and AP-1, thereby promoting the transcription of specific genes that lead to T cell proliferation and differentiation. In this system, genes involved in multiple signaling pathways cooperated and constituted a complicated network that governed the fate of T cells.

To perform multiplexed screening, Jurkat cells with stable Cas9 expression (also known as Jurkat-Cas9 cells) were transduced with the expression vector of the 4-gRNA combinations (FIG. 6A). After 10 days of cell culture and antibiotic selection, the cells were stimulated with T cell activators. Twenty-four hours after stimulation, cell viability was assessed based on surface CD69 expression, and the surface CD69 expression indicated the activation status of T cells. Cell populations sorted in the top 25% (CD69⁺) and bottom 25% (CD69⁻) were collected by cell sorter (FIG. 8). NGS sequencing libraries were generated using genomic DNAs collected from the cell populations pre-stimulation (Control), CD69⁺ (Top 25%) post-stimulation and CD69⁻ (Bottom 25%) post-stimulation cell populations (FIG. 6A, FIG. 8). The distributions of the 4-gRNA combinations were validated and compared between libraries (FIG. 10). Compared to the vector library, a small number of 4-gRNA combinations were eliminated from the control samples after puromycin selection (0.19%, 120/6236) (FIG. 9).

To discover combinations that potentially interrupted the cellular signal transduction of TCR activation, the present inventors focused on comparisons between CD69⁺ and CD69⁻ post-stimulation samples. The ratio of normalized read counts of each combination between the CD69⁺ and CD69⁻ samples were used to evaluate the perturbation to the TCR signal transduction. Firstly, the ratios from the combinations from the TCR signaling pathway, the salivary secretion pathway and pre-designed non-targeting controls were first examined (FIG. 6B, the grey area of FIG. 11). As expected, combinations involved in the TCR signaling pathway largely shifted towards to the low CD69⁺/CD69⁻ ratio side, which means cells edited by these combinations enriched in the CD69⁻ sample. Meanwhile, combinations involved in the salivary secretion pathway, which has no known interaction with the TCR signaling transmitting, exhibited a tight and uniform distribution (FIG. 6B). Moreover, the combinations that targeting subunits of the TCR complex, were also enriched in the CD69⁻ sample (FIG. 11). Together, these results suggested that the multiplexed 4gRNA-comb CRISPR/Cas9 perturbation effectively identified gene combinations that directly disrupted the TCR signaling transmitting functions.

### (2) Identification and Validation of Candidate Gene Combinations

Next, the present inventors ranked the ratios across all combinations to identify top candidates. The present inventors calculated the ratios of normalized read counts of each combination between CCD69⁺ and CD69⁻ cell populations, and assigned p-values under a negative binomial model (FIG. 6A). Among the top 10 combinations that were enriched in CD69-cell populations, 3 of them were predicted to be essential to T-cell activation signaling transduction, as they were either relevant to the T cell differentiation pathway or contained subunits of the TCR complex. To validate the screened candidates, the present inventors individually cloned the other seven 4-gRNA combinations, plus two other combinations with the consideration of both enrichment score and p-value. the inventors repeated the T cell activation experiment in 2˜4 replicates in two independent Jurkat-Cas9 clones. Most of combinations showed reduction in terms of the percentage of activated T cells compared to the control, and five of them show statistical significance (FIG. 5D and FIG. 12), which demonstrated the effectiveness of the multiplexed CRISPR screening strategy of the present invention.

To further analyze the synergistic behavior of multiple genes in the same combination, the present inventors dissected the top candidate “PSMF1-PSMD11-ROCK1-HRAS” into the following subsets: six 2-gRNA combinations and four single gRNA, and repeated the activation experiment. This was to test whether the down-regulation to the Jurkat activation was due to the incorporated behavior of the four genes, or due to incorporated behavior of a dominant subset. Among all 2-gRNA subsets, only “PSMD11-PSMF1” reduced the Jurkat activation level with statistical significance but not as effective as the 4-gene combination (FIG. 13). Interestingly, “PSMD11-PSMF1” was also subunit of two other 4gRNA-comb in the multiplexed CRISPR libraries (i.e., “PSMF1-PSMA3-PSMD11-PSMC5” and “PSMB2-PSMD6-PSMD11-PSMF1”), but only significantly impacted the Jurkat activation when coordinated with ROCK1 and HRAS. PSMF1 and PSMD11 are components of the ubiquitin mediated proteolysis, which is one of the downstream pathways of the TCR signaling pathway. ROCK1 and HRAS co-occurred in multiple pathways, including chemokine signaling pathway, in which the chemokine signal was transduced by GPCR that expressed on the immune cells (including T cell). Although there was no direct functional connection between the genes, the results obtain in the invention indicated potential synergistic effects among these genes.

Finally, the present inventors defined a set of “highly impacting” 4-gene combinations, they were enriched more than 2-fold in the CD69-cell populations, and the present inventors attempted to find other subsets that were essential to T cell activation. The present inventors defined a synergy score to quantify the contribution of each subset. Since the occurrences of 3-gene or 4-gene subsets was limited to the library of the present invention, the present inventors calculated scores for the 2-gene combination subsets. The present inventors validated the top candidate “ATP6V1D-KDELR1” and confirmed that the simultaneous knockout of these two genes reduced the activation rate of Jurkat cells (FIG. 14). The “ATP6V1D-KDELR1” subset was from the Vibrio cholerae infection pathway, ATP6V1D encoded a component of vacuolar ATPase (V-ATPase), that mediated the acidification of intracellular organelles in eukaryotic cells, and KDELR1 encoded endoplasmic reticulum protein retention receptor. To the knowledge of the present inventors, these two genes had no known influence on the signal transduction of T cell receptors.

Overall, these data demonstrated that the multiplexed CRISPR perturbation of the present invention was an effective strategy to identify functional and combinatorial gene sets that were responsible for phenotypic outcomes.

The experimental results of this example proved that the screening library of pair-specific multiplexed gRNA combinations constructed by the present invention was effective in the screening of cell signaling pathways, and could perform high-throughput screening for the effect of specific multi-gene combinations in mediating the biological state and behavior of cells, thereby studying the role of multi-gene combinational functions in cell regulation; while traditional gRNA libraries were only designed for a single gene, and transducing multiple gRNAs at the same time was low in efficiency and random in combination, so that it was impossible to perform high-throughput screening for specific multi-gene combinations that medicated biological state and behavior of cells.

### Example 9: Validation Experiment of the Use of CRISPR Library of Multiplexed gRNA Combinations to Screen Preferred gRNA for Specific Sites in Prime Editor System

The present inventors used the strategy of CRISPR library of multiplexed gRNA combinations to construct a pegRNA library for the Prime Editor system. The spacer part and the PBS+ reverse transcription template part in gRNA were designed in two oligonucleotide libraries, respectively. Using the aforementioned in-library ligation protocol, a screening library capable of testing gRNA editing efficiency was constructed (FIG. 15).

### Example 10: In Vivo Screen for Combinatorial Checkpoint Blockades to Boost T Cells

To identify potential candidates for a combined immunotherapy, we applied a 4-gRNA multiplexed library in an in vivo screen for boosted tumor-infiltrating T cells (TILs). Following a multiplexed CRISPR library construction strategy, as further detailed below, we genetically engineered CD8+ T cells collected from OT-1 mice. To investigate synergistic or additive anti-tumor efficacies of multiple gene knockouts, we engineered the T cells with a library of four gRNAs simultaneously.

The engineered T cells were screened for activation capability in a tumor environment. The engineered T cells were injected into recipient mice inoculated with Hepa1-6 cells with stable H2Kb-OVA₂₅₇₋₂₆₄ expression (FIG. 16A and FIG. 17). At the endpoint of the screening, the engineered T cells that were present in the tumors in the mice were isolated by Fluorescence-activated Cell Sorting (FACS) and subjected to NGS (next generation sequencing) characterization.

More specifically, the in vivo screening library was designed to target six checkpoint genes (Btla, Pdcd1, Tigit, Ctla4, Havcr2 and Adora2a) and included all fifty-six possible combinations, composed of fifteen 4-gRNA combinations, twenty 3-gRNA combinations, fifteen 2-gRNA combinations and six single-gRNA combinations (denoted as “CP group” herein). Moreover, for each combination, we used non-targeting control gRNAs to fill the unoccupied positions if the number of the targeting gRNAs is less than 4. For example, for the six single-gRNA combinations, each of them contains three non-targeting control gRNAs to fill the unoccupied positions. For comparison, we also included combinations targeting two other groups of genes: one included four genes (Lat, Zap70, Cd3e, and CD247) involved in the first signaling of T cell activation (fifteen combinations, denoted as “TCR group” herein), the other included five co-stimulatory molecules (Il2ra, Tnfrsf9, Tnfrsf4, Tnfrsf18 and CD28) involved in the secondary signaling of T cell (thirty combinations, denoted as “CS group” herein). T cells engineered by combinations from the TCR group and CS group should be incapable of T cell activation. All together, we included 101 distinct combinations targeting one to four genes of the CP group, the TCR group, and the CS group. For each distinct combination, we designed a group of six gRNA-combos in the library to eliminate biases of individual guide RNA. Another eighty-four combinations included only non-targeting control gRNAs, which served as negative controls (denoted as “NT group” herein). The sequences of the gRNA combinations are listed in SEQ ID NOs: 14-703. The screening was conducted in three independent batches.

We calculated log 2 transformed fold-change (log 2FC) values to show the relative abundance of each gRNA combination in the tumor infiltrated lymphocytes (TIL) relative to the engineered T cells before being injected into the recipient mice (“SR,” representing “starting reference”) (further described below). It was contemplated that the T cells enriched in the tumors gained functions relevant to anti-tumor immunity, which were reflected by the gRNA combinations with high log 2FC values. As shown in FIG. 18, most T cells did not successfully get enriched in the tumors and showed negative log 2FC values (FIG. 18). Among the four groups (CP, TCR, CS, and NC), more T cells from the CP group showed a positive log 2FC value (FIGS. 16B and 18). These results indicated the effectiveness of the screening model.

We ranked all gRNA combinations based on the corresponding T cell enrichments in the tumors from three screening batches and identified a top candidate of 3-gRNA combination that simultaneously targets Pdcd1, Adora2a and Ctla4 (denoted as “PAC” herein) (FIG. 19). Among all the gRNA combinations, the PAC combination exhibited the best reproducibility across three independent batches of screen and different groups of gRNAs. We also examined other 4gRNA-combinations that included gRNAs targeting these three genes and found that only this specific combination maximumly activated the infiltrated T cells in tumors (FIG. 20). These results indicate the importance of identifying the precise combination of targets, as the anti-tumor ability of T cells may not be positively strengthened by knocking out more checkpoint genes.

Next, we performed validation experiments to confirm the screen results. We prepared T cells knocked out only at the Pdcd1 loci (denoted as “PNN”), at Pdcd1 and Ctla4 loci (denoted as “PCN”), as well as Pdcd1, Ctla4, and Adora2a loci (“PAC”). The knockout efficiencies of the gRNAs were confirmed (FIG. 21). We also shuffled gRNAs when making combos to eliminate biases from individual gRNA. The gRNAs used in the validation experiment were randomly picked from the gRNAs used in the screening experiment. For example, there were six different gRNA combinations targeting the PAC in the screening library. In the validation experiment, an individual gRNA was picked randomly from those gRNAs that have been used in the screening library. Thus, the specific gRNA combination to target PAC in the validation experiments were not present in the screening library.

The engineered T cells were injected intravenously into the recipient mice inoculated with Hepa1-6 cancer cells expressing H2Kb-OVA₂₅₇₋₂₆₄. After the T cell therapy, the weight loss of the mice and the tumor size was monitored for eight weeks. We found that the growth of tumor size of the other two groups (PNN and PCN) was all controlled at different levels. T cells engineered by the PAC combination showed the best anti-tumor immune responses compared to T cells engineered by PCN or PNN, which were reflected by the tumor size and the survival rate of the mice (FIGS. 16C-16D & 22). The rates of tumor growth in the PCN and PNN groups were slower compared to the CTRL group, in which non-engineered T cells were used.

These results indicated that the multiplexed CRISPR screen is an effective way to look for candidates for potential combinatorial immune checkpoint blockades, and for other potential combinatorial pathway blockades.

### 10.1 Screen Library and Vector Design

An in vivo screen library was designed and constructed via in-library ligation and vector library construction as illustrated in FIG. 2.

As noted above, for the check point blockade screening library, we included a group of six immune checkpoint genes (CP group), a group of four genes involved in the first signaling of T cell activation (TCR group) and a group of five co-stimulatory molecules involved in the secondary signaling (CS group). Within each group, all possible 4 gRNA combinations, 3 gRNA combinations, 2 gRNA combinations, and single gRNA construct were designed. For each construct, the unoccupied position was placed with non-targeting control gRNA if the number of the targeting gRNAs is less than four. Further, all combinations were represented by six groups of gRNAs that are distinct from each other. Additionally, 84 combinations containing only the non-targeting control gRNAs (NT group) were included and served as negative control. This screen library composed a total of 101 gene combinations represented by 606 gRNA groups and 84 negative control combinations including on non-targeting gRNAs.

For the screening part, a multiplexed CRISPR knockout vector that contained a 4-sgRNA tandem cassette (as illustrated in FIG. 2) and a mKate2 reporter was generated. Using this vector, up to 4 checkpoints (e.g., shown as gRNA1, gRNA2, gRNA3, and gRNA4 illustrated in FIG. 2) could be disrupted in single cell. A mKate2 reporter was used in order to separate the engineered T cells that infiltrated into the tumors.

For the validation part, a multiplexed CRISPR knockout vector that contained a Pdcd1-Adora2a-Ctla4 gRNA tandem cassette and a mKate2 reporter was generated (SEQ ID NO: 704). A vector that contained a Pdcd1-NTC-NTC gRNA tandem cassette and a BFP reporter were created as control, in which one NTC gRNAs replaced the Adora2a gRNA, one NTC gRNA replaced the Ctla4 gRNA, and a BFP reporter replaced the mKate2 reporter. A vector that contained a Pdcd1-Ctla4-NTC sgRNA tandem cassette and a BFP reporter was created as a control, in which one NTC gRNAs replaced the Adora2a gRNA and a BFP reporter replaced the mKate2 reporter.

TABLE 5

| | |
|---|---|
| Spacer of | SEQ ID NO: 1: |
| Pdcd1 | CAGCTTGTCCAACTGGTCGG |
| Spacer of | SEQ ID NO: 2: |
| Adora2a | AGCACACAAGCACGTTACCC |
| Spacer of | SEQ ID NO: 3: |
| Ctla4 | GGACTGAGAGCTGTTGACAC |
| F-BsrDI-1 | SEQ ID NO: 4: |
| | GACCGCGTCTCACACCG |
| R-BsrDI- | SEQ ID NO: 5: |
| 1-biotin | CTGCGCTCCACGAGCCCGACGCAATG |
| F-BsrDI- | SEQ ID NO: 6: |
| 2-biotin | CTGGCGTGGTCGCGTGCTCGGCAATG |
| F-BsrDI-2 | SEQ ID NO: 7: |
| | GATCAGGGCCGTCTCGAAAC |
| Murine | SEQ ID NO: 8: |
| nest-F | GGACTATCATATGCTTACCG |
| Murine | SEQ ID NO: 9: |
| nest-R | GCCCAGAattcTCGCATTC |
| Fwd- | SEQ ID NO: 10: |
| Libseq-G12 | AATGATACGGCGACCACCGAgatctACACT |
| | ATAGCCTACACTCTTTCCCTACACGACGCT |
| | CTTCCGATCTTGTGGAAAGGACGAAACAC |
| Rev- | SEQ ID NO: 11: |
| Libseq-G12 | CAAGCAGAAGACGGCATACGAGATGGAGAT |
| | GAGTGACTGGAGTTCAGACGTGTGCTCTTC |
| | CGATCTTGCTAGGACCGGCCTTAAAG |
| Fwd- | SEQ ID NO: 12: |
| Libseq-G23 | AATGATACGGCGACCACCGAGATCTACACT |
| | CTTTCCCTACACGACGCTCTTCCGATCTGC |
| | TGGTTCCATGGTGTA |
| Rev- | SEQ ID NO: 13: |
| Libseq-G23 | CAAGCAGAAGACGGCATACGAGATCGAGTA |
| | ATGTGACTGGAGTTCAGACGTGTGCTCTTC |
| | CGATCTCCTTAGCCGCTAATAGGTGAGC |

### 10.2 Screen and Validation Experiments

### 10.2.1 Tumor Cells

Hepa1-6 cells were transduced with H-2K^{b}-OVA₂₅₇₋₂₆₄-expressing lentivirus. And the H-2K^{b}-OVA₂₅₇₋₂₆₄ expression in a mono-clone was validated via flow cytometry. The resulted cell line was named as Hepa1-6-H-2K^{b}-OVA₂₅₇₋₂₆₄. The established Hepa1-6-H-2K^{b}-OVA₂₅₇₋₂₆₄ cells were further transduced with a lentiviral vector (lenti-EF-1α-luciferase-T2A-BSD) for luciferase stable expression.

### 10.2.2 Mice Models

Primary T cells were isolated from OT-1 or Cas9+OT-1 mice, which were bred from OT-1 and Cas9 mouse obtained from the Jackson Laboratory. The tumor was inoculated to the NOD-Prkdc^{scid} Il2rg^{null}/Shjh mice purchased from Shanghai Jihui Laboratory Animal Care. The T cell donor mice were 10-12 weeks old. The tumor recipient mice were 6-8 weeks old. All mice were housed in standard individually ventilated and pathogen-free conditions in the laboratory facility of the Westlake University, under that animal protocol (AP #21-016-MLJ). All mice were used in accordance with Institutional Animal Care and Use Committee (IACUC) guidelines for Westlake University.

10.2.3 T Cell Isolation and Culture Spleens were isolated from the Cas9+OT-1 mice, followed by mashing through 40 m filter and RBCs lysis (BD Pharm Lyse). CD8+T cells were purified by negative selection via CD8a+ T cell isolation Kit (Milteny). Cells were stimulated with 100U/ml recombinant human IL-2 (Peprotech), 1 g/ml anti-mouse CD3F (Ultraleaf, Clone 145-2C11, Biolegend) and 0.5 g/ml anti-mouse CD28 (Ultraleaf, Clone 37.51, Biolegend) and cultured in RPMI-1640 with 10% FBS, 10 mM HEPES (Gibco), 100 M non-essential amino acids (Gibco), 1 mM Sodium Pyruvate (Gibco), 50 μM β-mercaptoethanol (Sigma), 50 U/ml penicillin, and 50 μg/ml streptomycin (Gibco).

### 10.2.4 T Cell Transduction, Transduction Efficiency Test, and Gene Editing Efficiency Test

After ex vivo stimulation for 24h, CD8+T cells were transduced with lentivirus in the presence of polybrene at 8 μg/ml during spinfection at 2,000 g for 2h at 32° C. At 48h after transduction, T cells were collected for transduction efficiency test via flow cytometry and adoptive transfer.

In a validation experiment, CD8+T cells were transduced with lentivirus for 2 times at 24h and 48h after isolation. At 24h after second transduction, T cells were collected for transduction efficiency test via flow cytometry and adoptive transfer after sorting via FACS. The gene editing efficiency was tested in T cells with a Pdcd1-Adora2a-Ctla4 combined disruption. At 48h after second transduction, mKate2+ T cells were sorted via FACS and pelleted for gDNA extraction. Then, the sgRNA target sequences of each gene were amplified by 2-step PCR for NGS sequencing. The list of oligos used in gene editing efficiency test were included in Table 5.

### 10.2.5 Antigen Specificity Test for OT-I T Cells

OT-1 CD8+T cells were co-cultured with either Hepa1-6 cells or Hepa1-6 expressing H-2K^{b}-OVA₂₅₇₋₂₆₄ cells for 2h and 48h. In the 2h test, cells were co-cultured at the presence of anti-CD107 (Biolegend). After 2h, all cells were collected and stained with anti-CD8a (Biolegend) for degranulation analysis via flow cytometry (Cytoflex, Beckman). After 48h, all cells were collected and stained with anti-CD8a, PI and Annexin V (Biolegend) for target cell apoptosis analysis via flow cytometry (Cytoflex, Beckman). All FCM Data were analyzed by Flowjo.

### 10.2.6 Screening Experiment

Hepa1-6 cells expressing H-2K^{b}-OVA₂₅₇₋₂₆₄ were mixed with matrigel (1:1 volume) and injected subcutaneously into the right flank of NPSG mice at 1×10⁶/recipient. At d12 after tumor cell inoculation, 1×10⁷ CD8+ T cells with screening library transduction (5%˜10% mKate2+ cells in total cells) were adoptively transferred into each recipient via i.v. injection. Meanwhile, 2˜3×10⁶ CD8+ T cells with screening library transduction were frozen as a starting reference (SR). Weight loss and tumor size was measured at d0 and d7 after T cell injection. On d7 after injection, the tumor was collected and cut into small fragments. After consecutively mashing through 100 m and 40 m filters, RBCs in the cell suspension were lysed. Then, the tumor infiltrating CD8+T cells were enriched by density gradient centrifugation via Lymphprep (StenCell). Cells at the interface were carefully collected and washed by PBS. Then, the cells were re-suspended into PBS and stained with anti-mouse CD8a for 30 mins on ice. Finally, CD8+mKate2+ TILs were sorted via FACS (BD Fusion). A total of 20,000-40,000 CD8+mKate2+ TIL could be collected per tumor. TIL from 3-4 recipient mice were mixed together and pelleted with carrier cells (Raji cell) at 1:50 (CD8+ T cells: carrier cells) for genomic DNA extraction.

10.2.7 Genomic DNA Extraction and sgRNA Library PCR Amplification

Genomic DNA extraction was performed using TIANamp Genomic DNA kit (TIANGEN) and finally resuspended in 50 μl nuclease free water. To prepare the gRNA NGS library for the SR sample, all gDNA were amplified on thermocycling with parameters of 98° C. for 30 sec, 20˜22 cycles of (98° C. for 10 sec, 64° C. for 30 sec, 72° C. for 20 sec), 72° C. for 2 min. One NGS library generated amplicons covering the 1st and the 2nd gRNAs (G12 library), and another NGS library generated amplicons covering the 2nd and the 3rd gRNAs (G23 library). Primers of SEQ ID NO: 10 and SEQ ID NO: 11 were used as a pair of primers to amplify the G12 library. Primers of SEQ ID NO: 12 and SEQ ID NO: 13 were used as a pair of primers to amplify the G23 library. To prepare the gRNA NGS library for the TIL sample, two-step amplification was applied. In the 1st step, PCR reaction (400˜800 ng DNA input per reaction, 2˜4 reactions per sample) was performed using Ultra II Q5 Master Mix (NEB) with thermocycling parameters as 98° C. for 30 sec, 28-30 cycles of (98° C. for 10 sec, 60° C. for 30 sec, 72° C. for 20 sec), 72° C. for 2 min. Primers of SEQ ID NO: 8 was used as the forward primer and SEQ ID NO: 9 was used as the reverse primer. And the PCR condition and primers of the 2nd step follows the condition of the SR library preparation, but with 8-10 cycles.

The list of primers used in gene editing efficiency test were included in Table 5.

### 10.2.8 Validation of Candidates

Hepa1-6 cells expressing H-2Kb-OVA257-264 with luciferase were mixed with matrigel (1:1 volume) and injected subcutaneously into the right flank of NPSG mice at 1×106/recipient. On d11-d12 after tumor cell inoculation, 1×10⁶ mKate2+ or BFP+ CD8+ T cells were sorted via FACS and adoptively transferred into each recipient via intravenous injection. Weight loss and tumor size were measured every 3 days after T cell injection. Meanwhile, the biological signal of tumor was monitored weekly by in vivo imaging via PHOTON IMAGER™ OPTIMA, in which luciferin was administered intraperitoneally 5 minutes prior to signal collection.

### 10.3 Data Analysis

In order to find the effective 4-gRNA combinations that enhance the capacity of the CD8+T cell-mediated tumor elimination in vivo, the normalized read counts of each combination were used to compare their representatives between the TIL and SR libraries. Normalizations were conducted according to the depth of sequencing libraries. We calculated both the fold-change and the p-value for each 4-gRNA combination. The TIL and SR libraries were treated as two samples, and G12 library and G23 library of each sample were treated as technical replicates. We used the log 2 fold-change of G12 and G23 between the TIL and SR libraries to pick out combinations for validations, which can be explained as Log 2((Mean of TIL three batches g12+1)/(Mean of SR three batches g12+1)) and Log 2((Mean of TIL three batches g23+1)/(Mean of SR three batches g23+1)).

Those skilled in the art will further realize that the present invention may be embodied in other specific forms without departing from its spirit or central characteristics. Since the foregoing description of the present invention discloses only exemplary embodiments thereof, it is to be understood that other variations are considered to be within the scope of the present invention. Therefore, the present invention is not limited to the specific embodiments described in detail herein. Rather, reference should be made to the appended claims to indicate the scope and content of the present invention.

### REFERENCES

1 Pickar-Oliver, A. & Gersbach, C. A. The next generation of CRISPR-Cas technologies and applications. Nat Rev Mol Cell Biol 20, 490-507, doi:10.1038/s41580-019-0131-5 (2019).
2 Hanahan, D. & Weinberg, R. A. Hallmarks of cancer: the next generation. Cell 144, 646-674, doi:10.1016/j.cell.2011.02.013 (2011).
3 Wong, A. S., Choi, G. C., Cheng, A. A., Purcell, O. & Lu, T. K. Massively parallel high-order combinatorial genetics in human cells. Nat Biotechnol 33, 952-961, doi:10.1038/nbt.3326 (2015).
4 Wong, A. S. et al. Multiplexed barcoded CRISPR-Cas9 screening enabled by CombiGEM. Proc Natl Acad Sci USA 113, 2544-2549, doi:10.1073/pnas.1517883113 (2016).
5 Han, K. et al. Synergistic drug combinations for cancer identified in a CRISPR screen for pairwise genetic interactions. Nat Biotechnol 35, 463-474, doi:10.1038/nbt.3834 (2017).
6 Zhu, S. et al. Genome-scale deletion screening of human long non-coding RNAs using a paired-guide RNA CRISPR-Cas9 library. Nat Biotechnol 34, 1279-1286, doi:10.1038/nbt.3715 (2016).
7 Diao, Y et al. A tiling-deletion-based genetic screen for cis-regulatory element identification in mammalian cells. Nat Methods 14, 629-635, doi:10.1038/nmeth.4264 (2017).
8 Shen, J. P. et al. Combinatorial CRISPR-Cas9 screens for de novo mapping of genetic interactions. Nat Methods 14, 573-576, doi:10.1038/nmeth.4225 (2017).
9 Boettcher, M. et al. Dual gene activation and knockout screen reveals directional dependencies in genetic networks. Nat Biotechnol 36, 170-178, doi:10.1038/nbt.4062 (2018).
10 Liu, Y et al. Genome-wide screening for functional long noncoding RNAs in human cells by Cas9 targeting of splice sites. Nat Biotechnol, doi:10.1038/nbt.4283 (2018).
11 Najm, F. J. et al. Orthologous CRISPR-Cas9 enzymes for combinatorial genetic screens. Nat Biotechnol 36, 179-189, doi:10.1038/nbt.4048 (2018).
12 Chow, R. D. et al. In vivo profiling of metastatic double knockouts through CRISPR-Cpf1 screens. Nat Methods 16, 405-408, doi:10.1038/s41592-019-0371-5 (2019).
13 Zetsche, B. et al. Multiplex gene editing by CRISPR-Cpf1 using a single crRNA array. Nat Biotechnol 35, 31-34, doi:10.1038/nbt.3737 (2017).
14 Gier, R. A. et al. High-performance CRISPR-Cas12a genome editing for combinatorial genetic screening. Nat Commun 11, 3455, doi:10.1038/s41467-020-17209-1 (2020).
15 Nissim, L., Perli, S. D., Fridkin, A., Perez-Pinera, P. & Lu, T. K. Multiplexed and programmable regulation of gene networks with an integrated RNA and CRISPR/Cas toolkit in human cells. Mol Cell 54, 698-710, doi:10.1016/j.molcel.2014.04.022 (2014).
16 Xie, K., Minkenberg, B. & Yang, Y Boosting CRISPR/Cas9 multiplex editing capability with the endogenous tRNA-processing system. Proc Natl Acad Sci USA 112, 3570-3575, doi:10.1073/pnas.1420294112 (2015).
17 Knapp, D. et al. Decoupling tRNA promoter and processing activities enables specific Pol-II Cas9 guide RNA expression. Nat Commun 10, 1490, doi:10.1038/s41467-019-09148-3 (2019).
18 Kanehisa, M. & Goto, S. KEGG: kyoto encyclopedia of genes and genomes. Nucleic Acids Res 28, 27-30, doi:10.1093/nar/28.1.27 (2000).
19 Lappalainen, I. et al. The European Genome-phenome Archive of human data consented for biomedical research. Nat Genet 47, 692-695, doi:10.1038/ng.3312 (2015).
20 Shang, W. et al. Genome-wide CRISPR screen identifies FAM49B as a key regulator of actin dynamics and T cell activation. Proc Natl Acad Sci USA 115, E4051-E4060, doi:10.1073/pnas.1801340115 (2018).
21 Kiani, S. et al. Cas9 gRNA engineering for genome editing, activation and repression. Nat Methods 12, 1051-1054, doi:10.1038/nmeth.3580 (2015).
22 Breinig, M. et al. Multiplexed orthogonal genome editing and transcriptional activation by Cas12a. Nat Methods 16, 51-54, doi:10.1038/s41592-018-0262-1 (2019).
23 Truong, V. A. et al. CRISPRai for simultaneous gene activation and inhibition to promote stem cell chondrogenesis and calvarial bone regeneration. Nucleic Acids Res 47, e74, doi:10.1093/nar/gkz267 (2019).
24 Gruber, A. R., Lorenz, R., Bernhart, S. H., Neubock, R. & Hofacker, I. L. The Vienna RNA website. Nucleic Acids Res 36, W70-74, doi:10.1093/nar/gkn188 (2008).
25 Doench, J. G. et al. Optimized sgRNA design to maximize activity and minimize off-target effects of CRISPR-Cas9. Nat Biotechnol 34, 184-191, doi:10.1038/nbt.3437 (2016).
26 Martin, M. Cutadapt removes adapter sequences from high-throughput sequencing reads. EMBnet Journal 17, 10-12, doi:10.14806/ej.17.1.200 (2011).
27 Langmead, B. & Salzberg, S. L. Fast gapped-read alignment with Bowtie 2. Nat Methods 9, 357-359, doi:10.1038/nmeth.1923 (2012).
28 Love, M. I., Huber, W. & Anders, S. Moderated estimation of fold change and dispersion for RNA-seq data with DESeq2. Genome Biol 15, 550, doi:10.1186/s13059-014-0550-8 (2014).

1. A CRISPR library of pair-specific multi-gene combinations, wherein the CRISPR library comprises a plurality of vectors each carrying more than two kinds of gRNA sequences, and the more than two kinds of guide RNA (gRNA) sequences carried on each vector are capable of performing co-editing of more than two kinds of important molecules.

2. The CRISPR library of pair-specific multi-gene combinations according to claim 1, wherein the CRISPR library comprises a plurality of vectors each carrying 4 kinds of gRNA sequences, and the 4 kinds of gRNA sequences carried on each vector are capable of performing co-editing of 4 kinds of important molecules.

3. The CRISPR library of pair-specific multi-gene combinations according to claim 2, wherein each vector in the CRISPR library comprises an insert fragment shown in gRNA1-tRNA1-gRNA2-tRNA2-gRNA3-tRNA3-gRNA4, in which gRNA1, gRNA2, gRNA3 and gRNA4 are respectively directed to 4 different genes of any known sequences.

4. The CRISPR library of pair-specific multi-gene combinations according to claim 3, wherein the insert fragment shown in gRNA1-tRNA1-gRNA2-tRNA2-gRNA-tRNA3-gRNA4 further comprises a U6 promoter, preferably a human U6 promoter, at the N-terminus.

5. The CRISPR library of pair-specific multi-gene combinations according to claim 3, wherein the sequences of tRNA1, tRNA2 and tRNA3 are all the same, two are the same and one is different, or three are different.

6. A method for constructing a CRISPR library of pair-specific multiplexed gRNA combinations based on long overhang sequence ligation, the method comprising the following steps:
(1) designing a sequence library of pair-specific multiplexed gRNA combinations according to the pathway or gene family to be screened, and synthesizing a mixture of two or more oligonucleotide chain pools according to the sequences of the library, wherein each oligonucleotide sequence in each oligonucleotide chain pool comprises one or more kinds of gRNAs, wherein for 3′-end of each sequence in one oligonucleotide chain pool, there is only one kind of 5′-end sequence completely complementary thereto in another oligonucleotide chain pool, and the complementary portion has a sequence length of 2-100 nucleotides, such as 4-50, 10-40, 15-35, 20-30, preferably 21 nucleotides (21 nt);
(2) performing PCR amplification with the mixture of two or more oligonucleotide chain pools as templates, respectively, to obtain two or more corresponding library chain pools, respectively;
(3) using a nicking endonuclease to digest the two or more corresponding library chain pools obtained by PCR amplification in step (2) respectively to generate products each having one or two complementary long overhangs of 2-100 nucleotides, such as 4-50, 10-40, 15-35, 20-30, preferably 21 nt, and mixing the digested products from each of the library chain pools and performing ligation of the digested products from the library chain pools by annealing to generate linear gRNA library sequences, each of which comprises the pair-specific multiplexed gRNA combinations;
(4) inserting the linear gRNA library sequence obtained in step (3) into a vector to form a primary library vector; and
(5) sequentially inserting a tRNA sequence between two adjacent gRNAs in the primary library vector to form a complete library vector, wherein the complete library vector comprises pair-specific multiplexed gRNA combinations which comprise a tRNA sequence between any two adjacent gRNAs.

7. A method for constructing a CRISPR library of pair-specific multiplexed gRNA combinations based on long overhang sequence ligation, the method comprising the following steps:
(1) designing a sequence library of pair-specific multiplexed gRNA combinations according to the pathway or gene family to be screened, and synthesizing a mixture of oligonucleotide chain pools 1 and 2 according to the sequences of the library, wherein each oligonucleotide sequence in the oligonucleotide chain pool 1 comprises gRNA1 and gRNA2, and each oligonucleotide sequence in the oligonucleotide chain pool 2 comprises gRNA3 and gRNA4, wherein for the 3′ end of each sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt);
(2) performing PCR amplification with the mixture of oligonucleotide chain pools 1 and 2 as templates, respectively, to obtain library chain pools 1 and 2, respectively;
(3) using a nicking endonuclease to digest the library chain pools 1 and 2 obtained by PCR amplification in step (2) respectively to generate products each having a complementary long overhang of 2-100 nucleotides, such as 4-50, 10-40, 15-35, 20-30, preferably 21 nt, and performing ligation of the digested products from the library chain pools 1 and 2 by annealing to generate gRNA library sequences each of which is shown by gRNA1-gRNA2-gRNA3-gRNA4;
(4) inserting the gRNA library sequence obtained in step (3) into a vector to form a primary library vector; and
(5) sequentially inserting tRNA1, tRNA2 and tRNA3 sequences into the primary library vector to form a complete library vector, wherein the complete library vector comprises the insert fragment shown by gRNA1-tRNA1-gRNA2-tRNA2-gRNA3-tRNA3-gRNA4.

8. The method according to claim 7, wherein a reverse primer in a primer pair used for the amplification of the oligonucleotide chain pool 1 in step (2) is biotinylated, and a forward primer in a primer pair used for the amplification of the oligonucleotide chain pool 2 is biotinylated.

9. The method according to claim 7, wherein the nicking endonuclease used in step (3) is selected from Nb.BsrDI or Nt.BspQI nicking endonuclease.

10. The method according to claim 8, wherein in step (3), after nicking endonuclease cleavage and before annealing ligation, streptavidin magnetic beads are used to purify and remove biotin-carrying small fragments.

11. The method according to claim 7, wherein after the library chain pools 1 and 2 are digested with the nicking endonuclease and annealed in step (3), and before step (4), the method further comprises using T7 endonuclease I to digest poorly matched ligation products.

12. A method for constructing a CRISPR library of pair-specific multiplexed gRNA combinations based on long overhang sequence ligation, comprising the following steps:
(1) designing a sequence library of pair-specific multiplexed gRNA combinations according to the pathway or gene family to be screened, and synthesizing a mixture of oligonucleotide chain pools 1 and 2 according to the sequences of the library, wherein each oligonucleotide sequence in the oligonucleotide chain pool 1 comprises gRNA1 and gRNA2, and each oligonucleotide sequence in the oligonucleotide chain pool 2 comprises gRNA3 and gRNA4, wherein for the 3′ end of each sequence in the oligonucleotide chain pool 1, there is only one kind of 5′-end sequence completely complementary thereto in the oligonucleotide chain pool 2, and the complementary portion has a sequence length of 2-100 nucleotides, such as 4-50, 10-40, 15-35, 20-30, preferably 21 nucleotides (21 nt);
(2) performing PCR amplification with the mixture of oligonucleotide chain pools 1 and 2 as templates, respectively, to obtain library chain pools 1 and 2, respectively, wherein the reverse primer in the primer pair used for amplification of the oligonucleotide chain pool 1 is biotinylated, and the forward primer in the primer pair used for amplification of the oligonucleotide chain pool 2 is biotinylated;
(3) using a nicking endonuclease to digest the library chain pools 1 and 2 obtained by PCR amplification in step (2) respectively to generate products each having a complementary long overhang of 2-100 nucleotides, such as 4-50, 10-40, 15-35, 20-30, preferably 21 nt, performing ligation of the digested products from the library chain pools 1 and 2 by annealing after biotin-carrying small fragments are removed by purification with streptavidin magnetic beads, performing digestion of the ligation product with T7 endonuclease I (T7E1) to remove poorly matched double-stranded fragments and finally generate gRNA library sequences, each of which is shown by gRNA1-gRNA2-gRNA3-gRNA4;
(4) inserting the gRNA library sequence obtained in step (3) into a vector to form a primary library vector; and
(5) sequentially inserting tRNA1, tRNA2 and tRNA3 sequences into the primary library vector to form a complete library vector, wherein the complete library vector comprises the insert fragment shown by gRNA1-tRNA1-gRNA2-tRNA2-gRNA3-tRNA3-gRNA4.

13. A method for constructing a CRISPR library of pair-specific multiplexed gRNA combinations based on long overhang sequence ligation, comprising:
(1) designing a sequence library of pair-specific multiplexed gRNA combinations according to the pathway or gene family to be screened, and synthesizing a mixture A of two oligonucleotide chain pools A1 and A2 and a mixture B of two oligonucleotide chain pools B1 and B2, according to the sequences of the library, wherein each oligonucleotide sequence in each oligonucleotide chain pool comprises one or more kinds of gRNAs,
wherein for 3-end of each sequence in oligonucleotide chain pool A1, there is only one kind of 5′-end sequence completely complementary thereto in oligonucleotide chain pool A2, and the complementary portion has a sequence length of 2-100 nucleotides, such as 4-50, 10-40, 15-35, 20-30, preferably 21 nucleotides (21 nt);
wherein for 3-end of each sequence in oligonucleotide chain pool B1, there is only one kind of 5′-end sequence completely complementary thereto in oligonucleotide chain pool B2, and the complementary portion has a sequence length of 2-100 nucleotides, such as 4-50, 10-40, 15-35, 20-30, preferably 21 nucleotides (21 nt);
wherein for 3-end of each sequence in oligonucleotide chain pool A2, there is only one kind of 5′-end sequence completely complementary thereto in oligonucleotide chain pool B1, and the complementary portion has a sequence length of 2-100 nucleotides, such as 4-50, 10-40, 15-35, 20-30, preferably 21 nucleotides (21 nt);
(2) performing PCR amplification with the mixture A as templates, respectively, to obtain corresponding library chain pools A1 and A2, respectively; and performing PCR amplification with the mixture B as templates, respectively, to obtain corresponding library chain pools B1 and B2, respectively;
(3) using a nicking endonuclease to digest the library chain pools A1 and A2 obtained by PCR amplification in step (2) respectively to generate products each having one or two complementary long overhangs of 2-100 nucleotides, such as 4-50, 10-40, 15-35, 20-30, preferably 21 nt; using a nicking endonuclease to digest the library chain pools B1 and B2 obtained by PCR amplification in step (2) respectively to generate products each having one or two complementary long overhangs of 2-100 nucleotides, such as 4-50, 10-40, 15-35, 20-30, preferably 21 nt; and mixing the digested products from the library chain pools A1, A2, B1 and B2 and performing ligation of the digested products from the library chain pools A1, A2, B1 and B2 by annealing to generate linear gRNA library sequences A1-A2-B1-B2, each of which comprises the pair-specific multiplexed gRNA combinations;
(4) inserting the gRNA library sequence A1-A2-B1-B2 obtained in step (3) into a vector to form a primary library vector; and
(5) sequentially inserting a tRNA sequence between two adjacent gRNAs in the primary library vector to form a complete library vector, wherein the complete library vector comprises pair-specific multiplexed gRNA combinations which comprise a tRNA sequence between any two adjacent gRNAs.

14. The method according to claim 7, wherein the vector used in step (4) is a viral vector selected from a lentiviral vector, a retroviral vector, an adenoviral vector, an adeno-associated viral vector.

15. The method according to claim 7, wherein the vector used in step (4) is a lentiviral vector, and the method further comprises the following step after obtaining the complete library vector in step (5): (6) a step of packaging a lentivirus with the constructed library vector and detecting a lentivirus titer.

16. The method according to claim 7, wherein in step (5), tRNA1, tRNA2 and tRNA3 are sequentially introduced through golden gate assembly, wherein the sequences of tRNA1, tRNA2 and tRNA3 are all the same, two are the same and one is different, or all three are different.

17. The method according to claim 7, wherein the insert fragment shown by gRNA1-tRNA1-gRNA2-tRNA2-gRNA3-tRNA3gRNA4 is under the control of a U6 promoter, preferably under the control of a human U6 promoter.

18. The method according to claim 7, wherein in step (5), tRNA1, tRNA2 and tRNA3 are sequentially introduced by golden gate assembly, and different endonucleases are respectively used in the reactions for introducing tRNA1, tRNA2 and tRNA3.

19. A library construction method based on long overhang sequence ligation, the method comprising the steps of:
(1) designing and synthesizing a mixture of oligonucleotide chain pools 1 and 2, wherein each oligonucleotide in the oligonucleotide chain pool 1 has a length of 141 to 165 bp, and each oligonucleotide in the oligonucleotide chain pool 2 has a length of 150 bp; and, for the 3′ end of each kind of sequence in the oligonucleotide chain pool 1, there is only one kind of 5′-end sequence completely complementary thereto in the oligonucleotide chain pool 2, and the complementary portion has a sequence length of 15-35 nucleotides, preferably 20-30 nucleotides, more preferably 21 nucleotides (21 nt);
(2) performing PCR amplification with the mixture of oligonucleotide chain pools 1 and 2 as templates, respectively, to obtain library chain pools 1 and 2, respectively, wherein a reverse primer in a primer pair used for amplification of the oligonucleotide chain pool 1 is biotinylated, and a forward primer in a primer pair used for amplification of the oligonucleotide chain pool 2 is biotinylated;
(3) using a nicking endonuclease to digest the library chain pools 1 and 2 obtained by PCR amplification in step (2) respectively to generate products each having a complementary long overhang of 15-35 nucleotides, preferably 20-30 nucleotides, more preferably 21 nt, performing ligation of the digested products from the library chain pools 1 and 2 by annealing after biotin-carrying small fragments are removed by purification with streptavidin magnetic beads, performing digestion of the ligation product with T7 endonuclease I (T7E1) to remove poorly matched double-stranded fragments and finally generate insert sequences; and
(4) inserting the insert sequence obtained in step (3) into a vector to form a primary library vector.

20. The method according to claim 19, wherein the nicking endonuclease used in step (3) is selected from Nb.BsrDI or Nt.BspQI nicking endonuclease.

21. The method according to claim 19, wherein the vector used in step (4) is a viral vector selected from a lentiviral vector, a retroviral vector, an adenoviral vector or an adeno-associated viral vector.

22. The method according to claim 21, wherein the vector used in step (4) is a lentiviral vector, and, after obtaining the primary library in step (4), the method further comprises the following step: (5) a step of packaging a lentivirus with the constructed library and detecting a lentivirus titer.

23. A host cell transformed with the CRISPR library according to claim 1, wherein the host cell is a prokaryotic cell or a eukaryotic cell, preferably a bacterial cell, a fungal cell or a mammalian cell, more preferably a murine cell or a human cell.

24. A high-throughput method for combined screening of incorporated multiple genes, the method comprising using the CRISPR library according to claim 1.

25. The method according to claim 13, wherein the vector used in step (4) is a viral vector selected from a lentiviral vector, a retroviral vector, an adenoviral vector, an adeno-associated viral vector.

26. The method according to claim 13, wherein the vector used in step (4) is a lentiviral vector, and the method further comprises the following step after obtaining the complete library vector in step (5): (6) a step of packaging a lentivirus with the constructed library vector and detecting a lentivirus titer.
